(19) United States
(12) Patent Application Publication
(10) Pub. No.: US 2026/0279543 A1
(43) Pub. Date: Sep. 17, 2026

(54) CHILD EVALUATION SYSTEM AND METHOD OF USE

(71) Applicants: Susan O. Gunduz, Northport, NY (US); Stephanie Anne Denton, Birmingham, AL (US)

(72) Inventors: Susan O. Gunduz, Northport, NY (US); Stephanie Anne Denton, Birmingham, AL (US)

(73) Assignees: Susan O. Gunduz, Northport, NY (US); Stephanie Anne Denton, Birmingham, AL (US)

(21) Appl. No.: 19/669,173

(22) Filed: May 6, 2026

Related U.S. Application Data
(62) Division of application No. 19/344,768, filed on Sep. 30, 2025, now Pat. No. 12,670,981.
Application No. 19/344,768 is a division of application No. 18/768,976, filed on Jul. 10, 2024, now Pat. No. 12,462,919.

(51) Int. Cl. G16H 20/70; A61B 5/00; A61B 5/11; A61B 5/12

(52) U.S. Cl. CPC G16H 20/70; A61B 5/1124; A61B 5/123; A61B 5/4803

(57) ABSTRACT

Systems and methods for the diagnosis and treatment of developmental delay and/or disorders in children for the purpose of early diagnosis and treatment of children to promote the development of at least one of fine motor skills, gross motor skills, sound articulation and sound discrimination skills, receptive language, expressive language, phonological awareness, phonological processing to prevent reading failure, academic underachievement, color blindness, attention issues and social emotional dysregulation. The systems and methods resulting in the generation of a referral of the individual for services in areas where the evaluation results were deficient to promote at least one of fine motor skills, gross motor skills, sound articulation, and sound discrimination skills.

### TECHNICAL FIELD

The present disclosure generally relates to systems and methods for diagnosing and treating developmental delay and/or disorders in children, and more particular to systems and methods for the early diagnosis and treatment of children in early childhood to promote at least one of fine motor skills, gross motor skills, sound articulation, and sound discrimination skills, receptive language, expressive language, phonological awareness, phonological processing to prevent reading failure, academic underachievement, color blindness, attentional issues, social emotional dysregulation.

A portion of the disclosure of this patent document contains material which is subject to (copyright or mask work) protection. The (copyright or mask work) owner has no objection to the facsimile reproduction by anyone of the patent document of the patent disclosure, as it appears in the Patent and Trademark Office patent file or records, but otherwise reserves all (copyright or mask work) rights whatsoever.

### BACKGROUND

Many children with developmental delay and/or other disorders are not recognized or diagnosed until the child is of school age. That is, many developmental delays and/or disorders are not diagnosed until after a child begins school, and usually multiple years after the child begins school. Indeed, in many cases, the child's parents may not be aware that their child may have a developmental delay and/or disorder and therefore do not take any action to identify and/or diagnose and/or seek early intervention to treat the child's delay and/or disorder before the child begins school. In other cases, the child's parents may not be aware of what can be done to diagnose and/or treat a suspected delay and/or disorder and therefore do not take any action to diagnose and/or treat the child's delay and/or disorder before the child begins school. There is a focus on reactive, deficit-driven, ‘wait-to-fail’ model instead of on the development of preventative approaches. In still other cases, the child's parents may not have the financial means to hire a specialist to diagnose and/or treat a suspected delay and/or disorder and therefore do not take any action to diagnose and/or treat the child's disorder before the child begins school. Every state has early intervention programs that act on a medical provider's prescription to investigate a diagnosis of developmental delay. The wait time for Early intervention to act on this prescription may take up to 3 to 6 months for a diagnosis and for early intervention to start another 3 to 12 months. If a child has Medicaid, most private early intervention sites that a pediatrician may also prescribe as they wait for Early intervention state programs to start is close to nonexistent. Those who have commercial insurance intervention may be provided by private early intervention sites but usually allowed only 1 to 2 months of therapy which is not adequate time to help a child with their delay depending on the severity. In addition, while most state-run early intervention programs focus on under 3 years, at 3 many children who have deficits do not qualify for services due to services are only covered by school board budgets and therefore receive little to no intervention until these deficits become quite large. As a result, a child's delay and/or disorder is often not recognized and/or addressed until after the child begins school and often not until 3^{rd} to 4^{th} grade. At that time, the child's teachers and/or other school staff may observe warning signs that the child has one or more delays and/or disorder and then recommend that a school specialist meet with the child to address the suspected disorder(s). By that time, for example, the child is several grade levels behind in reading and it is difficult to catch up and 75% do not catch up and read at grade level. Of those children, 54% do not attend College. Children who present with speech and language delay are at risk for future reading struggles (dyslexia). Early intervention before school begins is effective at eliminating that risk or lessening it. Those from families who claim English as a second language need early exposure to the English language in order to successfully participate in school in which learning in English is done. Children from an economically disadvantaged background would greatly benefit from exposure before school to the necessary skills needed to successfully perform in school. 67 percent of all fourth graders in the US are reading below grade level and approximately 80% of fourth graders from low socio-economic backgrounds are reading below grade level. Any treatment would therefore only assist the child in significantly reducing reading failure, dyslexia risk, limited exposure to the English language and also those children from disadvantaged back grounds. This program will help every child succeed in school. If only 10 to 20 percent of the population is dyslexic, it does not explain the high 67% of children in 4^{th} grade read below grade level. Looking at the multifactorial reasons for reading and or academic difficulties will help all the children who might potentially struggle from the numerous reasons why.

Even in instances where a child is assessed and/or evaluated at an early stage, parents are not usually informed directly what specifically they need to work on the help their child succeed in school skills until a teacher conference which is given later in the school year and again in the spring when school is almost over. Before beginning school, the methods and/or tests used to evaluate children that are used today focus almost entirely on reading. Most schools presently teach reading by cueing based off contextual language and/or pictorial clues in order to guess what the words are. Guessing is a very inefficient technique and prone to errors. Good readers accurately decode words by manipulating the sounds in words to which over time creates fluent readers. As such, while such methods and/or tests may be somewhat effective in evaluating language, such methods and/or tests fail to evaluate speech, fine and gross motor skills and thus do not evaluate speech and language, fine and gross motor skills simultaneously, nor do they assess the social and emotional regulation required for success in an academic environment. This disclosure describes an improvement over these prior art technologies.

### SUMMARY

In one embodiment, in accordance with the principles of the present disclosure, a method for providing evaluation of input data of an individual obtained through administering a worksheet is provided. The method comprises: (a) having the individual draw a line and shape to generate a fine motor skill input data; (b) evaluating the fine motor skill input data against an answer key to generate a fine motor skill evaluation result, wherein the fine motor skill evaluation result is a first determination with respect to the presence or absence of fine motor skill deficiency; Determine the pencil grip if fisted and if so, teach parent or notify teacher to have child work on correct 3 point pencil grip. (c) having the individual do a gross motor skill and receptive language task to generate a gross motor skill input data; (d) evaluating the gross motor skill input data against an answer key to generate a gross motor skill evaluation result, wherein the gross motor skill evaluation result is a first determination with respect to the presence or absence of gross motor skill deficiency; evaluate ability to follow directions to assess receptive language skills and emotional regulation (e) having the individual say a first letter in at least one word to generate a sound articulation input data; (f) evaluating the sound articulation input data against an answer key to generate a sound articulation evaluation result, wherein the sound articulation skill evaluation result is a first determination with respect to the presence or absence of sound articulation deficiency; (g) having the individual determine if one or more letters or words are the same or different to generate a sound discrimination input data; (h) evaluating the sound discrimination input data against an answer key to generate a sound determination evaluation result, wherein the sound determination skill evaluation result is a first determination with respect to the presence or absence of sound determination articulation deficiency; and (i) generating a referral referring the individual for services in areas where the evaluation results were deficient to promote at least one of fine motor skills, gross motor skills, sound articulation, sound discrimination skills receptive language skills, and social emotional development.

In one embodiment, in accordance with the principles of the present disclosure, a method for providing evaluation of input data of an individual obtained through administering a worksheet is provided. The method comprises: (a) having the individual perform a test relating to receptive language skills to generate a receptive language skill input data; (b) evaluating the receptive language skills input data against an answer key to generate a receptive language skills evaluation result, wherein the receptive language skills evaluation result is a first determination with respect to the presence or absence of receptive language deficiency; (c) having the individual perform a test relating to sound discrimination skills to generate a sound discrimination skill input data; (d) evaluating the sound discrimination skill input data against an answer key to generate a sound discrimination skill evaluation result, wherein the sound discrimination skill result is a first determination with respect to the presence or absence of sound discrimination deficiency; (e) having the individual perform a test relating to sound order awareness skills to generate a sound order awareness skill input data; (f) evaluating the sound order awareness skill input data against an answer key to generate a sound order awareness skill evaluation result, wherein the sound order awareness skill evaluation result is a first determination with respect to the presence or absence of sound order awareness deficiency; (g) having the individual perform a test relating to fine motor skills to generate a fine motor skill input data; (h) evaluating the fine motor skill input data against an answer key to generate a fine motor skill evaluation result, wherein the fine motor skill evaluation result is a first determination with respect to the presence or absence of fine motor deficiency; drawing a shape and determining pencil grip (i) having the individual perform a test relating to letter naming and letter sounds to generate a letter naming and letter sound input data; (j) evaluating the letter naming and letter sound input data against an answer key to generate a letter naming and letter sound evaluation result, wherein the letter naming and letter sound evaluation result is a first determination with respect to the presence or absence of letter naming and letter sound deficiency; (k) having the individual perform a test relating to sound deletion to generate a sound deletion input data; (l) evaluating the sound deletion input data against an answer key to generate a sound deletion evaluation result, wherein the sound deletion evaluation result is a first determination with respect to the presence or absence of sound deletion deficiency; and (m) generating a referral referring the individual for services in areas where the evaluation results were deficient to promote at least one of receptive language, sound discrimination, sound order, fine motor, letter naming and letter sound and sound deletion skills, and social and emotional development.

In one embodiment, in accordance with the principles of the present disclosure, a method for providing evaluation of input data of an individual obtained through administering a worksheet is provided. The method comprises: (a) having the individual perform a test relating to receptive language skills to generate a receptive language skill input data; (b) evaluating the receptive language skills input data against an answer key to generate a receptive language skills evaluation result, wherein the receptive language skills evaluation result is a first determination with respect to the presence or absence of receptive language deficiency; (c) having the individual perform a test relating to fine motor skills to generate a fine motor skill input data; (d) evaluating the fine motor pencil grip skill input data against an answer key to generate a fine motor skill evaluation result, wherein the fine motor skill result is a first determination with respect to the presence or absence of fine motor deficiency; (e) having the individual perform a test relating to letter naming and letter sound skills to generate a letter naming and letter sound skill input data; (f) evaluating the letter naming and letter sound skill input data against an answer key to generate a letter naming and letter sound skill evaluation result, wherein the letter naming and letter sound skill evaluation result is a first determination with respect to the presence or absence of letter naming and letter sound deficiency; (g) having the individual perform a test relating to sound deletion to generate a sound deletion input data; (h) evaluating the word deletion and sound deletion input data against an answer key to generate a word deletion and sound deletion evaluation result, wherein the word deletion and sound deletion evaluation result is a first determination with respect to the presence or absence of word deletion and sound deletion deficiency; (i) having the individual perform a test relating to rhyming to generate a rhyming input data; (j) evaluating the rhyming input data against an answer key to generate a rhyming evaluation result, wherein the rhyming evaluation result is a first determination with respect to the presence or absence of rhyming deficiency; and (k) generating a referral referring the individual for services in areas where the evaluation results were deficient to promote at least one of receptive language, fine motor, letter naming and letter sound, word deletion, sound deletion, rhyming skill, and social emotional development or regulation.

In one embodiment, in accordance with the principles of the present disclosure, a method for providing evaluation of input data of an individual obtained through administering a worksheet is provided. The method comprises: (a) having the individual show one or more shapes to generate a receptive language input and color discrimination data; (b) evaluating the receptive language input data against an answer key to generate a receptive language evaluation result and color discrimination, wherein the receptive language evaluation result is a first determination with respect to the presence or absence of receptive language deficiency or color discrimination deficit; (c) having the individual do a fine motor skill to generate a fine motor skill input data; (d) evaluating the fine motor skill input data against an answer key to generate a fine motor skill evaluation result, wherein the fine motor skill evaluation result is a first determination with respect to the presence or absence of fine motor skill deficiency; (e) having the individual say letters and/or sounds to generate a letter/sound input data; (f) evaluating the letter/sound input data against an answer key to generate a letter/sound evaluation result, wherein the letter/sound evaluation result is a first determination with respect to the presence or absence of letter/sound deficiency; (g) having the individual take away sounds and/or letters from a word or words to generate a phoneme input data; (h) evaluating the phoneme input data against an answer key to generate a phoneme evaluation result, wherein the phoneme evaluation result is a first determination with respect to the presence or absence of phoneme deficiency; (i) having the individual perform rhyming exercises to generate a rhyming input data; (j) evaluating the rhyming input data against an answer key to generate a rhyming evaluation result, wherein the rhyming evaluation result is a first determination with respect to the presence or absence of rhyming deficiency; (k) having the individual read nonsense words to generate a nonsense word input data; (l) evaluating the nonsense word input data against an answer key to generate a nonsense word evaluation result, wherein the nonsense word evaluation result is a first determination with respect to the presence or absence of nonsense word deficiency; and (m) generating a referral referring the individual for services in areas where the evaluation results were deficient to promote at least one of receptive language, fine motor, letter/sound, phoneme (phonological processing), rhyming, color discrimination and emotional regulation and engagement and nonsense word skills.

In one embodiment, in accordance with the principles of the present disclosure, a method for providing evaluation of input data of an individual obtained through administering a worksheet is provided. The method comprises: (a) having the individual demonstrate fine motor skills to generate a fine motor skill input data; (b) evaluating the fine motor skill input data against an answer key to generate a fine motor skill evaluation result, wherein the fine motor skill evaluation result is a first determination with respect to the presence or absence of fine motor skill deficiency; (c) having the individual take away a sound or sounds to generate a phoneme elision input data; (d) evaluating the phoneme elision input data against an answer key to generate a phoneme elision evaluation result, wherein the phoneme elision evaluation result is a first determination with respect to the presence or absence of phoneme elision deficiency; (e) having the individual read a single word to generate a single word reading input data; (f) evaluating the single word reading input data against an answer key to generate a single word reading evaluation result, wherein the single word reading evaluation result is a first determination with respect to the presence or absence of single word reading deficiency; (g) having the individual read nonsense words to generate a nonsense word input data; (h) evaluating the nonsense word input data against an answer key to generate a nonsense word evaluation result, wherein the nonsense word evaluation result is a first determination with respect to the presence or absence of nonsense word deficiency; and (i) generating a referral referring the individual for services in areas where the evaluation results were deficient to promote at least one of fine motor, phoneme elision, single word reading, color discrimination, engagement and emotional regulation and nonsense word skills

### BRIEF DESCRIPTION OF THE DRAWINGS

The present disclosure will become more readily apparent from the specific description accompanied by the following drawings, in which:

FIG. 1 is one embodiment of a questionnaire used in connection with a first part of a method for evaluating the presence or absence of a delay and/or disorder and/or school readiness, social emotional, fine motor/gross motor in accordance with the principles of the present disclosure;

FIG. 2 is an example of one embodiment of a form/document used in connection with the first part of the method, in accordance with the principles of the present disclosure;

FIG. 3 is an example of one embodiment of a worksheet used in connection in the first part of the method, in accordance with the principles of the present disclosure;

FIG. 4 is an example of one embodiment of a worksheet used in connection in the first part of the method, in accordance with the principles of the present disclosure;

FIG. 5A is an example of one page of one embodiment of an assessment used in connection with the first part of the method, in accordance with the principles of the present disclosure;

FIG. 5B is an example of one embodiment of another page of the assessment shown in FIG. 5A;

FIG. 6A is an example of one page of one embodiment of an assessment used in connection with the first part of the method, in accordance with the principles of the present disclosure;

FIG. 6B is an example of one embodiment of another page of the assessment shown in FIG. 6A;

FIG. 7A is an example of one page from one embodiment of a milestone information sheet used in connection with the first part of the method, in accordance with the principles of the present disclosure;

FIG. 7B is an example of another page of the milestone information sheet shown in FIG. 7A, in accordance with the principles of the present disclosure;

FIG. 7C is an example of another page of the milestone information sheet shown in FIG. 7A, in accordance with the principles of the present disclosure;

FIG. 7D is an example of another page of the milestone information sheet shown in FIG. 7A, in accordance with the principles of the present disclosure;

FIG. 7E is an example of another page of the milestone information sheet shown in FIG. 7A, in accordance with the principles of the present disclosure;

FIG. 7F is an example of another page of the milestone information sheet shown in FIG. 7A, in accordance with the principles of the present disclosure;

FIG. 7G is an example of one embodiment of another page from the assessment shown in FIG. 7A, in accordance with the principles of the present disclosure;

FIG. 7H is an example of one embodiment of an assessment guided treatment flow chart, in accordance with the principles of the present disclosure;

FIG. 7I is an example of one embodiment of an assessment guided treatment flow chart, in accordance with the principles of the present disclosure;

FIG. 7J is an example of one embodiment of an assessment guided treatment flow chart, in accordance with the principles of the present disclosure;

FIG. 7K is an example of one embodiment of an assessment guided treatment flow chart, in accordance with the principles of the present disclosure;

FIG. 7L is an example of one embodiment of an assessment guided treatment flow chart, in accordance with the principles of the present disclosure;

FIG. 7M is an example of one embodiment of an assessment guided treatment flow chart, in accordance with the principles of the present disclosure;

FIG. 7N is an example of one embodiment of an assessment guided treatment flow chart, in accordance with the principles of the present disclosure;

FIG. 7O is an example of one embodiment of an assessment guided treatment flow chart, in accordance with the principles of the present disclosure;

FIG. 7P is an example of one embodiment of an assessment guided treatment flow chart, in accordance with the principles of the present disclosure;

FIG. 7Q is an example of one embodiment of an assessment guided treatment flow chart, in accordance with the principles of the present disclosure;

FIG. 8 is one embodiment of a questionnaire used in connection with a second part of the method, in accordance with the principles of the present disclosure;

FIG. 9A is an example of one embodiment of a form/document used in connection with the second part of the method, in accordance with the principles of the present disclosure;

FIG. 9B is one embodiment of a form/document used in connection with form/document shown in FIG. 9A, in accordance with the principles of the present disclosure;

FIG. 9C is one embodiment of a form/document used in connection with form/document shown in FIG. 9A, in accordance with the principles of the present disclosure;

FIG. 10A is an example of one embodiment of a form/document used in connection with the second part of the method, in accordance with the principles of the present disclosure;

FIG. 10B is one embodiment of a form/document used in connection with form/document shown in FIG. 10A, in accordance with the principles of the present disclosure;

FIG. 10C is one embodiment of a form/document used in connection with form/document shown in FIG. 10A, in accordance with the principles of the present disclosure;

FIG. 11A is an example of a page from one embodiment of a worksheet used in connection with the second part of the method, in accordance with the principles of the present disclosure;

FIG. 11B is one embodiment of another page from the worksheet shown in FIG. 11A, in accordance with the principles of the present disclosure;

FIG. 11C is one embodiment of another page from the worksheet shown in FIG. 11A, in accordance with the principles of the present disclosure;

FIG. 11D is one embodiment of another page from the worksheet shown in FIG. 11A, in accordance with the principles of the present disclosure;

FIG. 12A is an example of a page from one embodiment of a worksheet used in connection with the second part of the method, in accordance with the principles of the present disclosure;

FIG. 12B is one embodiment of another page from the worksheet shown in FIG. 12A, in accordance with the principles of the present disclosure;

FIG. 12C is one embodiment of another page from the worksheet shown in FIG. 12A, in accordance with the principles of the present disclosure;

FIG. 12D is one embodiment of another page from the worksheet shown in FIG. 12A, in accordance with the principles of the present disclosure;

FIG. 13A is an example of one embodiment of one page of an assessment used in connection with the second part of the method, in accordance with the principles of the present disclosure;

FIG. 13B is an example of one embodiment of another page from the assessment shown in FIG. 13A, in accordance with the principles of the present disclosure;

FIG. 13C is an example of one embodiment of another page from the assessment shown in FIG. 13A, in accordance with the principles of the present disclosure;

FIG. 13D is an example of one embodiment of another page from the assessment shown in FIG. 13A, in accordance with the principles of the present disclosure;

FIG. 13E is an example of one embodiment of another page from the assessment shown in FIG. 13A, in accordance with the principles of the present disclosure;

FIG. 13F is an example of one embodiment of another page from the assessment shown in FIG. 13A, in accordance with the principles of the present disclosure;

FIG. 13G is an example of one embodiment of another page from the assessment shown in FIG. 13A, in accordance with the principles of the present disclosure;

FIG. 13H is an example of one embodiment of another page from the assessment shown in FIG. 13A, in accordance with the principles of the present disclosure;

FIG. 13I is an example of one embodiment of another page from the assessment shown in FIG. 13A, in accordance with the principles of the present disclosure;

FIG. 13J is an example of one embodiment of another page from the assessment shown in FIG. 13A, in accordance with the principles of the present disclosure;

FIG. 13K is an example of one embodiment of another page from the assessment shown in FIG. 13A, in accordance with the principles of the present disclosure;

FIG. 13L is an example of one embodiment of another page from the assessment shown in FIG. 13A, in accordance with the principles of the present disclosure;

FIG. 13M is an example of one embodiment of another page from the assessment shown in FIG. 13A, in accordance with the principles of the present disclosure;

FIG. 13N is an example of one embodiment of another page from the assessment shown in FIG. 13A, in accordance with the principles of the present disclosure;

FIG. 13O is an example of one embodiment of another page from the assessment shown in FIG. 13A, in accordance with the principles of the present disclosure;

FIG. 13P is an example of one embodiment of another page from the assessment shown in FIG. 13A, in accordance with the principles of the present disclosure;

FIG. 14A is one embodiment of an example of one page from a milestone information sheet used in connection with the second part of the method, in accordance with the principles of the present disclosure;

FIG. 14B is an example one embodiment of another page from the milestone information sheet shown in FIG. 14A, in accordance with the principles of the present disclosure;

FIG. 14C is an example one embodiment of another page from the milestone information sheet shown in FIG. 14A, in accordance with the principles of the present disclosure;

FIG. 14D is an example one embodiment of another page from the milestone information sheet shown in FIG. 14A, in accordance with the principles of the present disclosure;

FIG. 14E is an example one embodiment of another page from the milestone information sheet shown in FIG. 14A, in accordance with the principles of the present disclosure;

FIG. 14F is an example one embodiment of another page from the milestone information sheet shown in FIG. 14A, in accordance with the principles of the present disclosure;

FIG. 14G is an example one embodiment of another page from the milestone information sheet shown in FIG. 14A, in accordance with the principles of the present disclosure;

FIG. 15A is one embodiment of a questionnaire used in connection with a third part of the method, in accordance with the principles of the present disclosure;

FIG. 15B is an example of one embodiment of a form/document used in connection with the third part of the method, in accordance with the principles of the present disclosure;

FIG. 15C is an example of one embodiment of a form/document used in connection with the third part of the method, in accordance with the principles of the present disclosure;

FIG. 16A is an example of one embodiment of a form/document used in connection with the third part of the method, in accordance with the principles of the present disclosure;

FIG. 16B is an example of one embodiment of a form/document used in connection with the third part of the method, in accordance with the principles of the present disclosure;

FIG. 17A is an example of one embodiment of one page of an assessment used in connection with the third part of the method, in accordance with the principles of the present disclosure;

FIG. 17B is an example of one embodiment of another page from the assessment shown in FIG. 17A, in accordance with the principles of the present disclosure;

FIG. 17C is an example of one embodiment of another page from the assessment shown in FIG. 17A, in accordance with the principles of the present disclosure;

FIG. 17D is an example of one embodiment of another page from the assessment shown in FIG. 17A, in accordance with the principles of the present disclosure;

FIG. 17E is an example of one embodiment of another page from the assessment shown in FIG. 17A, in accordance with the principles of the present disclosure;

FIG. 17F is an example of one embodiment of another page from the assessment shown in FIG. 17A, in accordance with the principles of the present disclosure;

FIG. 18A is an example of one embodiment of one page of an assessment used in connection with the third part of the method, in accordance with the principles of the present disclosure;

FIG. 18B is an example of one embodiment of another page from the assessment shown in FIG. 18A, in accordance with the principles of the present disclosure;

FIG. 18C is an example of one embodiment of one page of an assessment used in connection with the third part of the method, in accordance with the principles of the present disclosure;

FIG. 18D is an example of one embodiment of one page of an assessment used in connection with the third part of the method, in accordance with the principles of the present disclosure;

FIG. 18E is an example of one embodiment of one page of an assessment used in connection with the third part of the method, in accordance with the principles of the present disclosure;

FIG. 18F is an example of one embodiment of one page of an assessment used in connection with the third part of the method, in accordance with the principles of the present disclosure;

FIG. 19A is an example of one embodiment of one page of an assessment used in connection with the third part of the method, in accordance with the principles of the present disclosure;

FIG. 19B is an example of one embodiment of another page from the assessment shown in FIG. 19A, in accordance with the principles of the present disclosure;

FIG. 19C is an example of one embodiment of another page from the assessment shown in FIG. 19A, in accordance with the principles of the present disclosure;

FIG. 19D is an example of one embodiment of another page from the assessment shown in FIG. 19A, in accordance with the principles of the present disclosure;

FIG. 19E is an example of one embodiment of another page from the assessment shown in FIG. 19A, in accordance with the principles of the present disclosure;

FIG. 19F is an example of one embodiment of another page from the assessment shown in FIG. 19A, in accordance with the principles of the present disclosure;

FIG. 19G is an example of one embodiment of another page from the assessment shown in FIG. 19A, in accordance with the principles of the present disclosure;

FIG. 19H is an example of one embodiment of another page from the assessment shown in FIG. 19A, in accordance with the principles of the present disclosure;

FIG. 19I is an example of one embodiment of another page from the assessment shown in FIG. 19A, in accordance with the principles of the present disclosure;

FIG. 19J is an example of one embodiment of another page from the assessment shown in FIG. 19A, in accordance with the principles of the present disclosure;

FIG. 19K is an example of one embodiment of another page from the assessment shown in FIG. 19A, in accordance with the principles of the present disclosure;

FIG. 19L is an example of one embodiment of another page from the assessment shown in FIG. 19A, in accordance with the principles of the present disclosure;

FIG. 19M is an example of one embodiment of another page from the assessment shown in FIG. 19A, in accordance with the principles of the present disclosure;

FIG. 19N is an example of one embodiment of another page from the assessment shown in FIG. 19A, in accordance with the principles of the present disclosure;

FIG. 20A is one embodiment of an example of one page from a milestone information sheet used in connection with the third part of the method, in accordance with the principles of the present disclosure;

FIG. 20B is an example one embodiment of another page from the milestone information sheet shown in FIG. 20A, in accordance with the principles of the present disclosure;

FIG. 20C is an example one embodiment of another page from the milestone information sheet shown in FIG. 20A, in accordance with the principles of the present disclosure;

FIG. 20D is an example one embodiment of another page from the milestone information sheet shown in FIG. 20A, in accordance with the principles of the present disclosure;

FIG. 20E is an example one embodiment of another page from the milestone information sheet shown in FIG. 20A, in accordance with the principles of the present disclosure;

FIG. 20F is an example one embodiment of another page from the milestone information sheet shown in FIG. 20A, in accordance with the principles of the present disclosure;

FIG. 20G is an example one embodiment of another page from the milestone information sheet shown in FIG. 20A, in accordance with the principles of the present disclosure;

FIG. 21 is one embodiment of a questionnaire used in connection with a fourth part of the method, in accordance with the principles of the present disclosure;

FIG. 22A is an example of one embodiment of one page of a worksheet used in connection with the fourth part of the method, in accordance with the principles of the present disclosure;

FIG. 22B is one embodiment of another page of the worksheet shown in FIG. 22A, in accordance with the principles of the present disclosure;

FIG. 22C is one embodiment of another page of the worksheet shown in FIG. 22A, in accordance with the principles of the present disclosure;

FIG. 22D is one embodiment of another page of the worksheet shown in FIG. 22A, in accordance with the principles of the present disclosure;

FIG. 22E is one embodiment of another page of the worksheet shown in FIG. 22A, in accordance with the principles of the present disclosure;

FIG. 22F is one embodiment of another page of the worksheet shown in FIG. 22A, in accordance with the principles of the present disclosure;

FIG. 22G is one embodiment of another page of the worksheet shown in FIG. 22A, in accordance with the principles of the present disclosure;

FIG. 22H is one embodiment of another page of the worksheet shown in FIG. 22A, in accordance with the principles of the present disclosure;

FIG. 22I is one embodiment of another page of the worksheet shown in FIG. 22A, in accordance with the principles of the present disclosure;

FIG. 23A is an example of one embodiment of one page of a worksheet used in connection with the fourth part of the method, in accordance with the principles of the present disclosure;

FIG. 23B is one embodiment of another page of the worksheet shown in FIG. 23A, in accordance with the principles of the present disclosure;

FIG. 23C is one embodiment of another page of the worksheet shown in FIG. 23A, in accordance with the principles of the present disclosure;

FIG. 23D is one embodiment of another page of the worksheet shown in FIG. 23A, in accordance with the principles of the present disclosure;

FIG. 23E is one embodiment of another page of the worksheet shown in FIG. 23A, in accordance with the principles of the present disclosure;

FIG. 23F is one embodiment of another page of the worksheet shown in FIG. 23A, in accordance with the principles of the present disclosure;

FIG. 23G is one embodiment of another page of the worksheet shown in FIG. 23A, in accordance with the principles of the present disclosure;

FIG. 23H is one embodiment of another page of the worksheet shown in FIG. 23A, in accordance with the principles of the present disclosure;

FIG. 23I is one embodiment of another page of the worksheet shown in FIG. 23A, in accordance with the principles of the present disclosure;

FIG. 24A is an example of one embodiment of one page of an assessment used in connection with the fourth part of the method, in accordance with the principles of the present disclosure;

FIG. 24B is one embodiment of another page of the assessment shown in FIG. 24A, in accordance with the principles of the present disclosure;

FIG. 24C is one embodiment of another page of the assessment shown in FIG. 24A, in accordance with the principles of the present disclosure;

FIG. 24D is one embodiment of another page of the assessment shown in FIG. 24A, in accordance with the principles of the present disclosure;

FIG. 24E is one embodiment of another page of the assessment shown in FIG. 24A, in accordance with the principles of the present disclosure;

FIG. 24F is one embodiment of another page of the assessment shown in FIG. 24A, in accordance with the principles of the present disclosure;

FIG. 24G is one embodiment of another page of the assessment shown in FIG. 24A, in accordance with the principles of the present disclosure;

FIG. 24H is one embodiment of another page of the assessment shown in FIG. 24A, in accordance with the principles of the present disclosure;

FIG. 24I is one embodiment of another page of the assessment shown in FIG. 24A, in accordance with the principles of the present disclosure;

FIG. 24J is one embodiment of another page of the assessment shown in FIG. 24A, in accordance with the principles of the present disclosure;

FIG. 24K is one embodiment of another page of the assessment shown in FIG. 24A, in accordance with the principles of the present disclosure;

FIG. 24L is one embodiment of another page of the assessment shown in FIG. 24A, in accordance with the principles of the present disclosure;

FIG. 24M is one embodiment of another page of the assessment shown in FIG. 24A, in accordance with the principles of the present disclosure;

FIG. 24N is one embodiment of another page of the assessment shown in FIG. 24A, in accordance with the principles of the present disclosure;

FIG. 24O is one embodiment of another page of the assessment shown in FIG. 24A, in accordance with the principles of the present disclosure;

FIG. 25A is one embodiment of an example of one page from a milestone information sheet used in connection with the fourth part of the method, in accordance with the principles of the present disclosure;

FIG. 25B is an example one embodiment of another page from the milestone information sheet shown in FIG. 25A, in accordance with the principles of the present disclosure;

FIG. 25C is an example one embodiment of another page from the milestone information sheet shown in FIG. 25A, in accordance with the principles of the present disclosure;

FIG. 25D is an example one embodiment of another page from the milestone information sheet shown in FIG. 25A, in accordance with the principles of the present disclosure;

FIG. 25E is an example one embodiment of another page from the milestone information sheet shown in FIG. 25A, in accordance with the principles of the present disclosure;

FIG. 25F is an example one embodiment of another page from the milestone information sheet shown in FIG. 25A, in accordance with the principles of the present disclosure;

FIG. 25G is an example one embodiment of another page from the milestone information sheet shown in FIG. 25A, in accordance with the principles of the present disclosure;

FIG. 26 is one embodiment of a questionnaire used in connection with a fifth part of the method, in accordance with the principles of the present disclosure;

FIG. 27A is an example of one embodiment of one page of a worksheet used in connection with the fifth part of the method, in accordance with the principles of the present disclosure;

FIG. 27B is one embodiment of another page of the worksheet shown in FIG. 27A, in accordance with the principles of the present disclosure;

FIG. 27C is one embodiment of another page of the worksheet shown in FIG. 27A, in accordance with the principles of the present disclosure;

FIG. 27D is one embodiment of another page of the worksheet shown in FIG. 27A, in accordance with the principles of the present disclosure;

FIG. 27E is one embodiment of another page of the worksheet shown in FIG. 27A, in accordance with the principles of the present disclosure;

FIG. 28A is an example of one embodiment of one page of a worksheet used in connection with the fifth part of the method, in accordance with the principles of the present disclosure;

FIG. 28B is one embodiment of another page of the worksheet shown in FIG. 28A, in accordance with the principles of the present disclosure;

FIG. 28C is one embodiment of another page of the worksheet shown in FIG. 28A, in accordance with the principles of the present disclosure;

FIG. 28D is one embodiment of another page of the worksheet shown in FIG. 28A, in accordance with the principles of the present disclosure;

FIG. 28E is one embodiment of another page of the worksheet shown in FIG. 28A, in accordance with the principles of the present disclosure;

FIG. 29A is one embodiment of one page of an assessment used in connection with the fifth part of the method, in accordance with the principles of the present disclosure;

FIG. 29B is one embodiment of another page of the assessment shown in FIG. 29A, in accordance with the principles of the present disclosure;

FIG. 29C is one embodiment of another page of the assessment shown in FIG. 29A, in accordance with the principles of the present disclosure;

FIG. 29D is one embodiment of another page of the assessment shown in FIG. 29A, in accordance with the principles of the present disclosure;

FIG. 29E is one embodiment of another page of the assessment shown in FIG. 29A, in accordance with the principles of the present disclosure;

FIG. 29F is one embodiment of another page of the assessment shown in FIG. 29A, in accordance with the principles of the present disclosure;

FIG. 29G is one embodiment of another page of the assessment shown in FIG. 29A, in accordance with the principles of the present disclosure;

FIG. 29H is one embodiment of another page of the assessment shown in FIG. 29A, in accordance with the principles of the present disclosure;

FIG. 29I is one embodiment of another page of the assessment shown in FIG. 29A, in accordance with the principles of the present disclosure;

FIG. 29J is one embodiment of another page of the assessment shown in FIG. 29A, in accordance with the principles of the present disclosure;

FIG. 29K is one embodiment of another page of the assessment shown in FIG. 29A, in accordance with the principles of the present disclosure;

FIG. 29L is one embodiment of another page of the assessment shown in FIG. 29A, in accordance with the principles of the present disclosure;

FIG. 29M is one embodiment of another page of the assessment shown in FIG. 29A, in accordance with the principles of the present disclosure;

FIG. 30A is one embodiment of an example of one page from a milestone information sheet used in connection with the fifth part of the method, in accordance with the principles of the present disclosure;

FIG. 30B is an example one embodiment of another page from the milestone information sheet shown in FIG. 30A, in accordance with the principles of the present disclosure;

FIG. 30C is an example one embodiment of another page from the milestone information sheet shown in FIG. 30A, in accordance with the principles of the present disclosure;

FIG. 30D is an example one embodiment of another page from the milestone information sheet shown in FIG. 30A, in accordance with the principles of the present disclosure;

FIG. 30E is an example one embodiment of another page from the milestone information sheet shown in FIG. 30A, in accordance with the principles of the present disclosure;

FIG. 30F is an example one embodiment of another page from the milestone information sheet shown in FIG. 30A, in accordance with the principles of the present disclosure;

FIG. 30G is an example one embodiment of another page from the milestone information sheet shown in FIG. 30A, in accordance with the principles of the present disclosure;

FIG. 31 is one embodiment of a questionnaire used in connection with a sixth part of the method, in accordance with the principles of the present disclosure;

FIG. 32A is an example of one embodiment of one page of a worksheet used in connection with the sixth part of the method, in accordance with the principles of the present disclosure;

FIG. 32B is one embodiment of another page of the worksheet shown in FIG. 32A, in accordance with the principles of the present disclosure;

FIG. 32C is one embodiment of another page of the worksheet shown in FIG. 32A, in accordance with the principles of the present disclosure;

FIG. 32D is one embodiment of another page of the worksheet shown in FIG. 32A, in accordance with the principles of the present disclosure;

FIG. 32E is one embodiment of another page of the worksheet shown in FIG. 32A, in accordance with the principles of the present disclosure;

FIG. 32F is one embodiment of another page of the worksheet shown in FIG. 32A, in accordance with the principles of the present disclosure;

FIG. 33A is an example of one embodiment of one page of a worksheet used in connection with the sixth part of the method, in accordance with the principles of the present disclosure;

FIG. 33B is one embodiment of another page of the worksheet shown in FIG. 33A, in accordance with the principles of the present disclosure;

FIG. 33C is one embodiment of another page of the worksheet shown in FIG. 33A, in accordance with the principles of the present disclosure;

FIG. 33D is one embodiment of another page of the worksheet shown in FIG. 33A, in accordance with the principles of the present disclosure;

FIG. 33E is one embodiment of another page of the worksheet shown in FIG. 33A, in accordance with the principles of the present disclosure;

FIG. 33F is one embodiment of another page of the worksheet shown in FIG. 33A, in accordance with the principles of the present disclosure;

FIG. 34A is one embodiment of one page of an assessment used in connection with the sixth part of the method, in accordance with the principles of the present disclosure;

FIG. 34B is one embodiment of another page of the assessment shown in FIG. 34A, in accordance with the principles of the present disclosure;

FIG. 34C is one embodiment of another page of the assessment shown in FIG. 34A, in accordance with the principles of the present disclosure;

FIG. 34D is one embodiment of another page of the assessment shown in FIG. 34A, in accordance with the principles of the present disclosure;

FIG. 34E is one embodiment of another page of the assessment shown in FIG. 34A, in accordance with the principles of the present disclosure;

FIG. 34F is one embodiment of another page of the assessment shown in FIG. 34A, in accordance with the principles of the present disclosure;

FIG. 34G is one embodiment of another page of the assessment shown in FIG. 34A, in accordance with the principles of the present disclosure;

FIG. 34H is one embodiment of another page of the assessment shown in FIG. 34A, in accordance with the principles of the present disclosure;

FIG. 34I is one embodiment of another page of the assessment shown in FIG. 34A, in accordance with the principles of the present disclosure;

FIG. 34J is one embodiment of another page of the assessment shown in FIG. 34A, in accordance with the principles of the present disclosure;

FIG. 34K is one embodiment of another page of the assessment shown in FIG. 34A, in accordance with the principles of the present disclosure;

FIG. 34L is one embodiment of another page of the assessment shown in FIG. 34A, in accordance with the principles of the present disclosure;

FIG. 34M is one embodiment of another page of the assessment shown in FIG. 34A, in accordance with the principles of the present disclosure;

FIG. 35A is one embodiment of an example of one page from a milestone information sheet used in connection with the sixth part of the method, in accordance with the principles of the present disclosure;

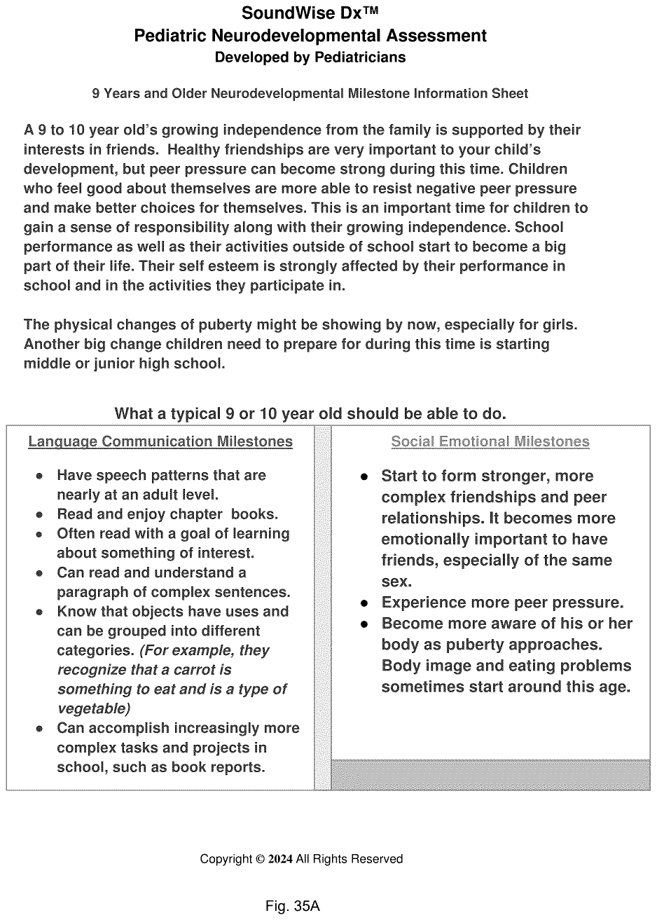

FIG. 35B is an example one embodiment of another page from the milestone information sheet shown in FIG. 35A, in accordance with the principles of the present disclosure;

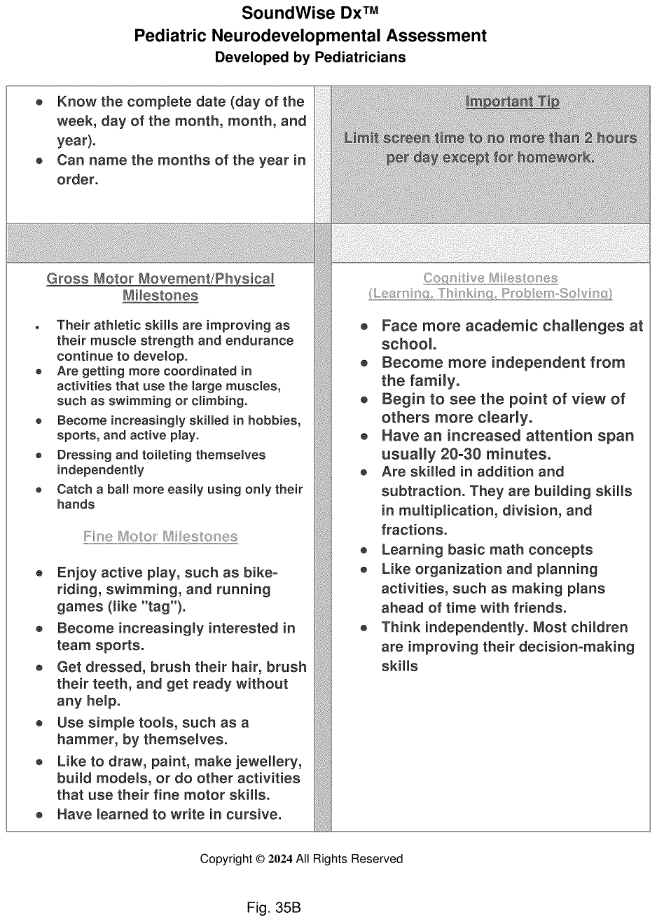

FIG. 35C is an example one embodiment of another page from the milestone information sheet shown in FIG. 35A, in accordance with the principles of the present disclosure;

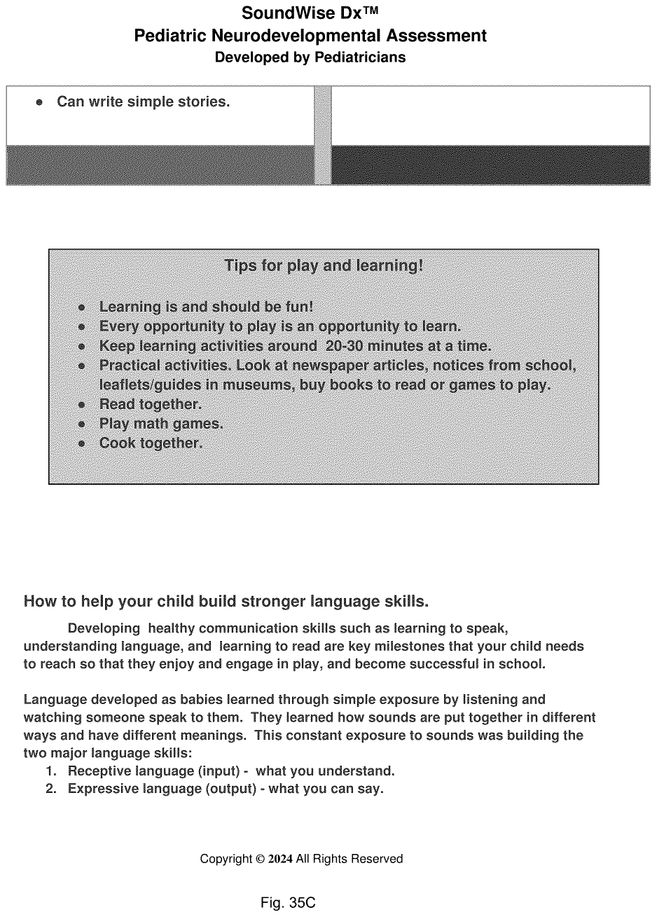

FIG. 35D is an example one embodiment of another page from the milestone information sheet shown in FIG. 35A, in accordance with the principles of the present disclosure;

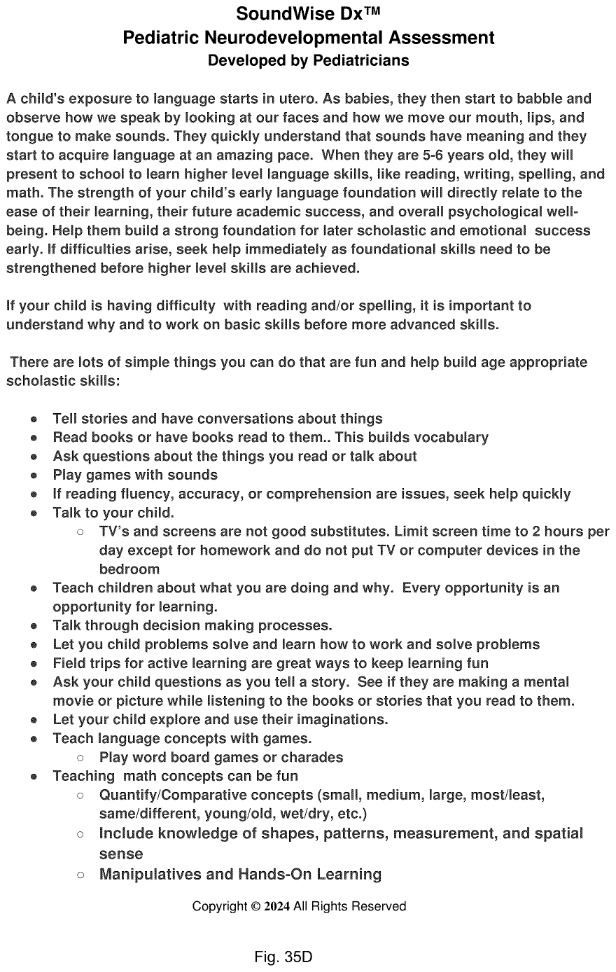

FIG. 35E is an example one embodiment of another page from the milestone information sheet shown in FIG. 35A, in accordance with the principles of the present disclosure;

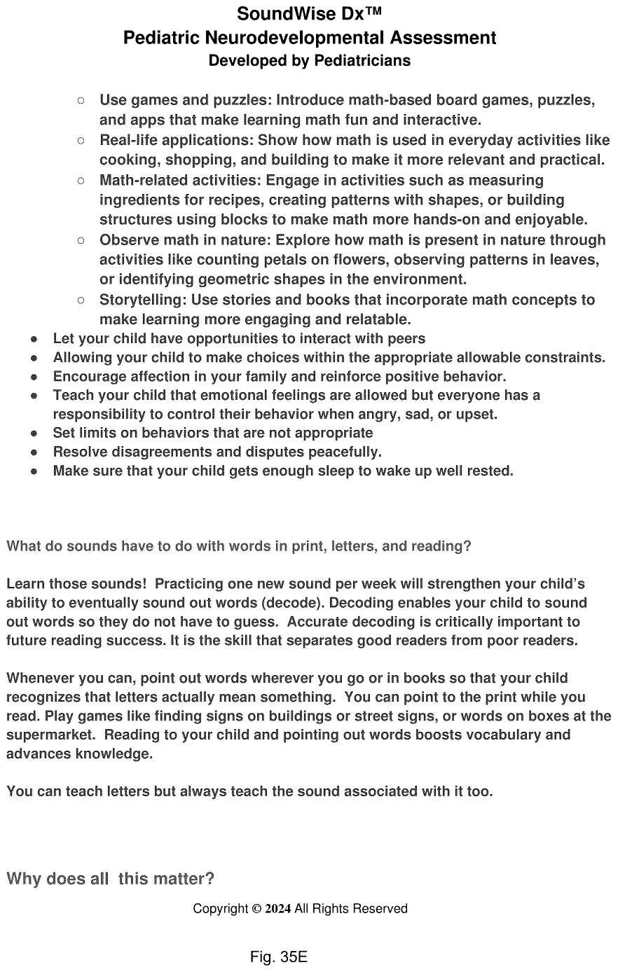

FIG. 35F is an example one embodiment of another page from the milestone information sheet shown in FIG. 35A, in accordance with the principles of the present disclosure;

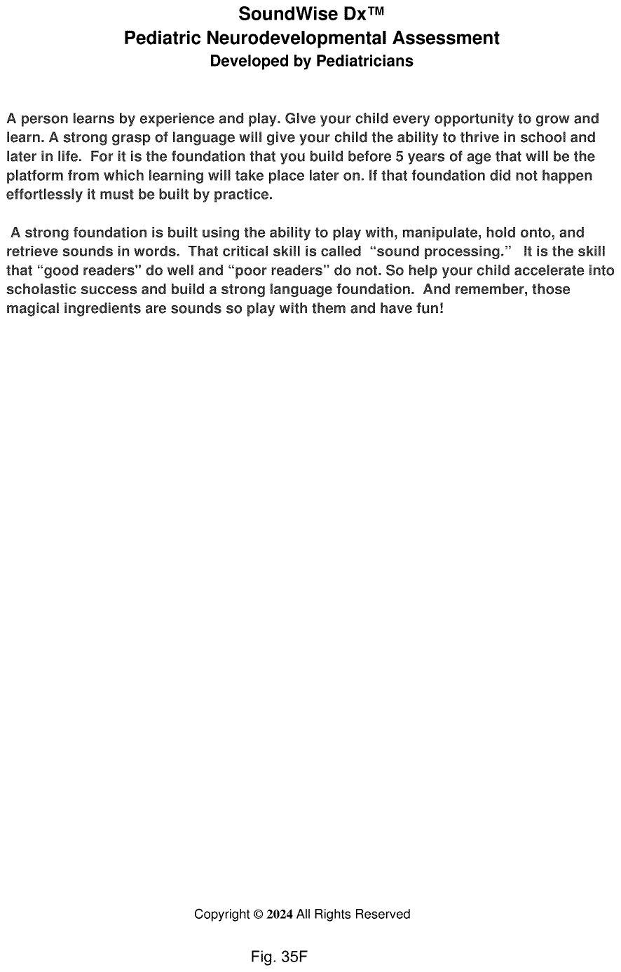

FIG. 36A is one page of an assessment report used and/or generated in connection with the first part of the method, in accordance with the principles of the present disclosure;

FIG. 36B is another page of the assessment report shown in FIG. 36A, in accordance with the principles of the present disclosure;

FIG. 37A is one page of an assessment report used and/or generated in connection with the second part of the method, in accordance with the principles of the present disclosure;

FIG. 37B is another page of the assessment report shown in FIG. 37A, in accordance with the principles of the present disclosure;

FIG. 37C is another page of the assessment report shown in FIG. 37A, in accordance with the principles of the present disclosure;

FIG. 38A is one page of an assessment report used and/or generated in connection with the third part of the method, in accordance with the principles of the present disclosure;

FIG. 38B is another page of the assessment report shown in FIG. 38A, in accordance with the principles of the present disclosure;

FIG. 38C is another page of the assessment report shown in FIG. 38A, in accordance with the principles of the present disclosure;

FIG. 39A is one page of an assessment report used and/or generated in connection with the fourth part of the method, in accordance with the principles of the present disclosure;

FIG. 39B is another page of the assessment report shown in FIG. 39A, in accordance with the principles of the present disclosure;

FIG. 39C is another page of the assessment report shown in FIG. 39A, in accordance with the principles of the present disclosure;

FIG. 40A is one page of an assessment report used and/or generated in connection with the fifth part of the method, in accordance with the principles of the present disclosure;

FIG. 40B is another page of the assessment report shown in FIG. 40A, in accordance with the principles of the present disclosure;

FIG. 41A is one page of an assessment report used and/or generated in connection with the sixth part of the method, in accordance with the principles of the present disclosure;

FIG. 41B is another page of the assessment report shown in FIG. 41A, in accordance with the principles of the present disclosure; and

Like reference numerals indicate similar parts throughout the figures.

### DETAILED DESCRIPTION

The present disclosure may be understood more readily by reference to the following detailed description of the disclosure taken in connection with the accompanying drawing figures, which form a part of this disclosure. It is to be understood that this disclosure is not limited to the specific systems, methods, conditions or parameters described and/or shown herein, and that the terminology used herein is for the purpose of describing particular embodiments by way of example only and is not intended to be limiting of the claimed disclosure.

Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” or “top” and “bottom” are relative and used only in the context to the other and are not necessarily “superior” and “inferior.”

Educational (literacy, academic achievement and ability to write with correct pencil grip), neurotypical social emotional learning, sports participation success for all children may be achieved through assessing early childhood development with usage of the system and method of the present disclosure. In particular, the system and method of the present disclosure deliver collaborative actionable skills, personalized, customizable and specific, for each child encompassing and not limited to speech and language, fine and gross motor skills, and social emotional learning.

In some embodiments, the system and method of the present disclosure provide an automated interactive scalable AI based assessment of selected duration (e.g., 15 minutes), scored by AI that can be configured to select personalized, customizable strategies for speech, language, fine and gross motor skills, color blindness, neurotypical social emotional learning. The system and method of the present disclosure can thus be utilized to foster a strong start for reading, social emotional interaction learning, and confidence in sports participation and other extracurricular activities. In some embodiments, the method of the present disclosure can begin when a child is three years old (or younger) and could be made available to pediatricians/medical providers, daycares, preschools, early intervention centers, elementary schools and children homeschooled, caregivers, and parents who want to work with their children in addition to going to school. While it is envisioned that the system and method of the present disclosure may be utilized with all children, that the system and method of the present disclosure may be especially beneficial for children with speech, language difficulties and phonological awareness/processing deficits (dyslexia), English as a second language children; disadvantaged and lack of opportunity; and fine and gross motor skill impairments and those with neurotypical social emotional impairments.

The system and method of the present disclosure are comprehensive by incorporating the critical components of early reading, writing, attention, processing (visual, sensory, auditorily), social emotional learning and motor skills integration simultaneously to identify students at risk. The system and method of the present disclosure are adapted to assure that every child has an appropriate neurodevelopmental course at each age. If they do not, the system and method of the present disclosure can be utilized to prescribe therapeutic treatment to help each child to improve on speech language, fine/gross motor color blindness and vision determination, neurotypical social emotional learning and behavioral regulation.

The system and method of the present disclosure may be utilized to assess children before or while they exhibit problems with speech and language, fine and gross motor, neurotypical social emotional learning in preschool/elementary schools or daycare settings, in pediatrician/provider offices, homeschoolers and for parents who believe their children exhibit developmental delay and are on long waiting list to get intervention or denied services. The system and method of the present disclosure may be utilized in provider offices to identify children during their yearly well child physical exams and when parents and teachers request a closer look at a child's developmental milestones to pick up if they have speech/language, fine/gross motor, neurotypical social emotional and any attention components that may cause a child to be delayed in the school setting including processing visual processing or activity deficits, color blindness, absence seizure, Anxiety, autism risk or emotional dysregulation.

In many instances, a child is not diagnosed and/or treated concerning their neurodevelopmental functional skills or cognitive function attributes until the child is in school or is about to begin school. This typically occurs when the child is approximately between 5 to six years old. A brief screen is done only delivering how their child compares to other children in the school. However, some speech/language disorders, such as, for example, the rise of reading struggles when discovered in early childhood will reduce the risk of future. Delaying treatment often makes the deficiencies or gaps so large that they are often insurmountable. Early intervention by school entry at age 6 prevents or greatly lessens the severity of struggles compared to waiting until a child is 8 years or older. It is therefore beneficial to diagnose and treat the child's language process deficits or phonological awareness deficits as early as possible to provide an opportunity for the child's language processing deficits to be treated and/or remediated by the time the child begins school. When “at risk” beginning readers receive intensive instruction, 56% to 92% of at-risk children across six studies reached the range of average reading ability. Sadly, children are typically not identified or diagnosed with a reading disability until the child is either in 3^{rd} or 4^{th} grade and by that time the child is several grade levels behind in reading and is difficult to catch up to grade level. It is financially very expensive for the parent to hire extra help after school to help the child catch up to grade level and it does cost the school district large amounts of tax dollars as well to help the child read at grade level. This also greatly applies to children from disadvantaged families and those who live in families where English is a second language. Seventy five percent of students who do not read proficiently by third grade never reach reading proficiency in future grades. There is a decrease of likelihood that struggling readers in third grade will attend college compared to their more proficient peers.

The system and method of the present disclosure is groundbreaking in that the system and method of the present disclosure integrate a medical approach together with an educational approach. The system and method of the present disclosure are preventive in that the system and method of the present disclosure prevent struggles or failures from happening instead of addressing them before they occur. When it comes to learning difficulties, we are largely focused on a reactive, deficit-driven, ‘wait to fail’ model instead of on the development of preventative approaches. The system and method of the present disclosure thus provide an early intervention approach. The system and method of the present disclosure are neurodevelopmental and transformative in the way that system and method of the present disclosure look at addressing early foundational milestone skills first. The system and method of the present disclosure are broad based and multifactorial in that the system and method of the present disclosure look at more than just reading. The system and method of the present disclosure are comprehensive by incorporating critical components of early reading, writing, attention, processing (visual, sensory, auditory), motor skills, and social emotional skills simultaneously to identify students at risk. The system and method of the present disclosure provide assessments and solutions that are based upon neurological (brain) science and the tenets of neuroplasticity (how the brain changes and learns). Best results are seen when the child is given intervention as early as possible. The system and method of the present disclosure is configured to incorporate the Medical-Home model allowing insurance coverage for treatment to be part of the solution. The system and method of the present disclosure are research-based with scientifically proven effectiveness in accordance with the guidelines and recommendations of the American Academy of Pediatrics as both inventors are active members. The system and method of the present disclosure are adapted for a child-centered, collaborative team approach that incorporates an array of adults (parents, teachers, pediatrician, counselors, and occupational, physical and speech therapists) working towards a common goal. The system and method of the present disclosure offer comprehensive solutions that provide customized personalized actionable enrichment plans. The system and method of the present disclosure are innovative in that the system and method of the present disclosure reshape the way school readiness is achieved by looking at multiple skills simultaneously instead of just looking at skills that are for reading. The system and method of the present disclosure are transformative in that early detection and intervention changes lives forever, reduce mental health disparities, build strong self-esteem and reduced absenteeism which is presently an increasingly large problem in the United States post covid pandemic. The system and method of the present disclosure provide universal neurodevelopmental assessments that offer equitable solutions that overcome many barriers like reading failure if risk of dyslexia is not picked up early, lack of access, lack of resources, English 2^{nd} language learners, geographic barriers and high cost of psychoeducational evaluations once failure has occurred. The system and method of the present disclosure are life-changing in that the system and method of the present disclosure provide children, educators, and families the tools and confidence needed to ensure children succeed scholastically, are healthy socially, emotionally and have competence and confidence in their ability to participate in sports, extra activity and thrive in academic life, employment, college and other avenues. The system and method of the present disclosure are adapted to build self-esteem in that a child who is successful in school is not as vulnerable to feelings of shame, failure, inadequacy, anxiety, depression, suicidal ideation, stronger reliance on social services, not completing their education and risk of incarceration in the future. Indeed, 93 million adults in the nation today read at or below basic levels, even though most living-wage jobs require proficient readers.

The system and method of the present disclosure layer foundational life and educational school readiness skills as a basis for scholastic success. The system and method of the present disclosure are adapted to focus on strengthening each foundational skills independently, simultaneously and as early as possible so that all layers can work well together to achieve a stronger and more successful end result. How each and every layer gets connected or integrated together determines how easily, effortlessly, and effectively a specific task can be performed. During early childhood, children develop skills from birth on and when a child is expected to develop which skill has long been studied and documented. Pediatricians study the various milestones specific for each age and during their examination at each well child examination, check for these skills and communicate to the caretakers of the child whether the child's milestones are up to date and if not, what action needs to take to help the child reach the correct milestone. When a child is not asked to demonstrate a skill, and we just rely on parental questionnaires, it is not always effective. A parent may not disclose their child's developmental delay due to fear of labelling culture beliefs, or lack of educational knowledge.

If a child is recommended to get therapy specific for speech, gross motor, fine motor, and/or behavior, then the provider will write a prescription for the specific therapy and send the patient to a state-run early intervention center to confirm the developmental needs of the child and if they agree if the child needs the therapy. If so, then if prescribed by the Pediatrician, the Early Intervention center sets up the therapy necessary for the child to undergo either at the child's home or at a day care facility or school. At the present time, the wait period from after the prescription is received by the Pediatrician to the State-run early intervention center is about 3 to 6 months and the time period to receive intervention is up to 6 to 12 months. A wait time this long in early childhood does not consider the neuroplasticity potential—the ability to build a greater number of connected neurocircuits (almost like building roads or more efficient highway system). Neuroplasticity means “brain change” or “brain learning.” The tenets of neuroplasticity are based upon five core principles: 1. Intensity 2. Frequency 3. Specificity (explicit instruction and methodology) 4. Duration 5. Neurodevelopmental hierarchy (i.e., training basic skills before advanced skills). Late identification of a developmental delay in early childhood and receiving intervention late results in the child not being able to catch up in the appropriate timetable compared to peers. Long waits allow fears to continue to increase.

When a child is discovered to have two developmental milestones affected, they require two separate therapists and therefore may not be given simultaneously, and the child will not have a coordinated effort to remediate their developmental delays. With a Pediatrician created interactive Neurodevelopmental assessment, that starts at age 3 years old and younger, the areas of speech/language (receptive, expressive, phonological awareness, fine motor, gross motor, behavioral, challenges, attention, engagement and emotional regulation engagement, and the ability to follow directions can be done based on normative data for each age group based on what has been studied for a neurotypical 3 year old, coexisting condition that mimic attentional disorder like staring spells seizures must be ruled out. The 3-year-old is asked to complete the interactive neurodevelopmental assessment using AI to guide the student and score the assessment. The assessment starts with asking the child to draw a straight line and to copy a circle. We observe the pencil grip and ability to do the two tasks. Although it may be developmental normal for a child to have a fisted pencil grip at this age, we expect that by age 4 years old that they have a three-point (tripod) pencil grip. This gives an opportunity to communicate to the parent or teacher of this child that although this finding is normal, this is your opportunity to work with your child towards a three-point pencil grip expected by age 4 years old. If the child is unable to draw a straight line and circle, we will then present enrichment modules using AI work on the skill of drawing a straight line and circle and other exercise similar to this. The child will then be given different assessments in 2 to 3 months depending on the AI evaluation, (however despite AI assessment we will evaluate no later than 3 months) of the enrichment exercise the child is doing to see if the child is proficient. If in three months the issue persists, then the child will be given more modules to work on this skill and an in-person Occupational therapist prescription will be given to the child to work together with our modules. The child is again evaluated in 3 months' time and further action depending on the score will be taken to help the child with this skill. The next part of the evaluation will be to check for gross motor ability and skill planning along with receptive language. The child is asked to follow directions where they are asked to use the large muscles of their body to demonstrate that they can move appropriately and follow directions by touching their finger to their nose for example. If the child is not able to follow through with these skills, the AI will determine if this is due to child not proficient in the English language. This is asked before the assessment is given and the parent will have the ability to have this part of the assessment given in their primary language to be sure they are able to complete this skill) or if it is due to a receptive language delay or gross motor delay. Also, we will determine if the child is following directions correctly but not fluent in using their hand to point to their nose.

A child not performing well in this skill will need enrichment modules in gross motor tasks and also in following receptive language exercise where they are asked to follow simple commands. In 2 to 3 months' time this will be reevaluated using a similar assessment to be sure this skill has been mastered. The next skill would be to tackle sound articulation. This skill exposes the child to phonological awareness—“Learning how to manipulate the sounds.” The child is asked to say the sound of a word, either the first sound, last or middle sound of each word. This skill is fundamental for starting to decode or read as a child must be able to manipulate, play with, hold onto, and retrieve sounds to read or spell well. The child is given several enrichment modules via AI to strengthen this skill and in 2 to 3 months' time will be given another assessment. The next step of this assessment is to assess sound discrimination. The child again is exposed to phonological awareness and is learning to play with sounds. They are asked to listen to three letter sounds said in a row and to determine if all these sounds are the same or different. This is another foundational skill for learning how to decode and later to read which is sound discrimination. The last part of the assessment is to evaluate the child's ability to engage in the assessment and how attentive they are and are they able to communicate back to the AI agent. Depending on the child's demeanor during the assessment, a child may need further evaluation for autism. Children with poor eye contact, lack of joint attention, lack of wanting to engage and sensory deregulation will need modules on simultaneous speech and Occupational therapies while simultaneously participating in our enrichment modules. Physician referral for a prescription for the child to receive urgent in-person speech and Occupational therapy. If the child is able to communicate effectively, able to engage in the assessment but has a staring spell during the assessment for several seconds, then a neurology referral for an EEG will be prescribed to rule out Absence staring spell seizures. If the child is crying and anxious during the assessment, the child will receive enrichment modules to gradually introduce these 3-year-old milestone skills slowly and in 2 to 3 months' time be able to be reevaluated with an assessment again. A questionnaire is given to the parent of the child before the assessment is given that will ask specific questions that will ask the parent about their knowledge of what skills they believe their child knows. It also gives valuable information as to how to help the parent engage in more experience-based learning opportunities. Each parent is given a detailed milestone information packet that will help them understand what a child at each age needs to be able to do and ideas on what they can do to help their child reach their milestones. Whether they answer some of the asks correctly or incorrectly, they are given enrichment modules to improve each day so that they the skills necessary for a 4-year-old child when the next assessment is given.

This novel approach considers all the developmental skills a child should have at a certain age and works on them simultaneously use artificial intelligence to make this scalable and the child is allowed to have this interactive assessment scored immediately and enrichment modules are delivered immediately. What is novel about this approach is it takes into consideration and asks the parent if the child has had a recent hearing and vision test, and through our platform we will be able to deliver a vision test including for color blindness in the older ages and hearing assessment if necessary. We also consider their engagement at the same time and therefore we will not allow a child with suspicion of autism, absence seizure, anxiety and possible attention disorders (ADHD/ADD) to have a delay in diagnosis. We will deliver the necessary enrichment modules to help the child improve and at the same time arrange for a prescription to be given for further evaluation and treatment as necessary. We are providing the necessary foundational developmental skills information needed at age 3 years old and younger building up for the necessary skills needed at age 4 years old and older, etc. We understand that about 67% thirds of our nation's 4^{th} graders are reading below grade level and approximately 80% of fourth graders are from low socio-economic backgrounds are reading below grade level about 75% of graduating High School students are not proficient in writing. If only 10 to 20 percent of the population are estimated to be dyslexic, then why do an additional 50% or more children struggle to read? The developmental assessments answer these and other reasons why. Each module is interactive and will go at the child's own pace depending on the ability, mood and demeanor of the child that day. The modules are personalized, customized and specific for each child based on the assessment and AI will act as the child's personal tutor. These interactive modules are given every day for short periods of time based on the child's age. The 4-year-old neurodevelopmental assessment starts with a receptive language skill and during this skill we are not only able to see the child's ability to follow directions and be proficient in receptive language skills, but we are also able to see if child knows their shapes and colors. If the child is unable to correctly identify colors and shapes, enrichment modules will be given to teach these skills. If the child is only having trouble with the colors red and green, color blindness tests will be done. Again, the appropriate modules are recommended after the interactive assessment is score for the 4-year-old. The next skill is for sound discrimination as explained above in the 3-year-old assessment, and next sound order awareness where the questions are harder than what a 3-year-old Is expected to know. The next skill is fine motor determination with tasks consistent with what a 4-year-old should perform. Each age level assessment is therefore adjusted for age expected performance. Also, fine motor skills assessment to determine if the child has correct 3-point pencil grip. Added also is the assessment of the ability to identify letters of the alphabet and their sounds. This skill is very important in learning the fundamentals of reading. The next skill is phoneme elision (deletion). This test has a very high positive predictive value for identifying who will later go on to struggle with reading as it is a very sensitive indicator of phonological awareness processing difficulties. Therefore, many critical language skills are addressed together and developed in a coordinated and specific fashion.

Neurodevelopmental skills are built layer upon layer starting with the most basic skills before advanced ones. From infancy, a baby starts to develop these skills in a certain progression and marked by critical milestones for which can be evaluated. For example, with gross motor milestones, an infant first learns to roll over, sit up, and crawl, way before walking and running. To understand sensory regulation based upon a neurodevelopmental model requires an understanding of how an individual regulates themselves within a sensory environment. While touch, smell, taste, sight, sound are senses, so are vestibular (how one responds with respect to their head with gravity), proprioceptive (body position, pressure and force of movements), and interoceptive (understanding the inner working of how a person feels from when they are upset to knowing when they need to go to the restroom). All neurodevelopmental skills integrate together as a child grows, matures, and learns. Learning happens through practice. Learning is the response of the brain to make new connections (new brain wiring in response to explicit learning activities or experiences). This is also called experience-based learning or neuroplasticity. Therefore, every experience from infancy and through childhood is an opportunity for growth. Neurodevelopmental milestones are grouped under different categories such as physical development (gross motor and fine motor), social-emotional and behavioral, cognitive and language.

While every child is different, experts have a clear idea about the range of normal development related to age. When deficits occur, early identification and treatment of deficits is imperative for the development of higher-level skills.

When a child is evaluated using the system and method of the present disclosure, skills of the child are evaluated to determine if the child has any skills that can be performed efficiently, or with “automaticity,” meaning you can do it without thinking about how you do it. For example, when learning to “ride a bike” or “drive a car,” it was not easy at first. You had to think about every step and how to integrate all those steps together to safely get from destination “A” to destination “B.” But now, after a significant amount of practice and repetition, it becomes “neurologic memory.” Your brain just knows how to do it without really thinking about it. Learning any new skill basically happens that same way. If a strong foundational platform exists, then all one needs will be the right practice opportunities to learn that skill until automaticity happens. The individual's strengths and weaknesses of their foundational skills will determine the end functional result but practice once those foundational skills are present is critical to end performance. This means that what was once hard and effortful now is quick, easy, and effortless. With that ease will mean that the activity is probably more enjoyable, less anxiety provoking because one does not have to work so hard to do it and one feels more competent in their ability to do it.

The system and method of the present disclosure are designed to help children enjoy and excel in school academically, socially & emotionally and interactively, but not only just with school (the workplace of a child), but also with sports, other extracurricular activities, and subsequently later with the future life skills. The system and method of the present disclosure was designed utilizing Pediatric neurodevelopmental science to provide the tools to not only successfully navigate children throughout their scholastic journey, but also their nonacademic performance like with the physical skills necessary for gym, sports, and other extracurricular activities. This improves their social and emotional well-being, meaning the ability to form relationships with peers, family, and adults. The system and method of the present disclosure are adapted to make learning fun by opening up the world for discovery and giving children strong self-esteem and confidence. Indeed, playing and exploration are some of the greatest ways to learn. Knowing what skills are critically important skills to a child's success are and then incorporating them into everyday play activities for a younger child changes the manner in which children are prepared for school readiness. Giving the parent the information on what skills their children need to improve allows them to be actively involved. Each parent wants the best for their child, and most do not know what their children are expected to know when they enter school. This information is not generally discussed at Pediatrician office well visits and the Pediatrician is usually asked to help the parent when the parent is told by the teacher that the child is falling behind in school. By that time, the child is several grade levels behind in reading and/or fine motor ability. It is important to have this evaluation given by all pediatricians during their well visits. When it is administered by AI, scored by AI and intervention plans are suggested by AI in accordance with the guidelines of the American Academy of Pediatrics, then Pediatricians are able to be the true medical home for each child and will be able to work in conjunction with teachers to give our children the best chance to succeed. When children enter kindergarten, they are given a basic screening test that does not include any testing of motor skills (fine or gross) and the score given to the parent is a percentage of how well their child is doing compared to other children in their classroom, school, county and state. They are not told what they need to work on with their children at home to improve their skills or how to work with them. Parents are only told their child's skills are progressive and instructed how to help. Only during parent conferences, which happen late in the school year and in the spring when school is almost over, does a discussion happen with the parents.

The system and method of the present disclosure are adapted to make children with weaker skills stronger and to make children with average skills stronger too. This means that if you create the right learning opportunities, every child will have stronger foundations from which to try, achieve, and excel in any endeavor they desire. The system and method of the present disclosure thus can act as a launching pad for learning successes from which each child can become healthy, happy, confident adults with careers they love and are successful at. Having the skills necessary for success in school and beyond reduces mental health disparities and removes the child from being punished for behavioral issues. Children often act out because they do not believe that they belong in school since they do not understand what is going on and others around them. Most children are expelled from the classroom for bad behavior starting at age 4 years old and getting the child to be better at their emotional regulations skills before age 4 years old will help reduce that fact. A lot of children would rather be in trouble than risk public embarrassment of not performing well in school. Also, there is a big problem with children not going to school and the large absentee rate is largely due to children not being able to engage most likely from not having the necessary skills necessary to perform in school and in the playgrounds/recess time. In some embodiments, the system and method of the present disclosure is in accordance with and follows the American Academy of Pediatric guidelines for identification of normal child development at each age and intervention strategies recommended.

The system and method of the present disclosure were designed to recognize that a medical approach is needed in assessing child development. Indeed, children in the elementary school years are in school to “learn to read.” If they do not master this skill by the end of the 2^{nd} grade, failure is imminent because they are now in school to “read to learn.” What starts as a reading gap, becomes an unsurmountable vocabulary and knowledge gap. Academic learning is now very difficult, and gaps can often become so large that they are unable to be closed. 75% of students who do not read proficiently by third grade never reach reading proficiency in future grades. Also, there is a 54% decrease in likelihood that struggling readers in third grade will attend college compared to their more proficient peers. In the United States 93 million adults in the nation today read at or below basic level, even though most living-wage jobs require proficient readers. Approximately ⅓ of all adults cannot read drug labels and low literacy costs the US 230 billion/year in health care costs. The US Department of labor estimates that illiteracy costs American businesses about $225 billion a year in lost productivity. Accordingly, the system and method of the present disclosure provide a medical approach by providing an early identification and early intervention problem solving approach. It means that if you identify a problem or issue early, that provides early intervention and change, then prevention is in the majority of cases possible. For example, you do not have to watch a train derail off a track if there is a way to redirect that train to a different path where a successful and safe arrival is achievable. The system and method of the present disclosure thus prevent a parent from watching the child they love struggle more and more each year as the grade level complexity increases. It is possible to change this trajectory, but it starts with understanding and trusting in “brain science” and knowing that the best outcomes are when you start early. Ideally, this would begin at birth, as it starts with talking to a child, allowing the baby to watch your lips form sounds and the baby soon learns that these sounds have meaning and reading to them. But much more can be done. Indeed, more fun, enjoyable, and bonding activities can be helpful and allowing parents to have this information in front of them in an interactive manner as AI can provide would help parents be able to do more for their children. This is especially also very helpful for day care personnel who are not all trained and not all knowledge in developmental milestones of children. The same goes for preschool teachers and elementary school teachers. 31% of teacher preparation programs devote no course work to reading science. Only 46 percent of undergraduate elementary education programs teach phonemic awareness and usually only a short time is devoted to this subject. The national Reading Panel identified five pillars of reading and only 39% of teacher undergraduate elementary education programs provide instruction in all five components of reading. The five components of reading identified by the national reading panel are 1. Phonemic Awareness is the ability to hear and perceive, identify and manipulate individual sounds (phonemes) in spoken words. 2. Phonics is the relationship between letters and sounds, and how to use this knowledge to decode words. 3. Fluency is the ability to read accurately, quickly and with expression 4. Vocabulary is the knowledge of words and their meaning. 5. Comprehension is the ability to understand and make meaning from what is read. The system and method of the present disclosure can guide children toward activities they need to make them successful with the skill of reading.

The system and method of the present disclosure were designed to recognize that while many of the causes of why a child can go on to struggle scholastically are preventable, some are not. Indeed, while some causes have treatments, others do not. No matter the disorder or condition, early diagnoses and effective intervention generate the best outcomes as earlier diagnosis will allow access to supportive therapies, helpful accommodations, and medical and scholastic supports to be in place.

The system and method of the present disclosure were designed to recognize that children may struggle scholastically, socially and emotionally and in extracurricular activities such as with sports and other activities for many reasons. One reason children may struggle scholastically is lack of opportunity, such as, example, English second language learners, foster care and adoption populations, and socioeconomic conditions affecting access and exposure to learning opportunities. Another reason children may struggle scholastically is attentional issues resulting in lack of adequate engagement/reception of learning instructions and opportunities. For instance, with severe ADHD, even though a child was in the classroom, if they did not adequately receive (attend and focus) on the intervention being given, you must consider that instruction as not to have happened and thus as lack of opportunity. Also, if a child has absence seizures (starring spells) for the moments when they have their seizures, they are unable to attend and listen and therefore will not learn. Thus, they miss bits and pieces of instruction. Children with this disorder may be misdiagnosed with ADHD/ADD. Another reason children may struggle scholastically is language disorders, which are both receptive (input) and expressive (output) or both. These children may not have the ability to understand language in general or have difficulties specifically with phonological awareness. For example, if a language disorder exists, vocabulary and receptive language must be built before reading and listening comprehension at higher levels can take place. Another reason children may struggle scholastically is specific learning disorders, which are sometimes referred to as specific learning disabilities. For example, neuro-developmental disorders that are characterized by a persistent impairment in at least one area, including, for example, impairment in reading (dyslexia), impairment in written expression with or without associated dysgraphia, and impairment in mathematics (dyscalculia). In cases with specific learning disorders or disabilities, they are unexpected and not caused because of a lack of intelligence. Another reason children may struggle scholastically are medical conditions that can impact learning in some way, such as, for example, seizure disorders, some genetic conditions, ADHD, prematurity, cerebral palsy, in utero drug or alcohol exposure, etc. Other reasons children may struggle scholastically is intellectual, such as, for example, cognitive disabilities whether genetic, environmental, or idiopathic. For example, cognitive neuro-developmental disorders are noticed early on with broad developmental delay characterized by intellectual difficulties as well as difficulties in adaptive skills like conceptual, social, and practical areas of living. They are characterized by low IQ scores and low adaptive behavioral functioning across multiple environments (school, home, and community).

The system and method of the present disclosure were designed to recognize that early intervention is important. Indeed, children are in our elementary schools to “learn to read.” After the 2^{nd} grade, they are in school to “read to learn.” If reading is not mastered by the 3^{rd} grade, academic gaps grow very quickly. It is important to treat as early as possible because you do not want a reading gap to become an academic gap that is difficult to close. Most children are unable to close that gap. Intervention at any stage is helpful, but the earlier the better. Therefore, it is important to look for and treat all the variables at play early in order to achieve the most successful outcomes. For some, they might just need adequate exposure (reteaching) while attentional focus is present. For others, who are dyslexic with weakness in phonological awareness, they will need explicit instruction to improve phonological awareness skills. For those who are unable to memorize sight words, they might need occupational therapy to help with the visual processing skills required for efficient sight word acquisition. As such, picking the right intervention specifically required by each child, in the most cost-effective and time-saving manner, to accomplish gap closure is very important for each and every child. Indeed, though learning to read may seem effortless, even natural, for some, reading and spelling are complex mental skills involving several different parts of the brain. A weakness in any one of these parts will cause the entire system to slow down and become laborious and frustrating. In order to be effective for all students, reading instruction must address all of the sensory-cognitive skills involved in reading and be informed by the relationship between them as revealed through scientific research.

The system and method of the present disclosure were designed to recognize that reading, writing and spelling are higher level language skills. Indeed, the ability to play and manipulate sounds. Learning to read the sounds is the building block required for language development at its most basic level, which will then affect all higher-level skills like reading. A foundational skill that children need to have before being able to sound words out is to be able to separate spoken words into their individual sounds. Indeed, many struggling readers have extremely poor foundational skills in this area with this being the “crack in the foundation” that makes easy and accurate reading and spelling difficult later on. For some struggling readers, these poor phonological processing skills (processing sounds in words) can prevent future reading success, even when these children are exposed to common place reading instruction as well as phonics programs or even multi-sensory Orton-Gillingham dyslexia methods. If one knows and applies the neurodevelopmental building blocks to assessment and treatment, then age-appropriate tests and interventions can be done to prevent reading and scholastic failure before it occurs. Every test utilized has to be standardized for performance compared to age specific norms. For example, what a kindergartener is expected to do is different than what you would expect an older student to do. The building blocks for foundational language, sensory, emotional regulation, and fine and gross motor skills are critically important and should and can be assessed easily given the right knowledge and tools. Thus, there is nothing random about the tests used which are testing very specific skills based on age expected norms.

In order to understand the complexity of language and why difficulties occur with reading and/or spelling, it is helpful to start at the most basic foundational level and to understand the terms used. The smallest meaningful unit of language is called a phoneme. In English there are 44 different phonemes that are represented by the 26 letters of the alphabet individually or in combination. Phonics instruction involves teaching the relationship between sounds and the letters used to represent them. With respect to spelling, there are hundreds of spelling alternatives that can be used to represent the 44 English phonemes.—Phonological processing (processing sounds in words) is the ability to be aware of, manipulate, and play with different sounds in words. This is the single most important skill that separates a good reader from a poor reader and can be tested for as early as 4 years old. Phonological processing is a combination of visual processing, sound processing, and oral motor movement processing.

While many reading interventions focus on general exposure to literature, phonics memory rules, or the memory of sight words, this is different from building stronger phonological processing. The natural development of phonological awareness skills happens over time and is not the result of rote memorization. Some children need to work many months on this skill depending on the severity of their phonological deficits. If a child has been exposed to previous common school interventions like Whole Language, Balanced Literacy, or even Orton-Gillingham methodology-based interventions and the reading gap has not closed, then the child still has difficulties with phonological processing. These cracks or difficulties in phonological awareness and processing should be tested for and effectively treated. Ideally, this would occur before a child starts school.

Having a child “try harder or just read more” is not an effective solution. Accurate reading is important for reading fluency (quickness and accuracy) and overall comprehension. Filling in unknown words based on contextual skills or guessing are not good alternatives. For many decades, teachers taught reading in elementary school by having children look at pictures or use contextual language to decipher unknown words instead of using their phonological awareness skills to accurately sound out (decode) a word. This method is starting to change, and teachers are beginning to learn how to use awareness and phonics to help children read accurately. This is important because accuracy fuels comprehension as it is a much better methodology than guessing. Developing the skills to accurately decode words is the best solution and should be the goal of any reading instruction and intervention. Early pre-literacy assessments that take a deep look into the separate skills of phonological processing can be very helpful in assessing the root cause of what would become future difficulties. Effective assessments aid to prevent reading failure from ever happening. The system and method of the present disclosure align as a way to focus on finding any weakness early so that reading failure or intervention for that failure does not have to occur.

The system and method of the present disclosure include language assessment portions and later enrichment activities that focus on various factors. One factor that the language assessment portions, and later enrichment activities focus on is strengthening auditory discrimination and phonological awareness. Another factor that the language assessment portions, and later enrichment activities focus on is based upon a neurodevelopmental language model (i.e., understanding how the brain learns simple language foundational skills before more advanced skills). Several brain functions are involved in reading. The visual cortex recognizes printed letters and words, the auditory cortex builds oral world understanding, angular gyrus associates letters with sounds and the frontal lobe produces speech and process meaning. Our methods include language fluency, phonological awareness, orthography, phoneme-grapheme correspondence and fluency in our enrichment modules to develop foundational skills associated with each brain function. There are physiological explanations for why it is critical for children to learn by the third grade. Growth in volume of white matter—(the neural pathways in a child's brain)—between kindergarten and third grade is one of the best predictors of how well a child learns how to read. Therefore, the quality of reading instruction is important and impacts a child's brain white matter development which is the neuropathways that connect areas of the brain. 56% of the variance in reading outcomes can be attributed to the change in volume of white matter during this important critical time in a child's reading trajectory. It is also important to be aware that areas in the brain that control language are remarkably close in proximity and somewhat overlap areas that control sensory and motor movements of the face, tongue, lips and fingers. This is why children with reading struggles like dyslexia often have problems with fine motor control and dysgraphia (difficulty with writing skills) and speech articulation issues due to oral kinesthetic movement difficulties with face and sensation and motor movements of the mouth, tongue and lips. In adult left hemispheric stroke patients (any patients with neurological insult to Broca and Wernicke's areas of the brain) present with expressive and receptive aphasia. Recovery is more successful if they receive therapy in speech/language, fine motor, and gross motor simultaneously. Therefore, in children with reading difficulties, allowing for therapy in speech/language and fine/gross motor simultaneously will allow a more rapid and full recovery which is especially true with children who present with Autism.

Another factor that the language assessment portions, and later enrichment activities focus on is practice in a neuro-rehabilitative model which incorporates the principles of neuroplasticity which means the ability of the brain to make new connections (new brain wiring in response to clear and direct learning activities or experiences). This is also called experience-based learning. Therefore, any program that is based on these specific principles will mean that the intervention is frequent, specific, intensive, based upon neurodevelopmental hierarchy (training basic skills before advanced ones), and occurs for the needed duration so that the child's skills are fully developed, mastered, and able to be performed independently without having to think about how to do it.

With the above considerations in mind, the system and method of the present disclosure include assessing a child as early as possible (using a medical approach) to assess his or her speech and language, fine and gross motor skills to provide a comprehensive analysis that considers various factors, including, for example, whether a skill can be performed with automaticity, whether there are outside reasons why a child may struggle scholastically, whether the child possesses appropriate foundational skills, etc. This allows the system to assess auditory discrimination and phonological awareness skills, neurodevelopmental language skills, experience-based learning skills, etc. In that regard, the system and method of the present disclosure includes administering a plurality of tests to a child relating to speech and language, fine and gross motor skills to assess the child's development and/or school readiness. How the child performs on the tests will determine whether or not the child should seek outside therapy (speech or occupational therapy), which can be recommended using the system and method of the present disclosure. How the child performs on the tests may also be used to assess/evaluate the child for ADHD, autism, anxiety, panic attacks, post-traumatic stress disorder (PTSD), sensor regulation issues depression, absence seizures, low self-esteem, etc.

The following discussion includes a description of a system and method for evaluating and/or assessing developmental disorders and/or school readiness, in accordance with the principles of the present disclosure. Alternate embodiments are also disclosed. Reference will now be made in detail to the exemplary embodiments of the present disclosure, which are illustrated in the accompanying figures. Turning to the FIGS., there are illustrated forms/documents that are used in connection with a system and method for evaluating and/or assessing developmental disorders and/or school readiness.

The system and method of the present disclosure includes a system and method for early evaluation of a child by implementing tests that determine whether the child has adequate speech and language, fine and gross motor skills, or not. That is, the system and method of the present disclosure evaluates a child for the presence or absence of a disorder or condition and/or for school readiness using tests that test the child's language skills, as well as other skills, such as for example, speech, fine and gross motor skills. As discussed in greater detail below, the system of the method of the present disclosure includes several portions or parts that are conducted over several years. For example, the system and method of the present disclosure may begin assessing a child by giving the child a first series of tests in a first part of the method when the child is 3 years old and then may continue periodically (e.g., annually) such that the child is given a second series of tests in a second part of the method when the child is 4 years old, is given a third series of tests in a third part of the test when the child is 5 years old, is given a fourth series of tests in a fourth part of the test when the child is 6 years old, is given a fifth series of tests in a fifth part of the test when the child is 7/8 or years old and is given a sixth series of tests in a sixth part of the test when the child is 9 years old or older. It is believed that early and continued implementation of the system and method of the present disclosure is important for assessing a disorder and treating the disorder effectively. Indeed, if the disorder is not assessed early enough, effective treatment and/or reversal of the disorder may not be possible.

### Method

In one embodiment, the method evaluates and/or assesses the presence or absence of a disorder and/or school, sports and emotional social readiness by providing evaluation of input data of an individual, such as, for example, a child. The input data is obtained through administering a worksheet, for example. That is, data concerning the child that is obtained by administering a variety of tests that evaluate speech and language, fine and gross motor skills is recorded on a worksheet, which is evaluated to assess the presence or absence of a disorder and/or school readiness. The evaluation of the worksheet can also be used to determine proper treatment for the child, such as, for example, the need for occupational therapy. The evaluation of the worksheet can further be used to assess/evaluate the child for ADHD, autism, anxiety, panic attacks, post-traumatic stress disorder (PTSD), depression, absence seizures, low self-esteem, etc.

### Method—Part One—Year Three

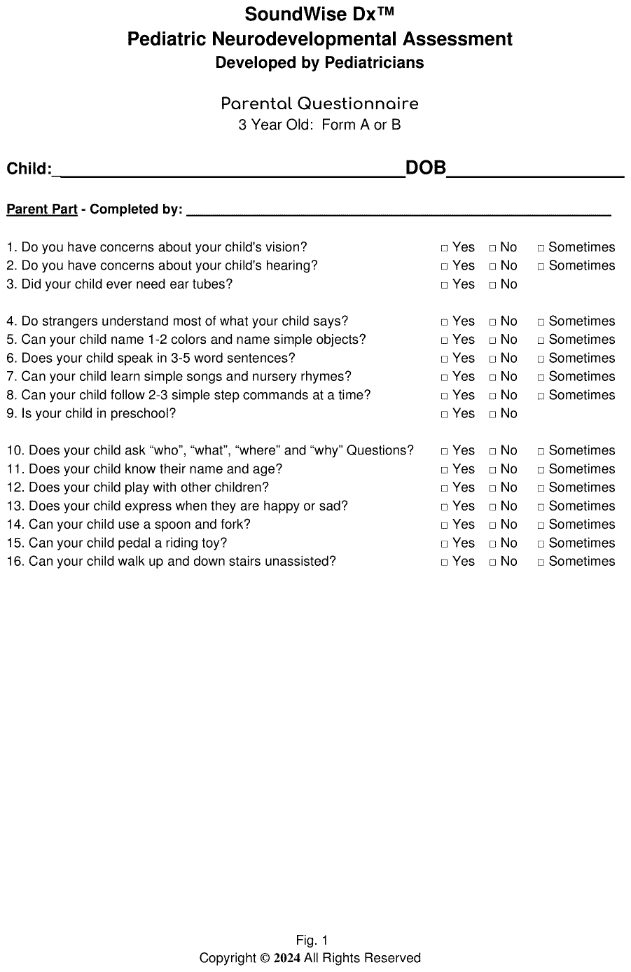

In a first part of the method, which should be administered as early as possible, such as, for example, when the child is approximately 3 years old or younger, the child's parent's is required to fill out a questionnaire that includes various questions about the child. One embodiment of the questionnaire is shown in FIG. 1. As shown in FIG. 1, the questionnaire can include questions about the child's physical abilities, such as, for example, the child's hearing and vision. The questionnaire can also include questions relating to the child's ability to read, write, comprehend information, perform simple tasks, socialize, etc. In some embodiments, the child is required to undergo a Vision Snell chart test and/or a colorblind test (e.g., The Ishihara, EnChroma, or any other standardized color-blind assessment or screen) prior to beginning the first part of the method.

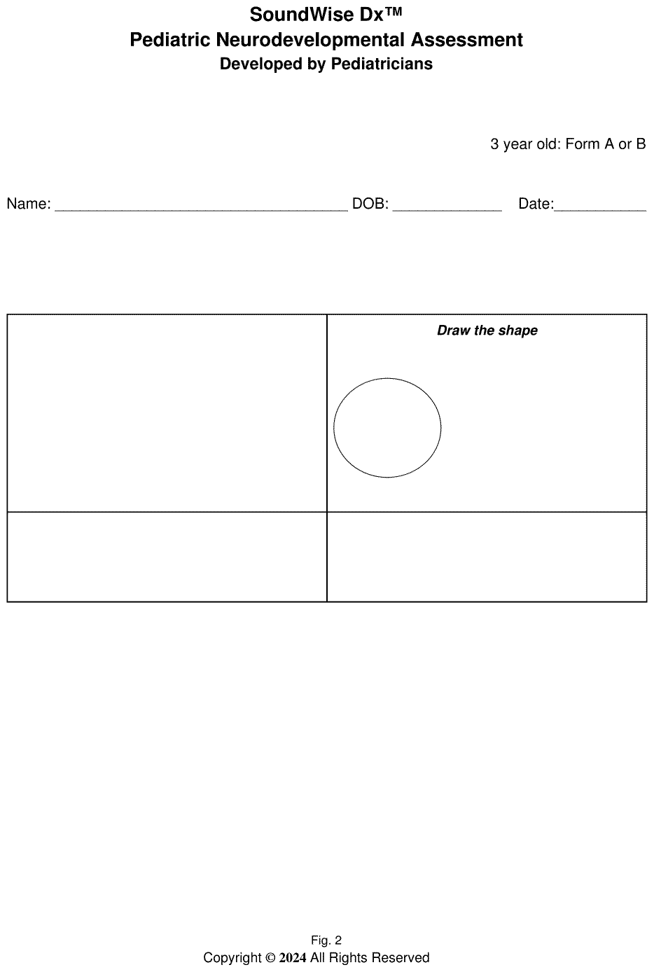

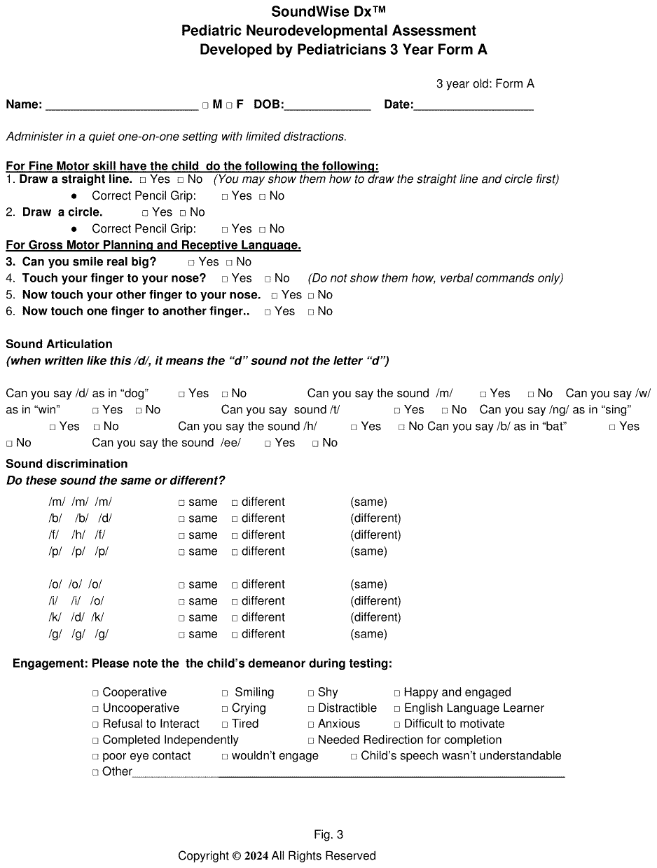

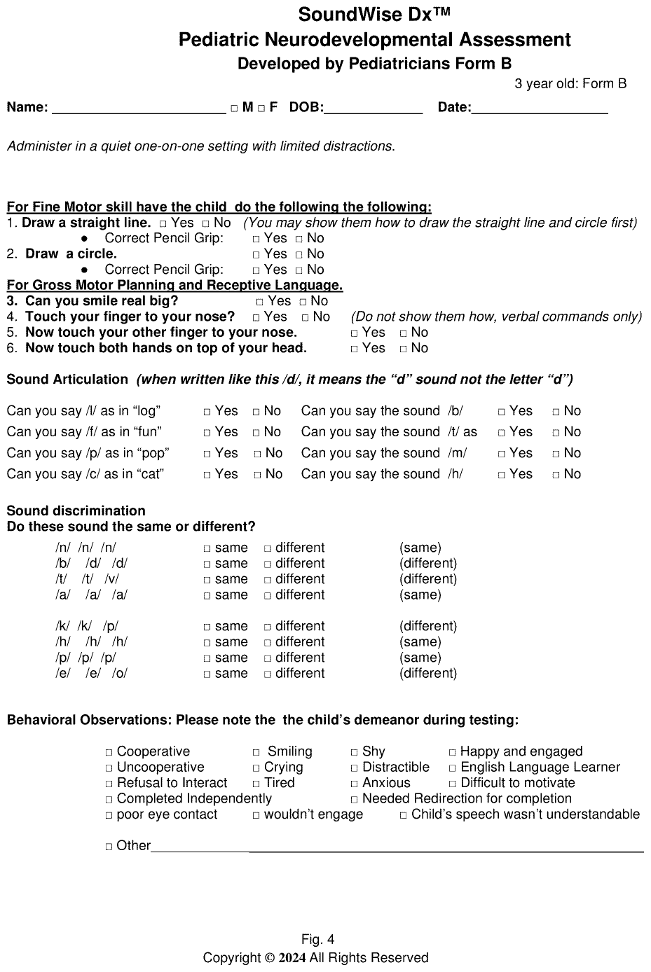

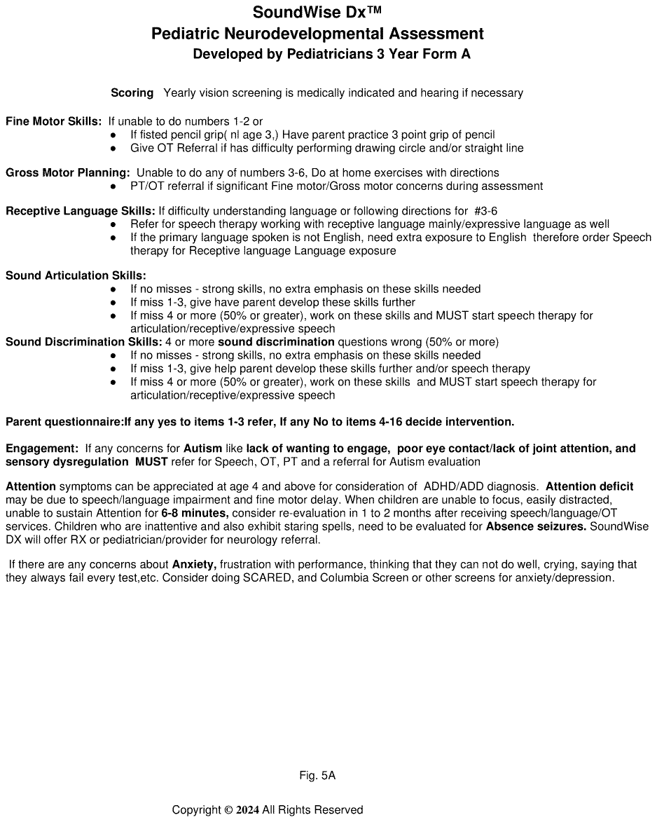

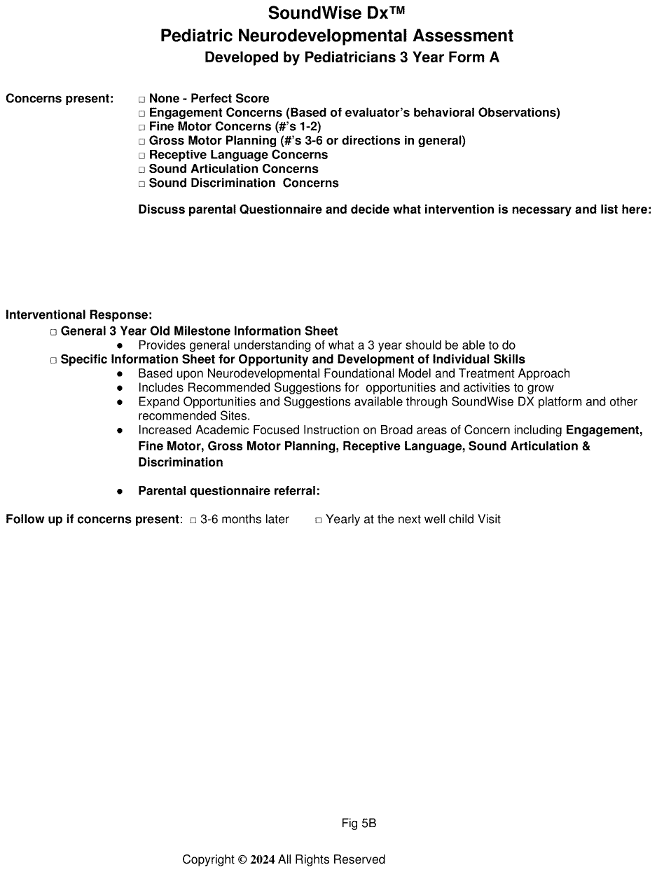

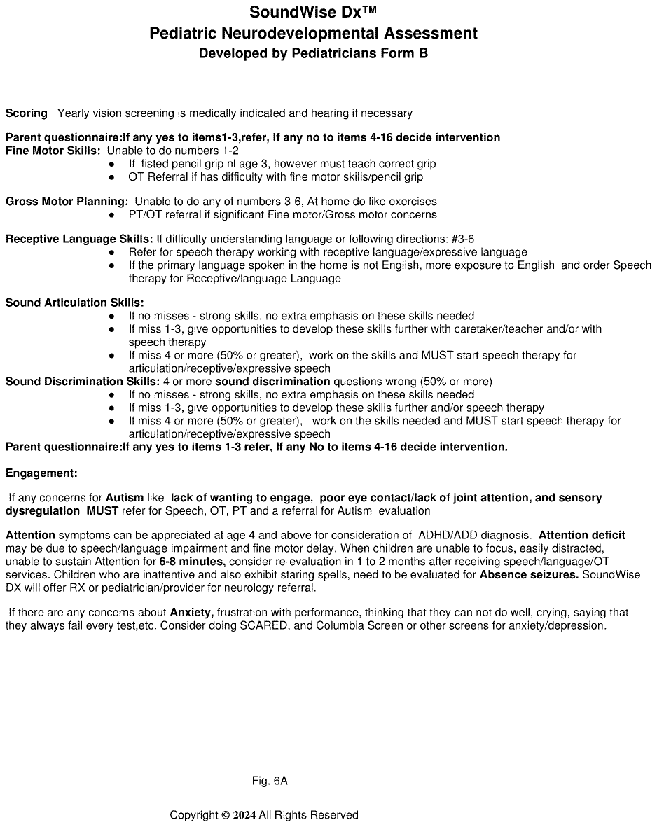

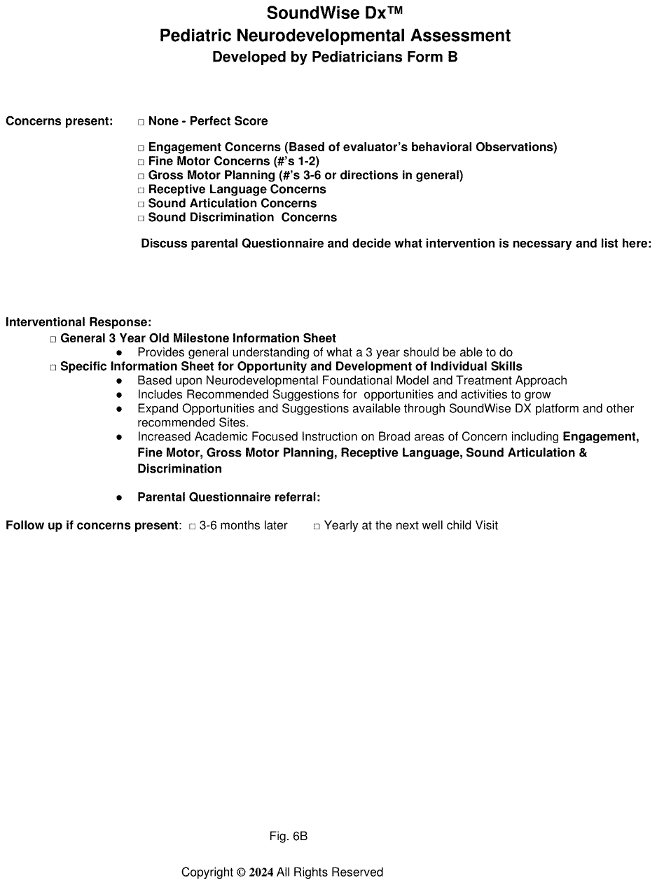

In the first part of the method, tests concerning speech and language, fine and gross motor skills are evaluated the presence or absence of a disorder and/or school readiness ability whether it be in the social, emotional, physical activity (i.e. sports) domain., In particular, in the first part of the method, a first test is administered in which the child has to draw a line or shape, as shown in FIG. 2. The line may be a straight line or a non-straight line, such as, for example, a squiggly line. The shape can be a shape, such as, for example, a circle, square, triangle, etc. The ability of the child to draw the line or shape asked is recorded to generate a fine motor skill input data. The ability of the child to draw the line or shape may be recorded in the worksheet. One embodiment of the worksheet is shown in FIG. 3. Another embodiment of the worksheet is shown in FIG. 4. The fine motor skill input data may include whether or not the child was able to draw the line or shape, how well the child was able to draw the line or shape, etc. In place of or in addition to testing the ability of the child to draw the line or shape, the first test may include a pencil grip test in which the child is required to grip a pencil and the ability to grip the pencil or not and/or the ability to grip the pencil correctly is recorded to generate the fine motor skill input data. For example, if the child uses a fisted grip to grip the pencil, an incorrect pencil grip may be recorded. The ability of the child to grip the pencil may be recorded in the worksheet, as shown in FIG. 4. The fine motor skill input data is evaluated against an answer key to generate a fine motor skill evaluation result. The fine motor skill evaluation result is a first determination with respect to the presence or absence of fine motor skill deficiency. In some embodiments, the fine motor skill evaluation result includes a score, such as, for example, a number score. In some embodiments, the fine motor skill evaluation result includes a written evaluation, such as, for example, words written in sentence or note form that explain what was observed by the practitioner during the first test. In some embodiments, the fine motor skill evaluation result is included in an assessment that evaluates at least each of the tests administered during the first part of the method. One embodiment of the assessment is shown in FIGS. 5A and 5B. Another embodiment of the assessment is shown in FIGS. 6A and 6B. In some embodiments, the first part of the method may be administered when the child is less than 3 years old, such as, for example, when the child is 1 year old, 18 months old, 2 years old, 30 months old, etc. In some embodiments, the child is required to draw both the line and shape in during the first test.

In the first part of the method, a second test is administered in which the child has to do a gross motor skill and receptive language task to generate a gross motor skill input data. The ability of the child to do a gross motor skill and receptive language task may be recorded in the worksheet (FIG. 3 or FIG. 4). In some embodiments, the gross motor skill and receptive language task may include having the child smile, such as, for example, smiling really big. In some embodiments, the gross motor skill and receptive language task may include having the child touch a first finger to his or her nose. In some embodiments, the gross motor skill and receptive language task may include having the child touch a second finger to his or her nose after touching the first finger to his or her nose. In some embodiments, the gross motor skill and receptive language task may include having the child touch a first finger to a second finger. In some embodiments, the gross motor skill and receptive language task may include having the child place both of his or her hands on top of his or her head. The gross motor skill input data is evaluated against an answer key to generate a gross motor skill evaluation result. The gross motor skill evaluation result is a first determination with respect to the presence or absence of gross motor skill deficiency. In some embodiments, the gross motor skill evaluation result is included in the assessment (FIGS. 5A and 5B, or FIGS. 6A and 6B). In some embodiments, the gross motor skill evaluation result uses AI or other software to recognize and/or transcribe the child's letter sound/word pronunciation, analyze context/structure where pronunciation varies due to accents/dialects and word/sentence structure comprehension and fluency.

In the first part of the method, a third test is administered in which the child has to say a first letter in at least one word to generate a sound articulation input data. In particular, the third test evaluates the child's ability to correctly pronounce some English sounds in words that are located at the beginning, middle, or end of a word because the ability to play and manipulate sounds in a word is a foundational building block needed to later read well. The ability of the child to say a first letter in at least one word may be recorded in the worksheet (FIG. 3 or FIG. 4). In some embodiments, having the child say a first letter in at least one word can include the childing saying a first letter in each of a plurality of words to generate the sound articulation input data. The words may include all short words, such as for example, three letter words and/or may include a combination of short words and longer words, such as, for example, three letter words and four-letter words. Alternatively, or in addition to having the child say a first letter in at least one word, the sound articulation input data may be generated by having the child say the sound of a letter or a combination of letters. For example, the sound articulation input data may be generated by having the child say the sound of the letter “M”, the letter “T,” the letter “H” and/or the letters “EE.” The sound articulation input data is evaluated against an answer key to generate a sound articulation evaluation result. The sound articulation skill evaluation result is a first determination with respect to the presence or absence of sound articulation deficiency. In some embodiments, the sound articulation skill evaluation result is included in the assessment (FIGS. 5A and 5B, or FIGS. 6A and 6B).

In the first part of the method, a fourth test is administered in which the child has to determine if one or more letters or words are the same or different to generate a sound discrimination input data. Sound discrimination is the ability to simply tell if sounds are similar or different. Understanding differences in sounds is part of the phonological (sound) awareness which is a skill that must be mastered well to later learn how to accurately decode (sound out) words. Phonological awareness is more than just knowing if sounds are different; it is actually a complicated brain skill of the combined ability of discriminating, manipulating, and changing sounds in words. How you process and understand sounds is what actually makes language meaningful. Phonological processing (the process of understanding sounds in words) is part manipulation and awareness but also the ability to hold on to and retrieve sounds in words. Phonological awareness is the single most important skill that separates good readers from poor readers. Phonological awareness is a combination of visual processing (seeing), auditory processing (hearing), and knowledge of oral mouth movements (feeling). In the fourth test, the child may be required to determine if the sound of the same letter repeated three times sound the same, to determine if the sound of two different letters wherein one of the letters is repeated twice sound the same and/or if the sound of three different letters sound the same. The ability of the child to determine if one or more letters or words are the same or different may be recorded in the worksheet (FIG. 3 or FIG. 4). The sound discrimination input data is evaluated against an answer key to generate a sound determination evaluation result. The sound determination skill evaluation result is a first determination with respect to the presence or absence of sound determination articulation deficiency. In some embodiments, the sound determination skill evaluation result is included in the assessment (FIGS. 5A and 5B, or FIGS. 6A and 6B).

In the first part of the method, a referral is generated referring the individual for services in areas where the evaluation results were deficient to promote at least one of fine motor skills, gross motor skills, sound articulation, and sound discrimination skills. In some embodiments, the assessment (FIGS. 5A and 5B, or FIGS. 6A and 6B), which can include the fine motor skill evaluation result, the gross motor skill evaluation result, the sound articulation evaluation result and/or the sound discrimination evaluation result is evaluated to generate the referral. In particular, if the fine motor skill evaluation result indicates that the child has proficient fine motor skills, the child is not referred for services, such as, for example, occupational therapy to promote advancement in fine motor skills. If, however, the fine motor skill evaluation result indicates that the child is deficient with his or her fine motor skills, the child is referred for services, such as, for example, occupational therapy to promote advancement in fine motor skills. In such cases, the recommended occupational therapy can include focusing on visual motor integration, oral motor kinesthetics, and proprioceptive skills.

Likewise, if the gross motor skill and receptive language evaluation result indicates that the child has proficient gross motor skill and receptive language, the child is not referred for services, such as, for example, occupational therapy to promote advancement in gross motor skill and speech therapy to promote advances in receptive language. If, however, the gross motor skill and receptive language result indicates that the child is deficient with his or her gross motor skill and receptive language, the child is referred for services, such as, for example, occupational therapy to promote advancement in gross motor skill and receptive language. In such cases, the recommended occupational therapy can include tasks to improve following directions, upper extremity movement and coordination, and proprioceptive work. Speech therapy is to strengthen receptive/expressive language skills, etc. In some embodiments, the recommended speech therapy can focus on motor integration, oral motor kinesthetics, and proprioceptive skills. In some embodiments, the recommended occupational therapy can focus on building strong receptive/expressive language skills.

If the sound articulation skill evaluation result indicates that the child has proficient sound articulation skills, the child is not referred for services, such as, for example, Speech therapy to promote advancement in sound articulation skills. If, however, the sound articulation skill evaluation result indicates that the child is deficient with his or her sound articulation skills, the child is referred for services, such as, for example, Speech therapy to promote advancement in sound articulation skills. In such cases, the recommended Speech therapy can include focusing on sound articulation and improving phonological awareness and phonological processing to build the foundational skills needed for reading.

If the sound discrimination skill evaluation result indicates that the child has proficient sound discrimination skills, the child is not referred for services, such as, for example, Speech therapy to promote advancement in sound discrimination skills. If, however, the sound discrimination evaluation result indicates that the child is deficient with his or her sound discrimination skills, the child is referred for services, such as, for example, Speech therapy to promote advancement in sound discrimination skills. In such cases, the recommended Speech therapy can include focusing on phonological processing skills. In some embodiments, the therapy recommendations can include occupational therapy for apraxia in additions to speech and language therapy focusing on improving phonological awareness and phonological processing/sound discrimination/receptive/expressive language.

In some embodiments, the first part of the method includes evaluating the child for ADHD, autism, anxiety, panic attacks, post-traumatic stress disorder (PTSD), depression, absence seizures, low self-esteem, etc. if the evaluation results were deficient to promote at least one of fine motor skills, gross motor skills, sound articulation, and sound discrimination skills. Concerning ADHD if the child is unable to keep their attention and/or unable to follow directions while completing the tests without being distracted and/or is unable to sit still long enough to complete the tests, the evaluation should include an ADHD evaluation. For example, at age 3 the child should be able to sit and attend for 6-8 minutes, at age 4 the child should be able to sit and attend for 8-12 minutes, at ages 5-6 the child should be able to sit and attend for 12-18 minutes, at ages 7-8 the child should be able to sit and attend for 16-24 minutes and at ages 9-10 the child should be able to sit and attend for 20-30 minutes.

The post ADHD evaluation's recommendations can include giving focused tips on a structured environment, making learning activities fun, multisensory activities, and short but repetitive opportunities for learning. If engagement is appropriate, but focus/attention to a task cannot be maintained for the age expected time for the child's age, then the method may refer the child to take a standardized ADHD measure such as the Vanderbilt or others) that includes ADHD/ADD age specific questions and consider all factors that can affect attention and sustained focus. Indeed, children with ADHD/ADD have a hard time paying attention, daydreaming, and often do not seem to listen. They are easily distracted from work and play and often do lack attention to details, are disorganized and do not follow through on directions. They are prone to losing a lot of important things/forgetting things and avoid doing things that require sustained mental effort. Having the hyperactivity component makes them prone not being able to stay seated, squirming a lot, talking too much, unable to play quietly and will jump, run and climb when not permitted to do so. They will demonstrate impulsivity, calling out with answers before the question being complete, acts or speaks without thinking, Interrupting others often, trouble taking turns, cannot wait for things and may run onto street without looking out for traffic. In some embodiments, if the child exhibits signs of ADHD/ADD, the method can be adapted to administer and score a Vanderbilt screen for parent and teacher (if applicable). These screens are diagnostic of ADHD/ADD.

Specific to visual attention and focus, the method can be adapted to derive objective and quantitative results, for example gaze direction, dwell time, and glance frequency off the assessment site to other objects and locations. While monitoring eye tracking, the system will measure where and for how long an individual looks in a certain direction or at a certain target, it is not a measure of actual visual attention. However, this information can be used to necessitate further exploration into attention or focus disorders like ADHD/ADD. The system and method can be further adapted to track and reflex to assess student attention while taking the test. Just as eye tracking is the go-to method when determining driver distraction via glance target classification, it can also be used to assess attention in the test itself. When problems with visual tracked perception processing are found then make in dept analysis optometry is warranted. When the above condition concerns are found, the system and method may be adapted to generate program modules and/or worksheets for evaluation and guidance for ADHD/ADD. It is envisioned that the system and method may also be adapted to generate further informational sheets on factors that can impact attention. These informational sheets can be given to the child's caregivers. Modules for scholastic accommodations such IEP, 504 and not limited to will also be given that can effectively be used within a child's Individual Educational Plan (IEP) or 504 Plan. When formal diagnosis of ADHD has been given, then information regarding evaluation for behavioral modification will be given through the system via the method. The system and method may also be adapted to indicate medication for ADHD/ADD when formal diagnosis of ADHD has been given, as indicated by the system. The system and method may also be adapted to recommend that blood pressure Pulse, EKG rhythm strip be administered through all digital devices, along with a telemedicine visit before medication is given. The system and method may also be adapted to provide classroom modifications and accommodation recommendations.

Concerning autism, the method includes evaluating whether the child has challenges with social communication and interaction, has poor eye contact, refuses to engage in tasks, and/or lacks joint attention back and forth while doing assessment tasks. For example, if the child demonstrates restrictive/repetitive behaviors, interests, and activities such as, but not limited to, flapping hands, spinning, lining up toys/objects, sensory issues (upset by loud noises/smell/touch), and challenges with change of routine during tasks, the evaluation should include an autism evaluation, while awaiting this evaluation. However, treatment must be started with speech and occupational enrichment modules every day until formal in-person Occupational and/or Speech therapy is started. These models should also be continued in conjunction with in-person therapy.

Autism presents challenges with social communication and interaction. Autistic children have speech and language delay and are not motivated to socially interact. They have challenges with eye contact and are unable to demonstrate joint attention, not able to play a back-and-forth game or do various assessment tasks. They show restrictive and repetitive behaviors, such as flapping their hands, spinning a lot, only interested in lining up toys or objects, sensory issues such as being upset with loud noises, wanting to smell things and not wanting to play pretend. Children with Autism also have difficulty with change of routine for example, having a new teacher, or driving a new route home and difficulty changing tasks during an assessment, such as, for example, one or more of the assessments made using the method of the present disclosure.

If the autism evaluation of the present method may recommend activities and/or worksheets that are adapted to address the specific concerns of lack of engagement/language delay affecting social interaction, high concerns for Autistic Spectrum Disorder (ASD), or ASD confirmed. This will include activities that focus on strengthening receptive, expressive, and pragmatic language skills; specific games and activities addressing language goals. To improve sensory regulation, the autism evaluation of the present method may recommend specific games and activities to decrease sensory triggers whether they be hypo(low) or hyper(high) sensory. To address sensory seeking behaviors and/or sensory avoidance behaviors, the autism evaluation may include recommending Occupational Therapy (OT) like play activities with OT therapy goals addressing visual/motor integration, sensory regulation, visual perceptual skills, executive function, and primitive reflex integration. To improve fine motor skills, the autism evaluation of the present method may recommend specific games and activities to improve any fine motor skills or hand skill ability like but not limited to the following (handwriting, cutting, folding, stringing beads, coloring, drawing, gluing, buttoning, using utensils, etc. To improve gross motor skills, the autism evaluation of the present method may recommend specific games and activities to strengthen gross motor movements that include focusing on balance, coordination, ball skills (dribbling, throwing, catching, etc.), hand eye coordination, same side and alternating side body movements, body schema activities (proprioceptive, vestibular, tactile, and body awareness) and/or Physical Therapy (PT) therapy like activities with PT gross motor goals. To improve social and pragmatic language skills, the autism evaluation of the present method may recommend specific group play therapy along with games and activities promoting opportunity for improved and guided social interaction. This can include Group play therapy, Applied Behavioral Analysis Therapy and Increased peer group interactions. In situations where anxiety or family connections are more calming, the method can be adapted to generate an avatar (computer generated face and/or voice used) that could be that of a person familiar to the individual. In some embodiments, one or more of the assessments, evaluations and/or recommendations are delivered to the child via the avatar.

The system and method of the present disclosure can be used to treat and strengthen skills along with in-person therapy especially when waiting for in-person therapy. The system and method of the present disclosure can recommend starting therapeutic advancement of identified skill deficits while at home when awaiting in-person therapies. Such therapeutic advancement can be used to augment and reinforce the actual future or current therapies in place like medically delivered Speech and Language Therapy, OT, Cognitive Behavioral Therapy, and PT. The system and method of the present disclosure can include engagement modules and/or worksheets that allow Early Intervention to immediately start while a child waits for in-person evaluation and/or in-person autism specific therapy. The system and method of the present disclosure can improve skills and reassessment will allow progress. Children should continue using the system and method of the present disclosure even after starting in-person therapy. Referral pursuant to the autism evaluation can include referring the child for an in-person Speech and Language Evaluation, referring the child for an in-person Sensory Integration Focused Occupational Therapy Evaluation, referring the child for an in-person Diagnostic Evaluation for Autism, referring the child for in-person Child Play Therapy.

The system and method of the present disclosure can be adapted to pursue all therapies covered by medical insurance while at the same time caregivers/teachers work on language skills, social interaction, developmental milestones, and sensory regulation using the system and method of the present disclosure for age-appropriate activities. Recommendations will include prescriptions for in-person services generated through the system and method of the present disclosure. Suggestions to seek specialized diagnostic evaluations when indicated by the system and method of the present disclosure will include follow up with the child's primary care medical provider, and specialty referrals. As part of the diagnostic process, the system and method of the present disclosure can be adapted to administer and score standardized validated screeners as age appropriate such as but not limited to the MCHAT, ASQ, PEDS Form, Vanderbilts, Denver Developmental assessments, Conners Forms, Scat, etc. While Autism may be the primary diagnosis or concern, other coexisting conditions like anxiety or ADHD and/or genetic disorders may exist. If a validated screening as discussed above elicits concerns for other disorders, the appropriate referral will be done through the system and method of the present disclosure.

Concerning anxiety, panic attacks, or PTSD, if the child is showing emotions of frustration with or without crying and/or demonstrating worry about their performance while completing the assessment, has an elevated heart rate and/or blood pressure and/or avoids behaviors when tasks are hard such as: complaining to end or get out of a task like, having headaches, having nausea, stomach aches, vomiting, having tiredness, yawning, falling asleep, having a tense posture, clenched shoulders or muscles, clenched teeth, pulling on eyelashes, biting fingers or fingernails, arguing, throwing the materials, leaving the test environment, saying they are thirsty, hungry, tired, sleepy, or having to all of a sudden got to go to the bathroom, complains of their heart racing, is selectively mute, or is unable to attempt to complete the tests in part or at all, the evaluation should include an anxiety, panic attack, or PTSD evaluation.

A diagnosis of Anxiety is when a child is worried or scared and it interferes with their everyday activities such as refusing to go to school, playing with friends, especially at recess or at social gatherings, getting to sleep and/or not being able to sleep alone in their bed. They may say their heart is beating too fast, or they have a stomachache and shaking, maybe having trouble breathing, start to sweat and suddenly are unable to sustain attention and want to find a safe place, like home or a caregiver. Anxiety may have been exhibited itself as shyness during the child's early years or occur after a traumatic event experienced/witnessed by the child or the child may be a victim of abuse by a caregiver or other. There are 4 types of Anxiety present in children. One type of anxiety is Social Anxiety wherein a child displays difficulty playing or being with others, especially in school, will speak softly not wanting to be heard and will not volunteer to raise hand in school or participate in group activities. Another type of anxiety with early onset is Separation Anxiety wherein a child is unable to separate from the caregiver, wanting to always be in their presence, not wanting to go to school or be on a playdate. Another type of anxiety is Selective Mutism—severe anxiety—wherein a child speaks at home but not anywhere else. Another type of anxiety is Generalized Anxiety wherein a child strives to be perfect, worries about the past, current events and the future a lot and worries about what may happen in school in their schoolwork or other activities. They may get diagnosed with ADHD; however, this child cannot pay attention due to worry, rather than attention. The child may demonstrate administration of the present method by being concerned about getting the assessment tasks correct or simply not speaking. They may cry and turn away and say they are afraid to continue

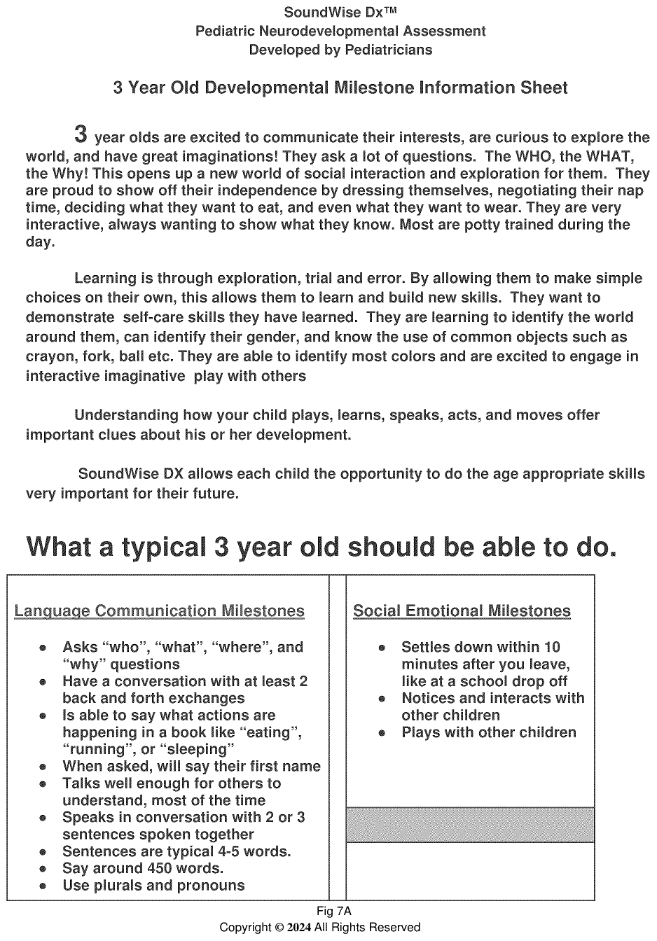

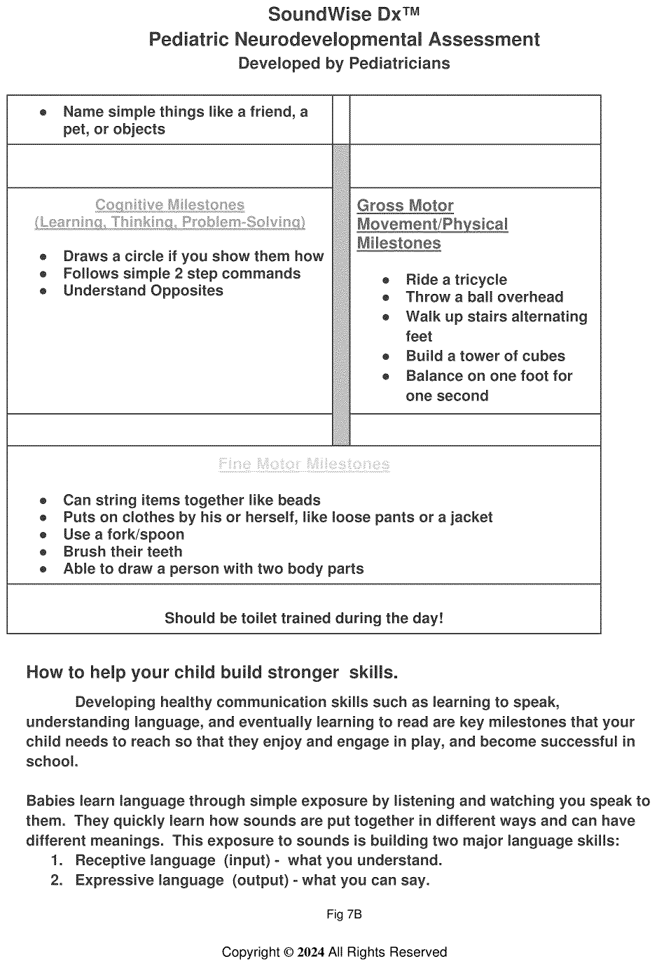

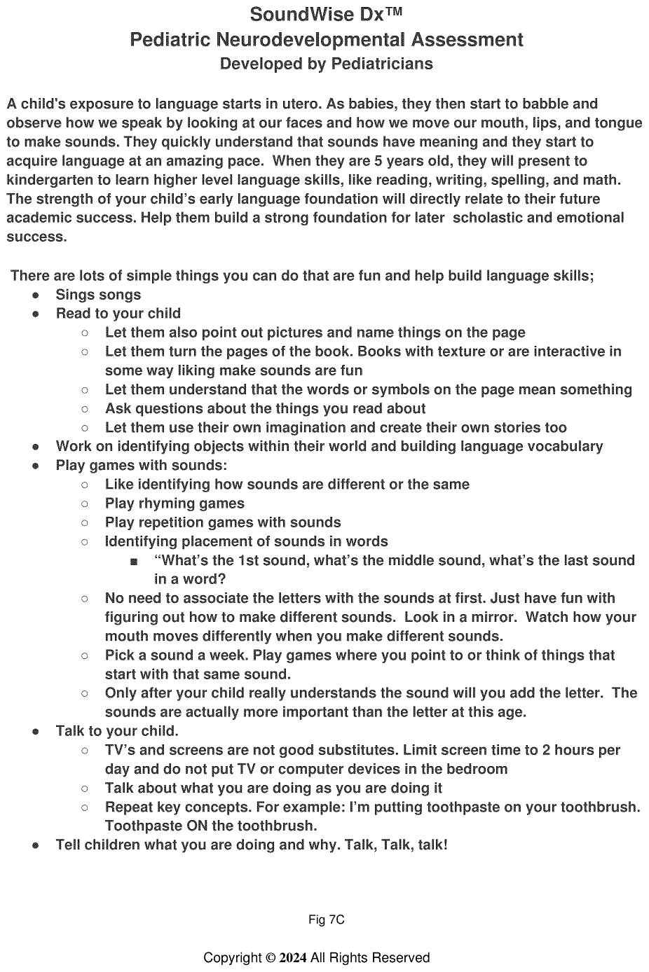

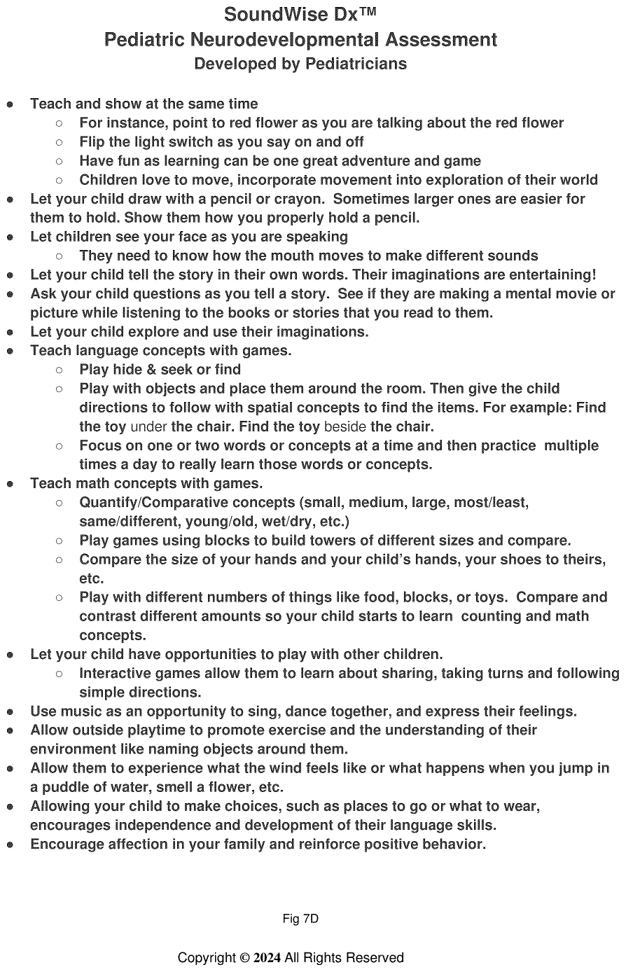

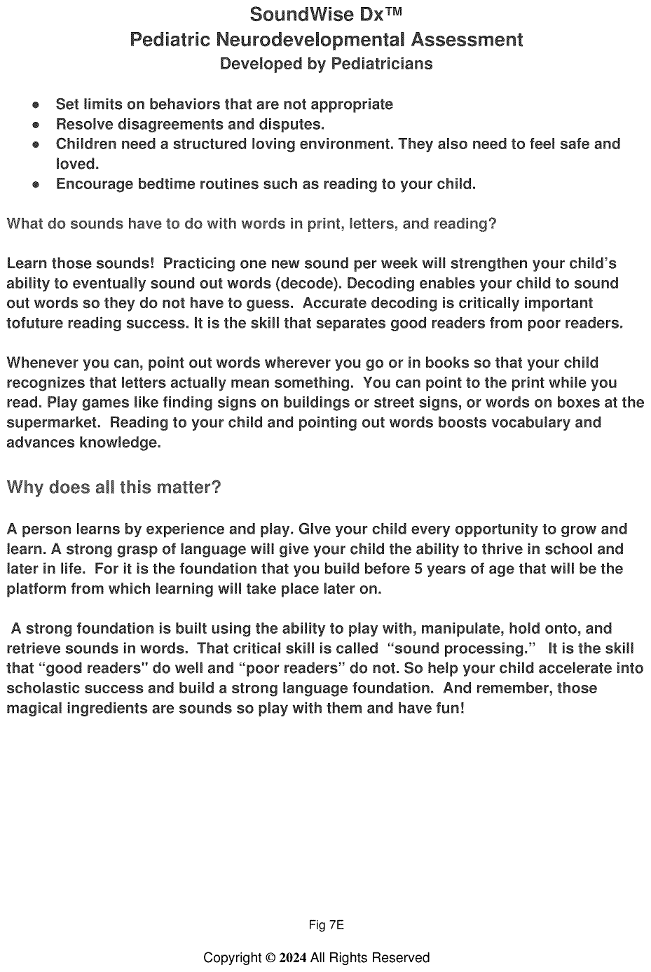

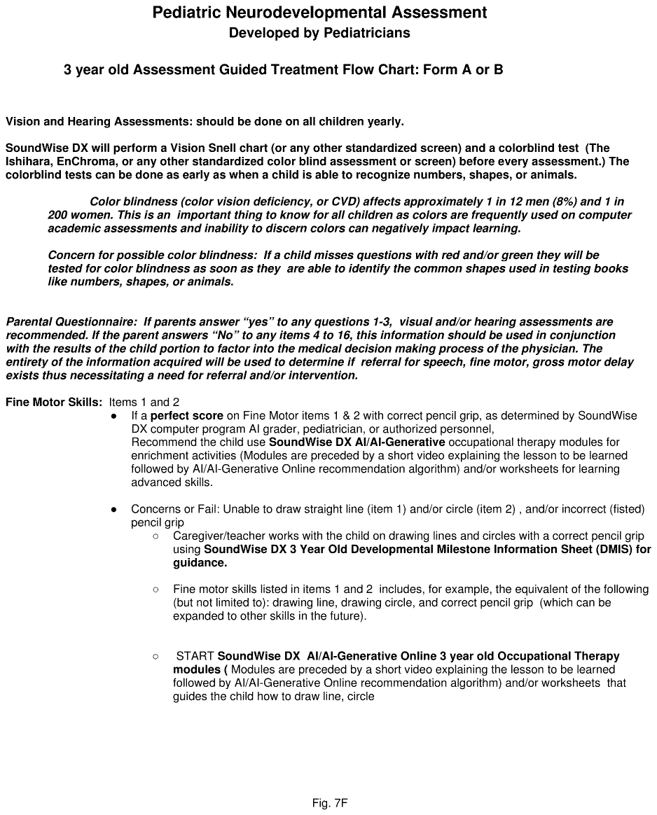

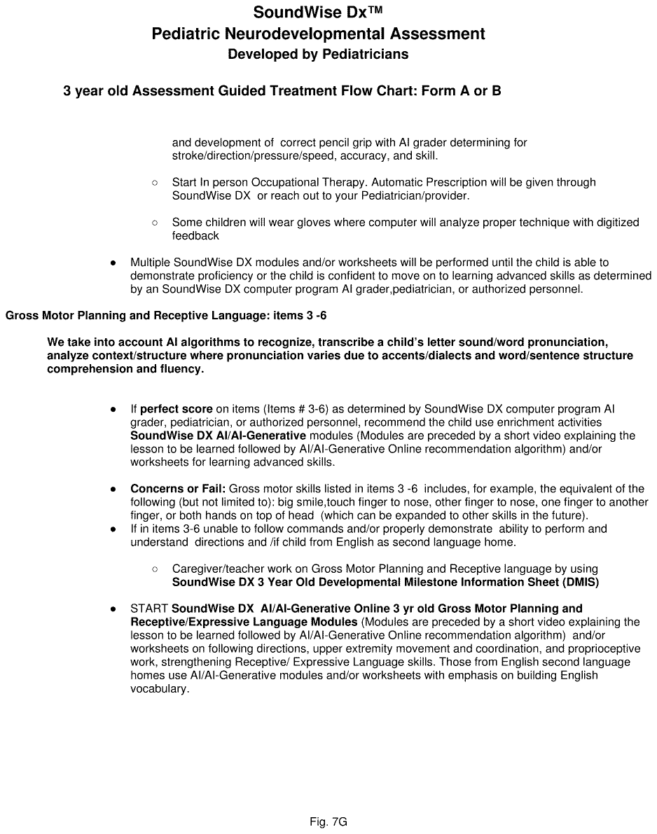

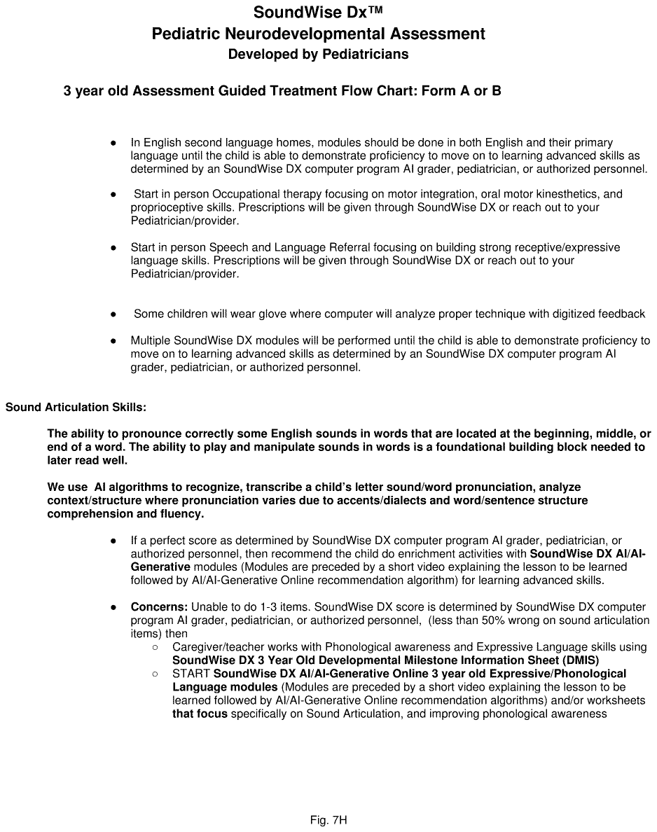

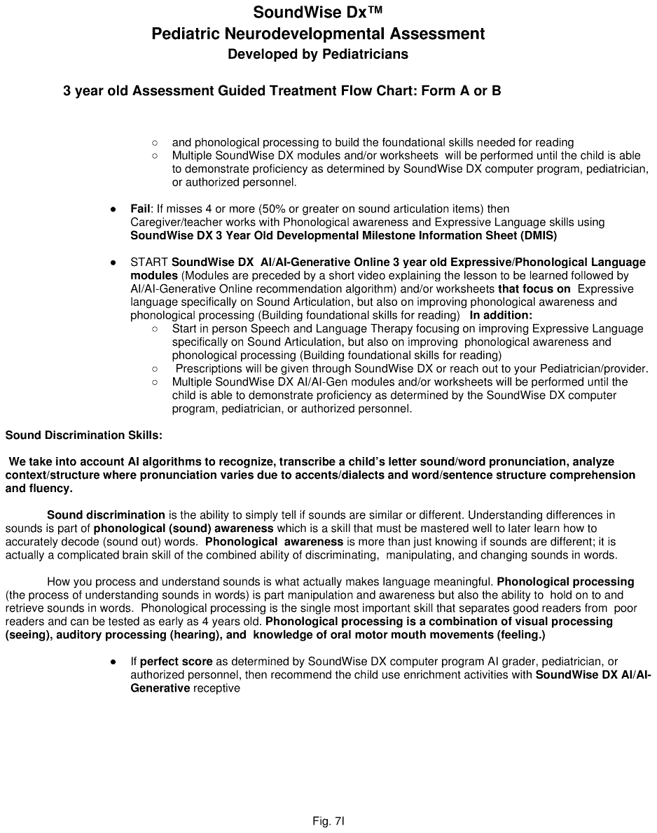

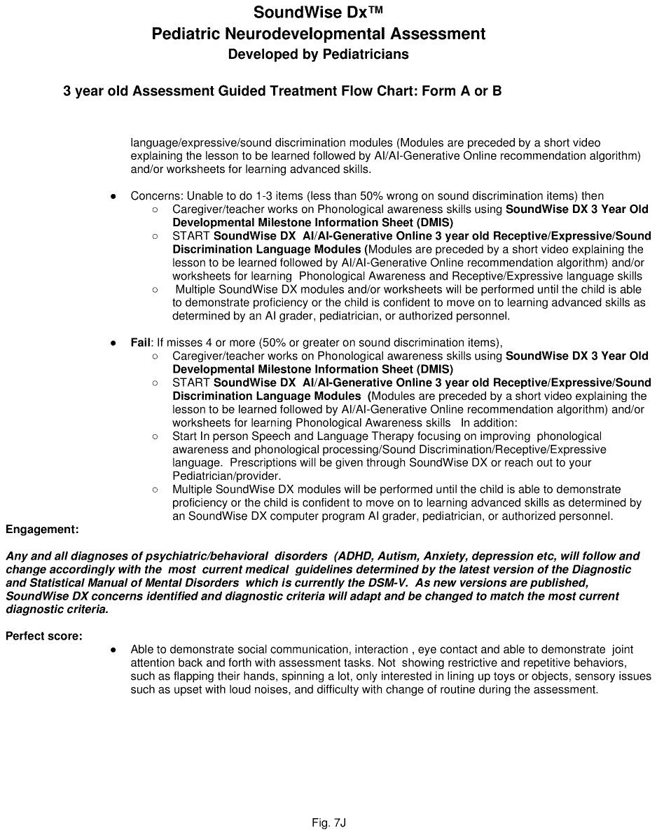

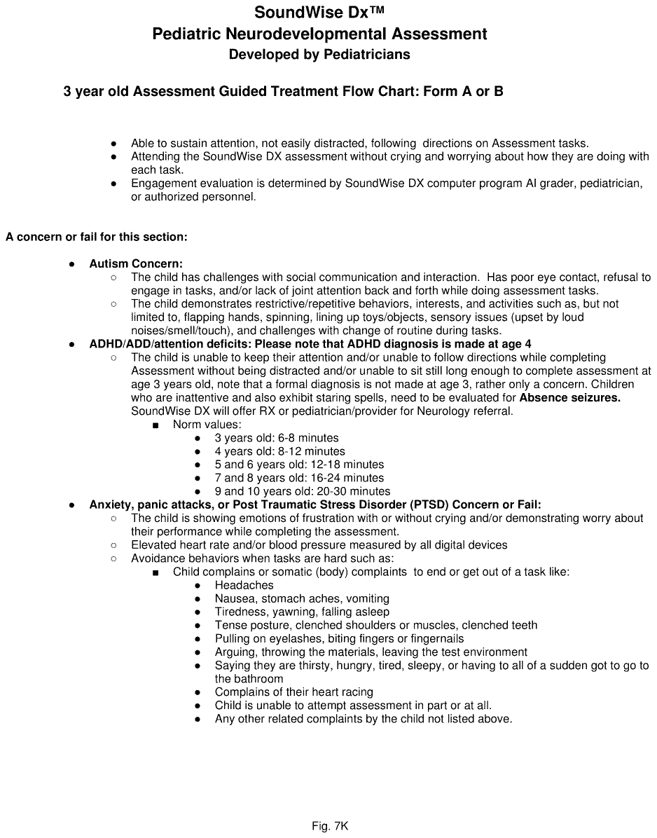

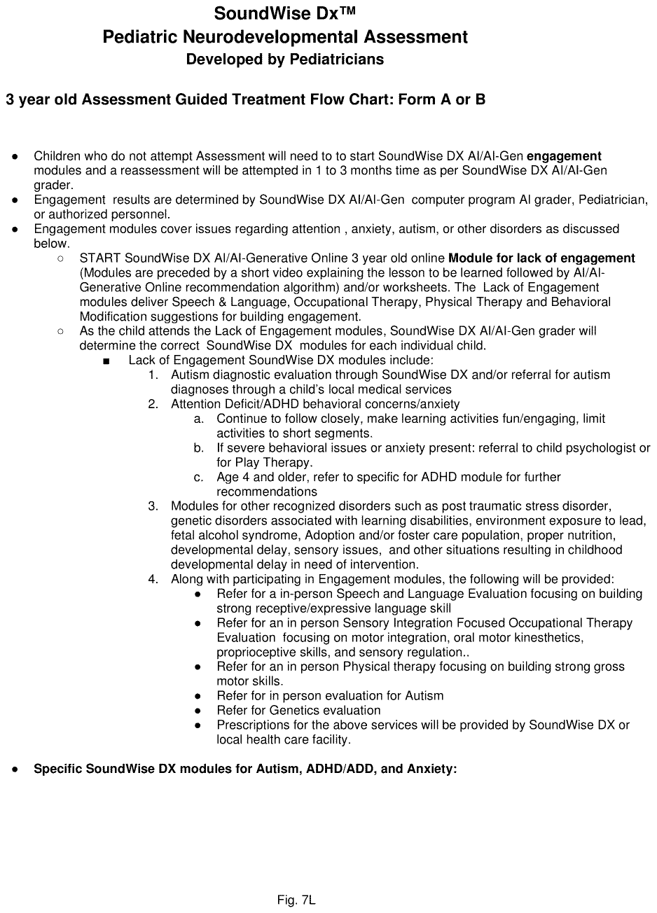

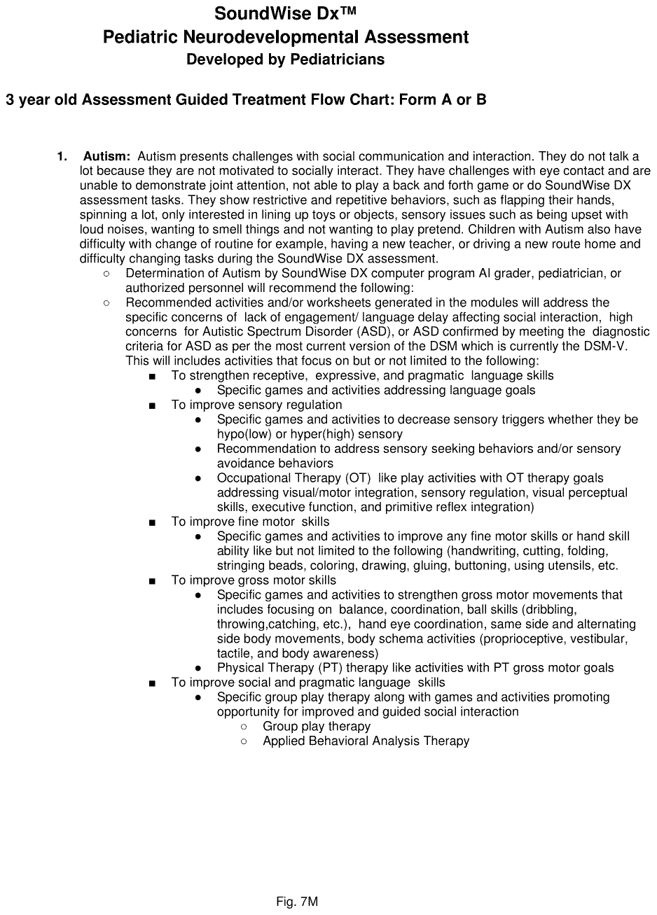

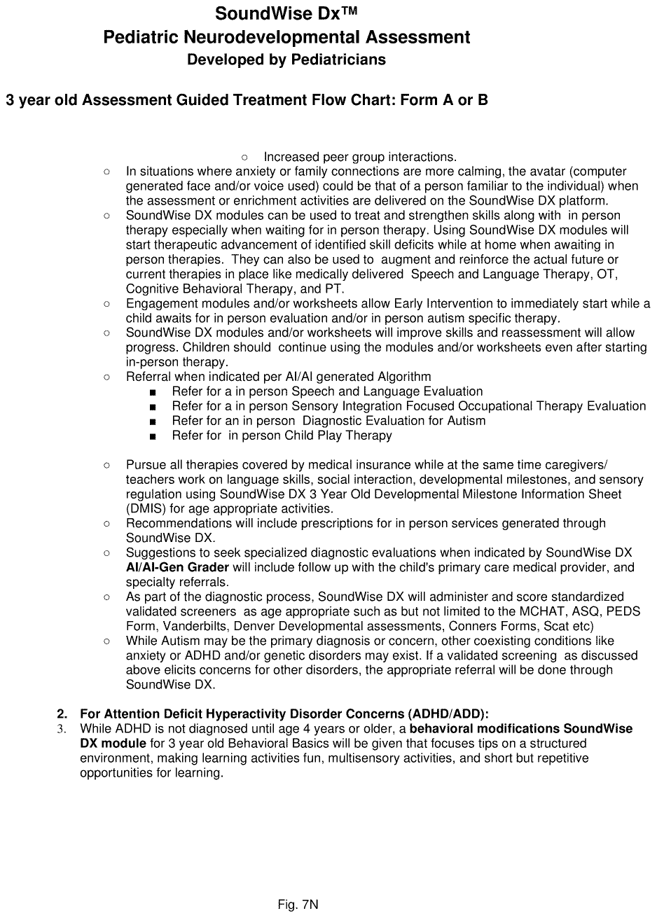

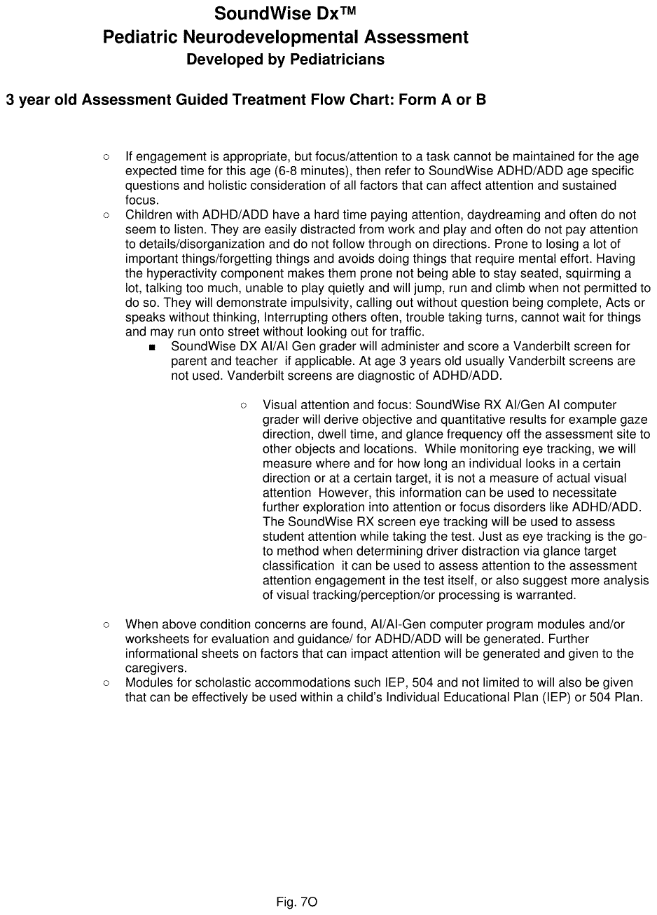

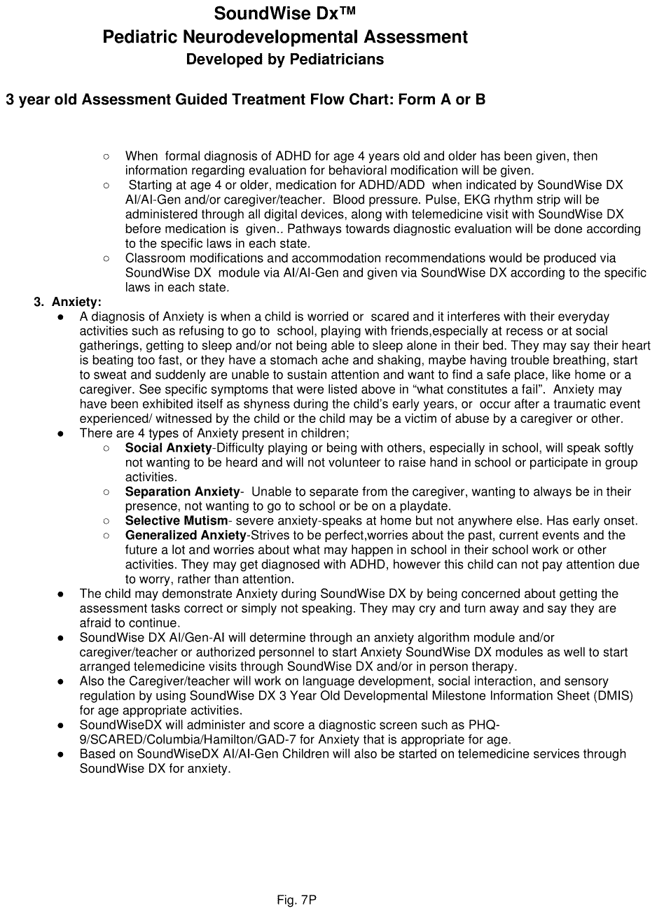

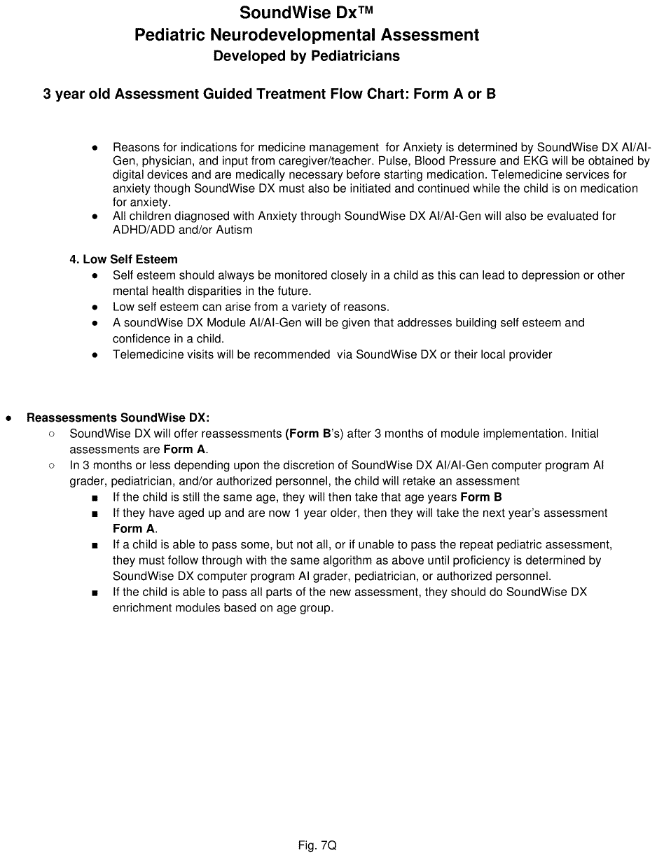

FIGS. 7F-7Q provide assessment guided treatment flow charts A and B which provide recommendations for treatment based on the assessments and answers to questions in the 3 year old questionnaire and neurodevelopmental assessments tasks described in FIGS. 1-6.

The system and method of the present disclosure may be configured to determine through an anxiety algorithm module and/or caregiver/teacher or authorized personnel to start Anxiety modules as well to start arranged telemedicine visits through the system and method of the present disclosure and/or in-person therapy. Also, the Caregiver/teacher will work on language development, social interaction, and sensory regulation by using a 3-Year-Old Developmental Milestone Information Sheet (DMIS) of the system and method of the present disclosure for age-appropriate activities. One example of a 3-Year-Old Developmental Milestone Information Sheet is shown in FIGS. 7A-7E. The system and method of the present disclosure may be configured to administer and score a diagnostic screen such as PHQ-9/SCARED/Columbia/Hamilton/GAD-7 for Anxiety that is appropriate for age. Based on the system and method of the present disclosure, Children will also be started on telemedicine services through the system and method of the present disclosure for anxiety.

Reasons for indications for medicine management for Anxiety is determined by the system and method of the present disclosure, physician, and input from caregiver/teacher. Pulse, Blood Pressure and EKG will be obtained by digital devices and are medically necessary before starting medication if indicated. Telemedicine services for anxiety though the system and method of the present disclosure must also be initiated and continued while the child is on medication for anxiety. All children diagnosed with Anxiety through the system and method of the present disclosure will also be evaluated for ADHD/ADD and/or Autism, if indicated.

Concerning low self-esteem, self-esteem should always be monitored closely in a child as this can lead to depression or other mental health disparities in the future. Low self-esteem can arise from a variety of reasons. The system and method of the present disclosure may include one or more modules that address building self-esteem and confidence in a child. Telemedicine visits will be recommended via the system and method of the present disclosure or a local provider. Anxiety will be less when a child can confidently and competently perform what is asked of him.

In the first part of the method, a referral may also generate referring the individual for services in areas where the evaluation results were sufficient to improve the child's skills, even though the child's skills are proficient for his or her age. For example, if the child demonstrates that he or she is able to demonstrate social communication, interaction, eye contact and able to demonstrate joint attention back and forth with assessment tasks, it may be indicative that the child's skills are not deficient, and the child needs to only to continue to build and/or improve his or her skills. Further, not showing restrictive and repetition behaviors, such as flapping their hands, spinning a lot, being only interested in lining up toys or objects, having sensory issue such as being upset with loud noises and difficulty with change of routine may also be indicative that the child's skills are not deficient, and the child needs to only to continue to build and/or improve his or her skills. Indeed, if the child is able to maintain attention, is not easily distracted, follows directions, etc. it may also be indicative that the child's skills are not deficient, and the child needs to only continue to build and/or improve his or her skills. Likewise, if the child is able to complete the tests without crying or worrying about how they are doing on the tests, it may also be indicative that the child's skills are not deficient, and the child needs to only to continue to build and/or improve his or her skills.

In some embodiments, the first part of the method further includes observing behavioral engagement skills of the child throughout the testing process, such as, for example, during the first test, during the second test, during the third test and/or during the fourth test. In some embodiments, the observations made throughout the testing process can include noting the child's demeanor, such as, for example, observations pertaining to whether the child is cooperative, uncooperative, refuses to interact, completed the tests independently, exhibited poor eye contact, smiled, cried, was tired, would not engage, was shy, was distractible, was anxious, needed redirection for completion, was happy and engaged, was an English language learner, was difficult to motivate, etc. In some embodiments, the observations made throughout the testing process are included in the worksheet (FIGS. 3 and 4) and/or are used to generate the referral. In some embodiments, information pertaining to the answers provided in the questionnaire (FIG. 1) is included in the worksheet and/or is used to generate the referral.

### Method—Part Two—Year Four

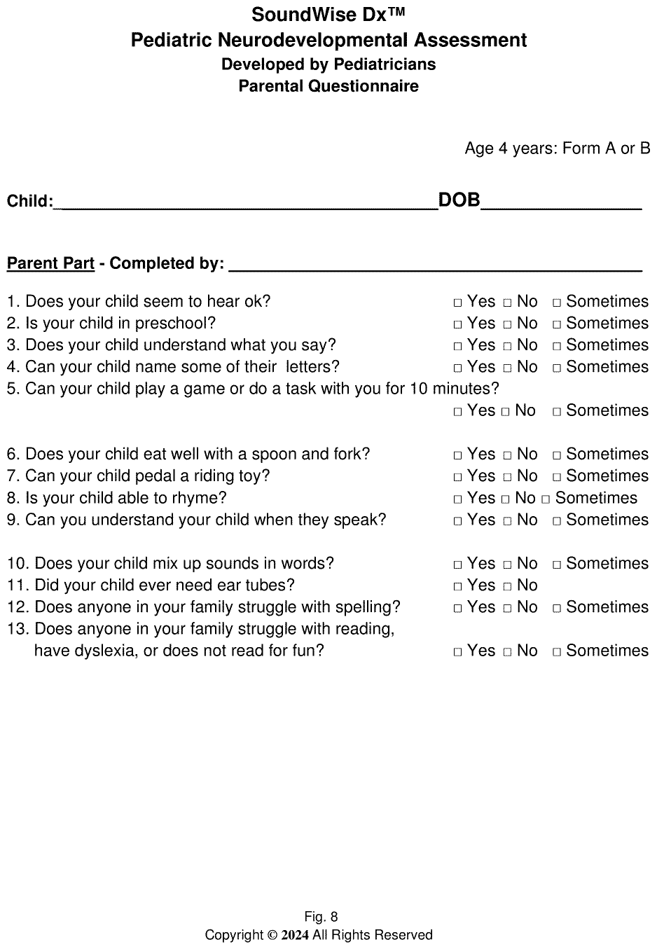

In a second part of the method, which should be administered after the first part of the method discussed above is administered to the child, when the child is approximately 4 years old, the child's parent's is required to fill out a questionnaire that includes various questions about the child. One embodiment of the questionnaire is shown in FIG. 8. As shown in FIG. 8, the questionnaire can include questions about the child's physical abilities, such as, for example, the child's hearing and vision. The questionnaire can also include questions relating to the child's ability to read, write, comprehend information, perform simple tasks, socialize, etc. In some embodiments, the child is required to undergo a Vision Snell chart test and/or a colorblind test (e.g., The Ishihara, EnChroma, or any other standardized color-blind assessment or screen) prior to beginning the second part of the method.

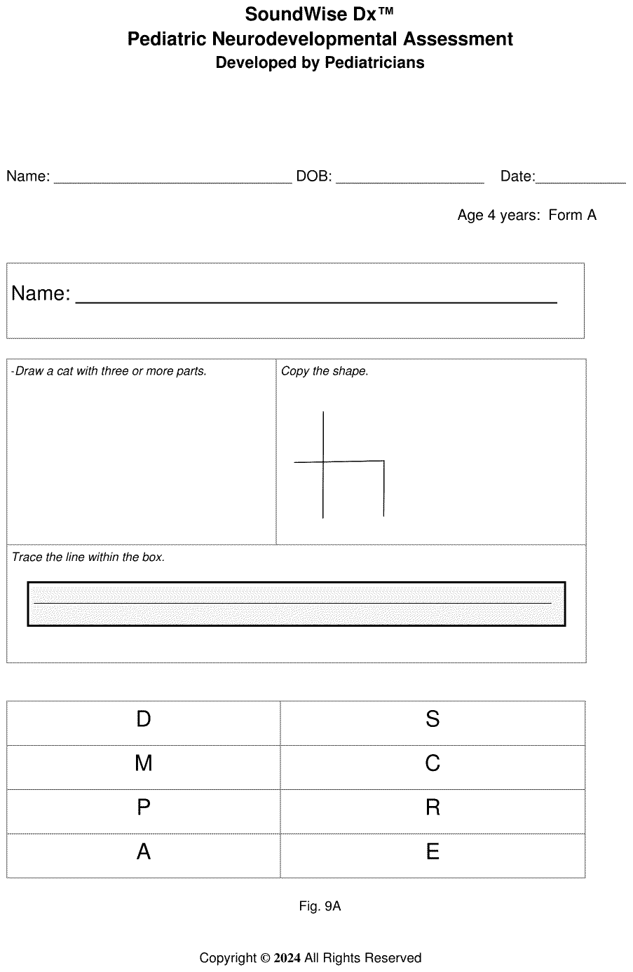

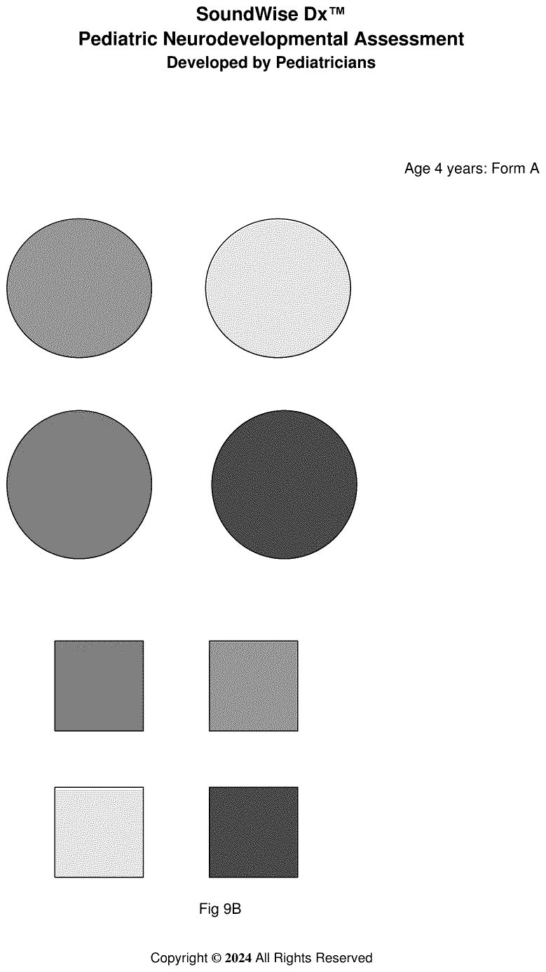

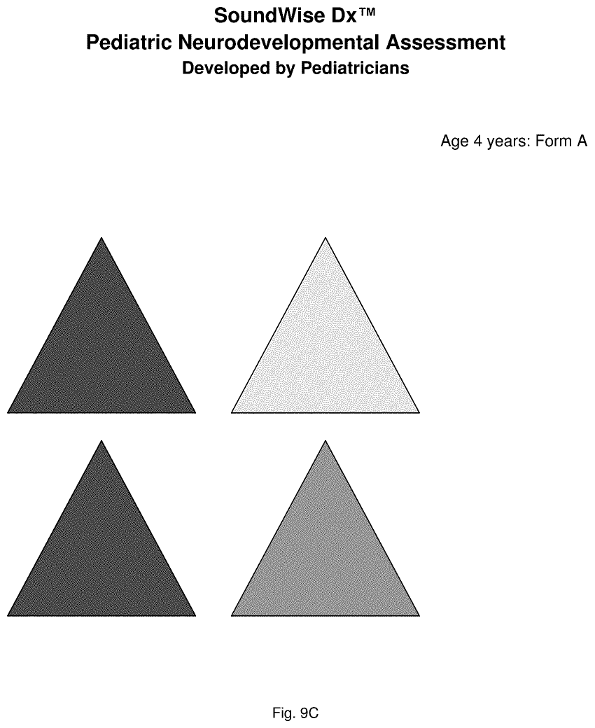

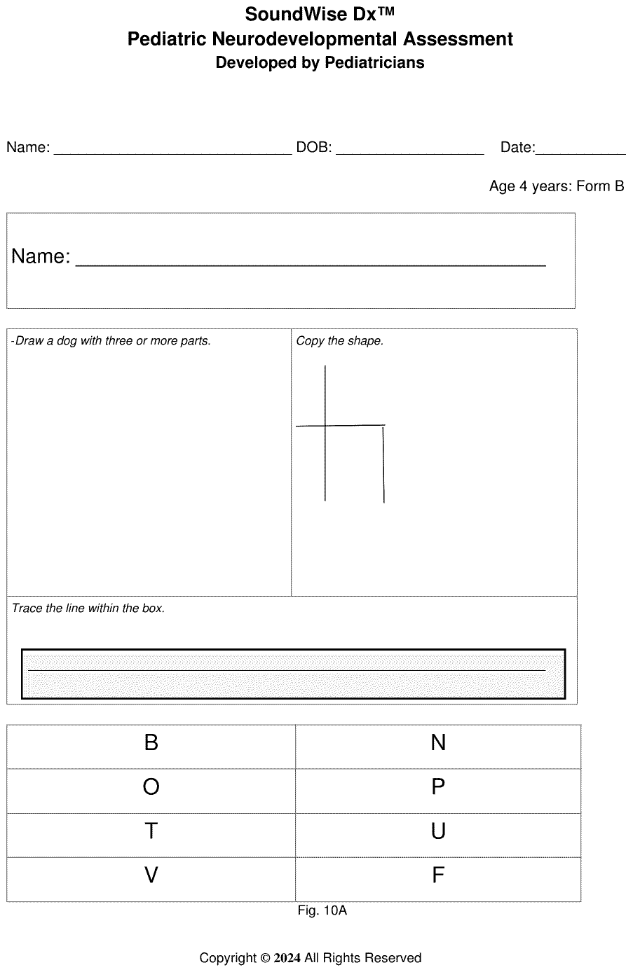

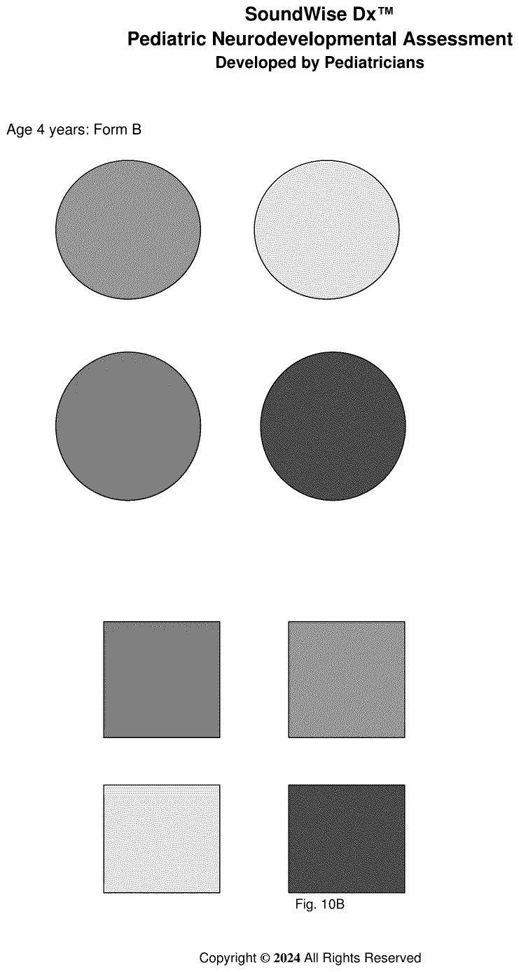

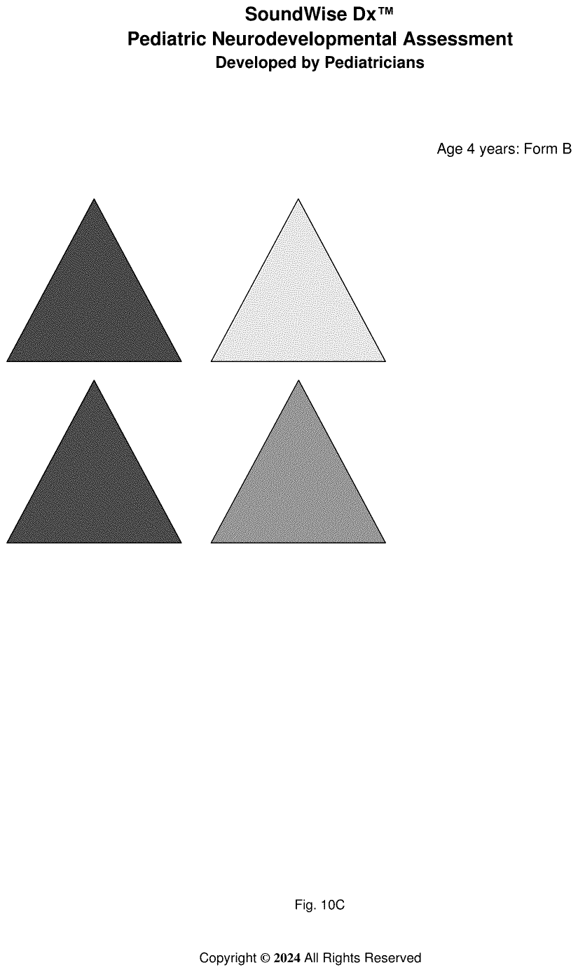

In the second part of the method, tests concerning speech and language, fine and gross motor skills are evaluated for the presence or absence of a disorder and/or school readiness. In particular, in the second part of the method, a first test is administered in which the child has to pick a picture that fits best. For example, in some embodiments, the child may be required to draw a cat with three or more parts, as shown in FIG. 9A. In some embodiments, the child may be required to copy a shape, as shown in FIG. 9A. In some embodiments, the child may be required to trace a line within a box, as shown in FIG. 9A. In some embodiments, the child may also be required to show a blue circle; point to a yellow square; show a green square; and/or show a red triangle, as shown in FIGS. 9B and 9C. In other embodiments, the child may be required to draw a dog with three or more parts, as shown in FIG. 10A. In some embodiments, the child may be required to copy a shape, as shown in FIG. 10A. In some embodiments, the child may be required to trace a line within a box, as shown in FIG. 10A. In some embodiments, the child may be required to pick a picture that depicts a red circle; point to a blue square; show a yellow square; and/or show a green triangle, as shown in FIGS. 10B and 10C.

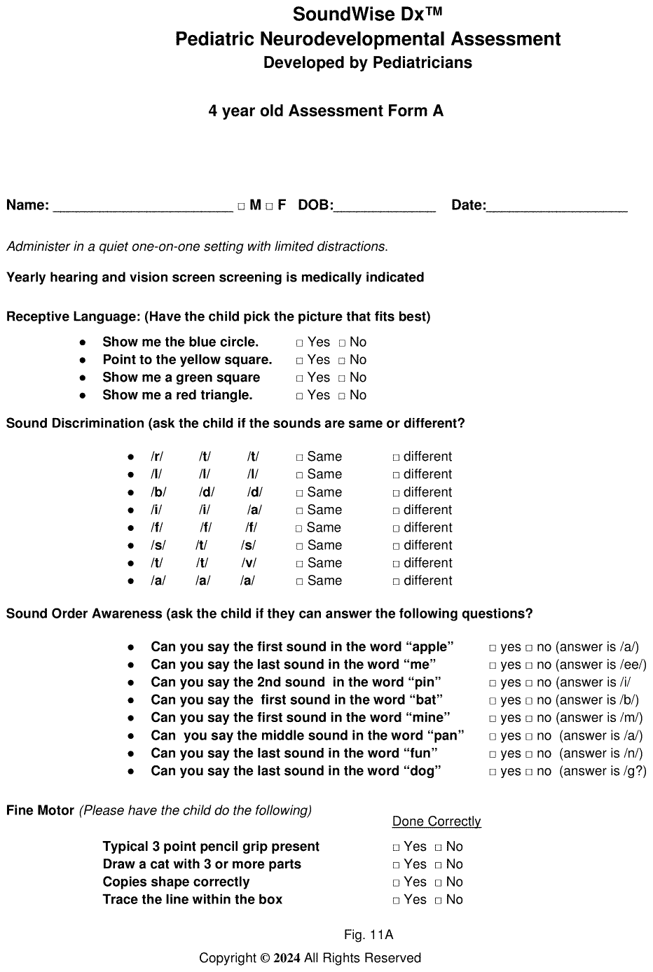

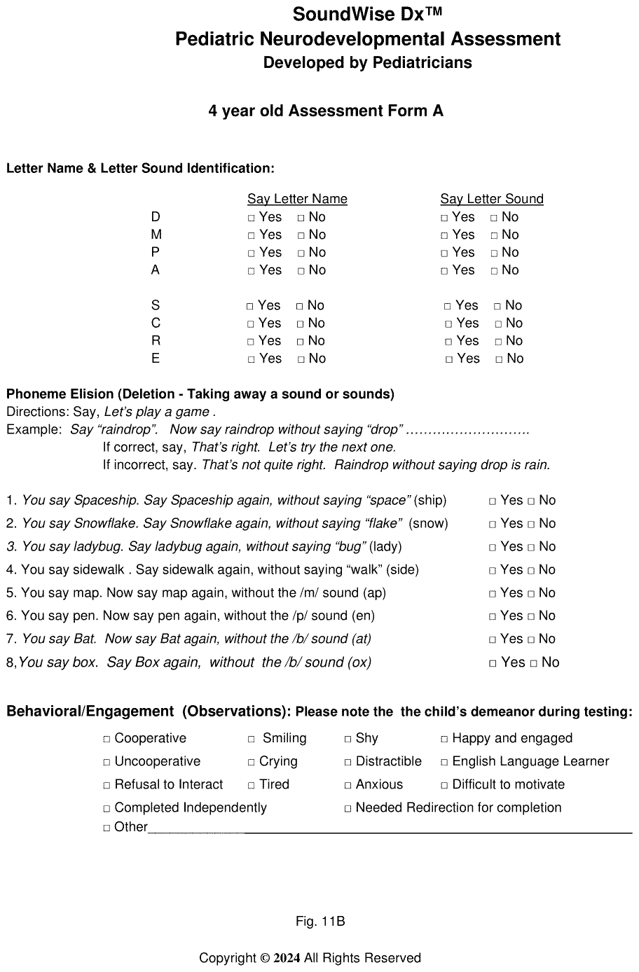

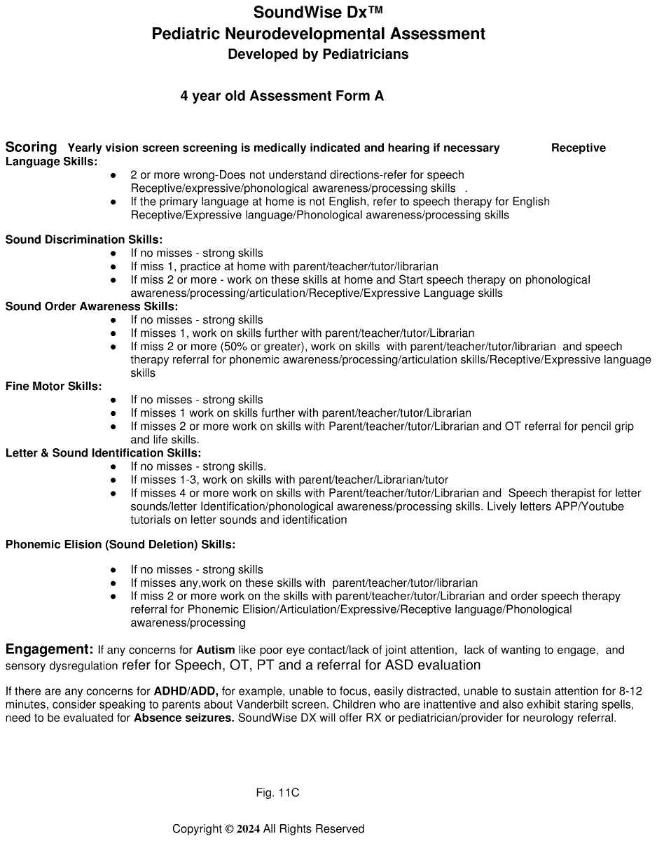

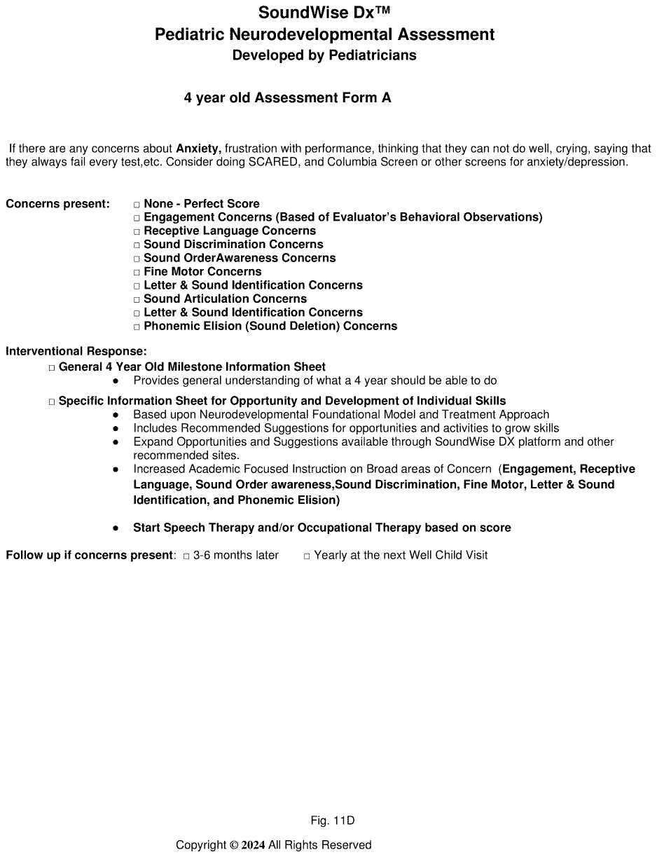

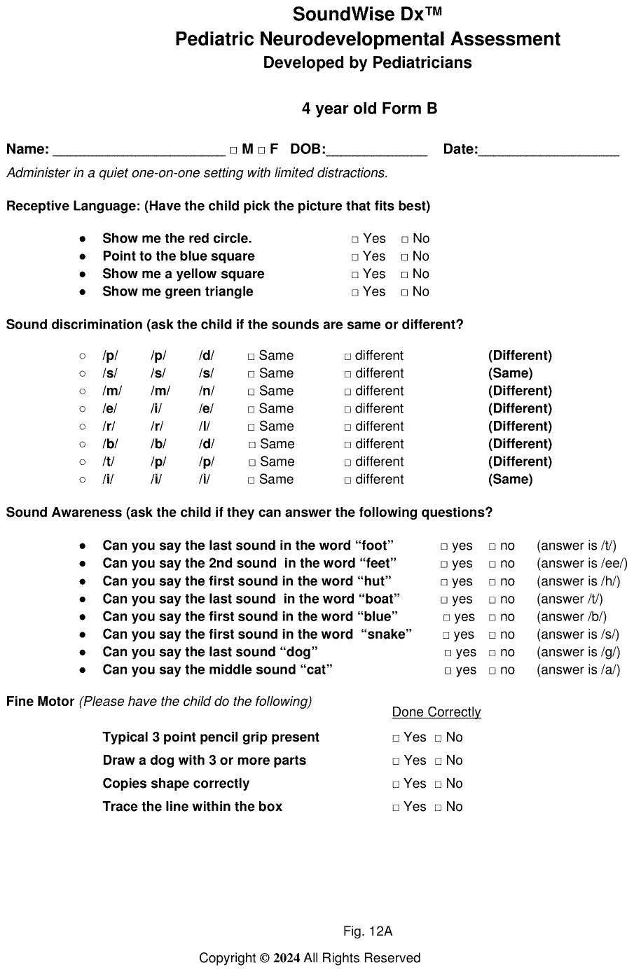

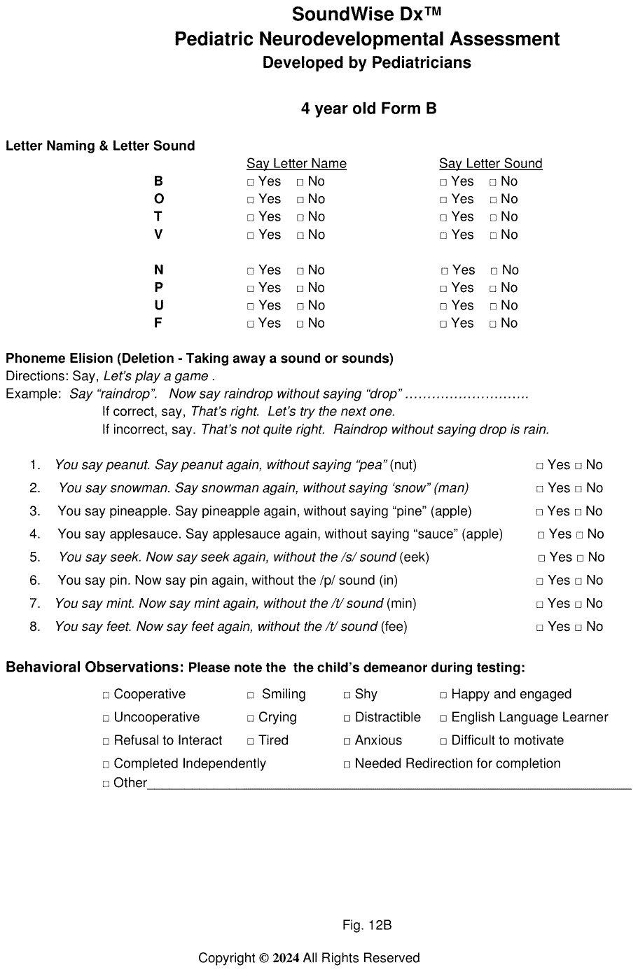

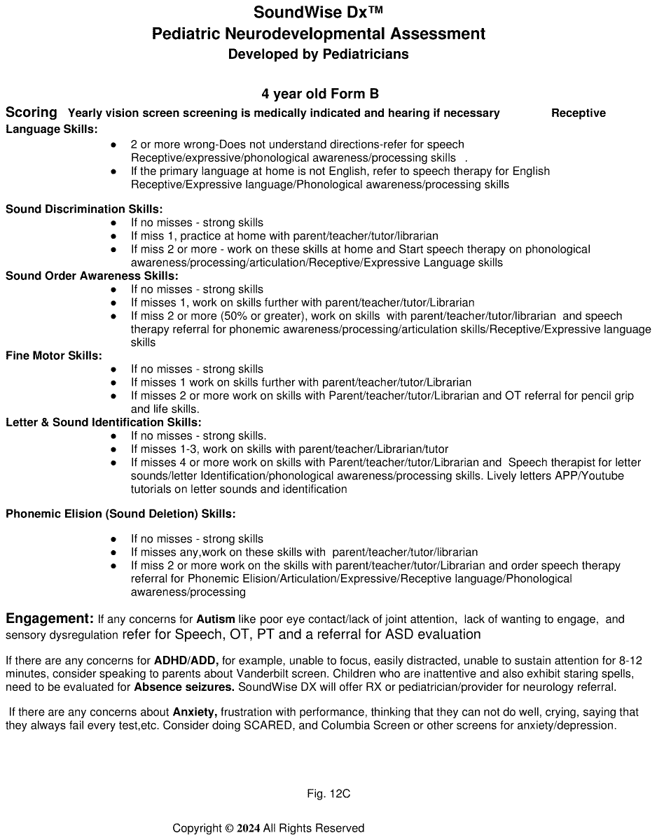

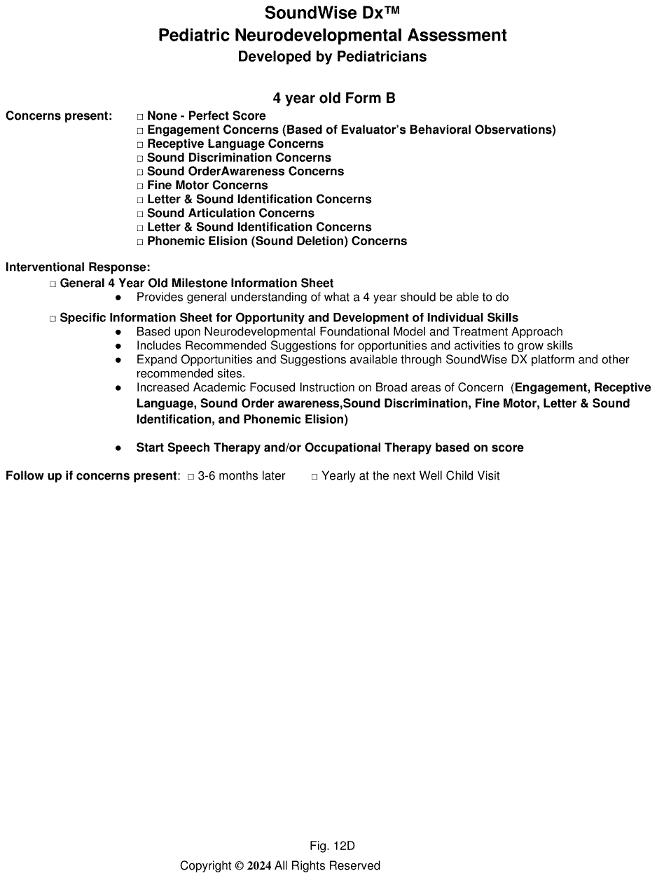

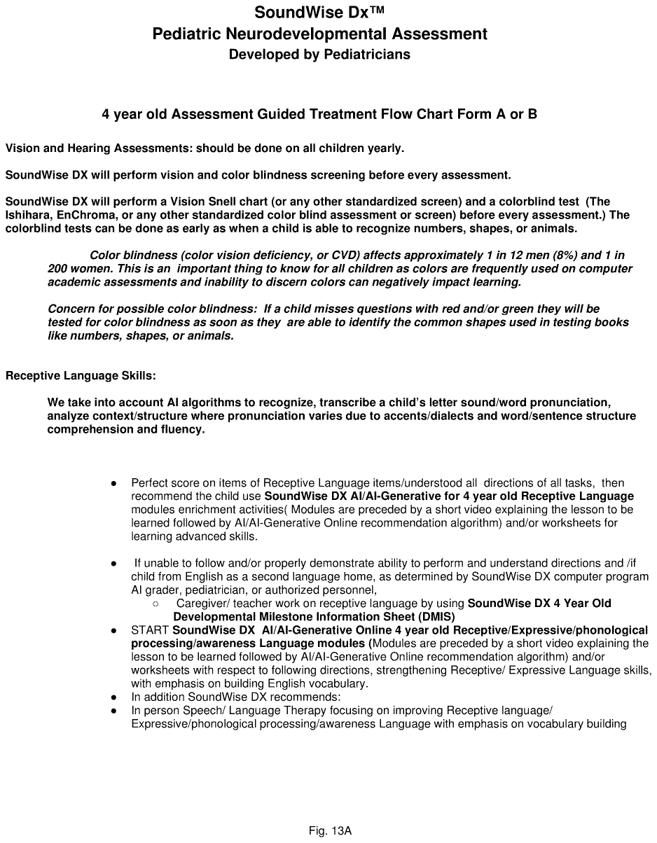

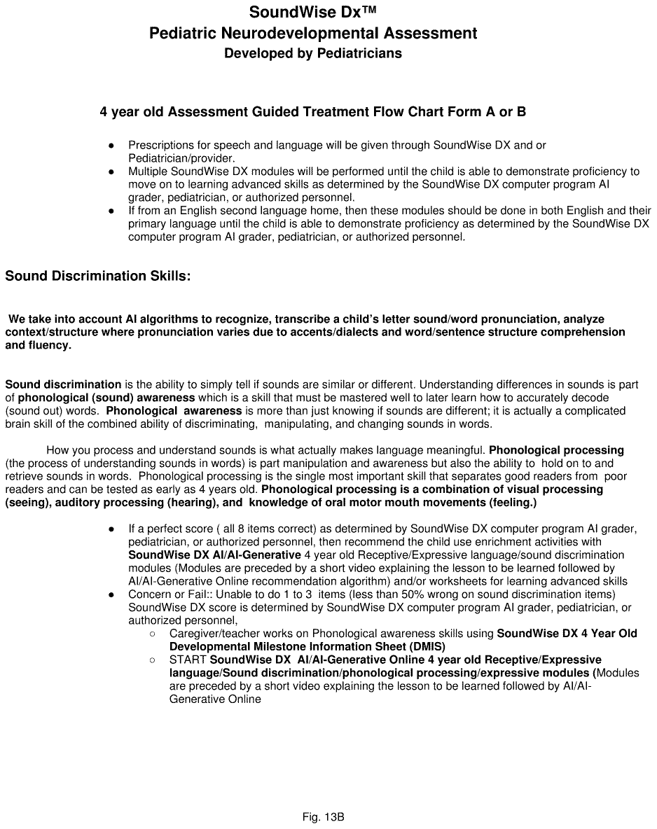

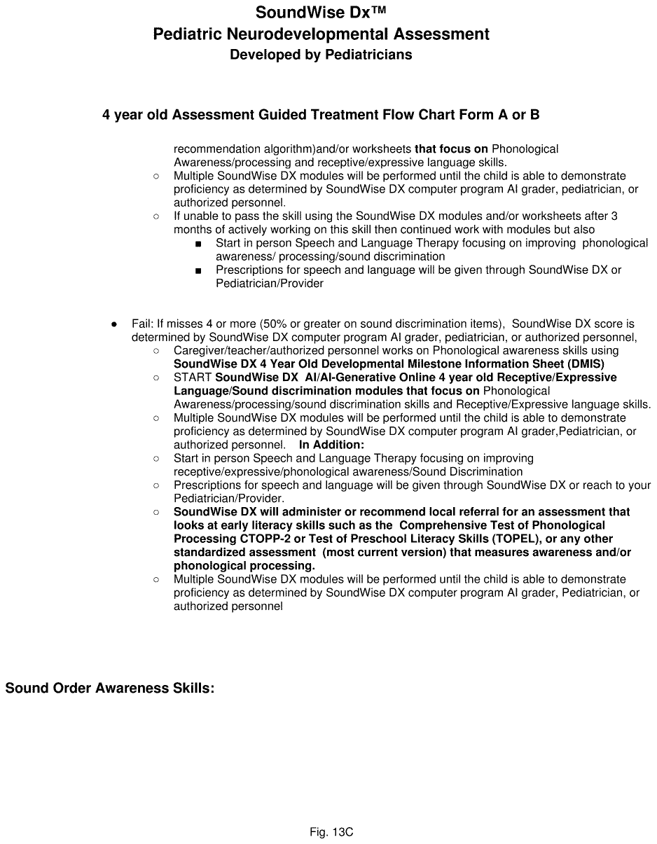

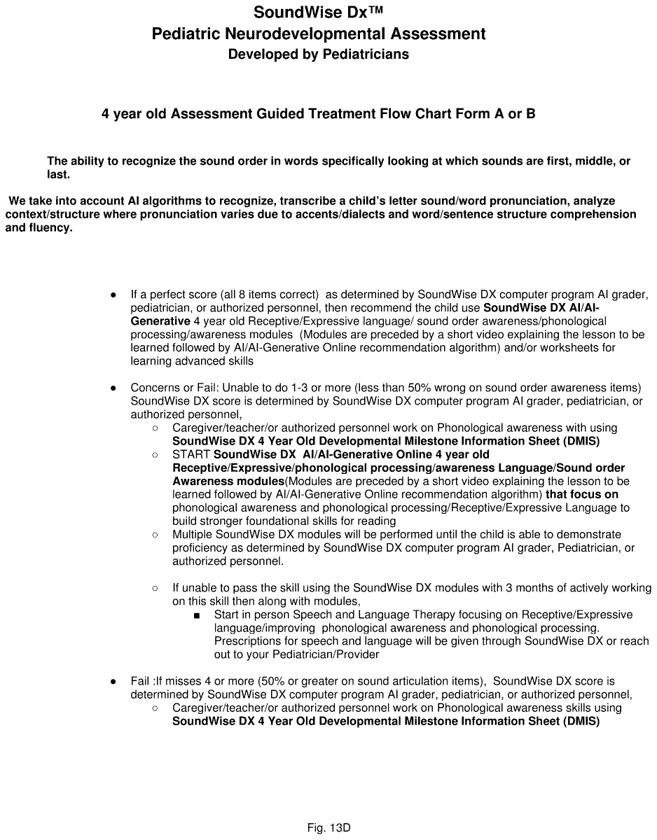

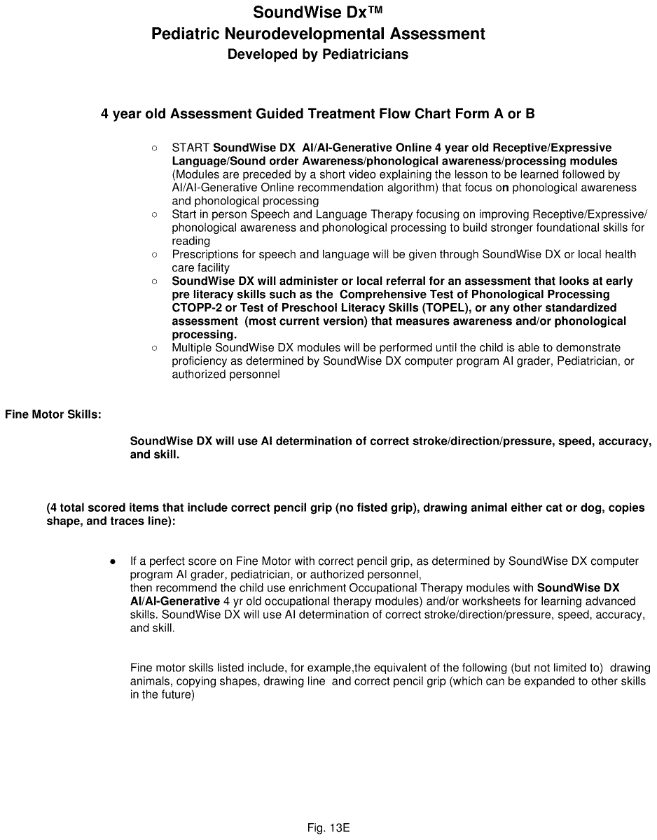

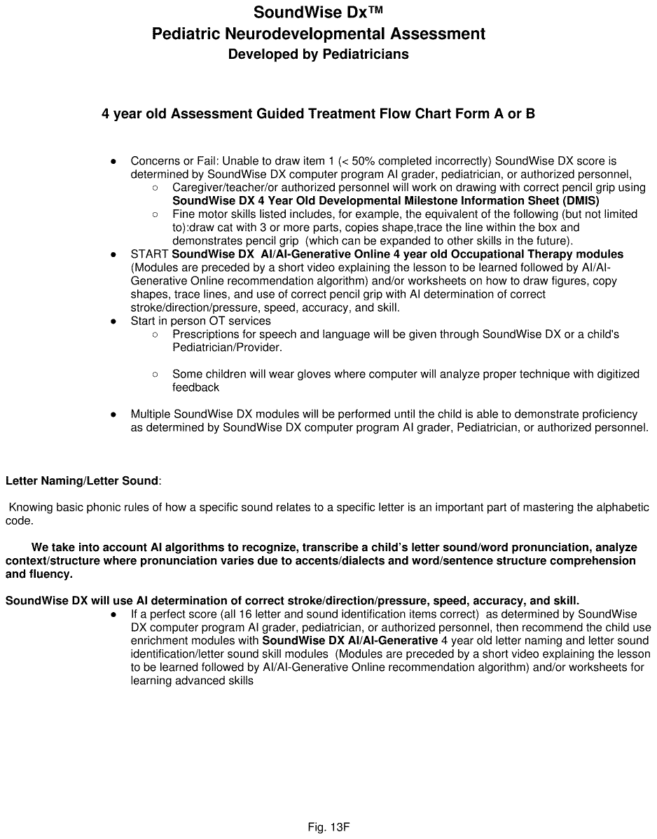

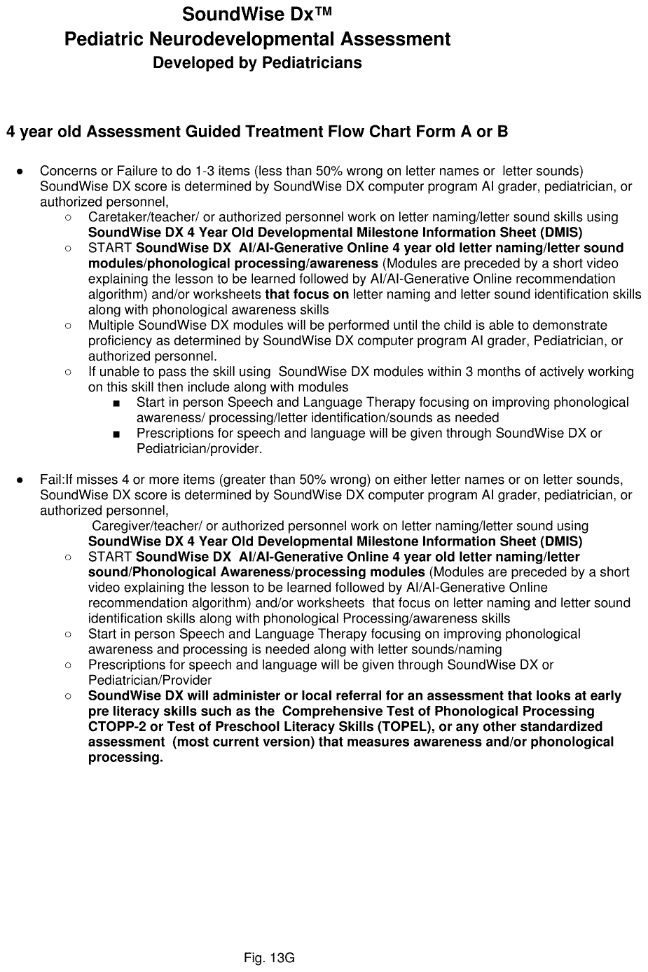

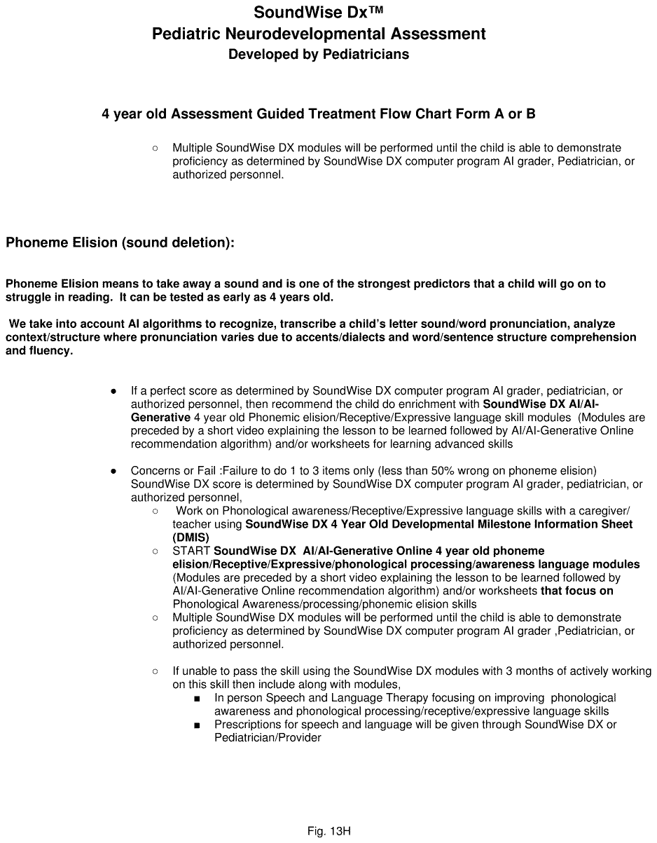

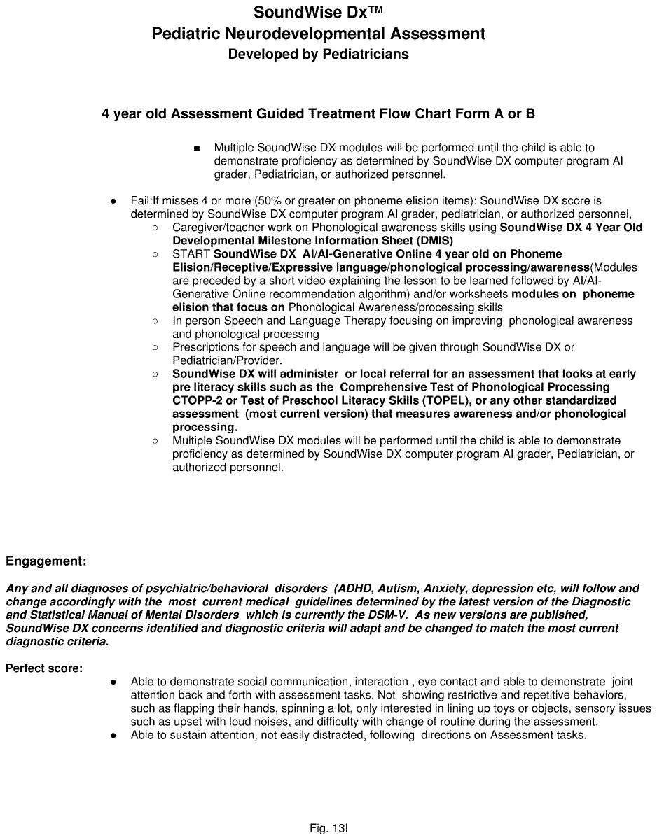

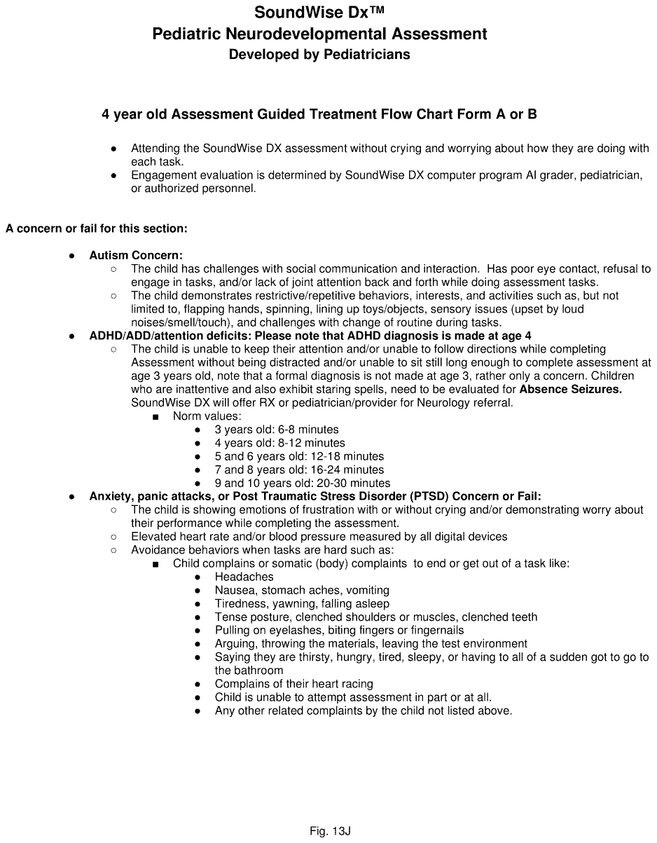

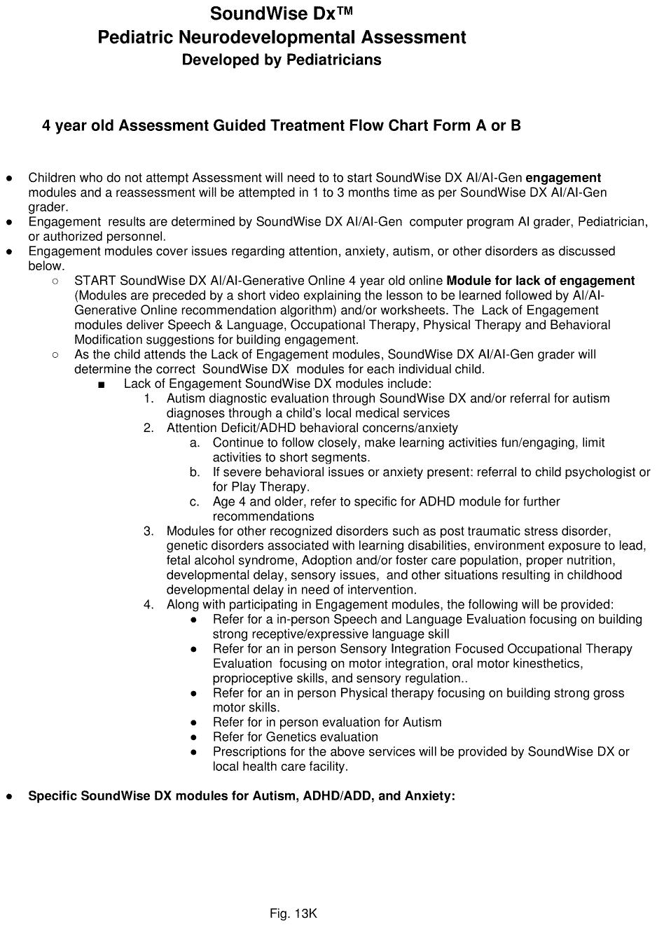

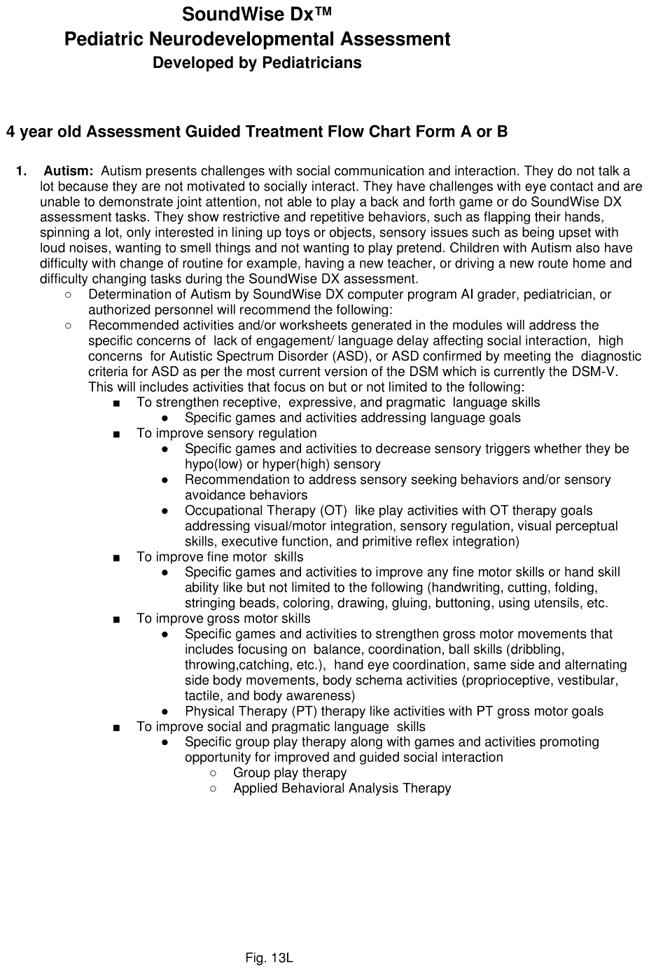

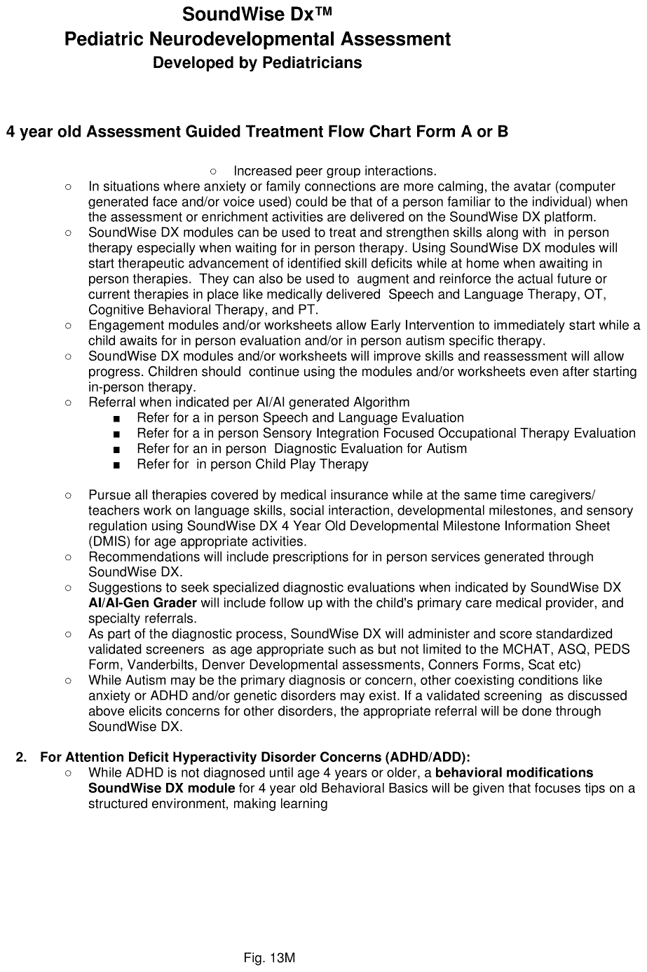

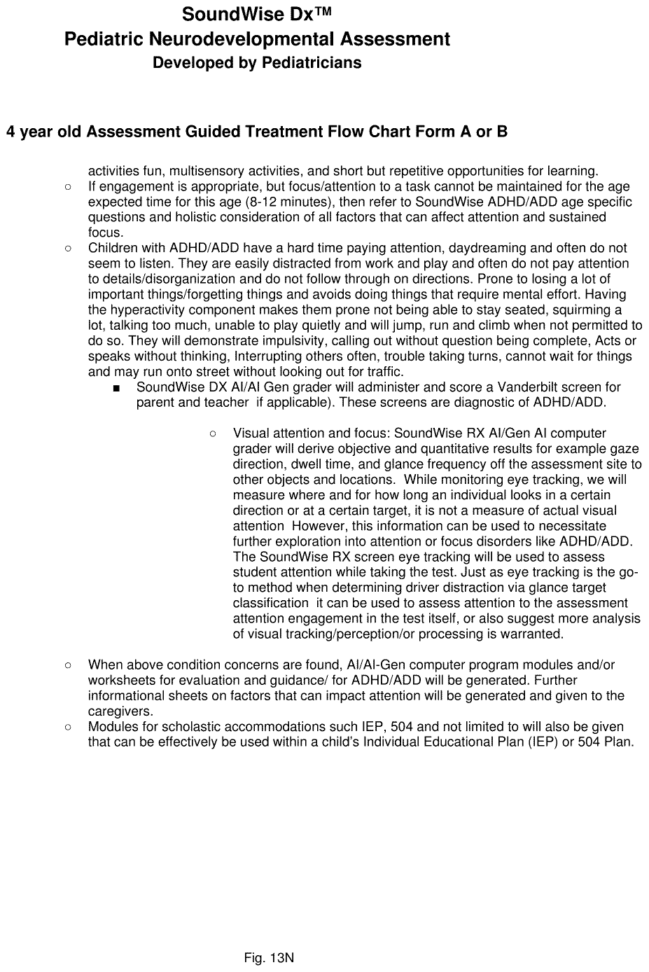

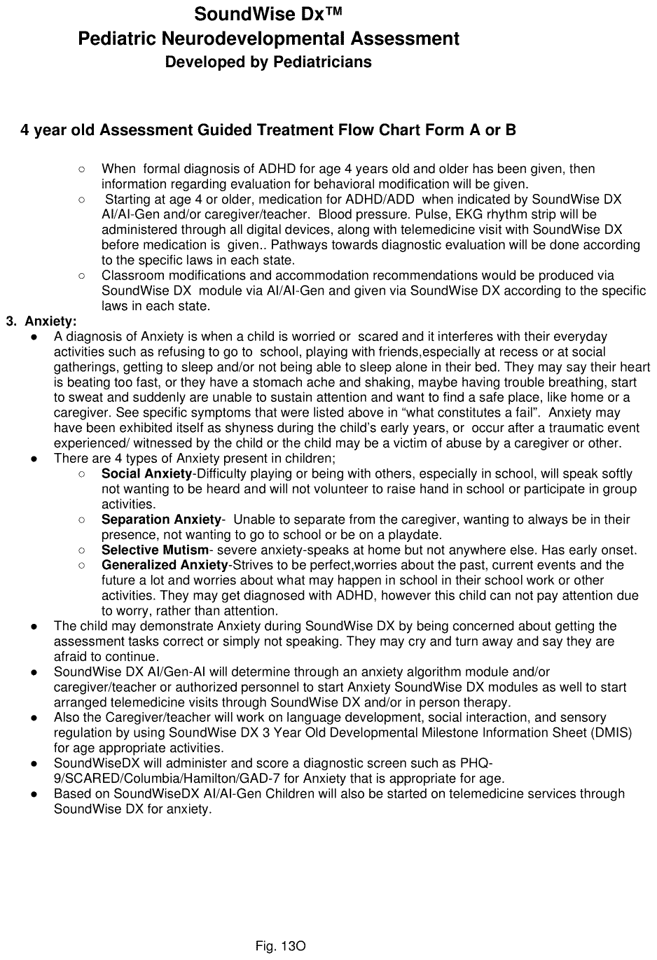

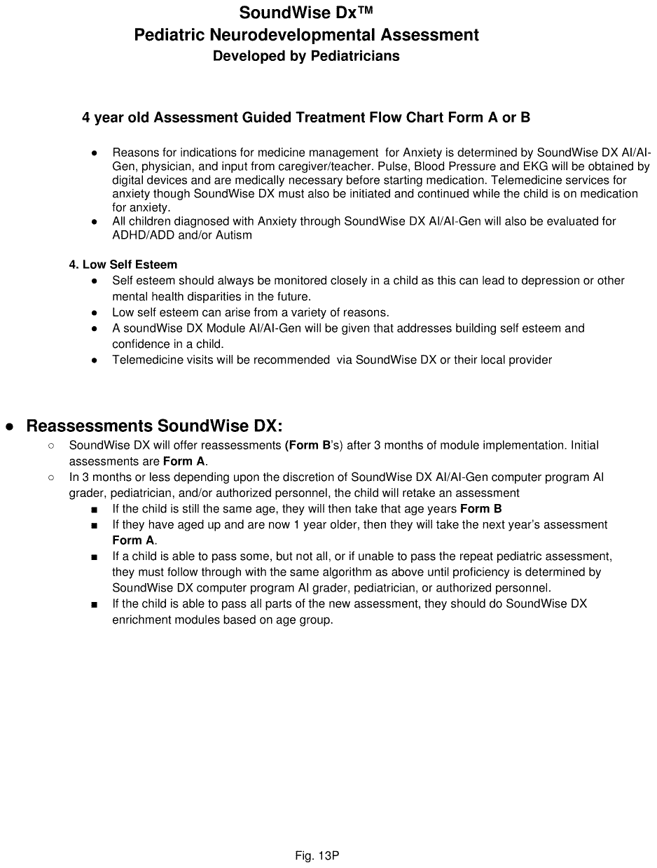

The ability of the child to draw or perform the requested tasks is recorded to generate receptive language input data. The ability of the child to perform one or more of the requested tasks may be recorded in a second worksheet. One embodiment of the second worksheet is shown in FIGS. 11A-11D. Another embodiment of the second worksheet is shown in FIGS. 12A-12D. The receptive language input data may include whether or not the child was able to perform required tasks. The ability of the child to perform the required tasks may be recorded in the second worksheet. The receptive language input data is evaluated against an answer key to generate a receptive language evaluation result. The receptive language evaluation result is another determination with respect to the presence or absence of receptive language deficiency, after the first determination with respect the presence or absence of receptive language deficiency conducted during the first part of the method. In some embodiments, the receptive language evaluation result includes a score, such as, for example, a number score. In some embodiments, the receptive language evaluation result includes a written evaluation, such as, for example, words written in sentence or note form that explain what was observed by the practitioner. In some embodiments, the receptive language result is included in an assessment that evaluates at least each of the tests administered during the second part of the method. One embodiment of the assessment is shown in FIGS. 13A-13Q.

In the second part of the method, a second test is administered in which the child has to do a sound discrimination task to generate a sound discrimination task input data. The ability of the child to do a sound discrimination task may be recorded in the second worksheet (FIG. 11A-11D or FIG. 12A-12E). In some embodiments, the sound discrimination task may include having the child say if sounds are the same or different. For example, the child may be required to say if two of the same letters sound different than another letter. In some embodiments, the sound discrimination evaluation result is included in the assessment (FIG. 11D, or FIG. 12A-12D). In some embodiments, the sound discrimination evaluation result uses AI or other software to recognize and/or transcribe the child's letter sound/word pronunciation, analyze context/structure where pronunciation varies due to accents/dialects and word/sentence structure comprehension and fluency.

In the second part of the method, a third test is administered in which the child has sound order awareness. The ability with sound order awareness may be recorded in the worksheet (FIG. 11A-11D or FIG. 12A-12E as sound order awareness input data. In some embodiments, administering the third test may include asking the child say the first wound in the word “apple”; having the child say the last sound in the word “me”; having the child say the second sound in the word “pin”; having the child say the first sound in the word “bat”; having the child say the first sound in the word “mine”; having the child say the middle sound in the word “pan”; having the child say the last sound in the word “fun”; and/or having the child say the last sound in the word “dog” (FIG. 11A). In some embodiments, administering the third test may include asking the child say the last sound in the word “foot”; having the child say the second sound in the word “feet”; having the child say the first sound in the word “hut”; having the child say the last sound in the word “boat”; having the child say the first sound in the word “blue”; having the child say the first sound in the word “snake”; having the child say the last sound “dog” and/or having the child say the middle sound “cat” (FIG. 12A).

In the second part of the method, a fourth test is administered in which the child has to determine if the child has appropriate fine motor skills. In the fourth test, the child may be required to perform a typical three point pencil grip; to draw a cat with three or more parts; to copy a shape correctly (FIG. 11A); and/or to trace a line within a box; and/or to perform a typical three point pencil grip test; to draw a dog with three or more parts; to copy shapes correctly; and/or to trace a line within a box (FIG. 12A). The ability of the child to perform may be recorded on the worksheet (FIG. 11A-D or FIG. 12A-E). The sound order awareness input data is evaluated against an answer key to generate a sound awareness evaluation result. The sound awareness evaluation result is a determination with respect to the presence or absence of sound order awareness foundational skills. In some embodiments, the sound order awareness evaluation result is included in the assessment (FIGS. 13A-13P).

In the second part of the method, a referral is generated referring the individual for services in areas where the evaluation results were deficient to promote at least one of fine receptive language, sound discrimination, sound order awareness and/or fine motor skills. In some embodiments, the assessment (FIGS. 13A-13P), which can include the receptive language result, the sound discrimination result, the sound order awareness result and/or the fine motor skill result is evaluated to generate the referral. In particular, if the fine motor or receptive language result indicates that the child has proficient fine receptive language skills, the child is not referred for services, such as, for example, occupational therapy to promote advancement in fine motor skills. If, however, the fine receptive language result indicates that the child is deficient with his or her fine receptive language skills, the child is referred for services, such as, for example, occupational therapy to promote advancement in fine motor skills.

Likewise, if the sound discrimination evaluation result indicates that the child has proficient sound discrimination, the child is not referred for services, such as, for example, occupational therapy to promote advancement in sound discrimination. If, however, the sound discrimination result indicates that the child is deficient with his or her sound discrimination, the child is referred for services, such as, for example, Speech therapy to promote advancement in sound discrimination.

If the sound order awareness evaluation result indicates that the child has proficient sound order awareness skills, the child is not referred for services, such as, for example, Speech therapy to promote advancement in sound order awareness. If, however, the sound order awareness result indicates that the child is deficient with his or her sound order awareness skills, the child is referred for services, such as, for example, Speech therapy to promote advancement in sound order awareness.

If the receptive language result indicates that the child has proficient receptive language skills, the child is not referred for services, such as, for example, Speech therapy to promote advancement in receptive language skills. If, however, the receptive language result indicates that the child is deficient with his or her receptive language skills, the child is referred for services, such as, for example, Speech therapy to promote advancement in receptive language skills.

In some embodiments, the second part of the method includes evaluating the child for ADHD, autism, anxiety, panic attacks, post-traumatic stress disorder (PTSD), depression, low self-esteem, etc. if the evaluation results in the second part of the method were deficient to promote at least one of fine receptive language, sound discrimination, sound order awareness and/or fine motor skills.

The ADHD evaluation in the second part of the method can include giving focused tips on a structured environment, making learning activities fun, multisensory activities, and short but repetitive opportunities for learning. If engagement is appropriate, but focus/attention to a task cannot be maintained for the age expected time for the child's age, then the method may refer the child to take a test that includes ADHD/ADD age specific questions and consider all factors that can affect attention and sustained focus. In some embodiments, if the child exhibits signs of ADHD/ADD, the method can be adapted to administer and score a Vanderbilt screen for parent and teacher (if applicable). These screens are diagnostic of ADHD/ADD.

Concerning autism, the second part of the method includes evaluating whether the child has challenges with social communication and interaction, has poor eye contact, refuses to engage in tasks, and/or lacks joint attention back and forth while doing assessment tasks. For example, if the child demonstrates restrictive/repetitive behaviors, interests, and activities such as, but not limited to, flapping hands, spinning, lining up toys/objects, sensory issues (upset by loud noises/smell/touch), and challenges with change of routine during tasks, the evaluation should include an autism evaluation.

If the autism evaluation of the present method may recommend activities and/or worksheets that are adapted to address the specific concerns of lack of engagement/language delay affecting social interaction, high concerns for Autistic Spectrum Disorder (ASD), or ASD confirmed. This will include activities that focus on strengthening receptive, expressive, and pragmatic language skills; specific games and activities addressing language goals. To improve sensory regulation, the autism evaluation of the present method may recommend specific games and activities to decrease sensory triggers whether they be hypo(low) or hyper(high) sensory. To address sensory seeking behaviors and/or sensory avoidance behaviors, the autism evaluation may include recommending Occupational Therapy (OT) like play activities with OT therapy goals addressing visual/motor integration, sensory regulation, visual perceptual skills, executive function, and primitive reflex integration.

The system and method of the present disclosure can be used to treat and strengthen skills along with in-person therapy especially when waiting for in-person therapy. The system and method of the present disclosure can recommend starting therapeutic advancement of identified skill deficits while at home when awaiting in-person therapies. Such therapeutic advancement can be used to augment and reinforce the actual future or current therapies in place like medically delivered Speech and Language Therapy, OT, Cognitive Behavioral Therapy, and PT. The system and method of the present disclosure can include engagement modules and/or worksheets that allow Early Intervention to immediately start while a child waits for in-person evaluation and/or in-person autism specific therapy. The system and method of the present disclosure can improve skills and reassessment will allow progress. Children should continue using the system and method of the present disclosure even after starting in-person therapy. Referral pursuant to the autism evaluation can include referring the child for an in-person Speech and Language Evaluation, referring the child for an in-person Sensory Integration Focused Occupational Therapy Evaluation, referring the child for an in-person Diagnostic Evaluation for Autism, referring the child for in-person Child Play Therapy.

Concerning anxiety, panic attacks, or PTSD, if the child is showing emotions of frustration with or without crying and/or demonstrating worry about their performance while completing the assessment, has an elevated heart rate and/or blood pressure and/or avoids behaviors when tasks are hard such as: complaining to end or get out of a task like, having headaches, having nausea, stomach aches, vomiting, having tiredness, yawning, falling asleep, having a tense posture, clenched shoulders or muscles, clenched teeth, pulling on eyelashes, biting fingers or fingernails, arguing, throwing the materials, leaving the test environment, saying they are thirsty, hungry, tired, sleepy, or having to all of a sudden got to go to the bathroom, complains of their heart racing, is selectively mute, or is unable to attempt to complete the tests in part or at all, the evaluation should include an anxiety, panic attack, or PTSD evaluation.

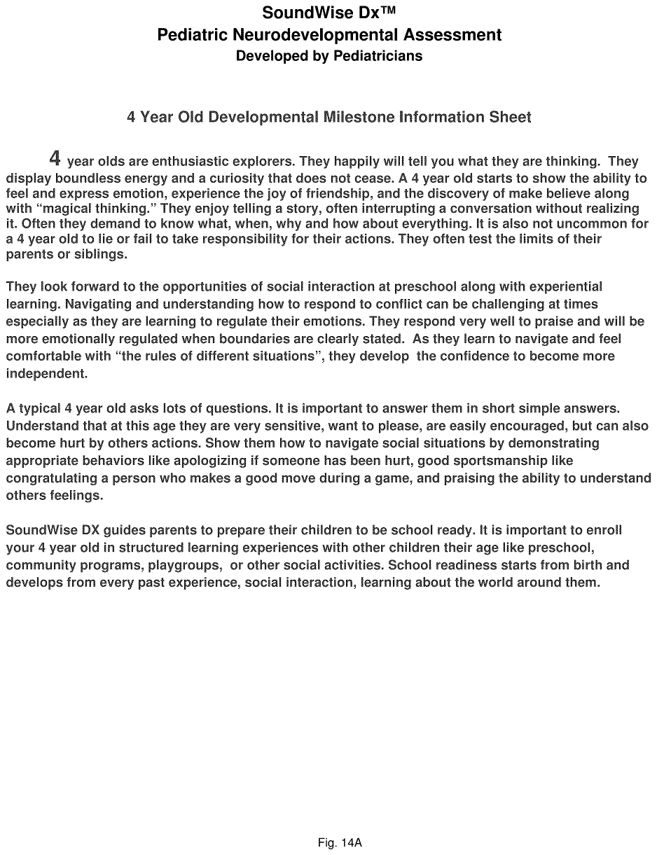

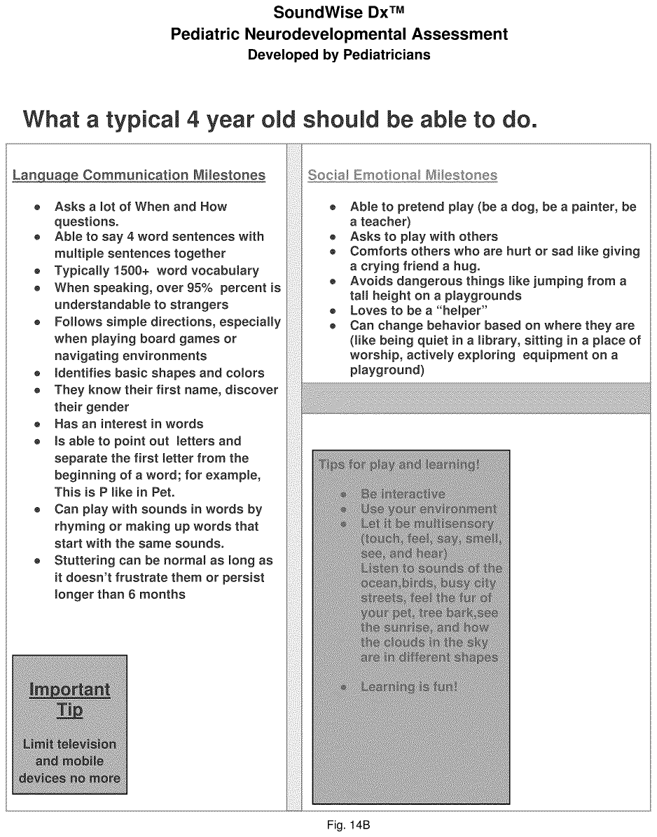

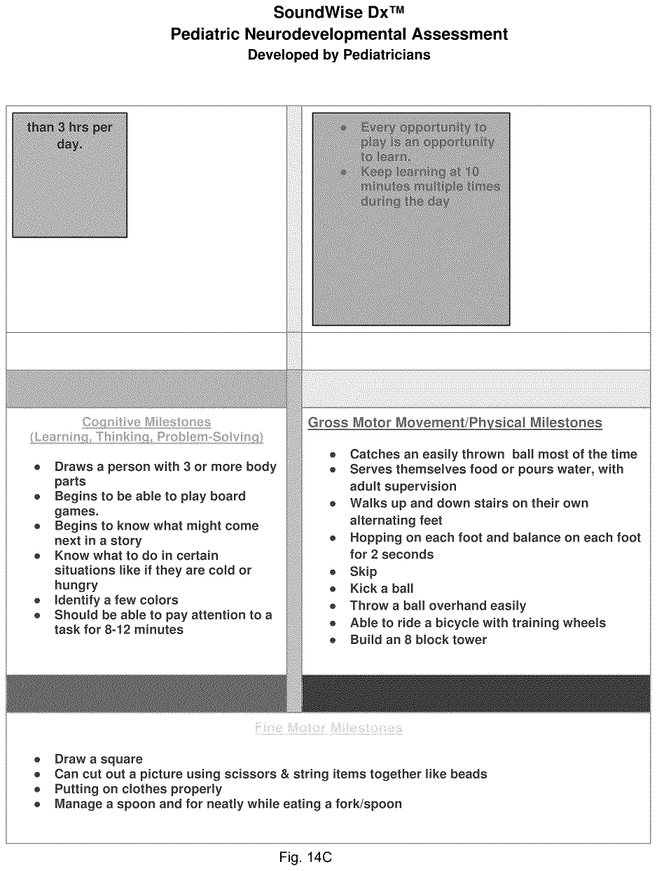

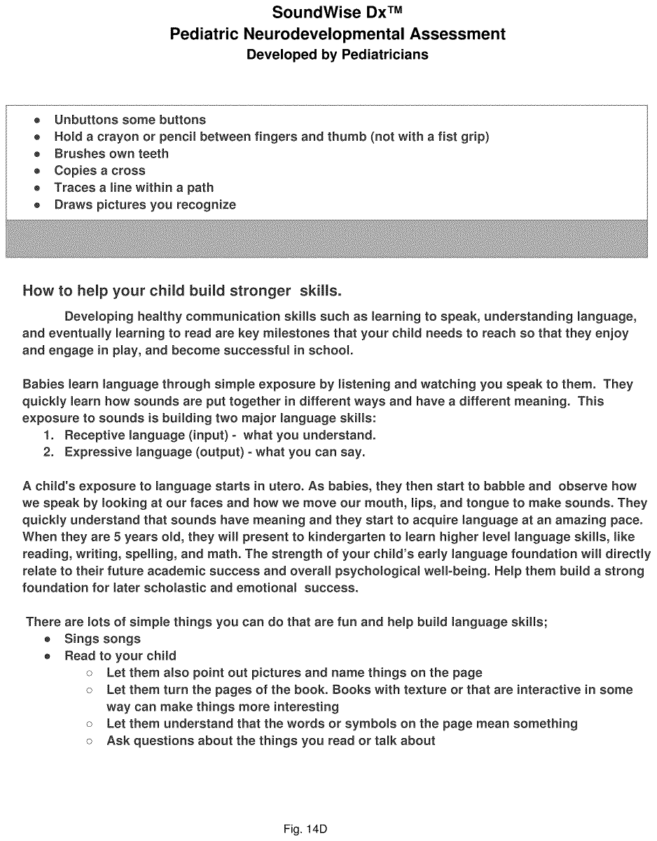

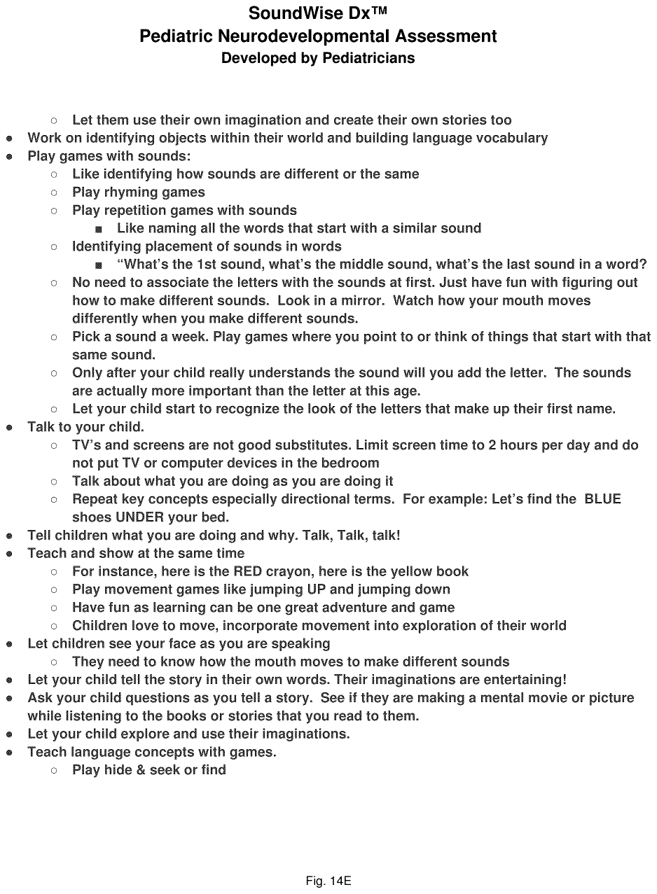

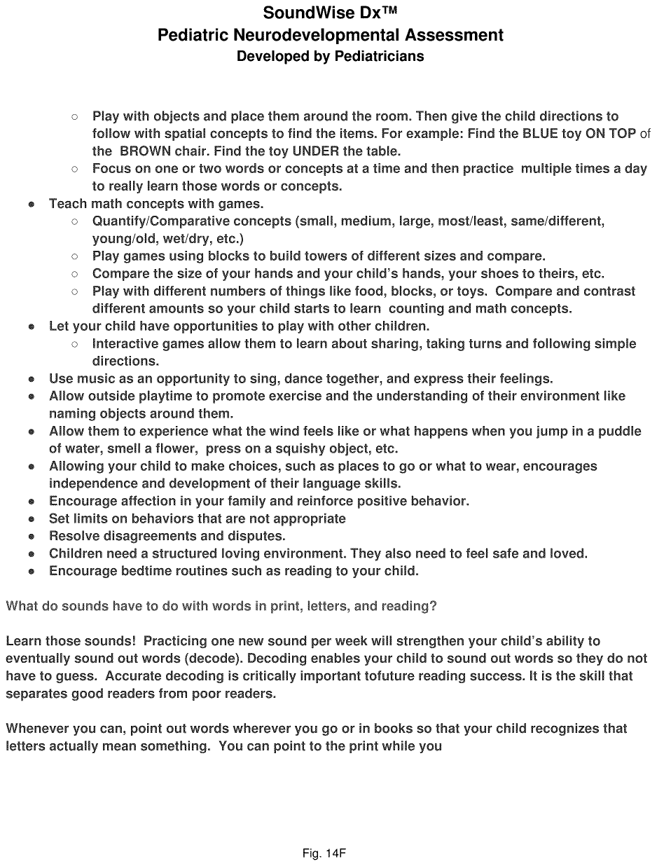

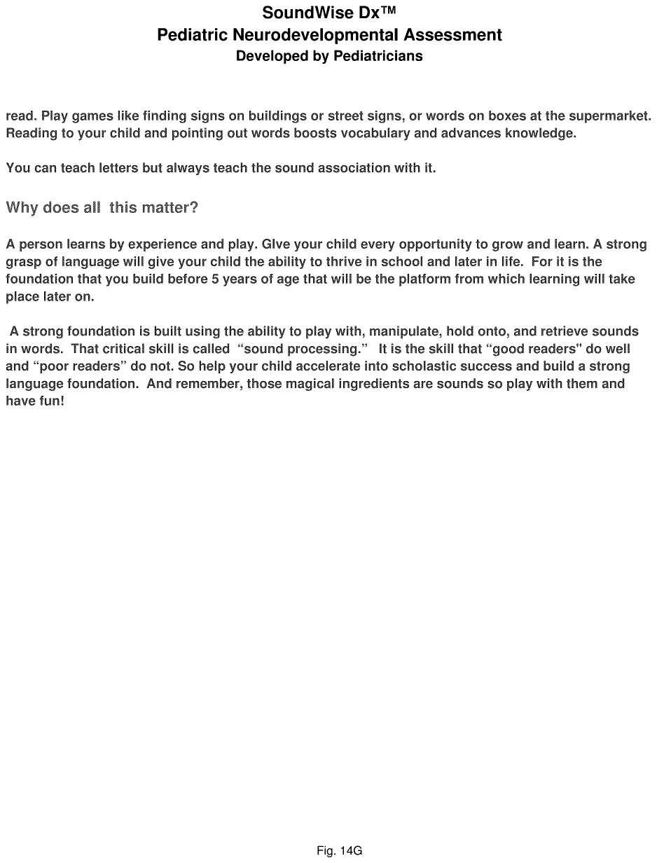

The system and method of the present disclosure may be configured to determine through an anxiety algorithm module and/or caregiver/teacher or authorized personnel to start Anxiety modules as well to start arranged telemedicine visits through the system and method of the present disclosure and/or in-person therapy. Also, the Caregiver/teacher will work on language development, social interaction, and sensory regulation by using a 4-Year-Old Developmental Milestone Information Sheet (DMIS) of the system and method of the present disclosure for age-appropriate activities. One example of a 4-Year-Old Developmental Milestone Information Sheet is shown in FIGS. 14A-14G. The system and method of the present disclosure may be configured to administer and score a diagnostic screen such as PHQ-9/SCARED/Columbia/Hamilton/GAD-7 or others for Anxiety that is appropriate for age. Based on the system and method of the present disclosure, Children will also be started on telemedicine services through the system and method of the present disclosure for anxiety.

Reasons for indications for medicine management for Anxiety is determined by the system and method of the present disclosure, physician, and input from caregiver/teacher. Pulse, Blood Pressure and EKG will be obtained by digital devices and are medically necessary before starting medication. Telemedicine services for anxiety though the system and method of the present disclosure must also be initiated and continued while the child is on medication for anxiety. All children diagnosed with Anxiety through the system and method of the present disclosure will also be evaluated for ADHD/ADD and/or Autism.

Concerning low self-esteem, self-esteem should always be monitored closely in a child as this can lead to depression or other mental health disparities in the future. Low self-esteem can arise from a variety of reasons. The system and method of the present disclosure may include one or more modules that address building self-esteem and confidence in a child. Telemedicine visits will be recommended via the system and method of the present disclosure or a local provider. Anxiety will be less when a child can confidently and competently perform what is asked of them.

In the second part of the method, a referral may also generate referring the individual for services in areas where the evaluation results were sufficient to improve the child's skills, even though the child's skills are proficient for his or her age. For example, if the child demonstrates that he or she is able to demonstrate social communication, interaction, eye contact and able to demonstrate joint attention back and forth with assessment tasks, it may be indicative that the child's skills are not deficient, and the child needs to only to continue to build and/or improve his or her skills. Further, not showing restrictive and repetition behaviors, such as flapping their hands, spinning a lot, being only interested in lining up toys or objects, having sensory issue such as being upset with loud noises and difficulty with change of routine may also be indicative that the child's skills are not deficient, and the child needs to only to continue to build and/or improve his or her skills. Indeed, if the child is able to maintain attention, is not easily distracted, follows directions, etc. it may also be indicative that the child's skills are not deficient, and the child needs to only continue to build and/or improve his or her skills. Likewise, if the child is able to complete the tests without crying or worrying about how they are doing on the tests, it may also be indicative that the child's skills are not deficient, and the child needs to only to continue to build and/or improve his or her skills.

In some embodiments, the second part of the method further includes observing behavioral engagement skills of the child throughout the testing process, such as, for example, during the first test of the second part of the method, during the second test of the second part of the method, during the third test of the second part of the method and/or during the fourth test of the second part of the method. In some embodiments, the observations made throughout the testing process of the second part of the method can include noting the child's demeanor, such as, for example, observations pertaining to whether the child is cooperative, uncooperative, refuses to interact, completed the tests independently, exhibited poor eye contact, smiled, cried, was tired, would not engage, was shy, was distractible, was anxious, needed redirection for completion, was happy and engaged, was an English language learner, was difficult to motivate, etc. In some embodiments, the observations made throughout the testing process of the second part of the method are included in the worksheet (FIG. 11A-11D or 12A-12E) and/or are used to generate the referral. In some embodiments, information pertaining to the answers provided in the questionnaire of the second part of the method (FIG. 8) is included in the worksheet and/or is used to generate the referral.

### Method—Part Three—Year Five

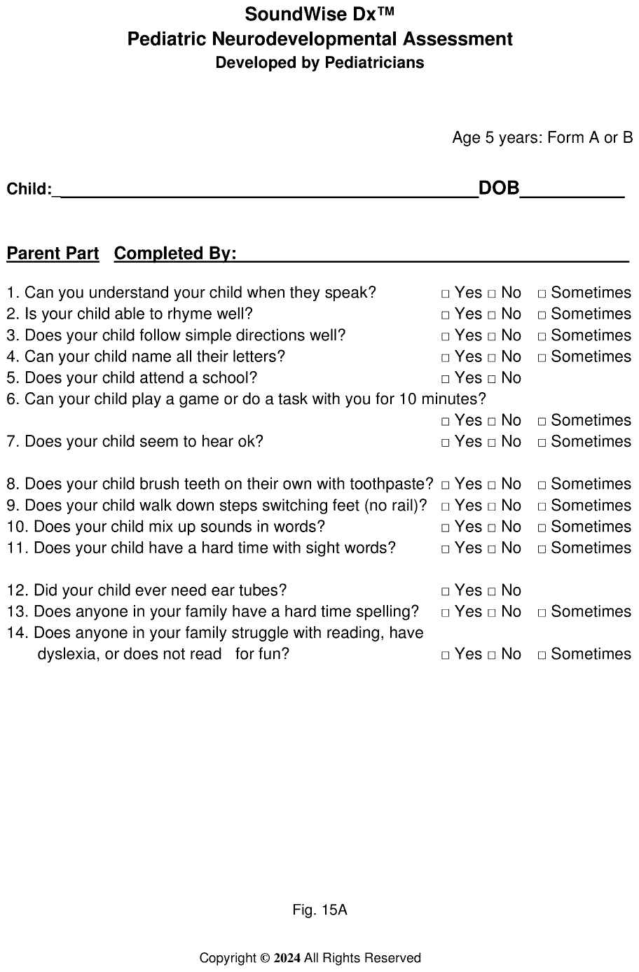

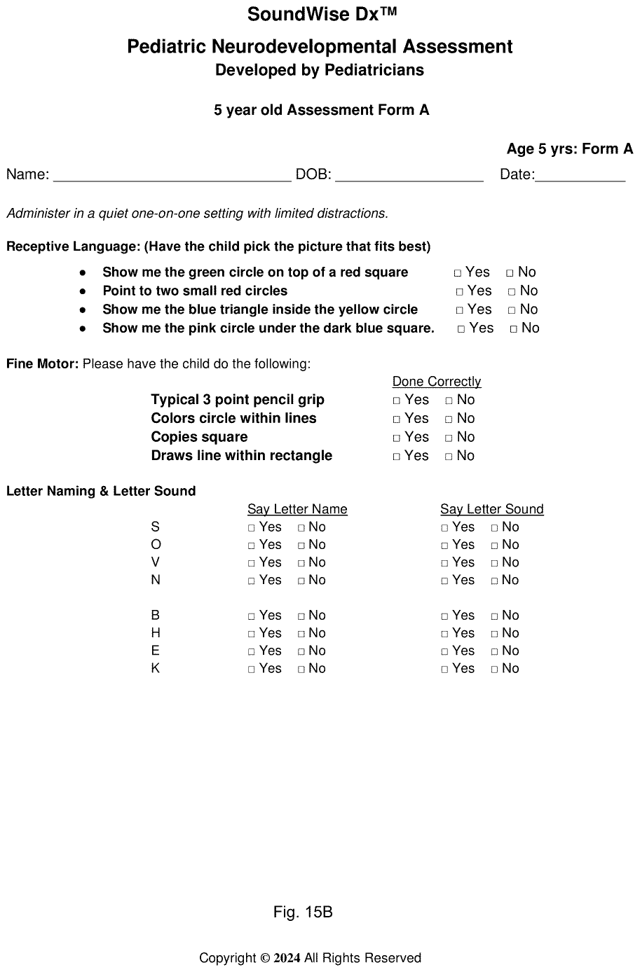

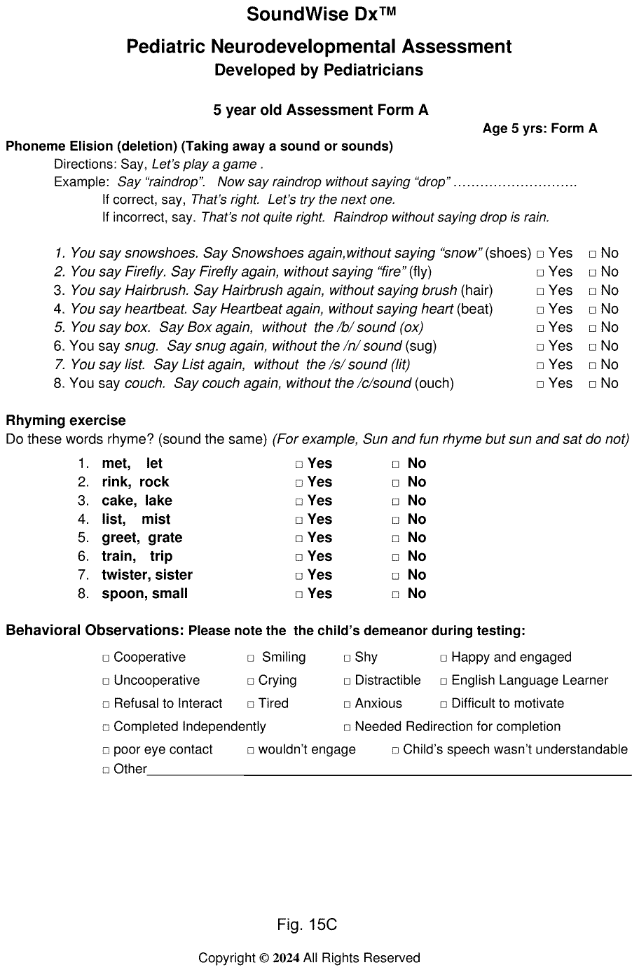

In a third part of the method, which should be administered after the second part of the method discussed above is administered to the child, when the child is approximately 5 years old, the child's parent's is required to fill out a questionnaire that includes various questions about the child. One embodiment of the questionnaire is shown in FIG. 15A. As shown in FIG. 15A, the questionnaire can include questions about the child's physical abilities, such as, for example, the child's hearing and vision. The questionnaire can also include questions relating to the child's ability to read, write, comprehend information, perform simple tasks, socialize, etc. For example, the questionnaire can include questions whether they can understand their child when the child speaks, whether the child is able to rhyme well, whether the child seems to hear OK, whether the child brushes his or her teeth with toothpaste, whether the child mixes up sounds in words, whether the child ever needed ear tubes, whether there is a family history of disorders, etc. (See, FIG. 15A).

In the third part of the method, the child may be required to perform tests relating to receptive language, fine motor and letter naming and letter sound skills. For example, as shown in FIG. 15B, the child may be required to show a green circle on top of a red square, to point to two small red circles, to show the blue triangle inside the yellow circle, to show the pink circle under the dark blue square. The child may also be required to perform a typical three-point pencil grip, color a circle within lines, copy squares and/or draw a line within a rectangle (FIG. 15B). The child may also be required to save various letter names and/or letter sounds (FIG. 15B). In some embodiments, the document shown in FIG. 15B is a worksheet that is used in connection with the third part of the method. For example, the document shown in FIG. 15B may be used as a worksheet to record the child's test results during the third part of the method.

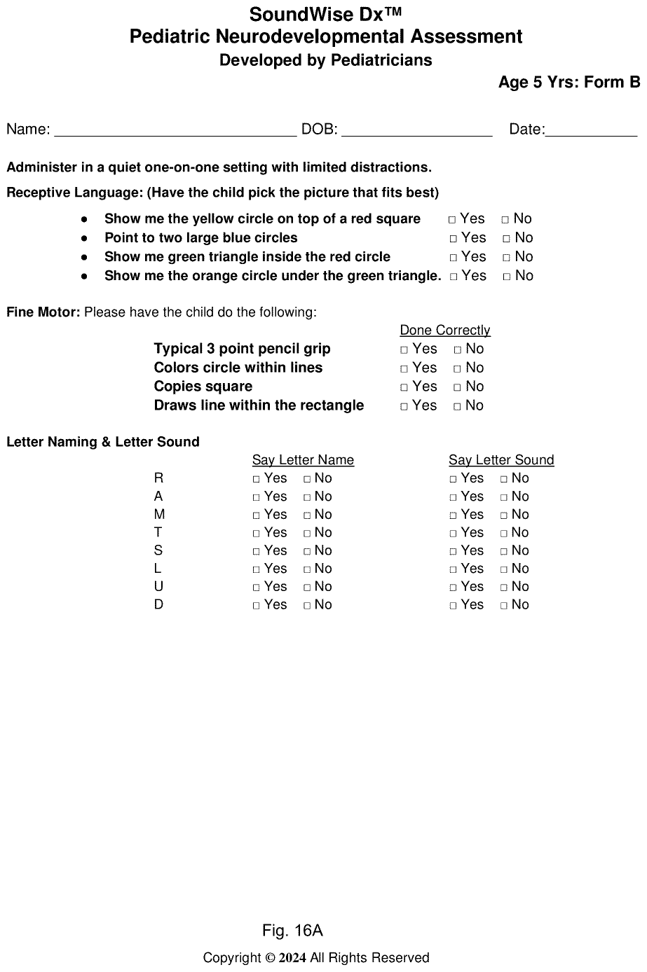

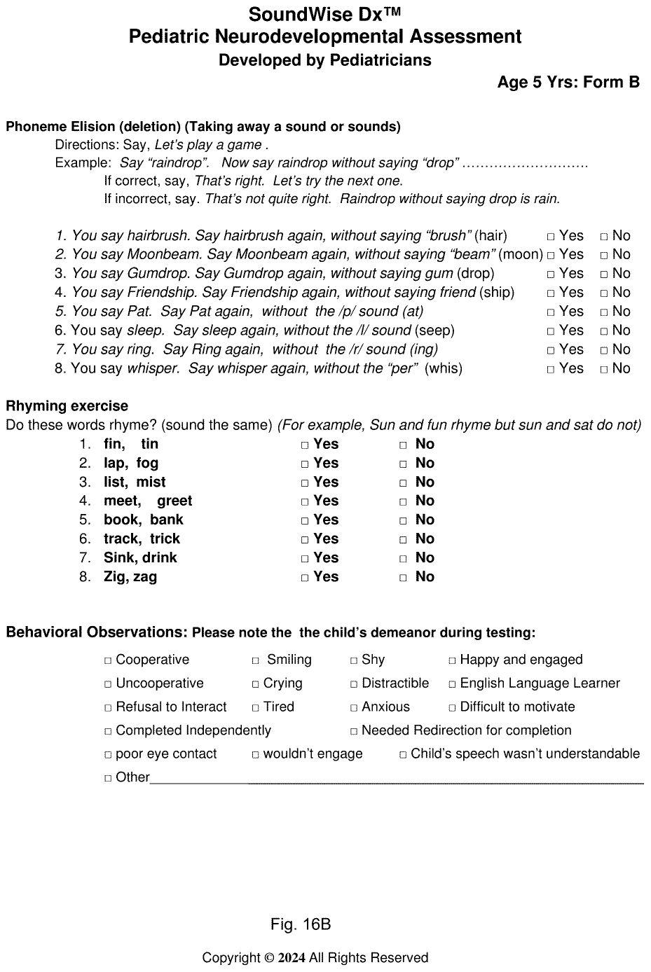

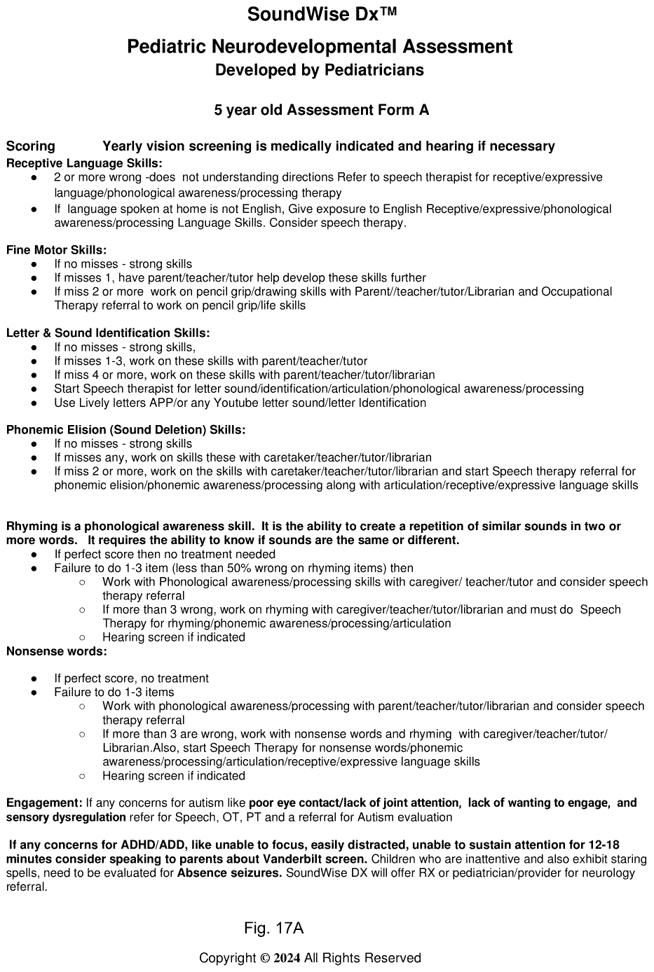

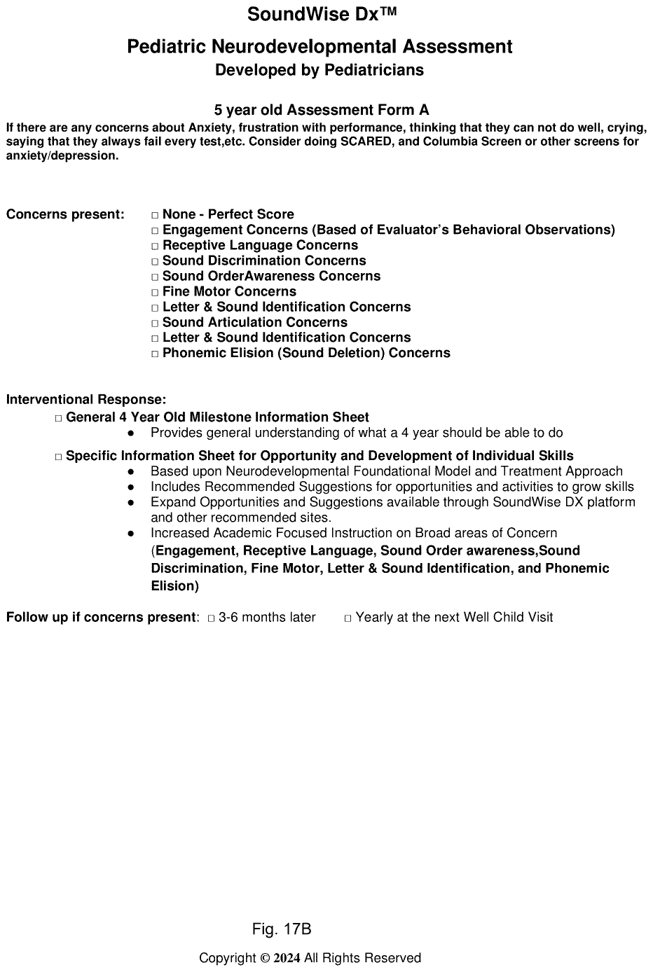

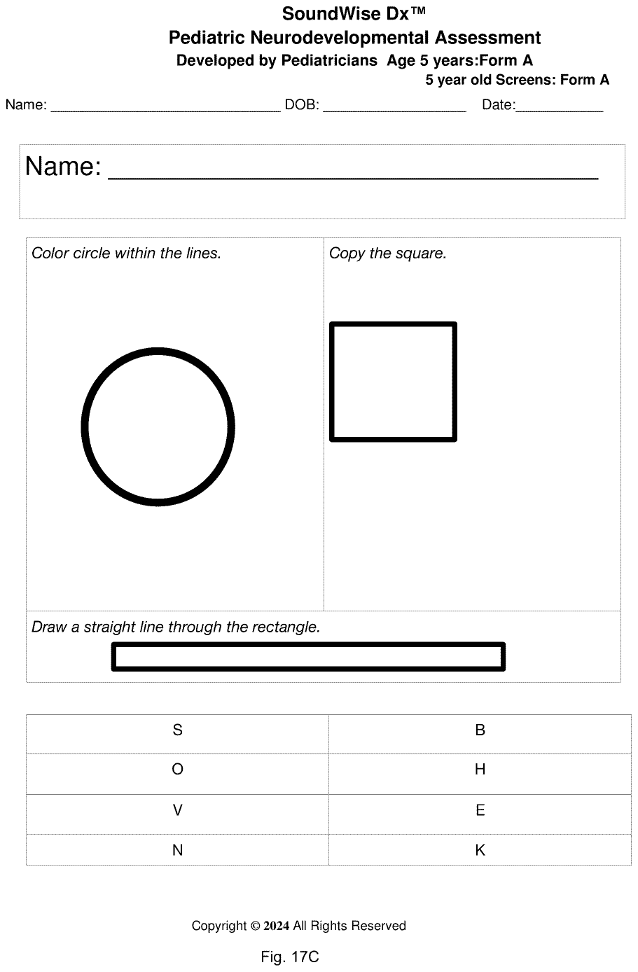

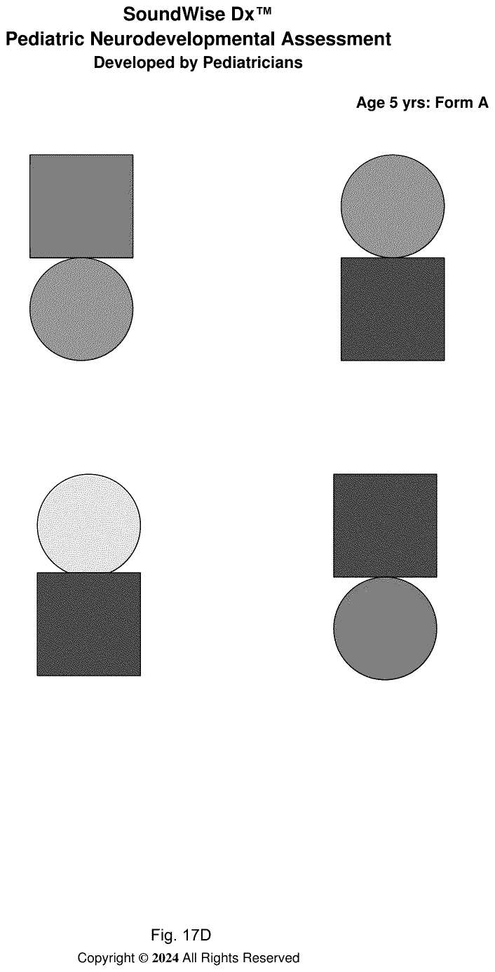

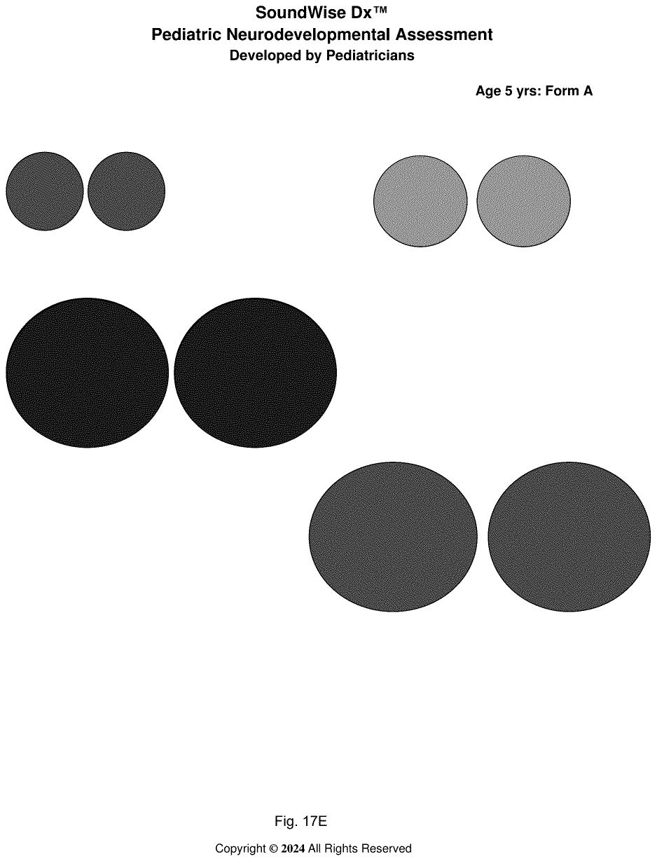

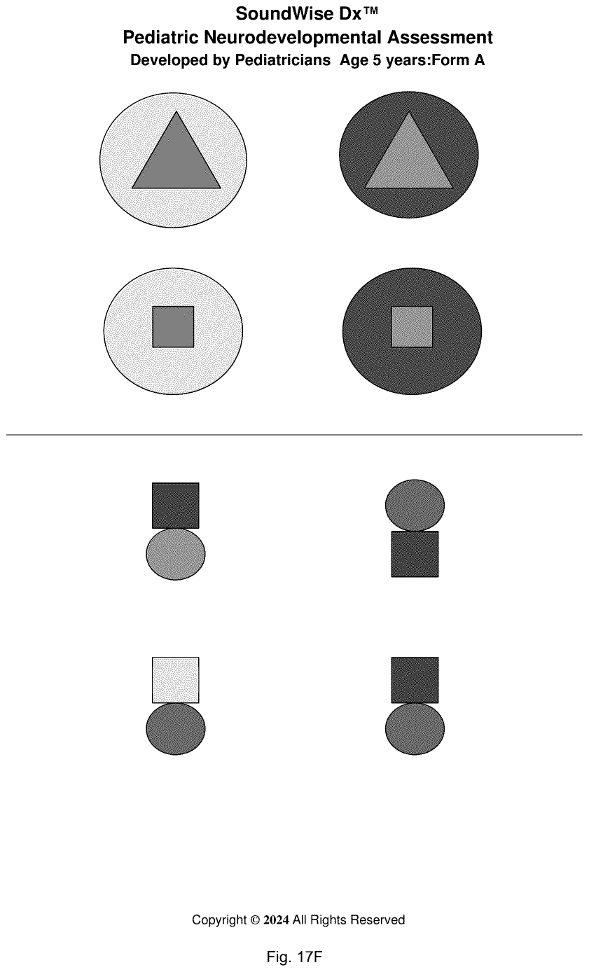

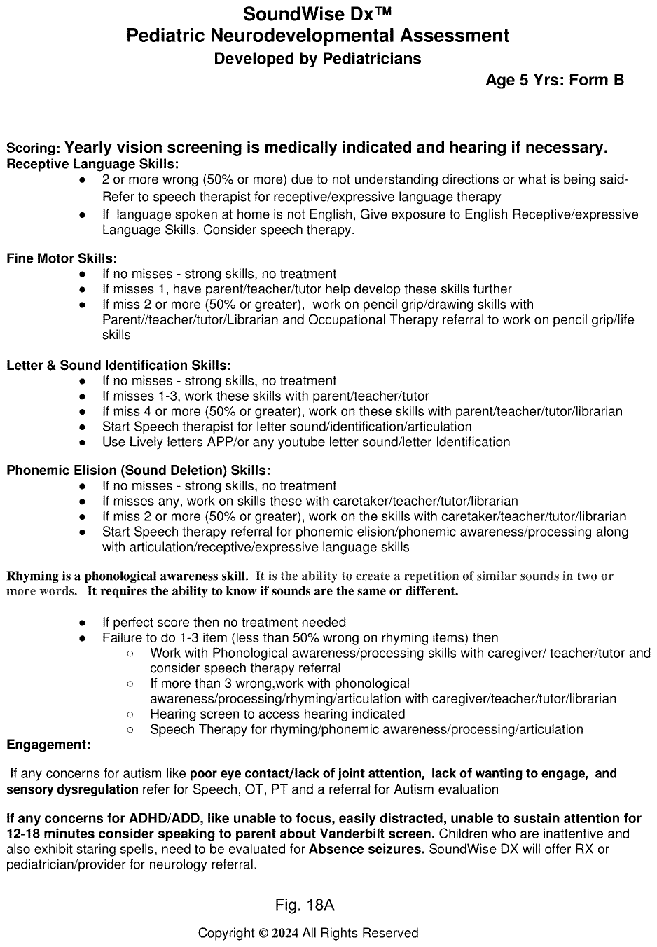

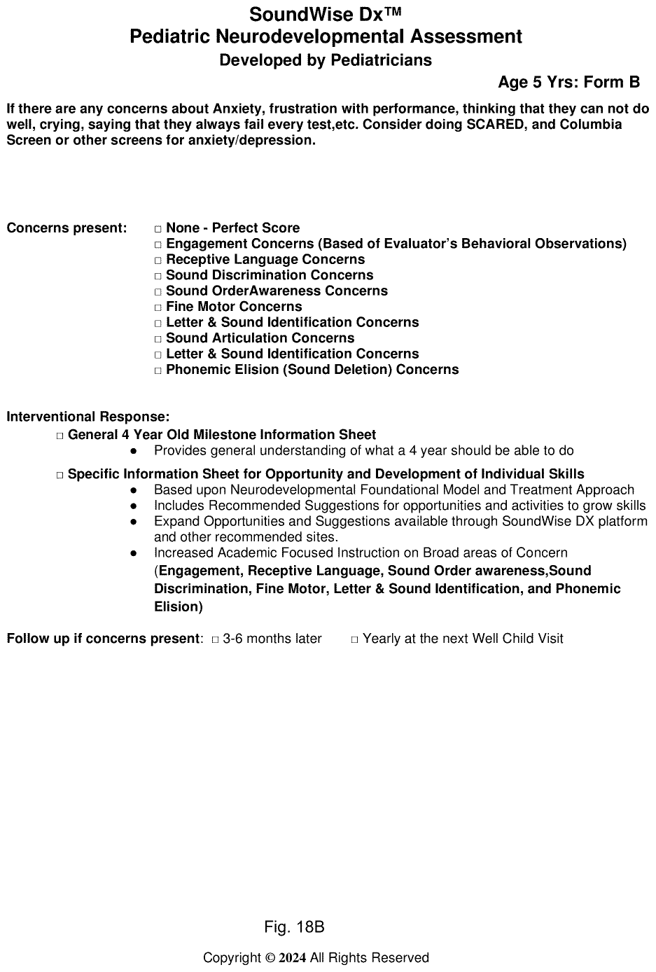

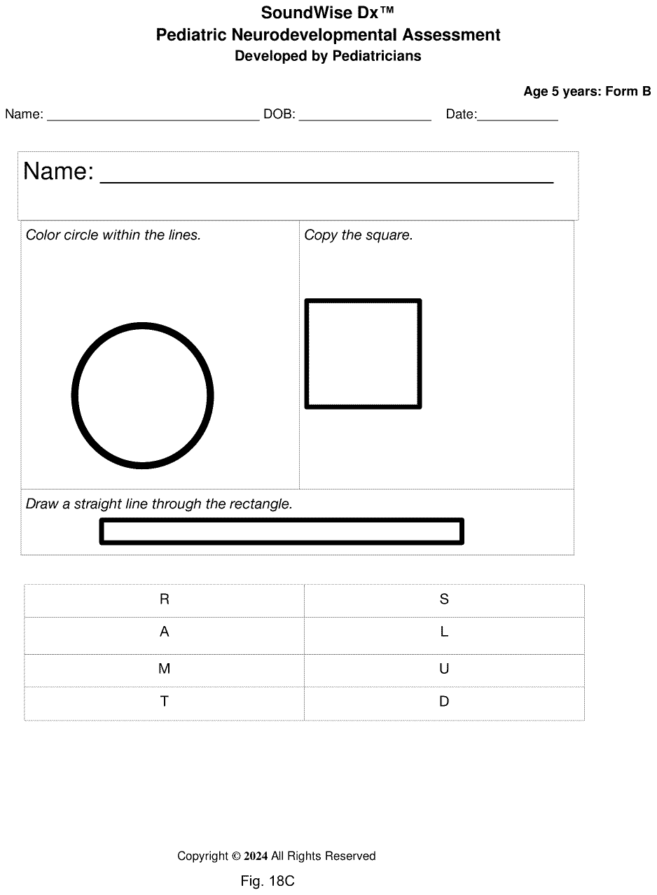

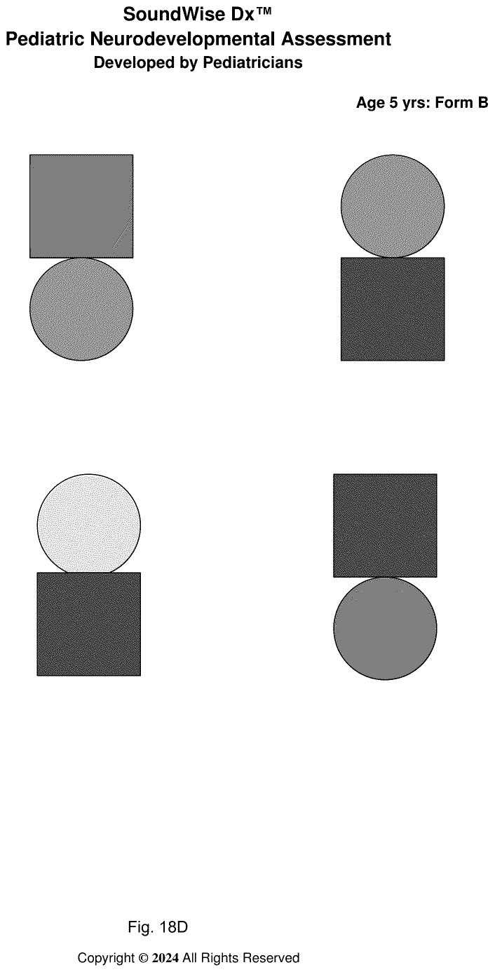

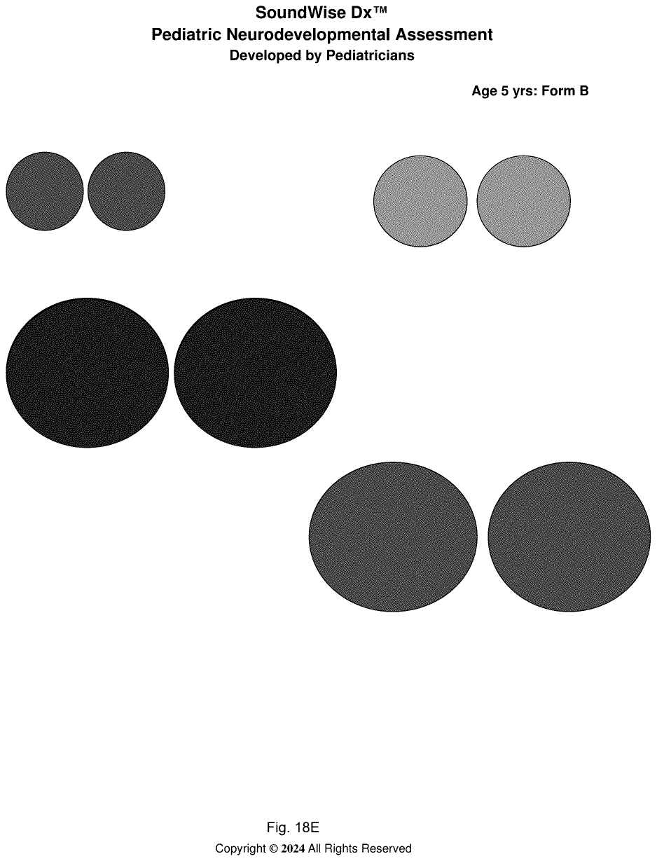

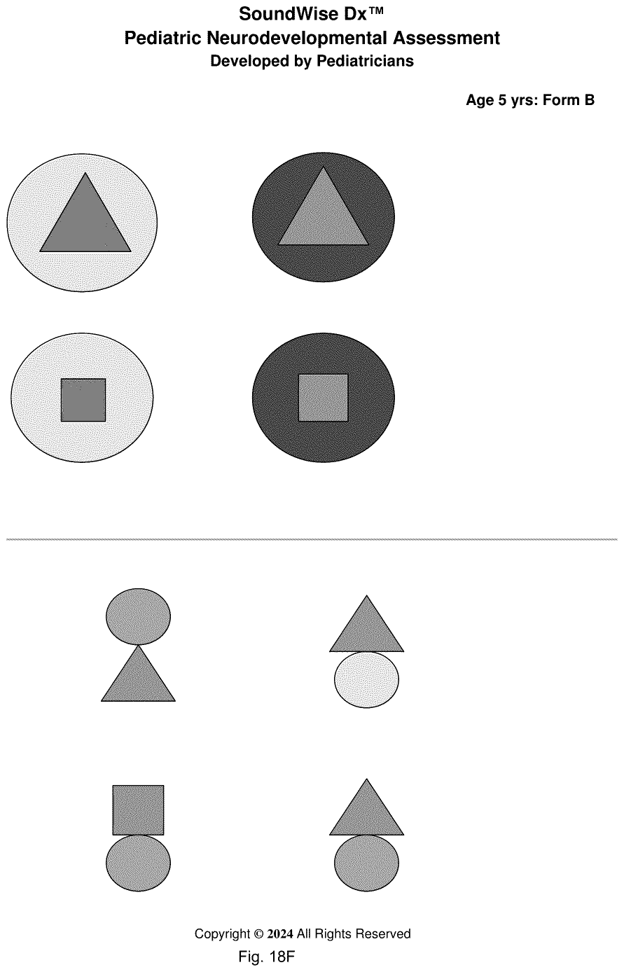

In the third part of the method, tests for perform tests relating to phoneme elision (deletion) and rhyming are performed (FIGS. 16A and 16B) prior to tests for fine motor skills, letter and sound identification skills, phonemic elision (sound deletion) skills (FIGS. 17A-F) and/or receptive language skills, fine motor skills, sound and letter identification skills and phonemic elision skills (FIGS. 18A-F). In some embodiments, the documents shown in FIGS. 16A and 16B can be part of a worksheet that is used in connection with the third part of the method. For example, the documents shown in FIGS. 16A and 16B may be used as worksheets to record the child's test results during the third part of the method. It is envisioned that the results of tests that are recorded in the documents shown in FIGS. 16A and 16B may be used to determine if the child is capable of understanding and following directions. For example, as shown in FIGS. 16A and 16B, the child may be required to play a game in which the child says a word, such as, for example, the word “raindrop,” and is then asked to say the word “raindrop” without saying “drop.” This may be repeated with other words, as shown in FIGS. 16A and 16B. In some embodiments, the child may be required to say whether or not two words rhyme, as also shown in FIGS. 16A and 16B. The child's behavior may be observed during these tests for considerations such as, for example, whether the child is cooperative, whether the child is uncooperative, whether the child refuses to interact, whether the child completed tasks independently, whether the child exhibited poor eye contact, whether the child smiled, whether the child cried, whether the child appeared tired, whether or not the child would engage, whether the child was shy, whether the child was distractible, whether the child was anxious, whether the child needed redirection to complete tasks, whether the child was happy and engaged, whether the child is an English language learner, whether the child was difficult whether the child's speech was understandable, etc., as shown in FIGS. 16A and 16B.

In the third part of the method, tests for fine motor skills, letter and sound identification skills, phonemic elision (sound deletion) skills (FIG. 17A) and/or receptive language skills, fine motor skills, sound and letter identification skills and phonemic elision skills (FIG. 18A) are evaluated for the presence or absence of a disorder and determination of school readiness. These tests may be administered after the tests discussed in the preceding paragraph and shown in FIGS. 16A and 16B. The ability of the child to perform the requested tasks is recorded to generate input data. The ability of the child to perform one or more of the requested tasks may be recorded in a third worksheet. One embodiment of the third worksheet is shown in FIGS. 17A-17F. Another embodiment of the third worksheet is shown in FIGS. 18A-18F.

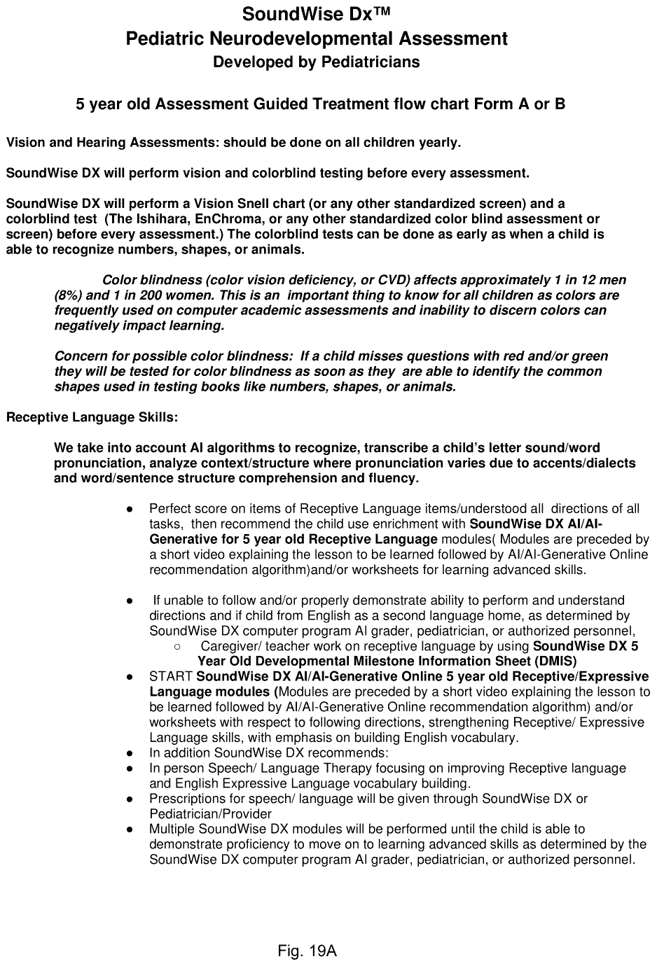

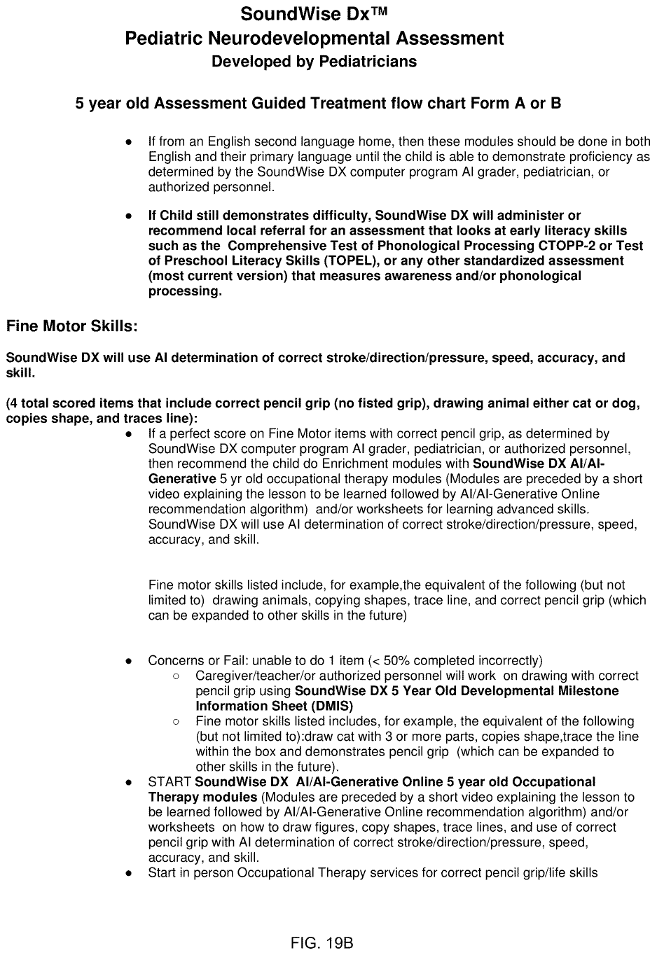

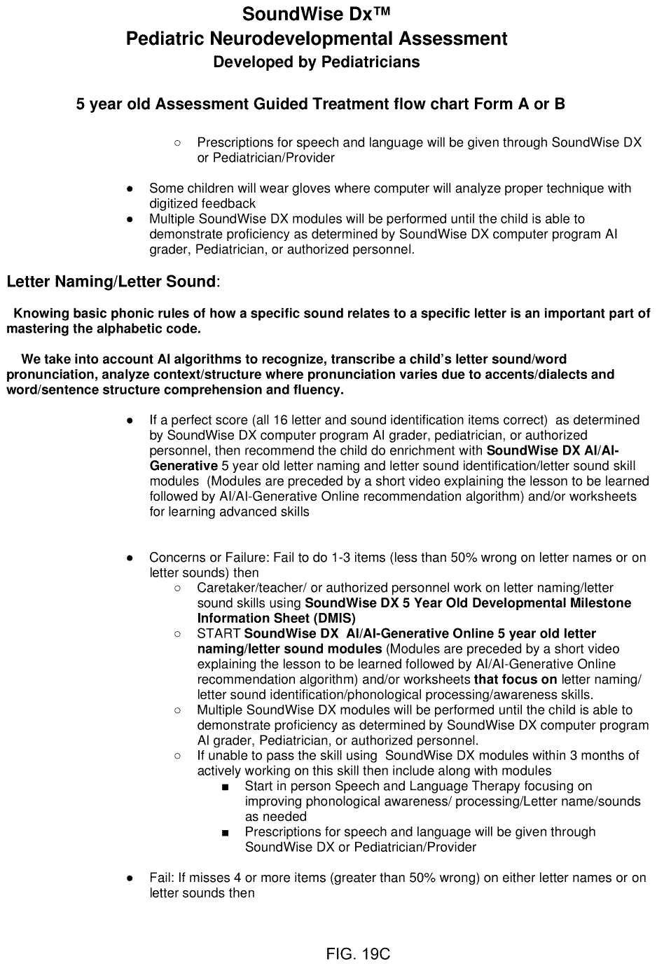

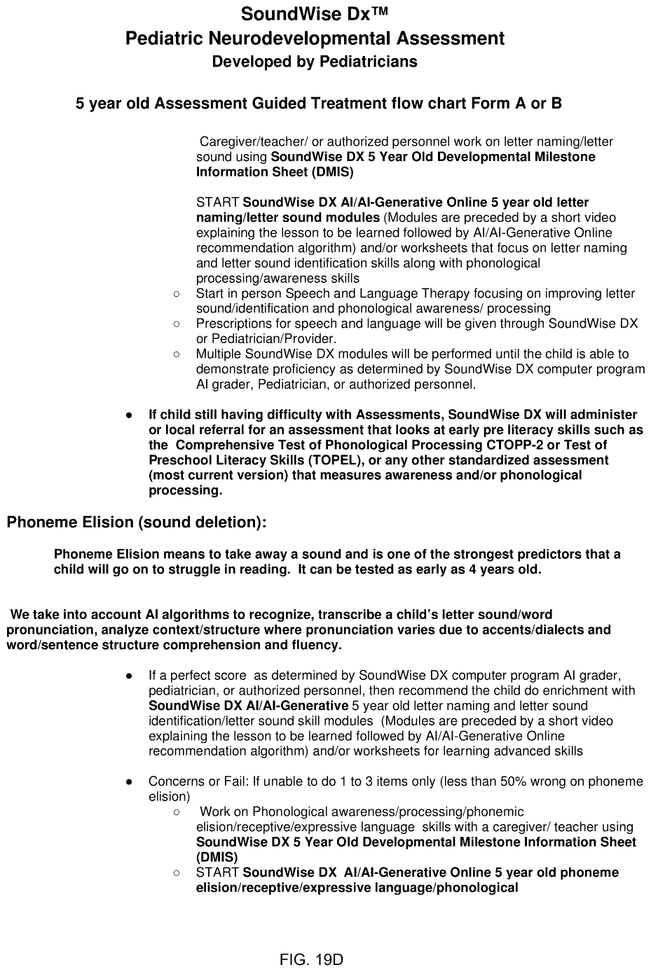

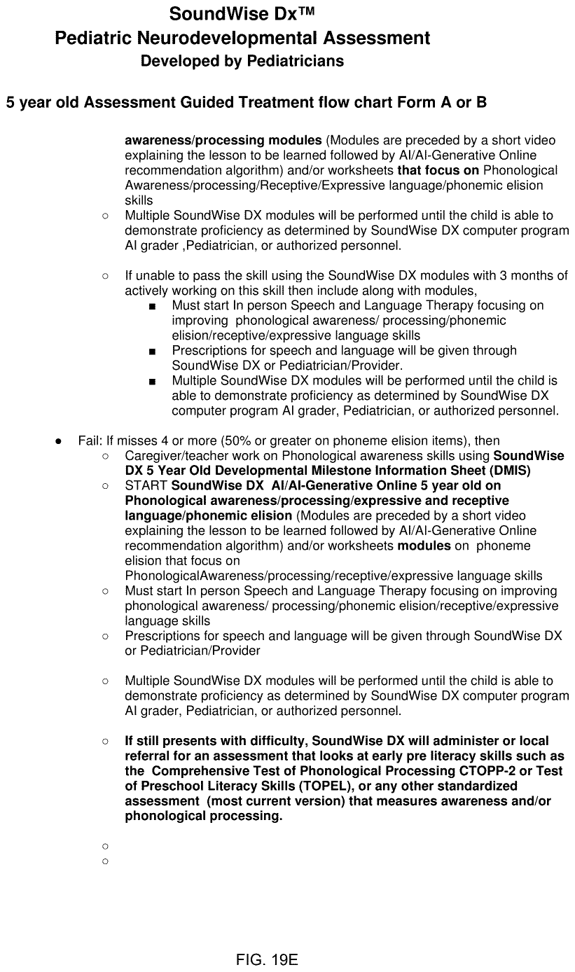

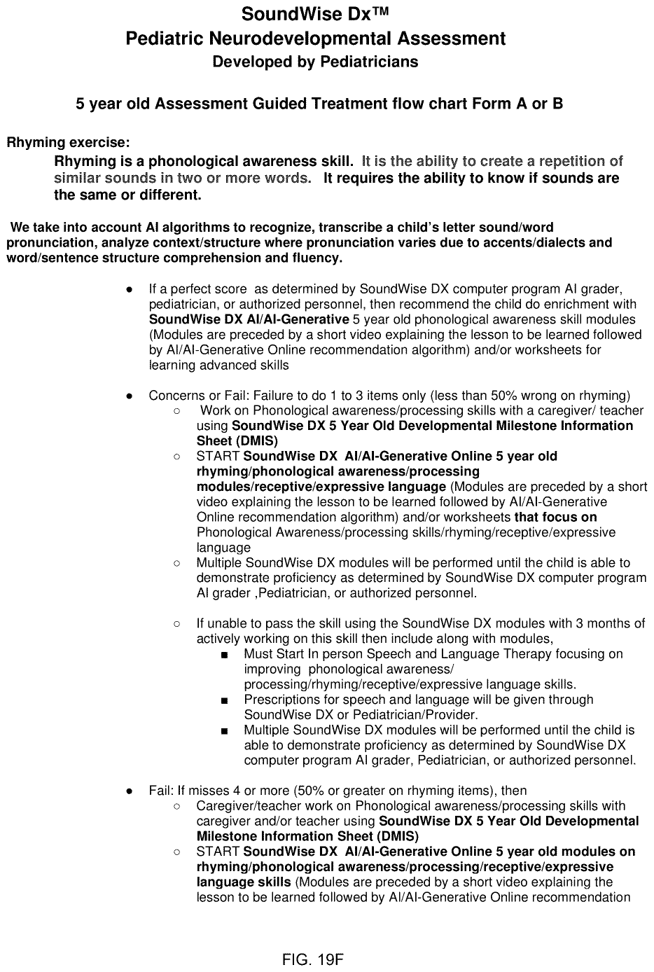

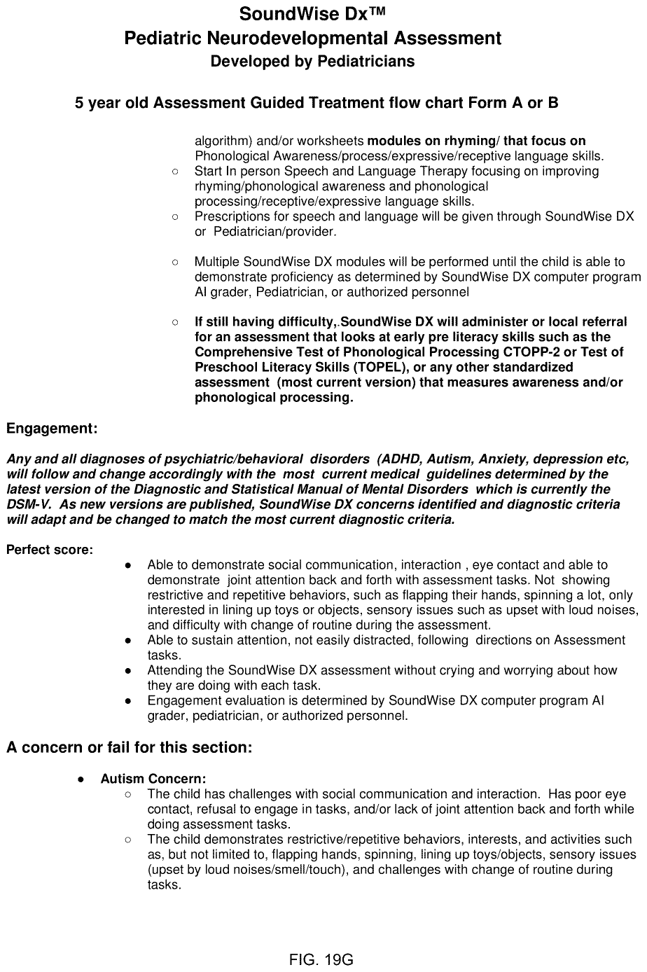

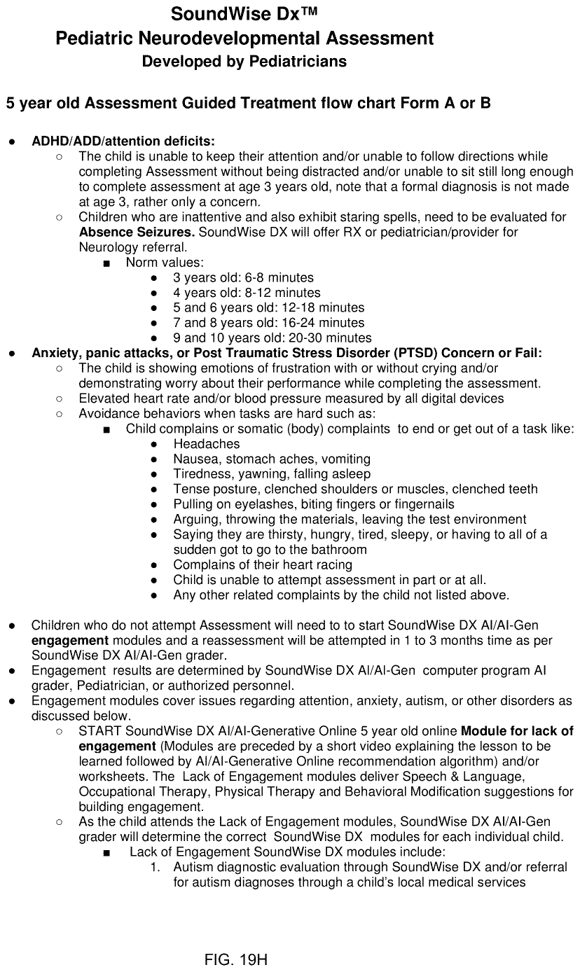

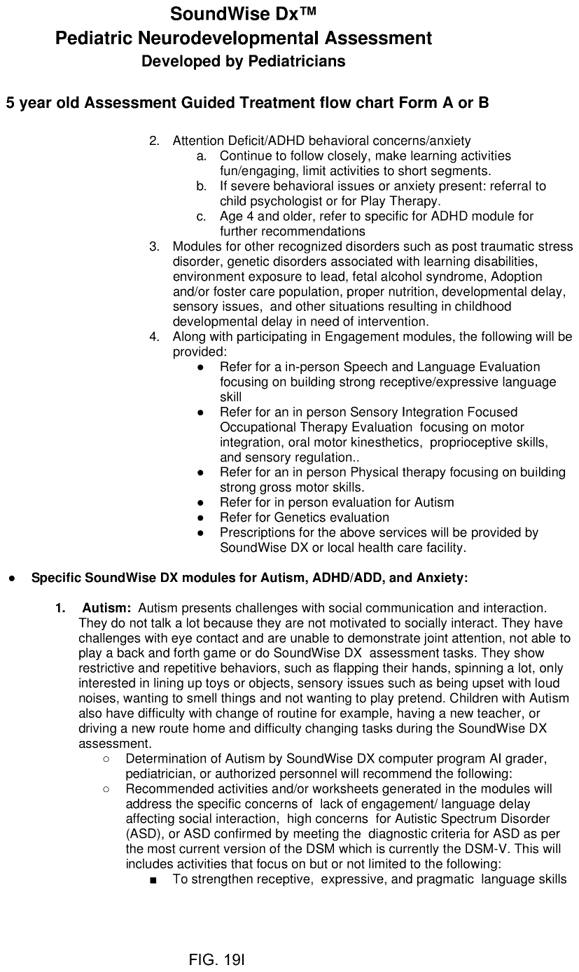

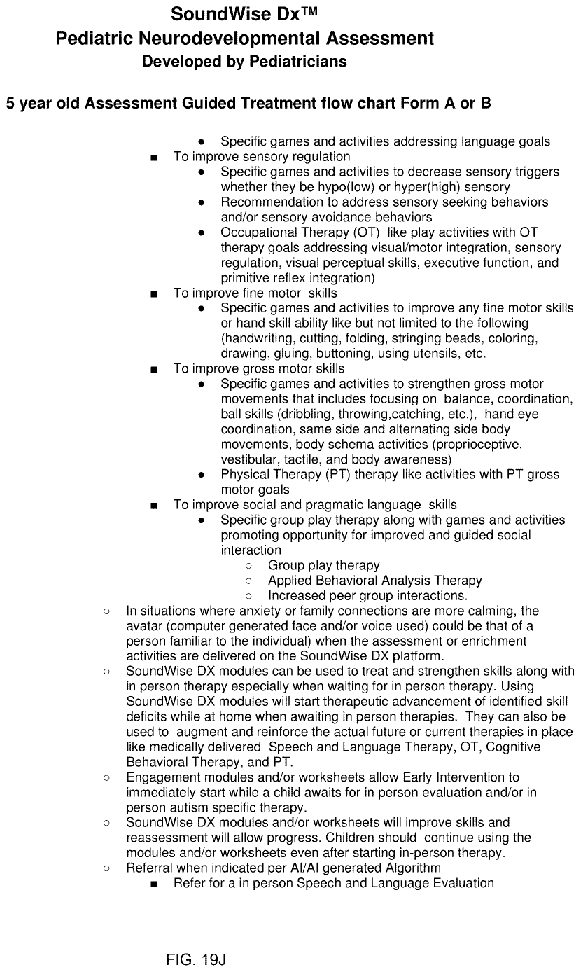

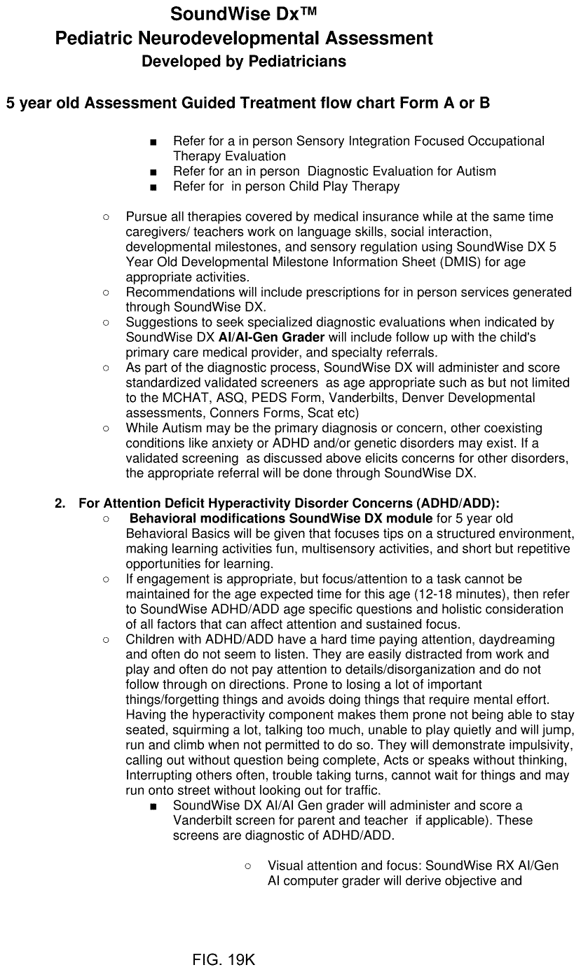

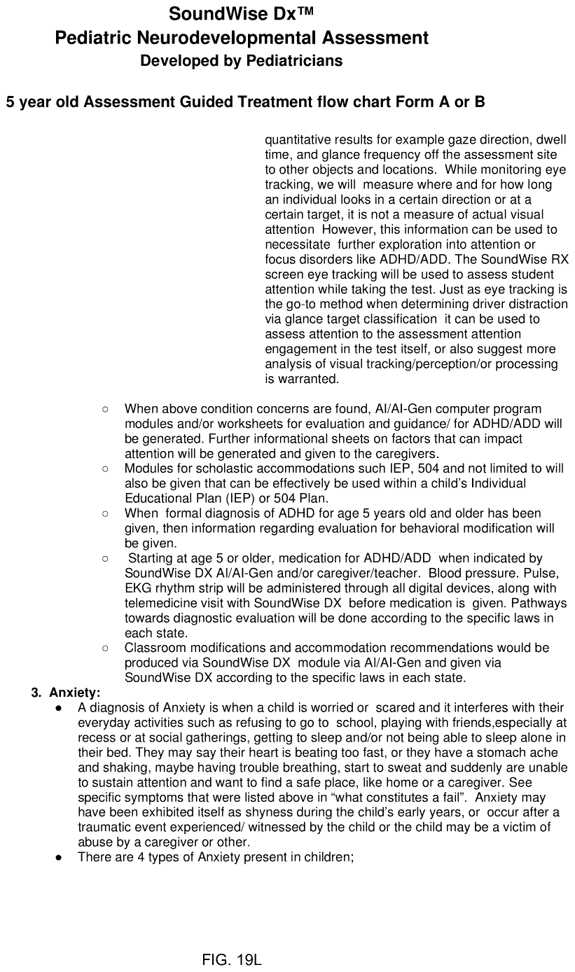

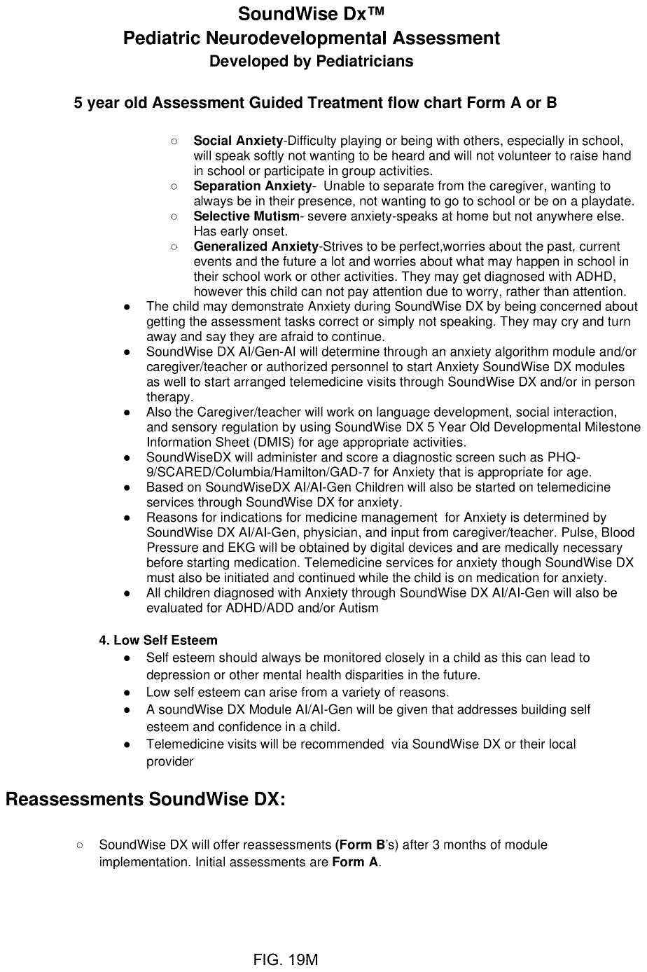

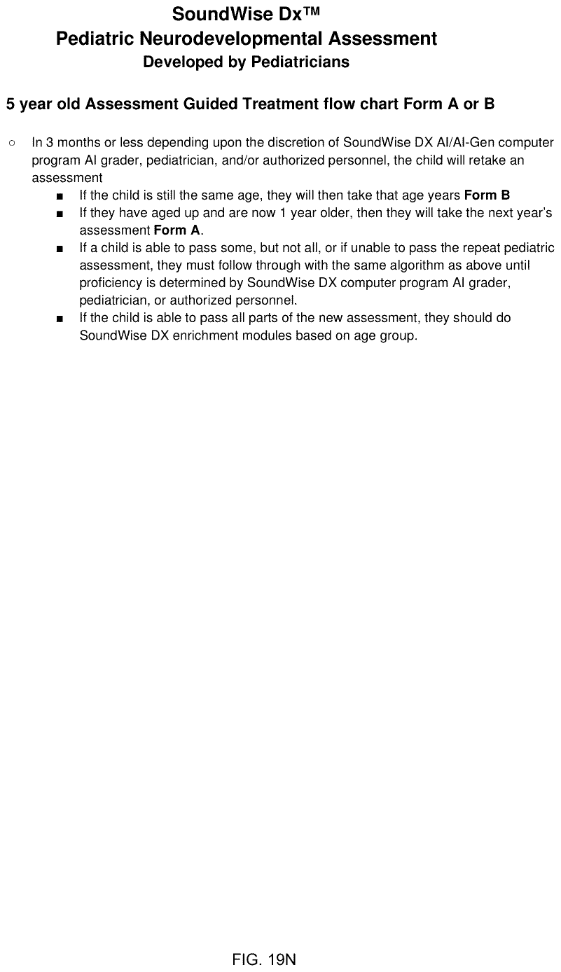

The fine motor skill input data may include whether or not the child was able to perform required tasks. The ability of the child to perform the required tasks may be recorded in the third worksheet. The fine motor input data is evaluated against an answer key to generate a fine motor evaluation result. The fine motor evaluation result is another determination with respect to the presence or absence of fine motor deficiency, after the second determination with respect the presence or absence of fine motor conducted during the first and second parts of the method. In some embodiments, the fine motor evaluation result includes a score, such as, for example, a number score. In some embodiments, the fine motor evaluation result includes a written evaluation, such as, for example, words written in sentence or note form that explain what was observed by the practitioner. In some embodiments, the fine motor result is included in an assessment that evaluates at least each of the tests administered during the third part of the method. One embodiment of the assessment is shown in FIGS. 19A-19N.

In the third part of the method, a second test is administered in which the child has to do a letter and sound identification task to generate a letter and sound identification task input data. The ability of the child to do a letter and sound identification task may be recorded in the third worksheet (FIG. 17A-17F or FIG. 18A-18F). The letter and sound identification input data are evaluated against an answer key to generate a letter and sound identification evaluation result. The letter and sound identification evaluation result are another determination with respect to the presence or absence of letter and sound identification deficiency, after the second determination with respect the presence or absence of letter and sound identification conducted during the first and second parts of the method. In some embodiments, the letter and sound identification evaluation result include a score, such as, for example, a number score. In some embodiments, the letter and sound identification evaluation result include a written evaluation, such as, for example, words written in sentence or note form that explain what was observed by the practitioner. In some embodiments, the letter and sound identification evaluation result is included in the assessment (FIGS. 19A-19N). In some embodiments, the letter and sound identification evaluation result uses AI or other software to recognize and/or transcribe the child's letter sound/word pronunciation, analyze context/structure where pronunciation varies due to accents/dialects and word/sentence structure comprehension and fluency. In the third part of the method, a third test is administered in which the child has phonemic elision (sound deletion) skills.

In the third part of the method, a third test is administered in which the child has to do a phonemic elision (sound deletion) task to generate a phonemic elision task input data. The ability of the child to do a phonemic elision task may be recorded in the third worksheet (FIG. 17A-17F or FIG. 18A-18F). The phonemic elision input data is evaluated against an answer key to generate a phonemic elision evaluation result. The phonemic elision evaluation result is another determination with respect to the presence or absence of phonemic elision deficiency, after the second determination with respect the presence or absence of phonemic elision deficiency conducted during the first and second parts of the method. In some embodiments, the phonemic elision evaluation result includes a score, such as, for example, a number score. In some embodiments, the phonemic elision evaluation result includes a written evaluation, such as, for example, words written in sentence or note form that explain what was observed by the practitioner. In some embodiments, the phonemic elision evaluation result is included in the assessment (FIGS. 19A-19N). In some embodiments, the phonemic elision evaluation result uses AI or other software to recognize and/or transcribe the child's letter sound/word pronunciation, analyze context/structure where pronunciation varies due to accents/dialects and word/sentence structure comprehension and fluency.

In the third part of the method, a fourth test is administered in which the child has to do a rhyming task to generate a rhyming task input data. The ability of the child to do a rhyming task may be recorded in the third worksheet (FIG. 17A-17F or FIG. 18A-18F). The rhyming task input data is evaluated against an answer key to generate a rhyming evaluation result. The rhyming evaluation result is another determination with respect to the presence or absence of rhyming deficiency, after the second determination with respect the presence or absence of rhyming deficiency conducted during the first and second parts of the method. Indeed, because rhyming is a phonological awareness skill, the fourth test will test the child's ability to create a repetition of similar sounds in to or more words, which requires the ability to know if the sounds are the same or different. In some embodiments, the rhyming evaluation result includes a score, such as, for example, a number score. In some embodiments, the rhyming evaluation result includes a written evaluation, such as, for example, words written in sentence or note form that explain what was observed by the practitioner. In some embodiments, the rhyming evaluation result is included in the assessment (FIGS. 19A-19N). In some embodiments, the rhyming evaluation result uses AI or other software to recognize and/or transcribe the child's letter sound/word pronunciation, analyze context/structure where pronunciation varies due to accents/dialects and word/sentence structure comprehension and fluency.

In one embodiment of the third part of the method, a fifth test is administered in which the child has to do a receptive language skill task to generate a receptive language skill input data. The ability of the child to do a receptive language skill may be recorded in the third worksheet (FIGS. 18A-18F). The receptive language skill input data is evaluated against an answer key to generate a receptive language skill evaluation result. The receptive language skill evaluation result is another determination with respect to the presence or absence of receptive language skills, after the second determination with respect the presence or absence of receptive language skill deficiency conducted during the first and second parts of the method. In some embodiments, the receptive language skill evaluation result includes a score, such as, for example, a number score. In some embodiments, the receptive language skill evaluation result includes a written evaluation, such as, for example, words written in sentence or note form that explain what was observed by the practitioner. In some embodiments, the receptive language skill evaluation result is included in the assessment (FIGS. 19A-19N). In some embodiments, the receptive language skill evaluation result uses AI or other software to recognize and/or transcribe the child's letter sound/word pronunciation, analyze context/structure where pronunciation varies due to accents/dialects and word/sentence structure comprehension and fluency.

In the third part of the method, a referral is generated referring the individual for services in areas where the evaluation results were deficient to promote at least one of the skills tested in the third part of the method. In some embodiments, the assessment (FIGS. 19A-19N), which can include one or more of the test results in the third part of the method are evaluated to generate the referral. In particular, if the results indicate that the child has proficient fine motor skills, the child is not referred for services, such as, for example, occupational therapy to promote advancement in fine motor skills. If, however, the test results indicate that the child is deficient with his or her fine motor skills, the child is referred for services, such as, for example, occupational therapy to promote advancement in fine motor skills. Likewise, if the results indicate that the child has proficient letter and sound identification skills, the child is not referred for services, such as, for example, Speech therapy to promote advancement in letter and sound identification skills. If, however, the test results indicate that the child is deficient with his or her letter and sound identification skills, the child is referred for services, such as, for example, Speech therapy to promote advancement in letter and sound identification skills. If the results indicate that the child has proficient phonemic elision skills, the child is not referred for services, such as, for example, Speech therapy to promote advancement in phonemic elision skills. If, however, the test results indicate that the child is deficient with his or her phonemic elision skills, the child is referred for services, such as, for example, Speech therapy to promote advancement in phonemic elision skills. If the results indicate that the child has proficient rhyming/phonological awareness skills, the child is not referred for services, such as, for example, Speech therapy to promote advancement in rhyming/phonological awareness skills. If, however, the test results indicate that the child is deficient with his or her rhyming/phonological awareness skills, the child is referred for services, such as, for example, Speech therapy to promote advancement in rhyming/phonological awareness skills. Lastly, if the results indicate that the child has proficient receptive language skills, the child is not referred for services, such as, for example, Speech therapy to promote advancement in receptive language skills. If, however, the test results indicate that the child is deficient with his or her receptive language skills, the child is referred for services, such as, for example, Speech therapy to promote advancement in receptive language skills.

In some embodiments, the third part of the method includes evaluating the child for ADHD, autism, anxiety, panic attacks, post-traumatic stress disorder (PTSD), depression, low self-esteem, etc. if the evaluation results in the third part of the method were deficient to promote at least one of the skills tested in the third part of the method.

The ADHD evaluation in the third part of the method can include giving focused tips on a structured environment, making learning activities fun, multisensory activities, and short but repetitive opportunities for learning. If engagement is appropriate, but focus/attention to a task cannot be maintained for the age expected time for the child's age, then the method may refer the child to take a test that includes ADHD/ADD age specific questions and consider all factors that can affect attention and sustained focus. In some embodiments, if the child exhibits signs of ADHD/ADD, the method can be adapted to administer and score a Vanderbilt screen for parent and teacher (if applicable). These screens are diagnostic of ADHD/ADD. Also, must ask and rule out absence seizures with EEG or another diagnostic device.

Concerning autism, the third part of the method includes evaluating whether the child has challenges with social communication and interaction, has poor eye contact, refuses to engage in tasks, and/or lacks joint attention back and forth while doing assessment tasks. For example, if the child demonstrates restrictive/repetitive behaviors, interests, and activities such as, but not limited to, flapping hands, spinning, lining up toys/objects, sensory issues (upset by loud noises/smell/touch), and challenges with change of routine during tasks, the evaluation should include an autism evaluation, if indicated.

If the autism evaluation of the present method may recommend activities and/or worksheets that are adapted to address the specific concerns of lack of engagement/language delay affecting social interaction, high concerns for Autistic Spectrum Disorder (ASD), or ASD confirmed. This will include activities that focus on strengthening receptive, expressive, and pragmatic language skills; specific games and activities addressing language goals. To improve sensory regulation, the autism evaluation of the present method may recommend specific games and activities to decrease sensory triggers whether they be hypo(low) or hyper(high) sensory. To address sensory seeking behaviors and/or sensory avoidance behaviors, the autism evaluation may include recommending Occupational Therapy (OT) like play activities with OT therapy goals addressing visual/motor integration, sensory regulation, visual perceptual skills, executive function, and primitive reflex integration.

The system and method of the present disclosure can be used to treat and strengthen skills along with in-person therapy especially when waiting for in-person therapy. The system and method of the present disclosure can recommend starting therapeutic advancement of identified skill deficits while at home when awaiting in-person therapies. Such therapeutic advancement can be used to augment and reinforce the actual future or current therapies in place like medically delivered Speech and Language Therapy, OT, Cognitive Behavioral Therapy, and PT. The system and method of the present disclosure can include engagement modules and/or worksheets that allow Early Intervention to immediately start while a child waits for in-person evaluation and/or in-person autism specific therapy. The system and method of the present disclosure can improve skills and reassessment will allow progress. Children should continue using the system and method of the present disclosure even after starting in-person therapy. Referral pursuant to the autism evaluation can include referring the child for an in-person Speech and Language Evaluation, referring the child for an in-person Sensory Integration Focused Occupational Therapy Evaluation, referring the child for an in-person Diagnostic Evaluation for Autism, referring the child for in-person Child Play Therapy.

Concerning anxiety, panic attacks, or PTSD, if the child is showing emotions of frustration with or without crying and/or demonstrating worry about their performance while completing the assessment, has an elevated heart rate and/or blood pressure and/or avoids behaviors when tasks are hard such as: complaining to end or get out of a task like, having headaches, having nausea, stomach aches, vomiting, having tiredness, yawning, falling asleep, having a tense posture, clenched shoulders or muscles, clenched teeth, pulling on eyelashes, biting fingers or fingernails, arguing, throwing the materials, leaving the test environment, saying they are thirsty, hungry, tired, sleepy, or having to all of a sudden got to go to the bathroom, complains of their heart racing, going selectively mute, or is unable to attempt to complete the tests in part or at all, the evaluation should include an anxiety, panic attack, or PTSD evaluation.

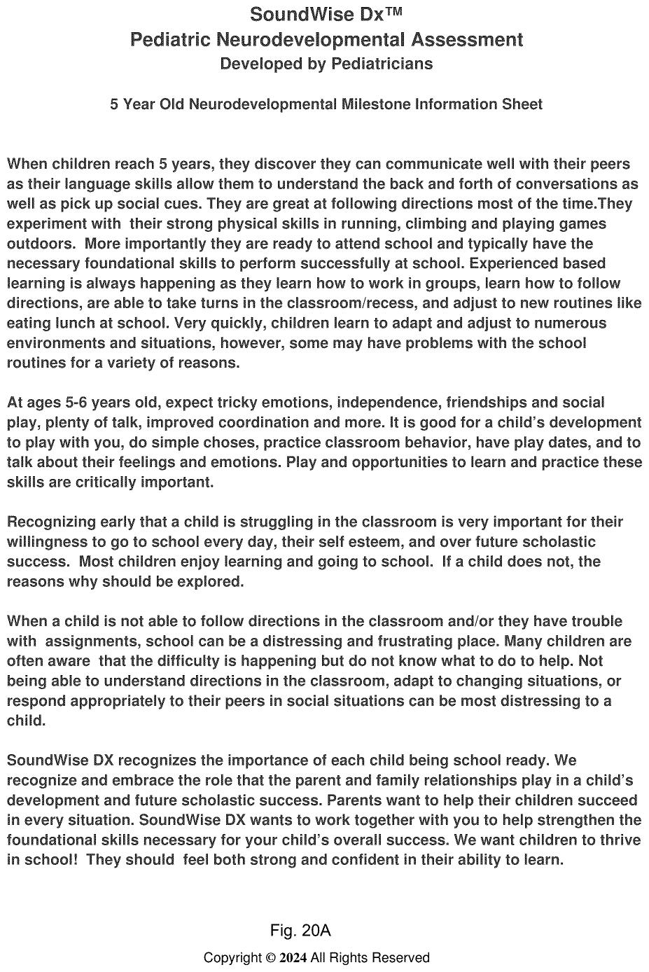

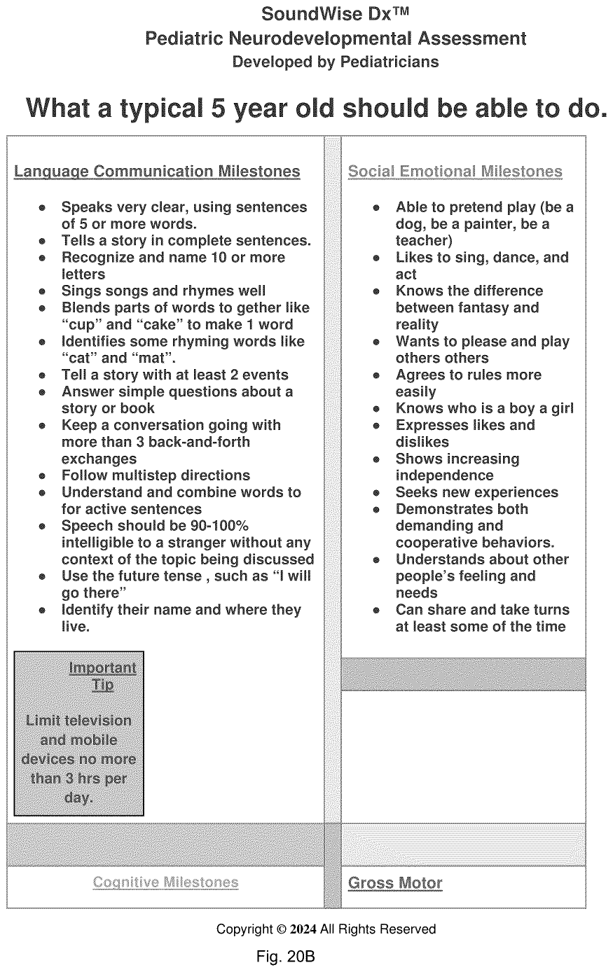

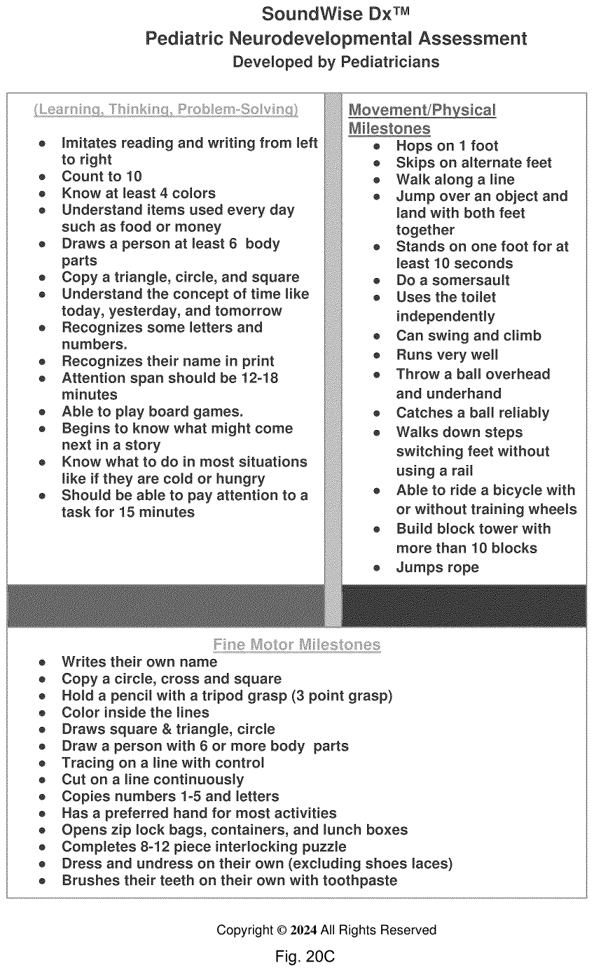

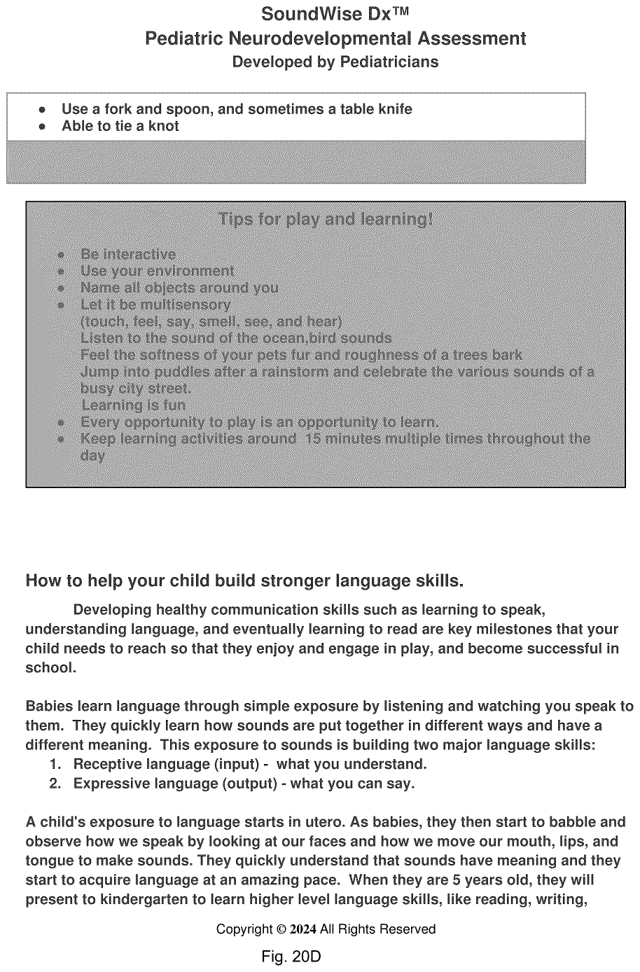

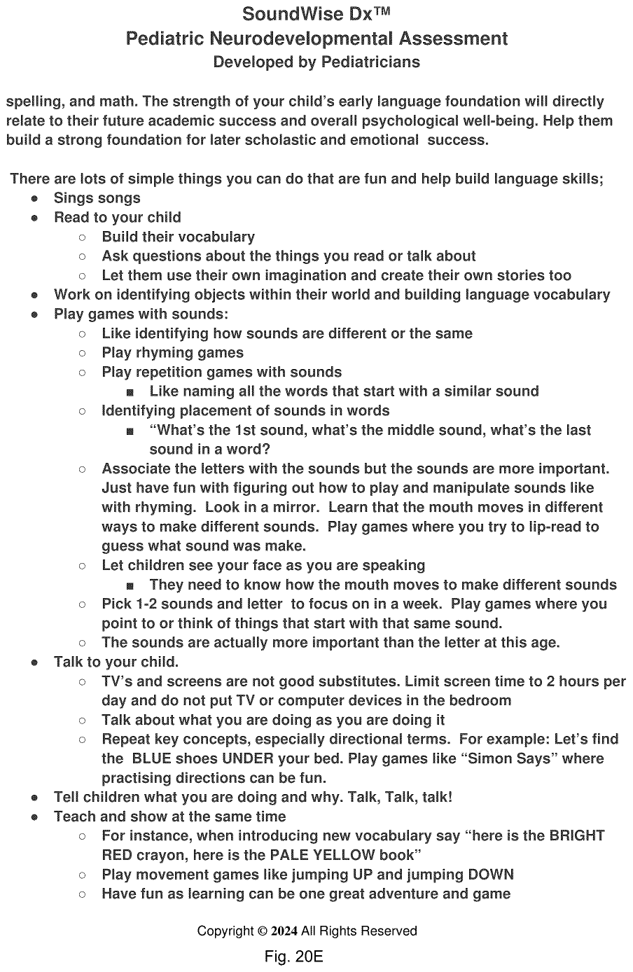

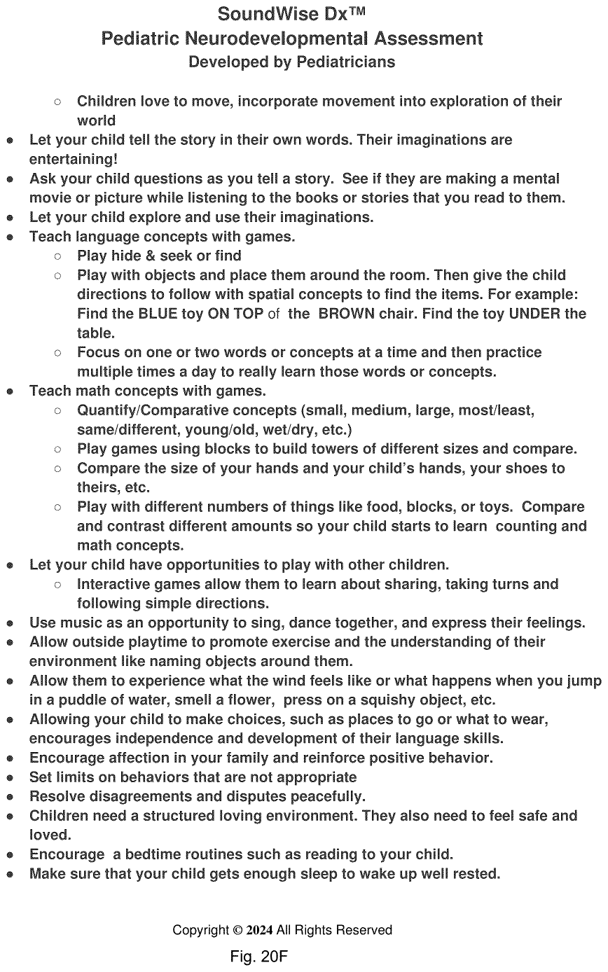

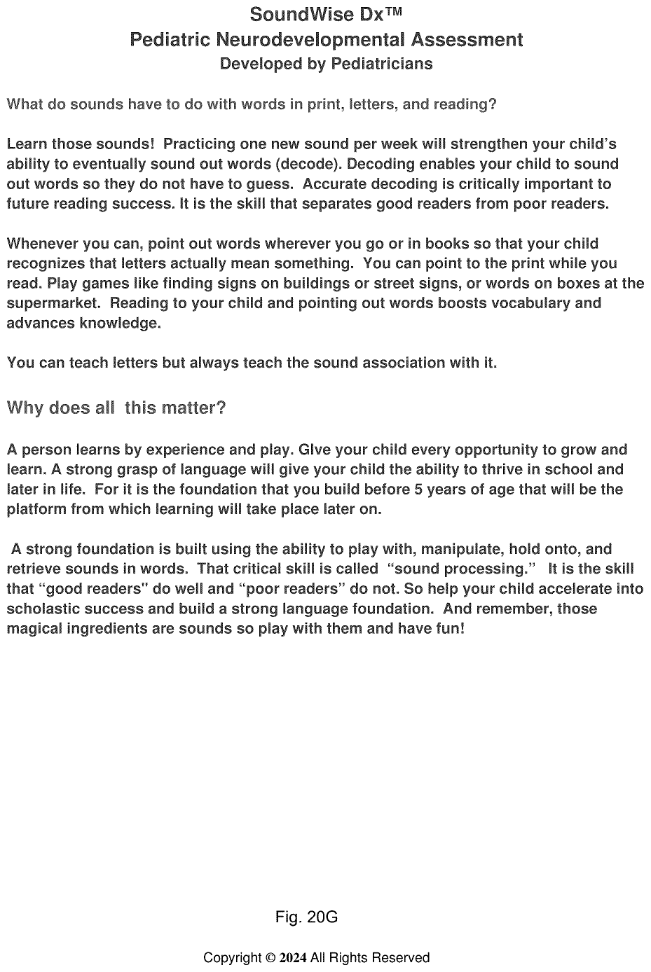

The system and method of the present disclosure may be configured to determine through an anxiety algorithm module and/or caregiver/teacher or authorized personnel to start Anxiety modules as well to start arranged telemedicine visits through the system and method of the present disclosure and/or in-person therapy. Also, the Caregiver/teacher will work on language development, social interaction, and sensory regulation by using a 5-Year-Old Developmental Milestone Information Sheet (DMIS) of the system and method of the present disclosure for age-appropriate activities. One example of a 5-Year-Old Developmental Milestone Information Sheet is shown in FIGS. 20A-20G. The system and method of the present disclosure may be configured to administer and score a diagnostic screen such as PHQ-9/SCARED/Columbia/Hamilton/GAD-7 or others for Anxiety that is appropriate for age. Based on the system and method of the present disclosure, Children will also be started on telemedicine services through the system and method of the present disclosure for anxiety.

Reasons for indications for medicine management for Anxiety is determined by the system and method of the present disclosure, physician, and input from caregiver/teacher. Pulse, Blood Pressure and EKG will be obtained by digital devices and are medically necessary before starting medication. Telemedicine services for anxiety though the system and method of the present disclosure must also be initiated and continued while the child is on medication for anxiety. All children diagnosed with Anxiety through the system and method of the present disclosure will also be evaluated for ADHD/ADD and/or Autism.

Concerning low self-esteem, self-esteem should always be monitored closely in a child as this can lead to depression or other mental health disparities in the future. Low self-esteem can arise from a variety of reasons. The system and method of the present disclosure may include one or more modules that address building self-esteem and confidence in a child. Telemedicine visits will be recommended via the system and method of the present disclosure or a local provider.

In the third part of the method, a referral may also generate referring the individual for services in areas where the evaluation results were sufficient to improve the child's skills, even though the child's skills are proficient for his or her age. For example, if the child demonstrates that he or she is able to demonstrate social communication, interaction, eye contact and able to demonstrate joint attention back and forth with assessment tasks, it may be indicative that the child's skills are not deficient, and the child needs to only to continue to build and/or improve his or her skills. Further, not showing restrictive and repetition behaviors, such as flapping their hands, spinning a lot, being only interested in lining up toys or objects, having sensory issue such as being upset with loud noises and difficulty with change of routine may also be indicative that the child's skills are not deficient, and the child needs to only to continue to build and/or improve his or her skills. Indeed, if the child is able to maintain attention, is not easily distracted, follows directions, etc. it may also be indicative that the child's skills are not deficient, and the child needs to only continue to build and/or improve his or her skills. Likewise, if the child is able to complete the tests without crying or worrying about how they are doing on the tests, it may also be indicative that the child's skills are not deficient, and the child needs to only to continue to build and/or improve his or her skills.

In some embodiments, the third part of the method further includes observing behavioral engagement skills of the child throughout the testing process, such as, for example, during the first test of the third part of the method, during the second test of the third part of the method, during the third test of the third part of the method, during the fourth test of the third part of the method and/or during the fifth test of the third part of the method. In some embodiments, the observations made throughout the testing process of the third part of the method can include noting the child's demeanor, such as, for example, observations pertaining to whether the child is cooperative, uncooperative, refuses to interact, completed the tests independently, exhibited poor eye contact, smiled, cried, was tired, would not engage, was shy, was distractible, was anxious, needed redirection for completion, was happy and engaged, was an English language learner, was difficult to motivate, etc. In some embodiments, the observations made throughout the testing process of the third part of the method are included in the worksheet (FIG. 17A-17F or 18A-18F) and/or are used to generate the referral. In some embodiments, information pertaining to the answers provided in the questionnaire of the third part of the method (FIG. 16A or 16B) is included in the worksheet and/or is used to generate the referral.

### Method—Part Four—Year Six

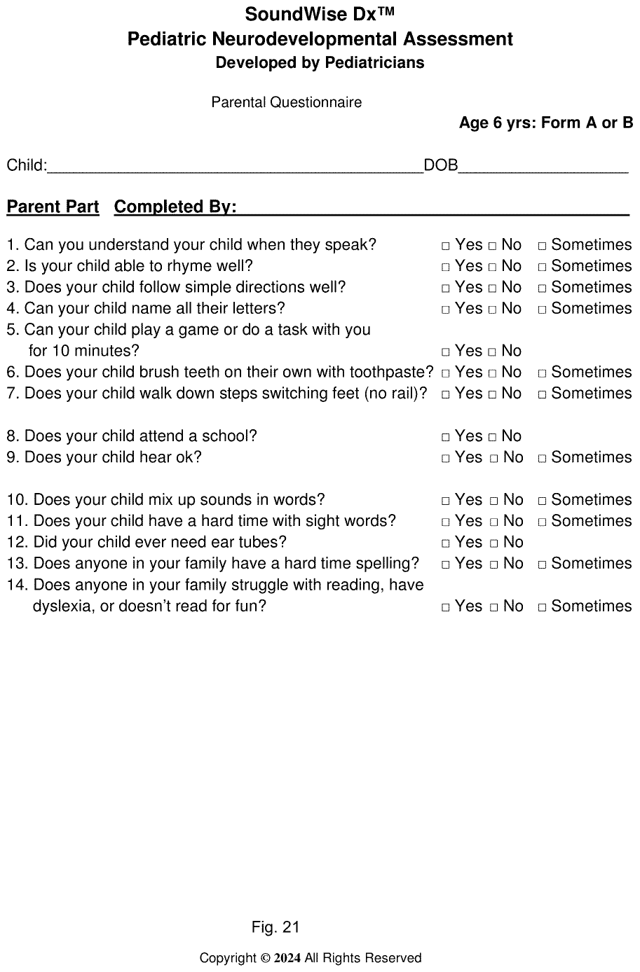

In a fourth part of the method, which should be administered after the third part of the method discussed above is administered to the child, when the child is approximately 6 years old, the child's parent's is required to fill out a questionnaire that includes various questions about the child. One embodiment of the questionnaire is shown in FIG. 21. As shown in FIG. 21, the questionnaire can include questions about the child's physical abilities, such as, for example, the child's hearing and vision. The questionnaire can also include questions relating to the child's ability to read, write, comprehend information, perform simple tasks, socialize, etc. For example, the questionnaire can include questions whether they can understand their child when the child speaks, whether the child is able to rhyme well, whether the child attends school, whether the child brushes his or her teeth with toothpaste, whether the child mixes up sounds in words, whether the child ever needed ear tubes, whether there is a family history of disorders, etc. (See, FIG. 21).

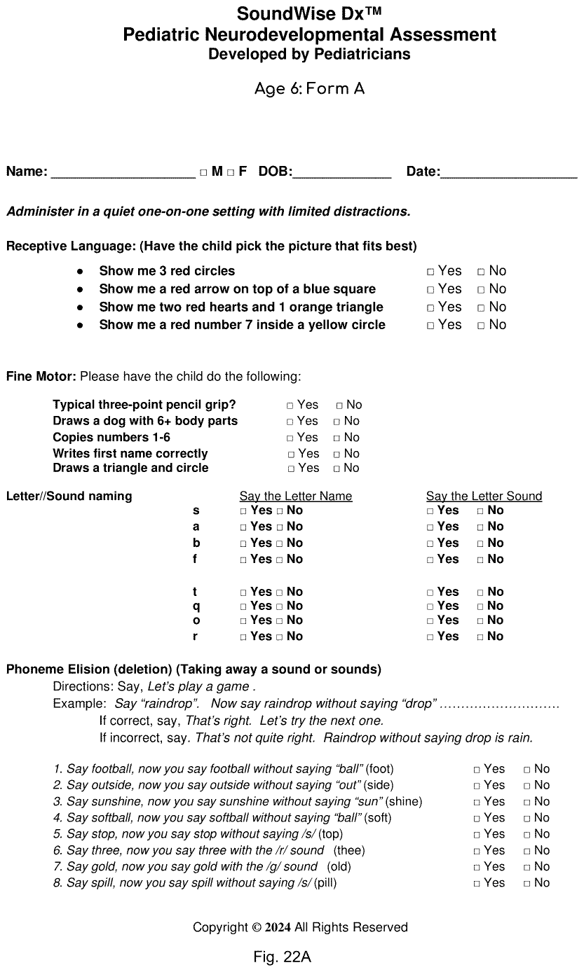

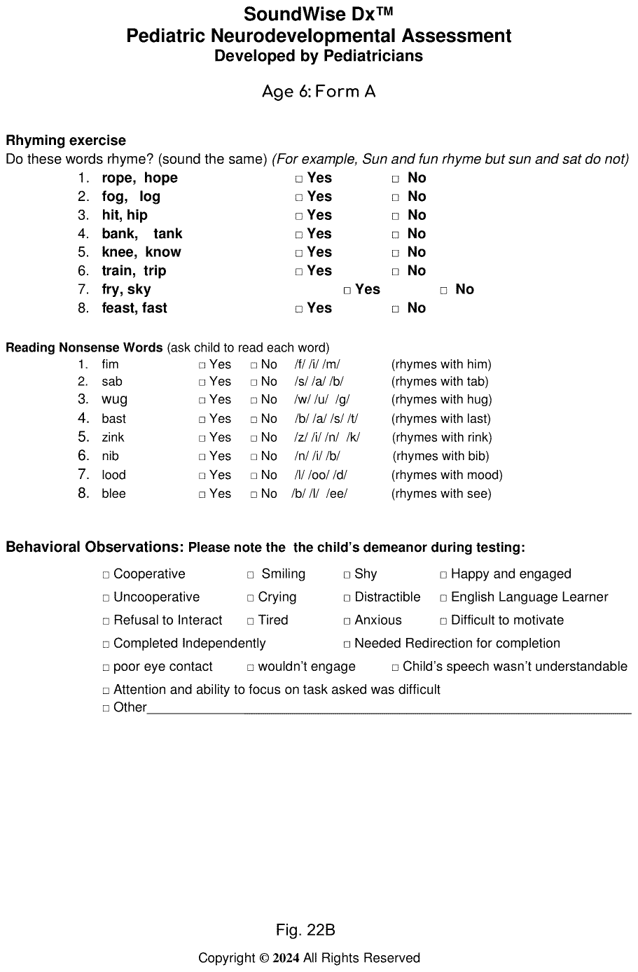

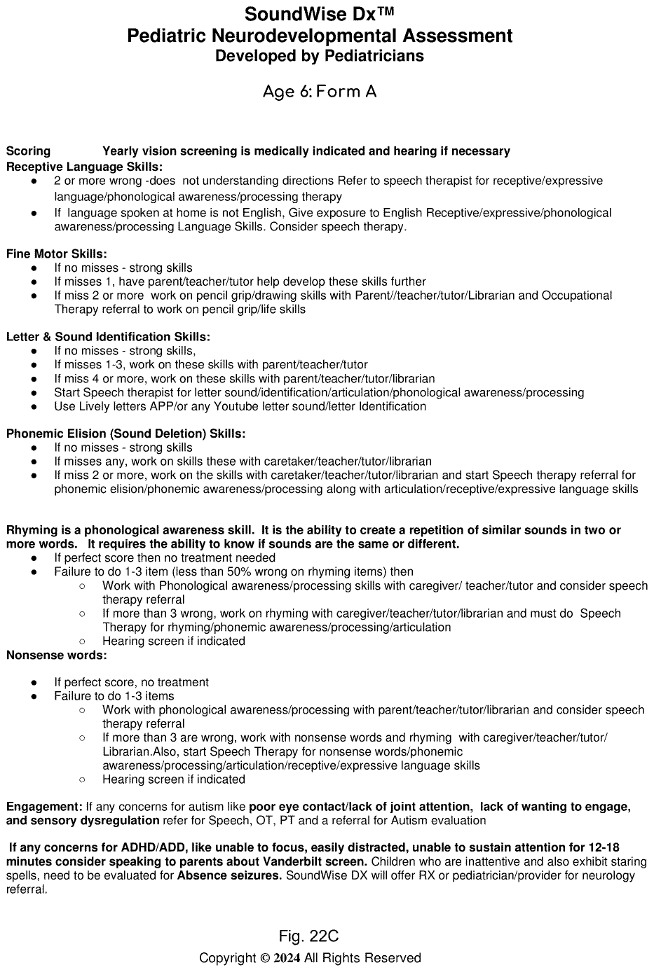

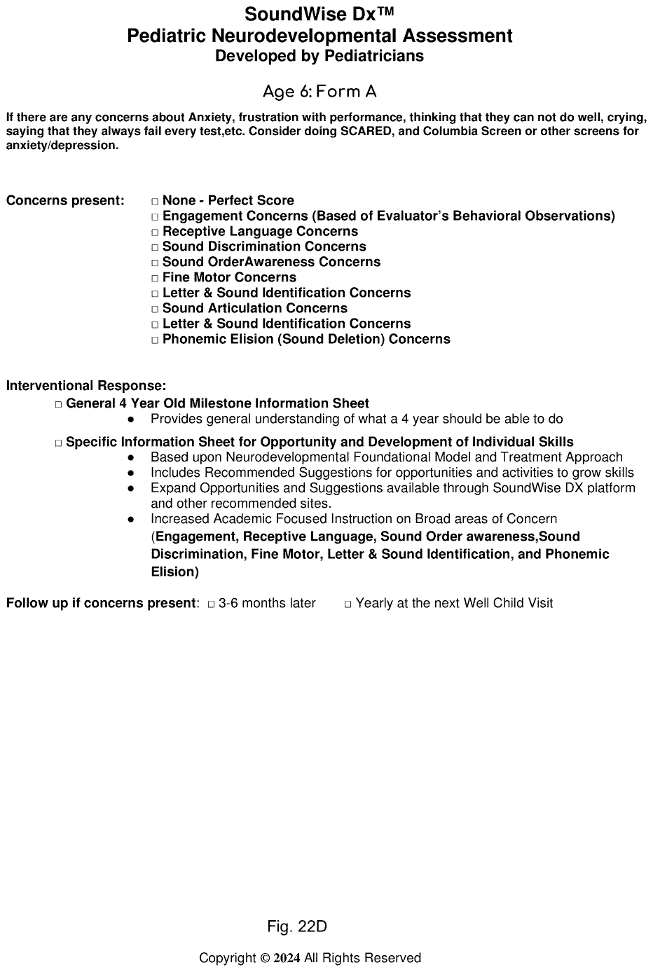

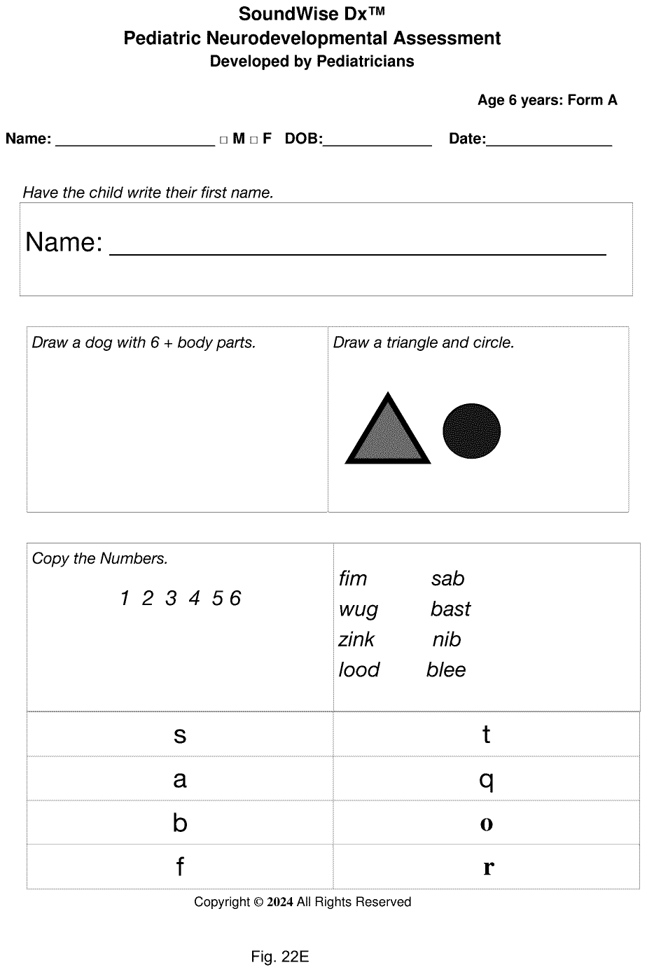

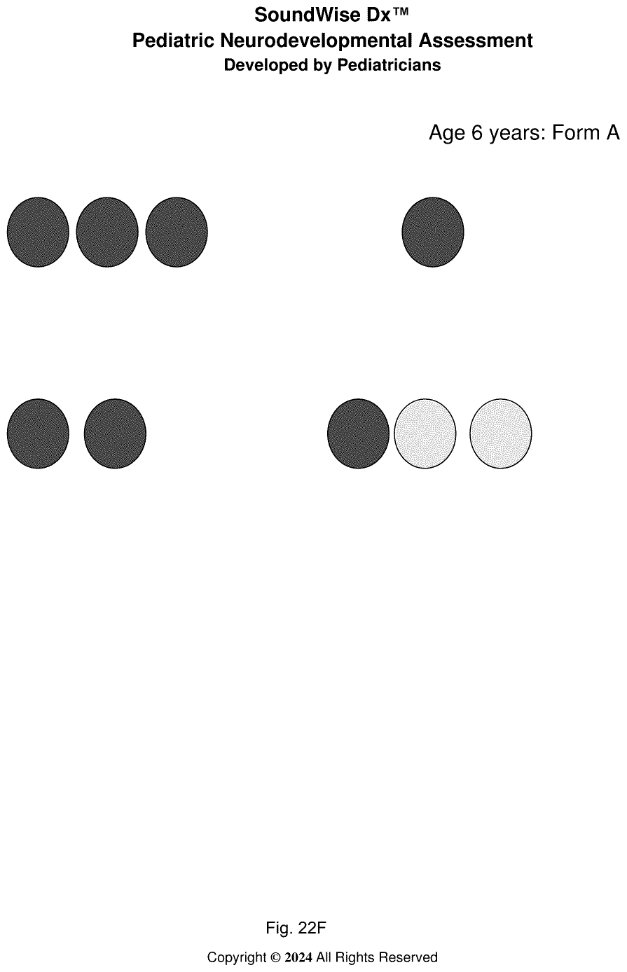

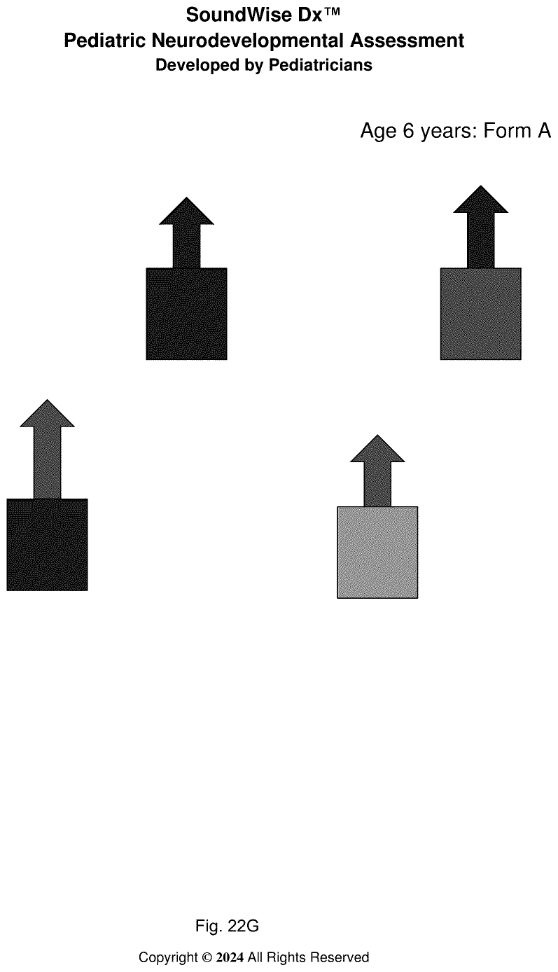

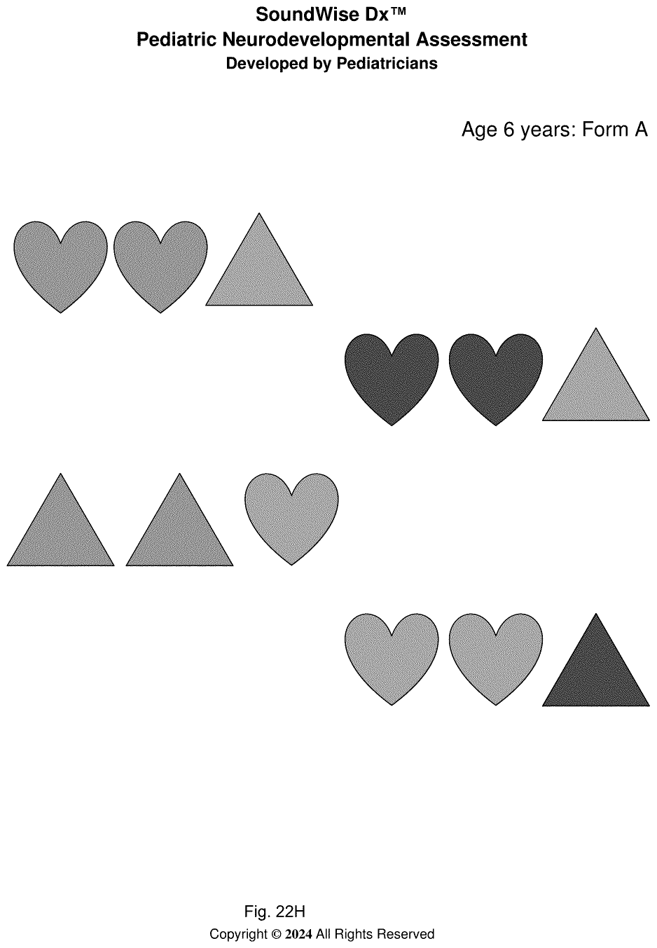

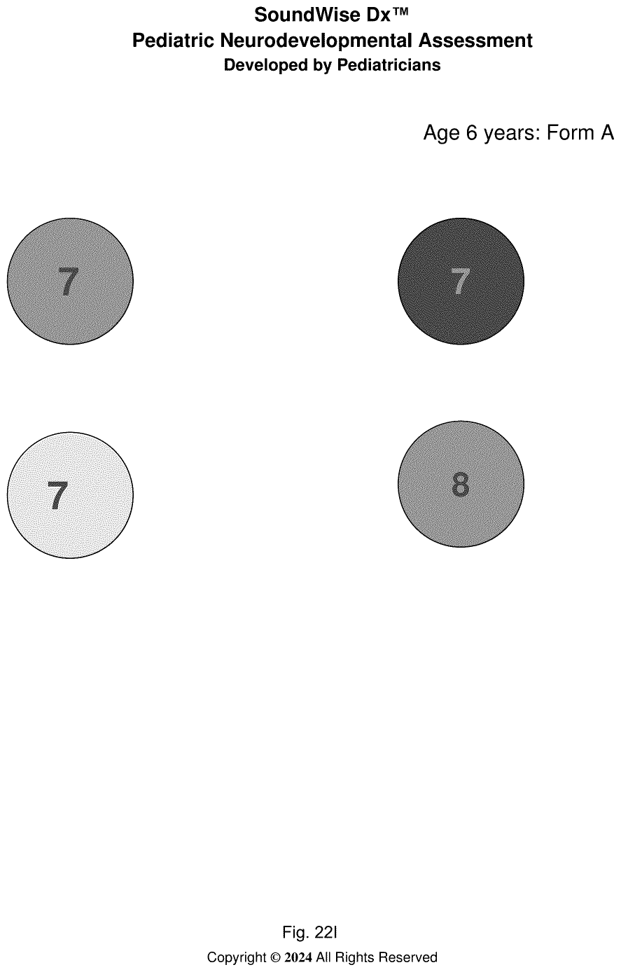

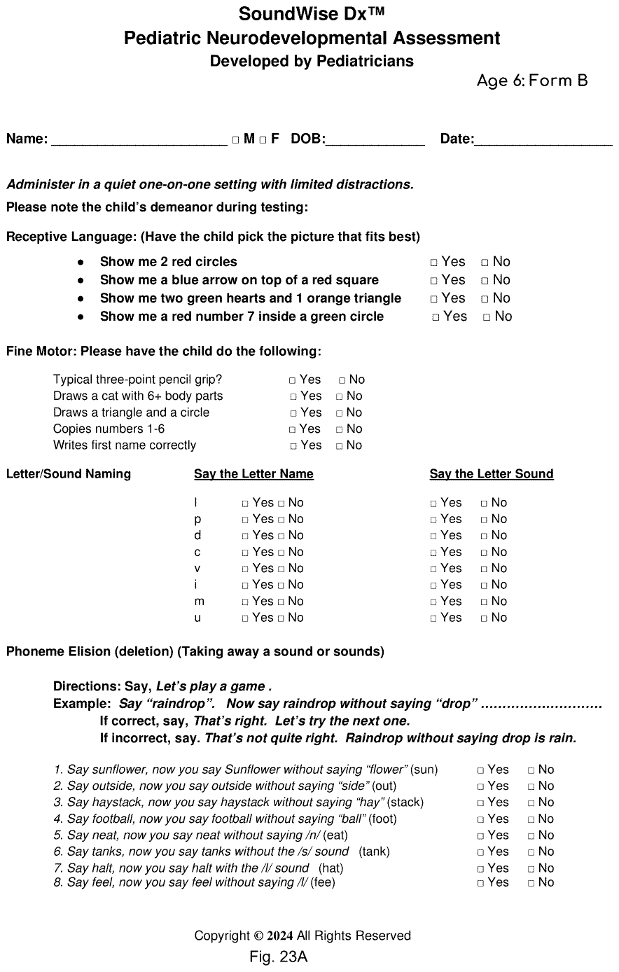

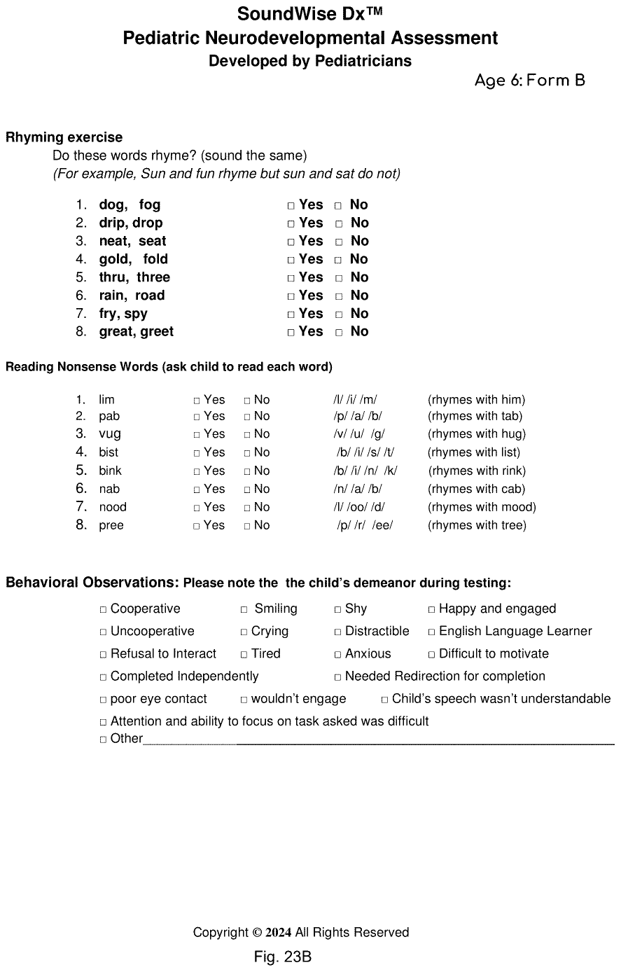

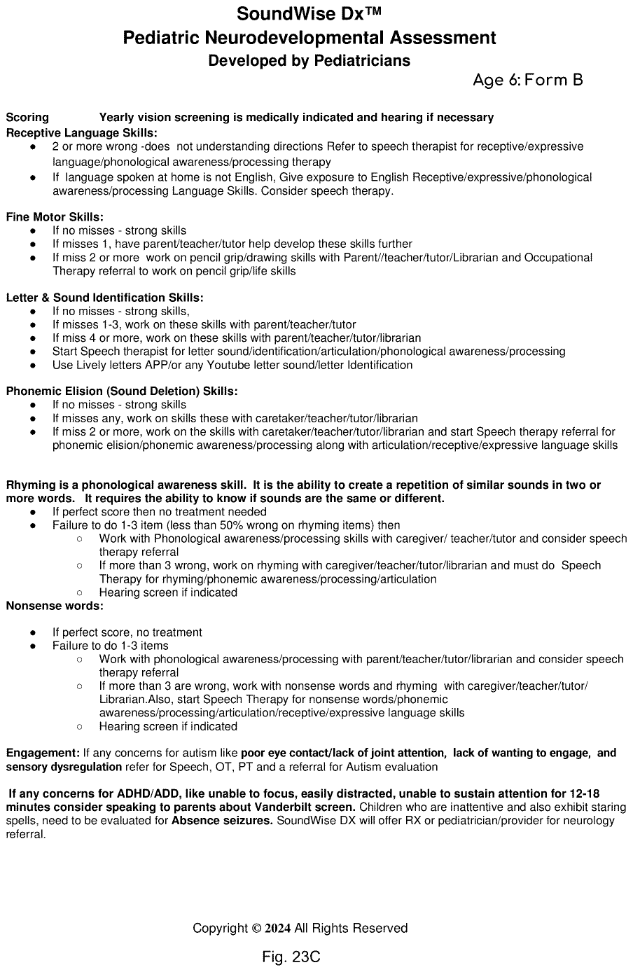

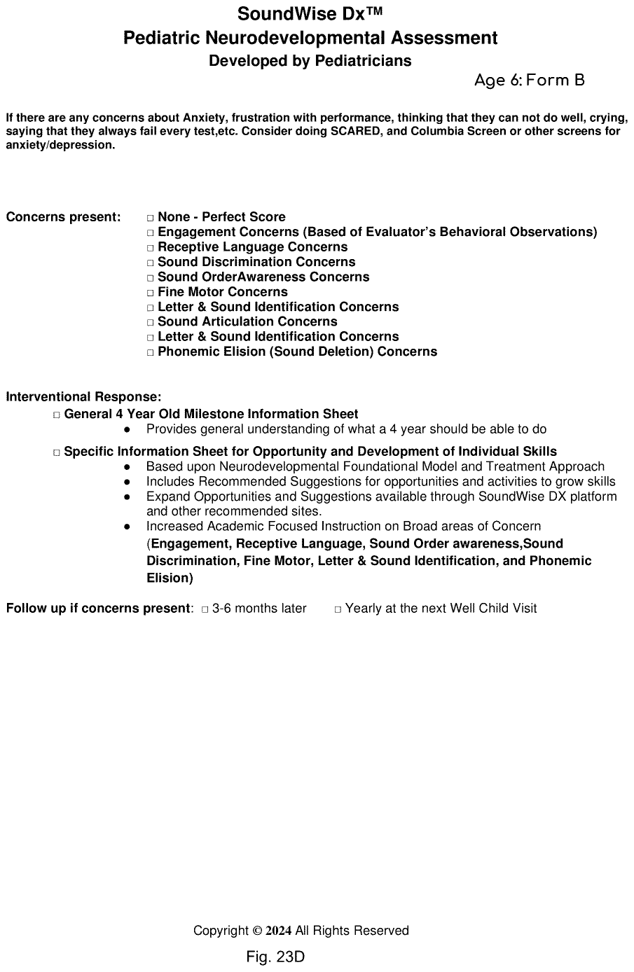

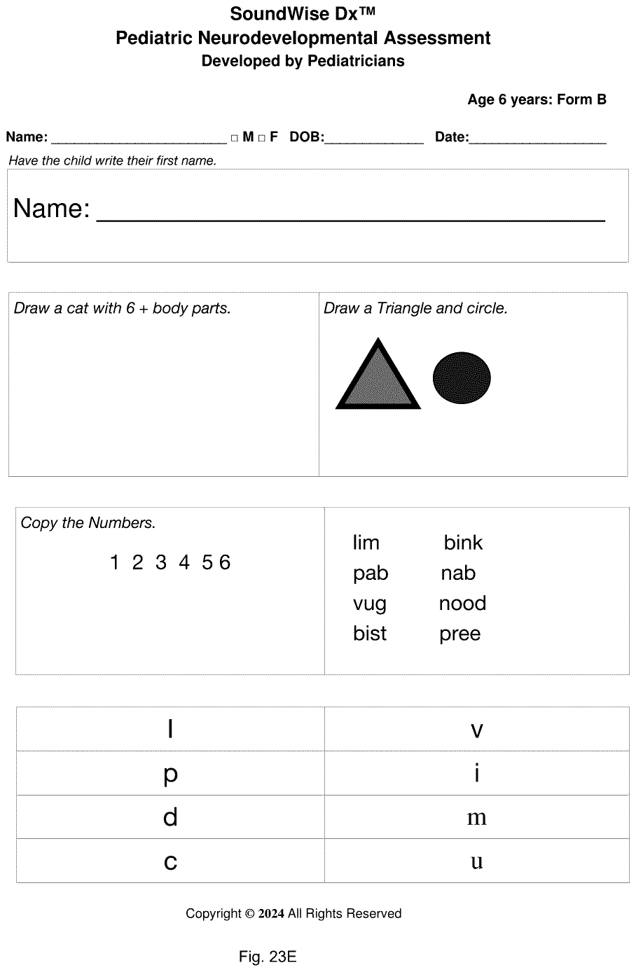

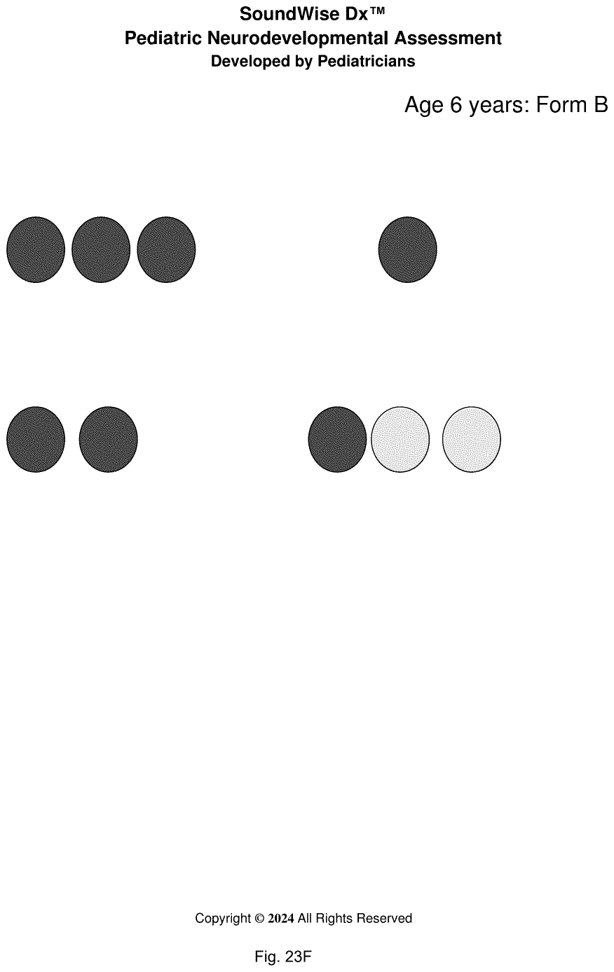

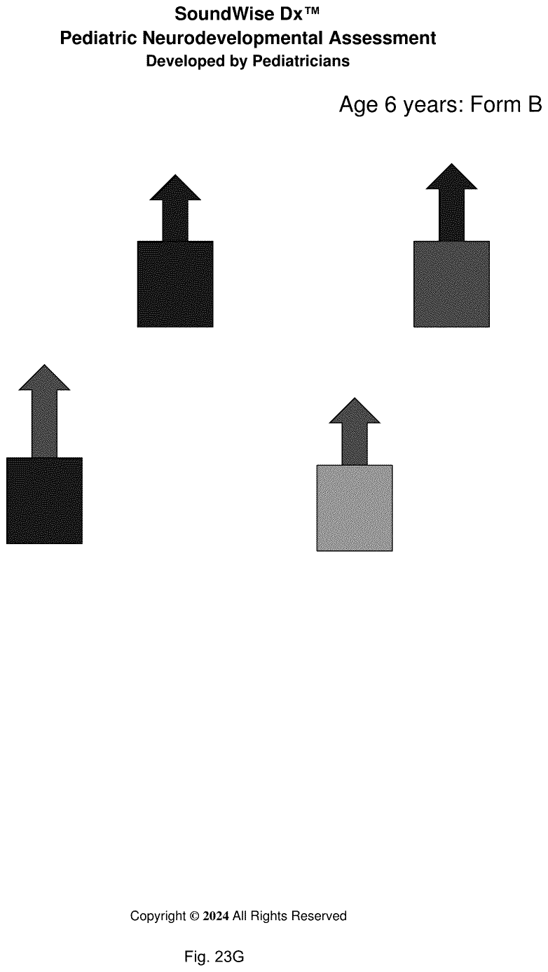

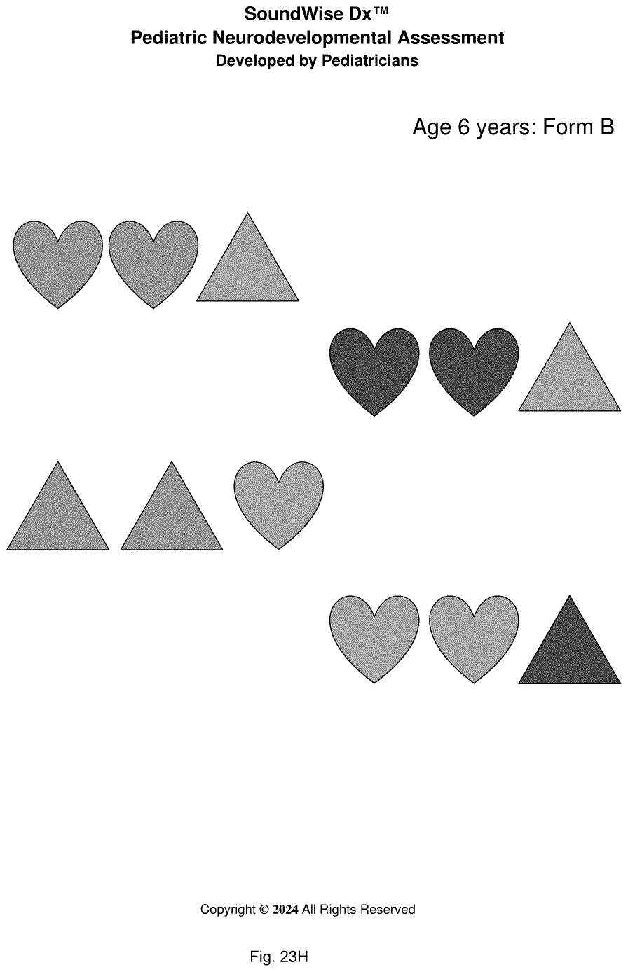

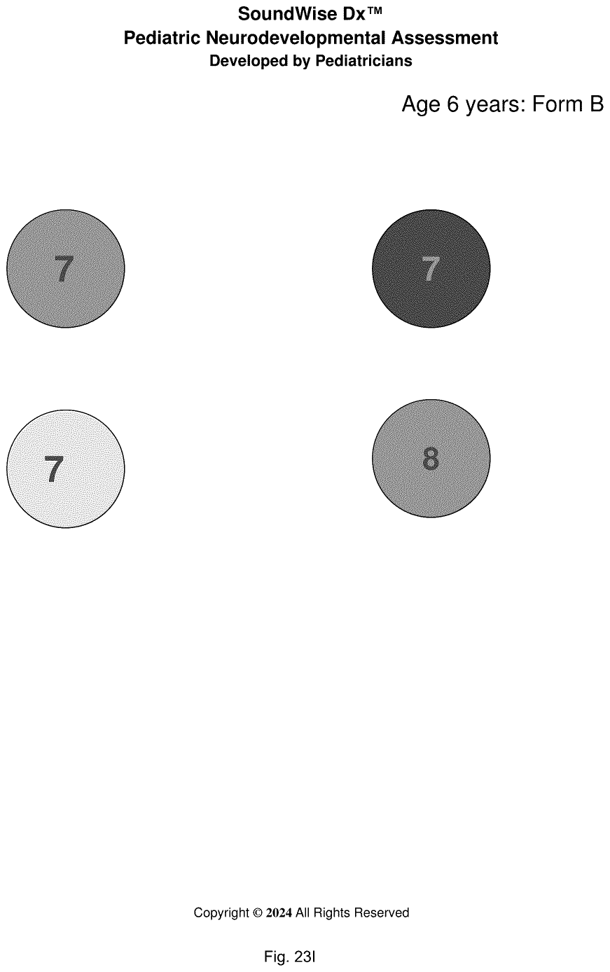

In the fourth part of the method, the child may be required to perform tests relating to receptive language, fine motor, letter naming and letter sound, phoneme elision (deletion), rhyming and/or reading skills (See, e.g., FIGS. 22A-22I; and FIGS. 23A-23I. For example, as shown in FIGS. 22A and 22B, the child may be required to show a show three red circles, to show a red arrow on top of a blue square, to show two red hearts and one orange triangle, to show a red number seven inside a yellow circle, etc. The child may also be required to perform a typical three-point pencil grip, draw a dog with six or more body parts, copy numbers one through six, write his or her first name and/or draw a triangle and circle (FIG. 22A). The child may also be required to say various letter names and/or letter sounds and/or perform a game relating to phoneme elision (deletion) similar to that discussed above in which the child is asked to take away a sound or sounds from a word (FIG. 22A). In one embodiment, the child may also be required to perform a rhyming exercise in which the child indicates whether or not two words rhyme (FIG. 22B). In one embodiment, the child may also be required to read nonsense or nonreal words in which the child indicates whether or not the nonsense or nonreal word rhymes with an existing or proper word (FIG. 22B). In some embodiment the document shown in FIGS. 22A-22I and/or the document shown in FIGS. 23A-23I may be used as a worksheet to record results of the tests taken by the child during the fourth part of the method.

In one embodiment, shown in FIGS. 23A-23I, the child may be required to show a show two red circles, to show a blue arrow on top of a red square, to show two green hearts and one orange triangle, to show a red number seven inside a green circle, etc. The child may also be required to perform a typical three-point pencil grip, draw a dog with six or more body parts, copy numbers one through six, write his or her first name and/or draw a triangle and circle (FIG. 23A). The child may also be required to say various letter names and/or letter sounds and/or perform a game relating to phoneme elision (deletion) similar to that discussed above in which the child is asked to take away a sound or sounds from a word (FIG. 23A). In one embodiment, the child may also be required to perform a rhyming exercise in which the child indicates whether or not two words rhyme (FIG. 23B). In one embodiment, the child may also be required to read nonsense or nonreal words in which the child indicates whether or not the nonsense or nonreal word rhymes with an existing or proper word (FIG. 23B).

In the fourth part of the method, the tests discussed above and shown in FIGS. 22A-22I and/or FIGS. 23A-23I relate to fine motor skills, letter and sound identification skills, phonemic elision (sound deletion) skills and rhyming skills (FIGS. 22C and 22D and/or FIGS. 23C and 23D). The child's behavior may be observed during these tests for considerations such as, for example, whether the child is cooperative, whether the child is uncooperative, whether the child refuses to interact, whether the child completed tasks independently, whether the child exhibited poor eye contact, whether the child smiled, whether the child cried, whether the child appeared tired, whether or not the child would engage, whether the child was shy, whether the child was distractible, whether the child was anxious, whether the child needed redirection to complete tasks, whether the child was happy and engaged, whether the child is an English language learner, whether the child was difficult whether the child's speech was understandable, etc., as shown in FIGS. 22B and 23B.

In the third part of the method, tests for fine motor skills, letter and sound identification skills, phonemic elision (sound deletion) skills and rhyming skills (FIG. 22C) and/or receptive language skills, fine motor skills, sound and letter naming skills, phonemic elision skills and rhyming skills (FIG. 23C) are evaluated for the presence or absence of a disorder and/or school readiness. The ability of the child to perform the requested tasks is recorded to generate input data. The ability of the child to perform one or more of the requested tasks may be recorded in a fourth worksheet. One embodiment of the fourth worksheet is shown in FIGS. 22A-22I. Another embodiment of the fourth worksheet is shown in FIGS. 23A-23I.

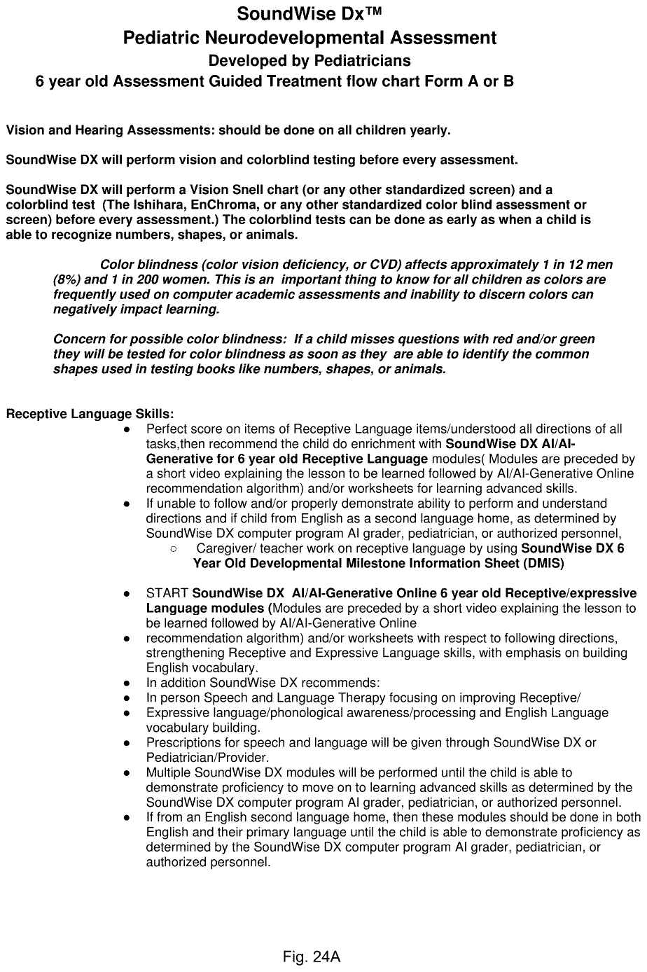

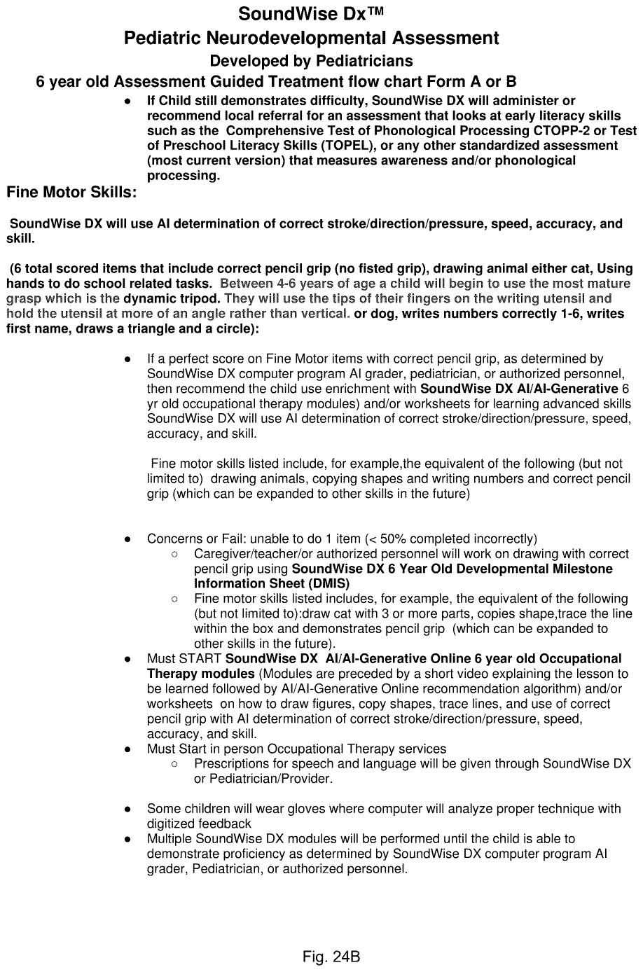

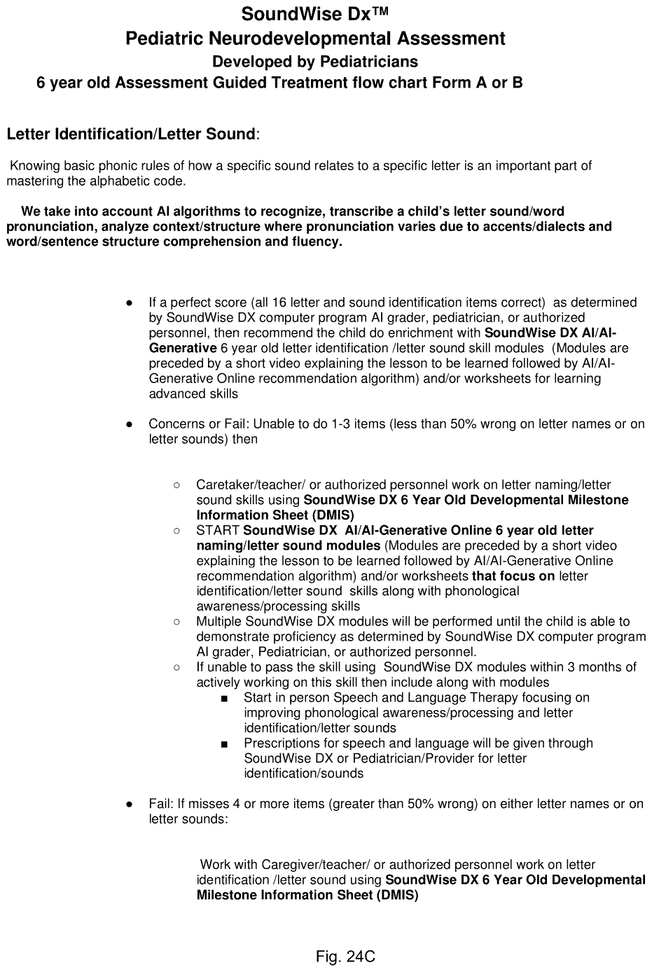

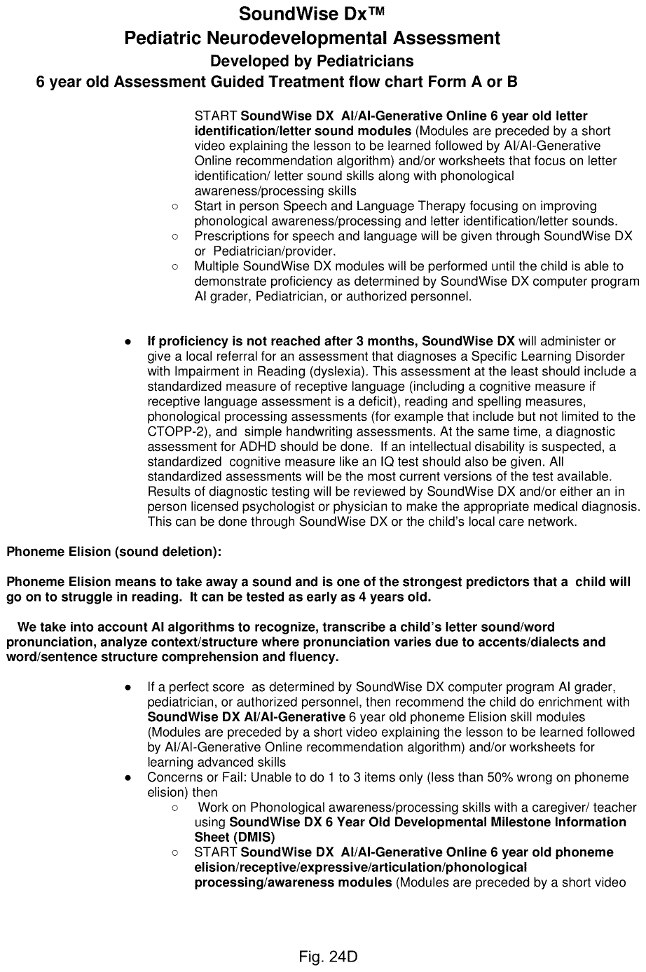

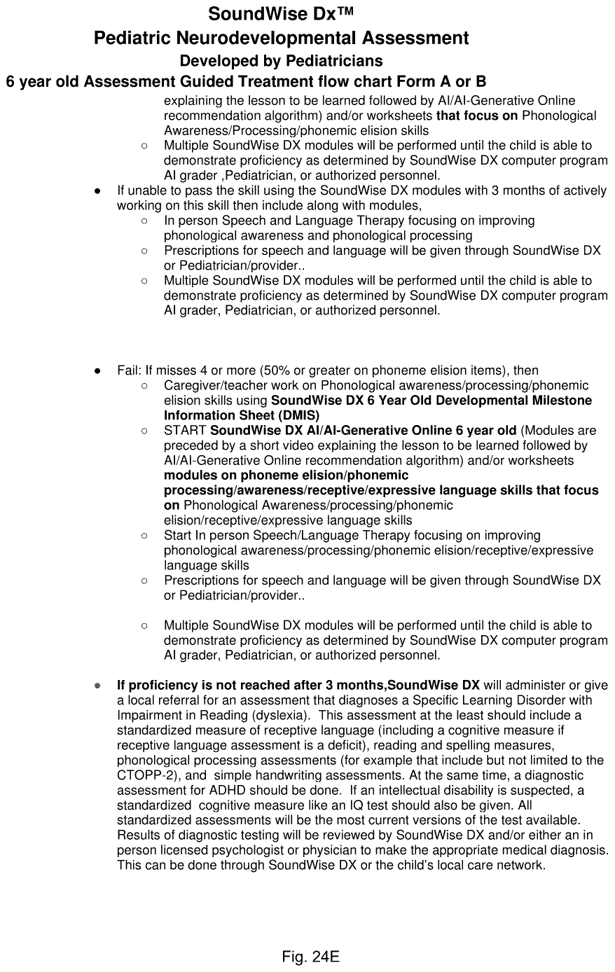

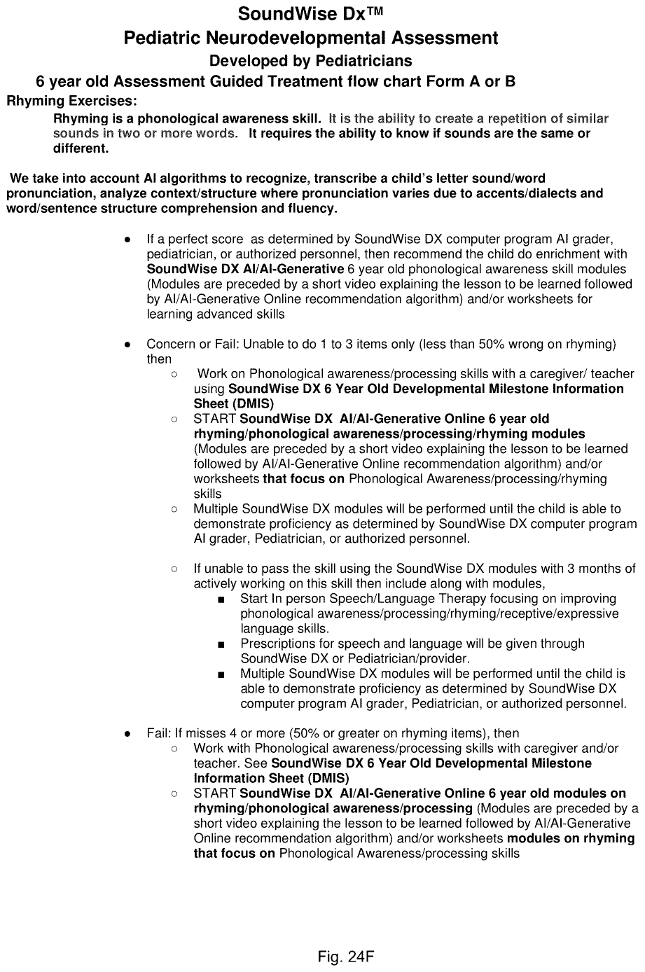

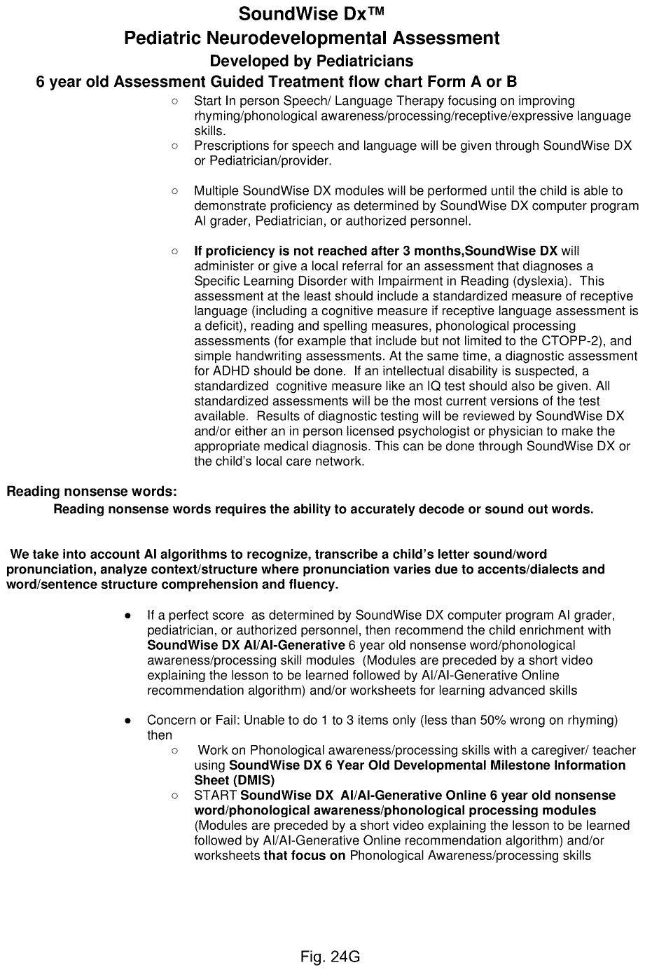

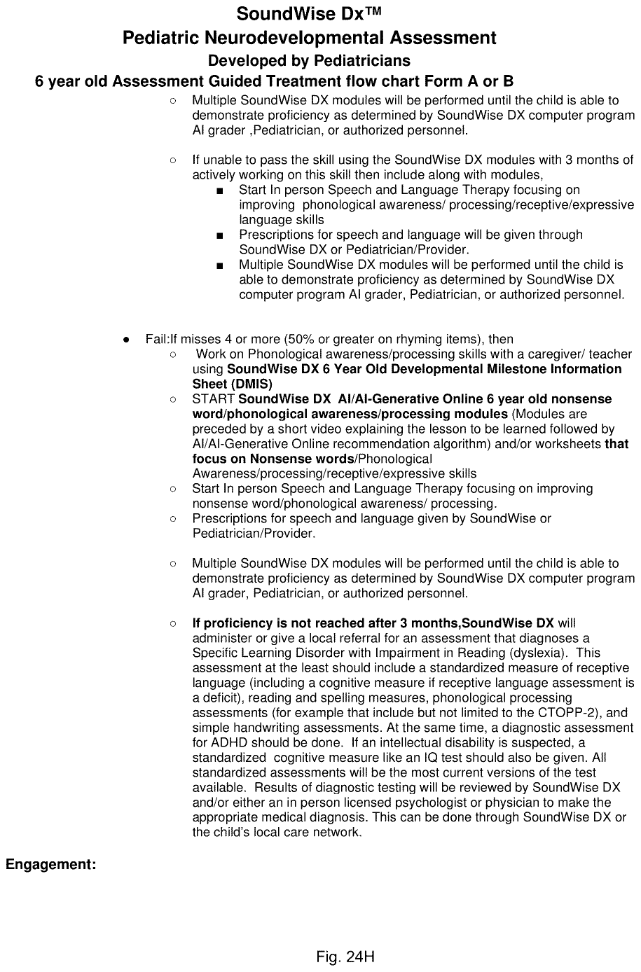

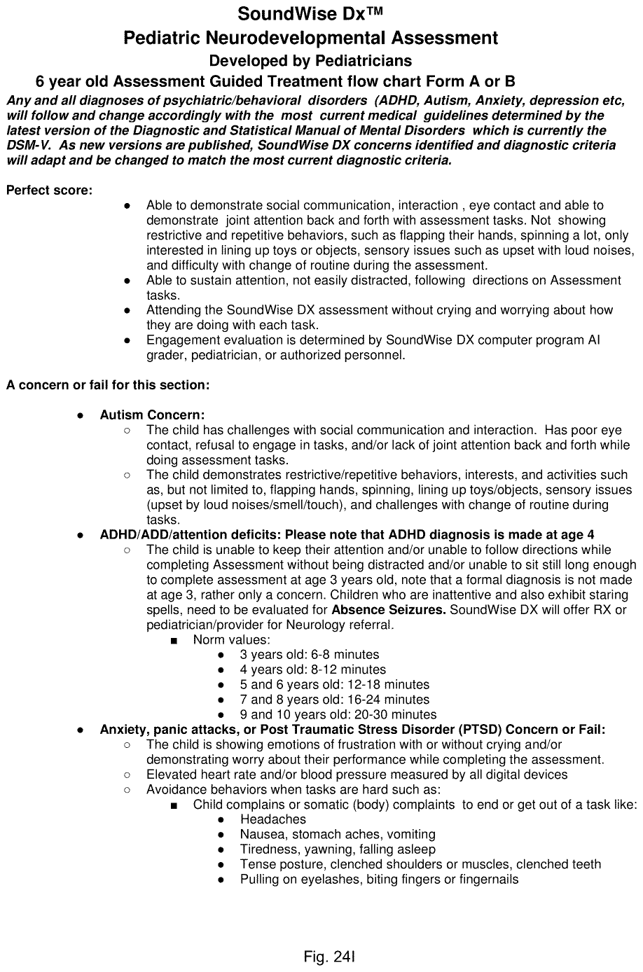

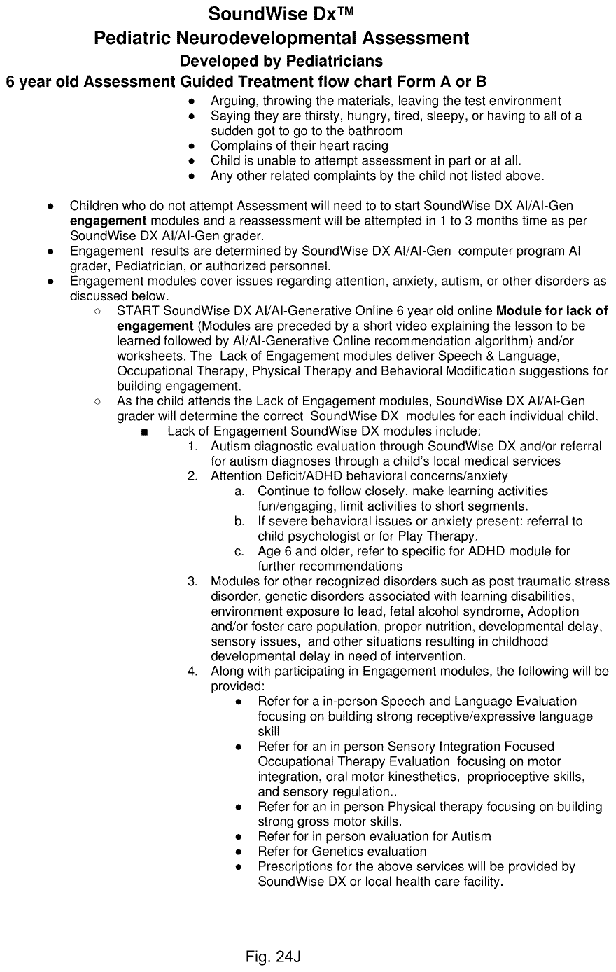

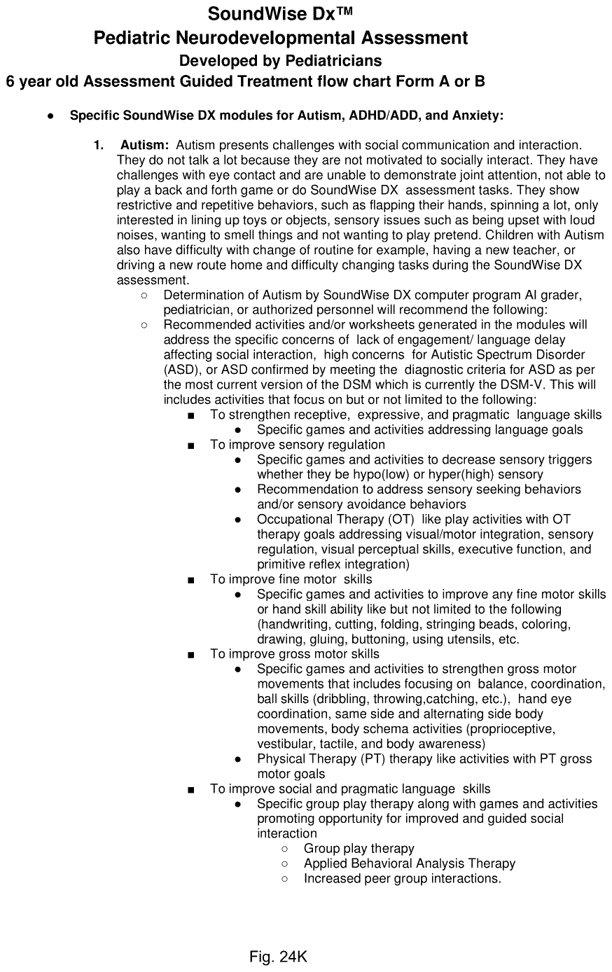

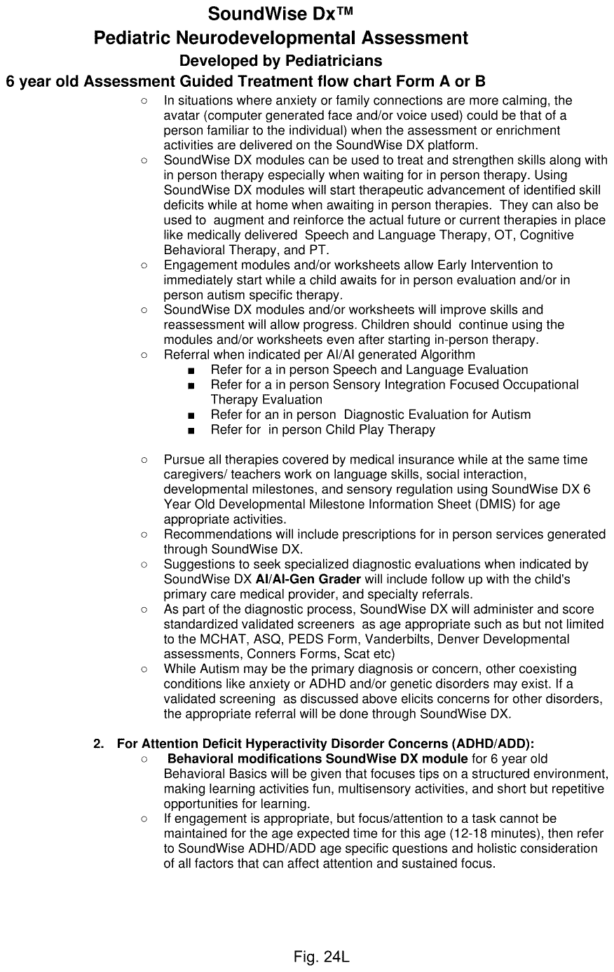

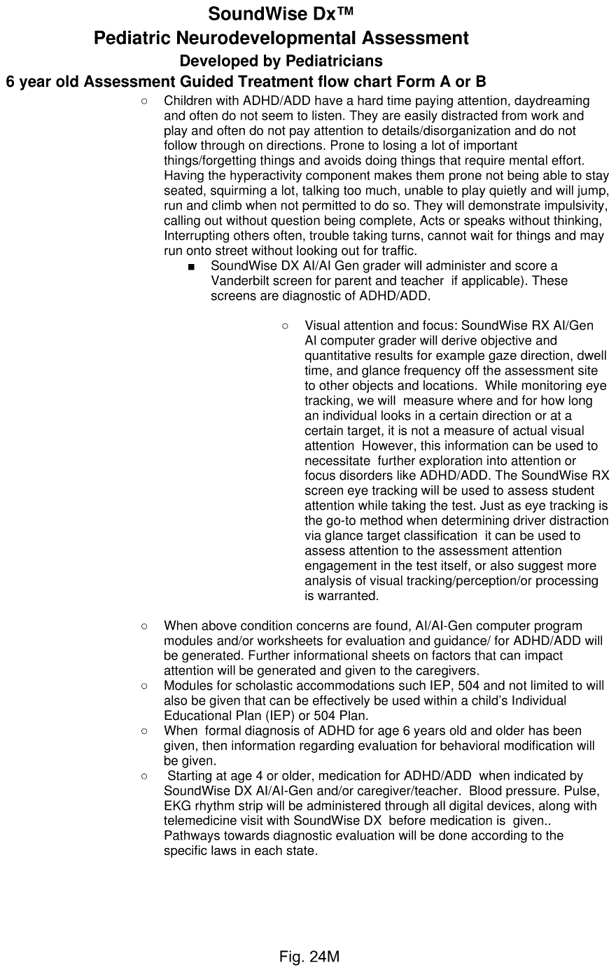

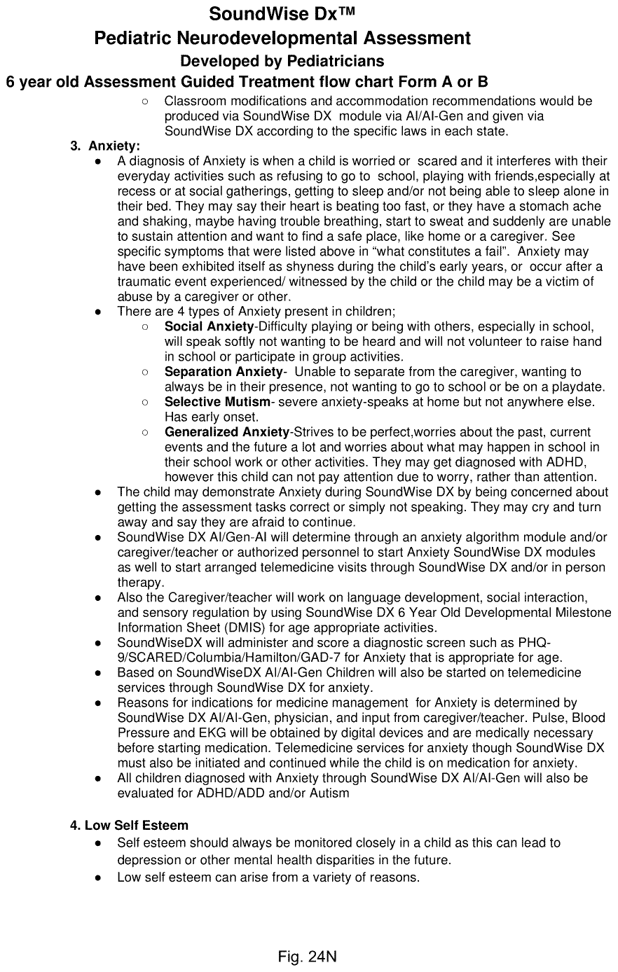

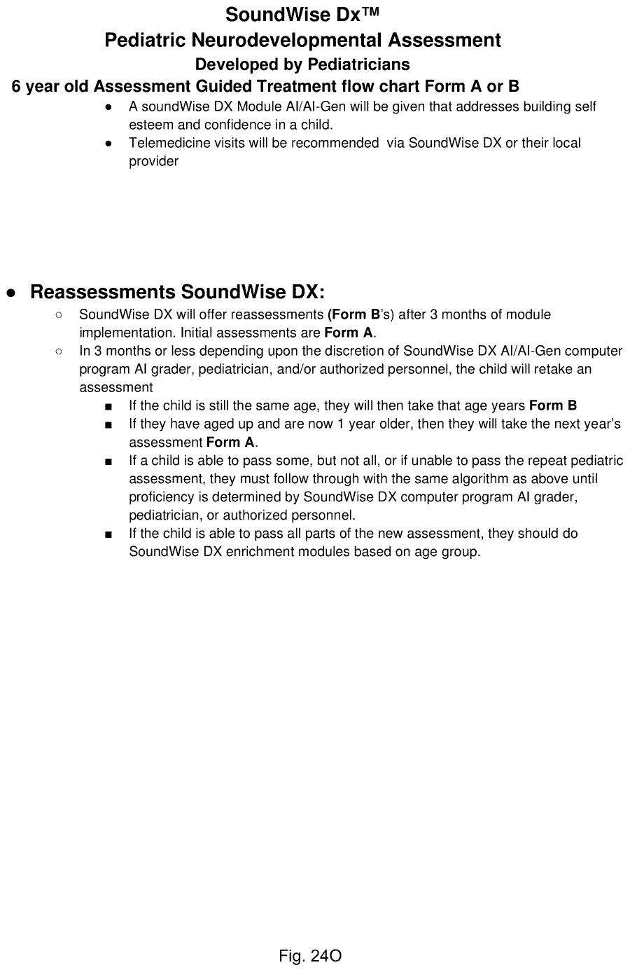

The fine motor skill input data may include whether or not the child was able to perform required tasks. The ability of the child to perform the required tasks may be recorded in the fourth worksheet. The fine motor input data is evaluated against an answer key to generate a fine motor evaluation result. The fine motor evaluation result is another determination with respect to the presence or absence of fine motor deficiency, after the third determination with respect the presence or absence of fine motor conducted during the third part of the method. In some embodiments, the fine motor evaluation result includes a score, such as, for example, a number score. In some embodiments, the fine motor evaluation result includes a written evaluation, such as, for example, words written in sentence or note form that explain what was observed by the practitioner. In some embodiments, the fine motor result is included in an assessment that evaluates at least each of the tests administered during the fourth part of the method. One embodiment of the assessment is shown in FIGS. 24A-24O.

In the fourth part of the method, a second test is administered in which the child has to do a letter and sound identification task to generate a letter and sound identification task input data. The ability of the child to do a letter and sound identification task may be recorded in the third worksheet (FIG. 22A-22I or FIG. 23A-23I). The letter and sound identification input data is evaluated against an answer key to generate a letter and sound identification evaluation result. The letter and sound identification evaluation result is another determination with respect to the presence or absence of letter and sound identification deficiency, after the second determination with respect the presence or absence of letter and sound identification conducted during the third part of the method. In some embodiments, the letter and sound identification evaluation result include a score, such as, for example, a number score. In some embodiments, the letter and sound identification evaluation result include a written evaluation, such as, for example, words written in sentence or note form that explain what was observed by the practitioner. In some embodiments, the letter and sound identification evaluation result is included in the assessment (FIGS. 24A-24O). In some embodiments, the letter and sound identification evaluation result uses AI or other software to recognize and/or transcribe the child's letter sound/word pronunciation, analyze context/structure where pronunciation varies due to accents/dialects and word/sentence structure comprehension and fluency.

In the fourth part of the method, a third test is administered in which the child has to do a phonemic elision (sound deletion) task to generate a phonemic elision task input data. The ability of the child to do a phonemic elision task may be recorded in the fourth worksheet (FIG. 22A-22I or FIG. 23A-23I). The phonemic elision input data is evaluated against an answer key to generate a phonemic elision evaluation result. The phonemic elision evaluation result is another determination with respect to the presence or absence of phonemic elision deficiency, after the second determination with respect the presence or absence of phonemic elision deficiency conducted during the third part of the method. In some embodiments, the phonemic elision evaluation result includes a score, such as, for example, a number score. In some embodiments, the phonemic elision evaluation result includes a written evaluation, such as, for example, words written in sentence or note form that explain what was observed by the practitioner. In some embodiments, the phonemic elision evaluation result is included in the assessment (FIGS. 24A-24O). In some embodiments, the phonemic elision evaluation result uses AI or other software to recognize and/or transcribe the child's letter sound/word pronunciation, analyze context/structure where pronunciation varies due to accents/dialects and word/sentence structure comprehension and fluency.

In the fourth part of the method, a fourth test is administered in which the child has to do a rhyming task to generate a rhyming task input data. The ability of the child to do a rhyming task may be recorded in the third worksheet (FIG. 22A-22I or FIG. 23A-23I). The rhyming task input data is evaluated against an answer key to generate a rhyming evaluation result. The rhyming evaluation result is another determination with respect to the presence or absence of rhyming deficiency, after the third determination with respect the presence or absence of rhyming deficiency conducted during the third part of the method. Indeed, because rhyming is a phonological skill, the fourth test will test the child's ability to create a repetition of similar sounds in to or more words, which requires the ability to know if the sounds are the same or different. In some embodiments, the rhyming evaluation result includes a score, such as, for example, a number score. In some embodiments, the rhyming evaluation result includes a written evaluation, such as, for example, words written in sentence or note form that explain what was observed by the practitioner. In some embodiments, the rhyming evaluation result is included in the assessment (FIGS. 24A-24O). In some embodiments, the rhyming evaluation result uses AI or other software to recognize and/or transcribe the child's letter sound/word pronunciation, analyze context/structure where pronunciation varies due to accents/dialects and word/sentence structure comprehension and fluency.

In one embodiment of the fourth part of the method, a fifth test is administered in which the child has to do a nonsense words exercise that compares nonsense words with real words to generate a nonsense word skill input data. The ability of the child to do a nonsense word skill may be recorded in the third worksheet (FIG. 22A-22I or FIG. 23A-23I). The nonsense word skill input data is evaluated against an answer key to generate a nonsense word skill evaluation result. The nonsense word skill evaluation result is another determination with respect to the presence or absence of nonsense word skills. In some embodiments, the nonsense word skill evaluation result includes a score, such as, for example, a number score. In some embodiments, the nonsense word skill evaluation result includes a written evaluation, such as, for example, words written in sentence or note form that explain what was observed by the practitioner. In some embodiments, the nonsense word skill evaluation result is included in the assessment (FIGS. 22A-22P). In some embodiments, the nonsense word skill evaluation result uses AI or other software to recognize and/or transcribe the child's letter sound/word pronunciation, analyze context/structure where pronunciation varies due to accents/dialects and word/sentence structure comprehension and fluency.

In the fourth part of the method, a referral is generated referring the individual for services in areas where the evaluation results were deficient to promote at least one of the skills tested in the fourth part of the method. In some embodiments, the assessment (FIGS. 24A-24O), which can include one or more of the test results in the fourth part of the method are evaluated to generate the referral. In particular, if the results indicate that the child has proficient fine motor skills, the child is not referred for services, such as, for example, occupational therapy to promote advancement in fine motor skills. If, however, the test results indicate that the child is deficient with his or her fine motor skills, the child is referred for services, such as, for example, speech therapy to promote advancement in fine motor skills. Likewise, if the results indicate that the child has proficient letter and sound identification skills, the child is not referred for services, such as, for example, occupational therapy to promote advancement in letter and sound identification skills. If, however, the test results indicate that the child is deficient with his or her letter and sound identification skills, the child is referred for services, such as, for example, Speech therapy to promote advancement in letter and sound identification skills. If the results indicate that the child has proficient phonemic elision skills, the child is not referred for services, such as, for example, Speech therapy to promote advancement in phonemic elision skills. If, however, the test results indicate that the child is deficient with his or her phonemic elision skills, the child is referred for services, such as, for example, Speech therapy to promote advancement in phonemic elision skills. If the results indicate that the child has proficient rhyming/phonological awareness skills, the child is not referred for services, such as, for example, Speech therapy to promote advancement in rhyming/phonological awareness skills. If, however, the test results indicate that the child is deficient with his or her rhyming/phonological awareness skills, the child is referred for services, such as, for example, Speech therapy to promote advancement in rhyming/phonological awareness skills. Lastly, if the results indicate that the child has proficient nonsense word skills, the child is not referred for services, such as, for example, speech therapy to promote advancement in nonsense word skills. If, however, the test results indicate that the child is deficient with his or her nonsense word skills, the child is referred for services, such as, for example, Speech therapy to promote advancement in nonsense word skills.

In some embodiments, the fourth part of the method includes evaluating the child for ADHD, autism, anxiety, panic attacks, post-traumatic stress disorder (PTSD), depression, low self-esteem, etc. if the evaluation results in the fourth part of the method were deficient to promote at least one of the skills tested in the fourth part of the method.

The ADHD evaluation in the fourth part of the method can include giving focused tips on a structured environment, making learning activities fun, multisensory activities, and short but repetitive opportunities for learning. If engagement is appropriate, but focus/attention to a task cannot be maintained for the age expected time for the child's age, then the method may refer the child to take a test that includes ADHD/ADD age specific questions and consider all factors that can affect attention and sustained focus. In some embodiments, if the child exhibits signs of ADHD/ADD, the method can be adapted to administer and score a Vanderbilt screen for parent and teacher (if applicable). These screens are diagnostic of ADHD/ADD. Absence seizure must be ruled out by EEG or another medical device.

Concerning autism, the fourth part of the method includes evaluating whether the child has challenges with social communication and interaction, has poor eye contact, refuses to engage in tasks, and/or lacks joint attention back and forth while doing assessment tasks. For example, if the child demonstrates restrictive/repetitive behaviors, interests, and activities such as, but not limited to, flapping hands, spinning, lining up toys/objects, sensory issues (upset by loud noises/smell/touch), and challenges with change of routine during tasks, the evaluation should include an autism evaluation.

If the autism evaluation of the present method may recommend activities and/or worksheets that are adapted to address the specific concerns of lack of engagement/language delay affecting social interaction, high concerns for Autistic Spectrum Disorder (ASD), or ASD confirmed. This will include activities that focus on strengthening receptive, expressive, and pragmatic language skills; specific games and activities addressing language goals. To improve sensory regulation, the autism evaluation of the present method may recommend specific games and activities to decrease sensory triggers whether they be hypo(low) or hyper(high) sensory. To address sensory seeking behaviors and/or sensory avoidance behaviors, the autism evaluation may include recommending Occupational Therapy (OT) like play activities with OT therapy goals addressing visual/motor integration, sensory regulation, visual perceptual skills, executive function, and primitive reflex integration.

The system and method of the present disclosure can be used to treat and strengthen skills along with in-person therapy especially when waiting for in-person therapy. The system and method of the present disclosure can recommend starting therapeutic advancement of identified skill deficits while at home when awaiting in-person therapies. Such therapeutic advancement can be used to augment and reinforce the actual future or current therapies in place like medically delivered Speech and Language Therapy, OT, Cognitive Behavioral Therapy, and PT. The system and method of the present disclosure can include engagement modules and/or worksheets that allow Early Intervention to immediately start while a child waits for in-person evaluation and/or in-person autism specific therapy. The system and method of the present disclosure can improve skills and reassessment will allow progress. Children should continue using the system and method of the present disclosure even after starting in-person therapy. Referral pursuant to the autism evaluation can include referring the child for an in-person Speech and Language Evaluation, referring the child for an in-person Sensory Integration Focused Occupational Therapy Evaluation, referring the child for an in-person Diagnostic Evaluation for Autism, referring the child for in-person Child Play Therapy.

Concerning anxiety, panic attacks, or PTSD, if the child is showing emotions of frustration with or without crying and/or demonstrating worry about their performance while completing the assessment, has an elevated heart rate and/or blood pressure and/or avoids behaviors when tasks are hard such as: complaining to end or get out of a task like, having headaches, having nausea, stomach aches, vomiting, having tiredness, yawning, falling asleep, having a tense posture, clenched shoulders or muscles, clenched teeth, pulling on eyelashes, biting fingers or fingernails, arguing, throwing the materials, leaving the test environment, saying they are thirsty, hungry, tired, sleepy, or having to all of a sudden got to go to the bathroom, complains of their heart racing, goes selectively mute, or is unable to attempt to complete the tests in part or at all, the evaluation should include an anxiety, panic attack, or PTSD evaluation.

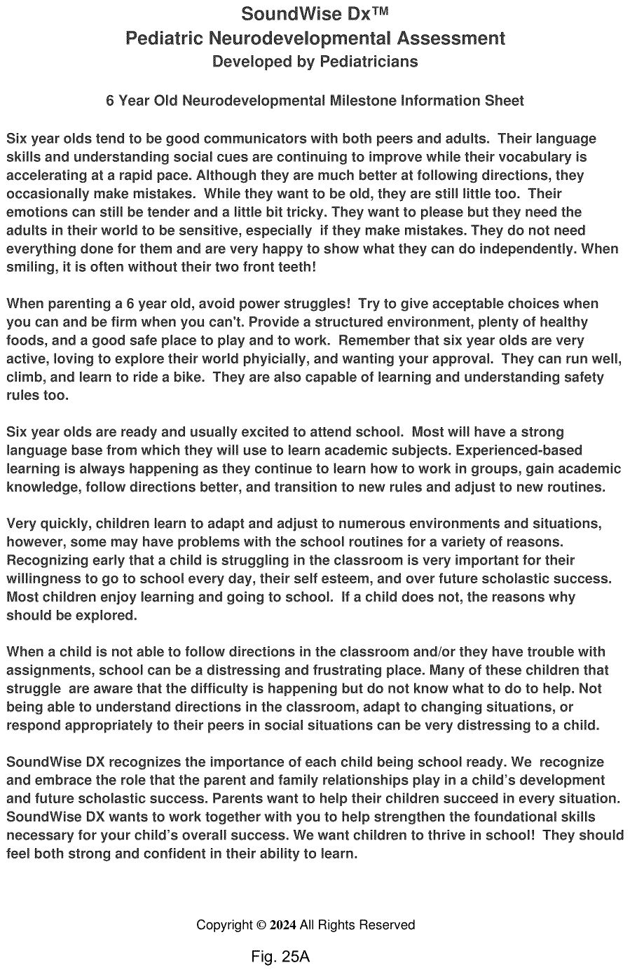

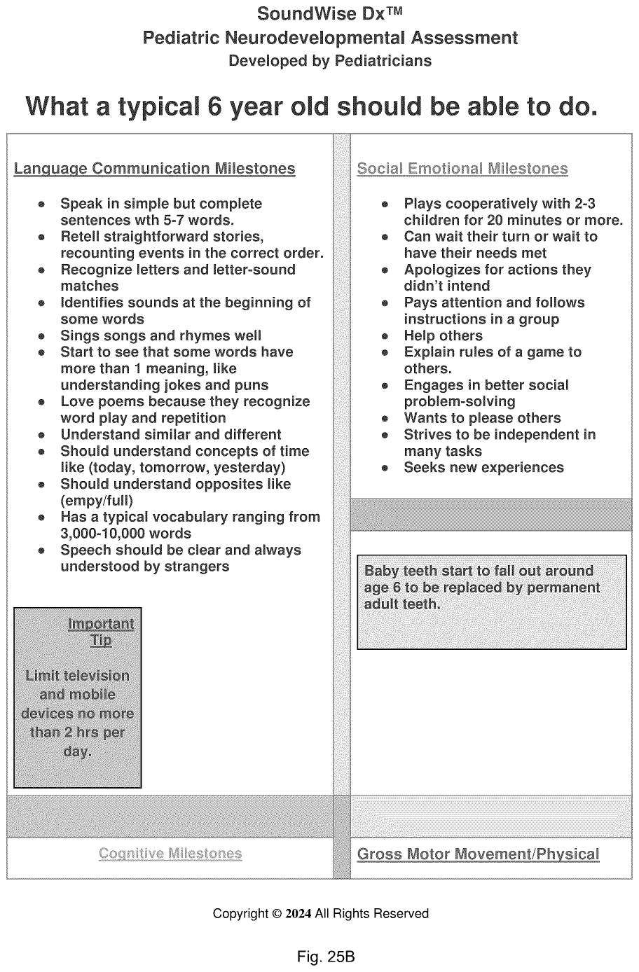

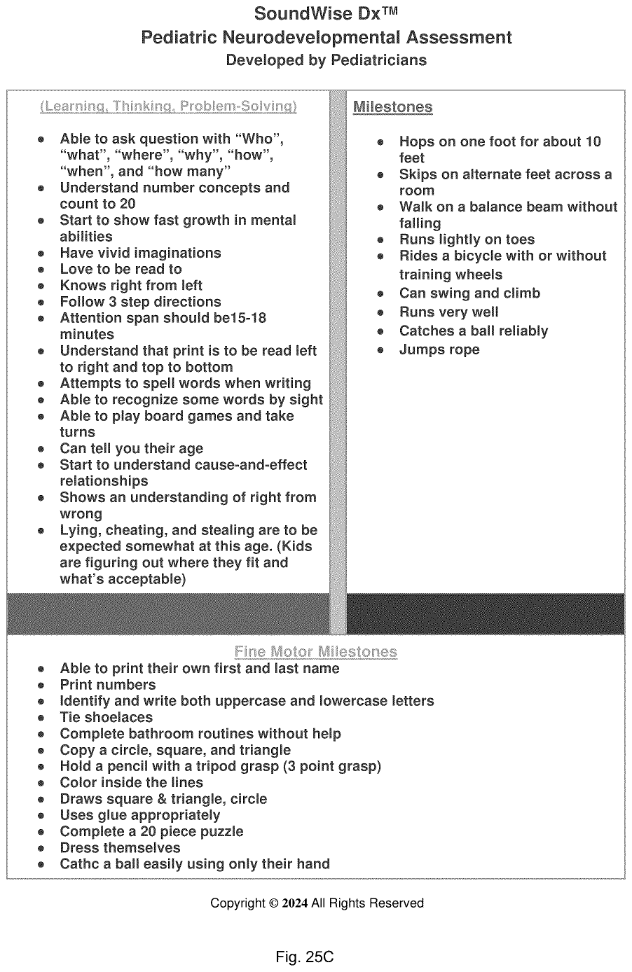

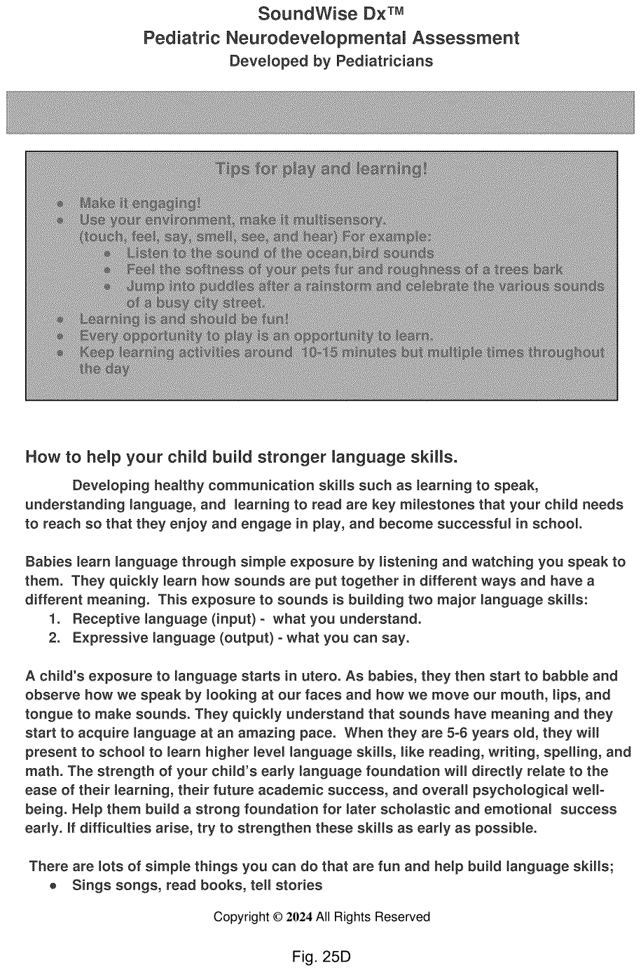

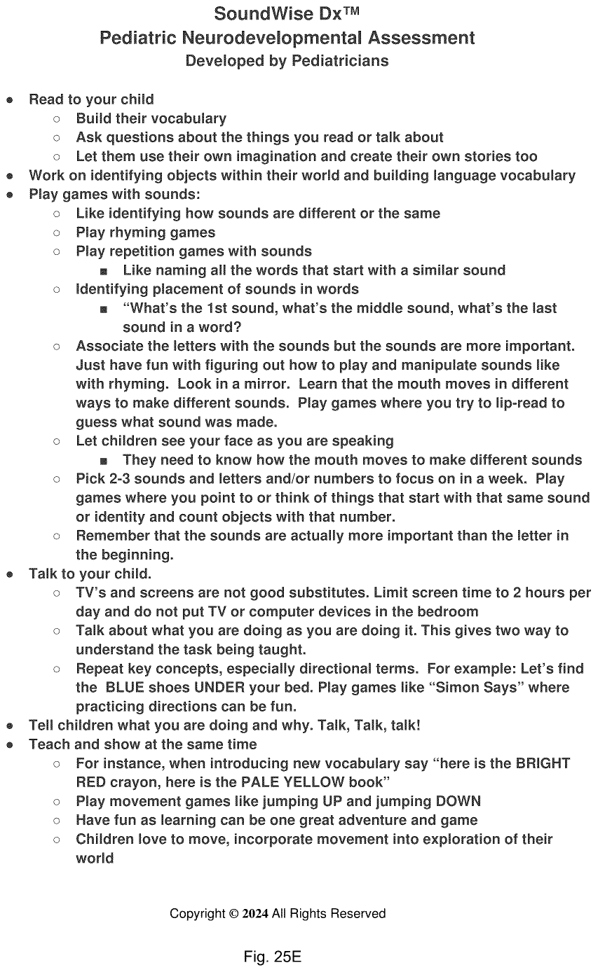

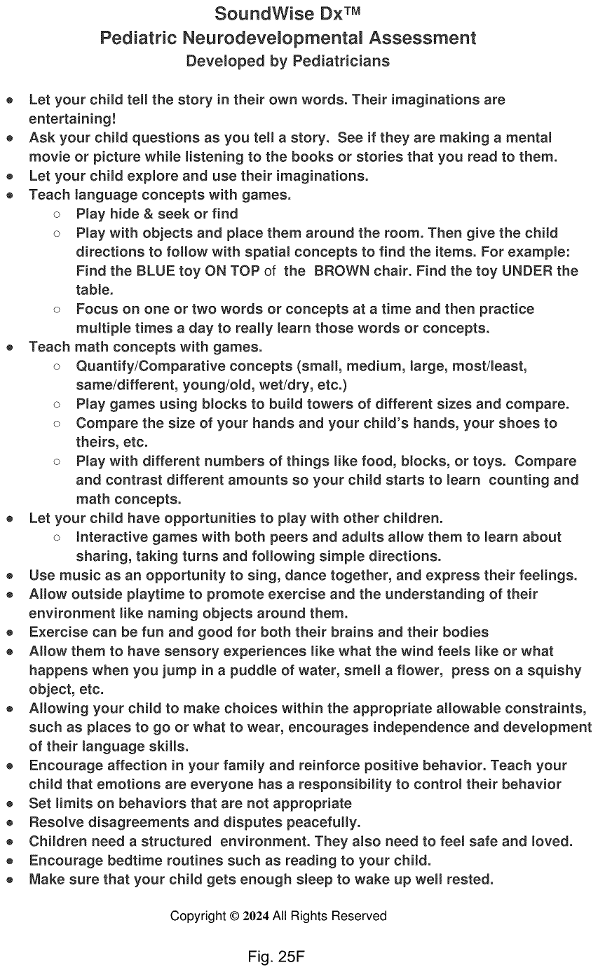

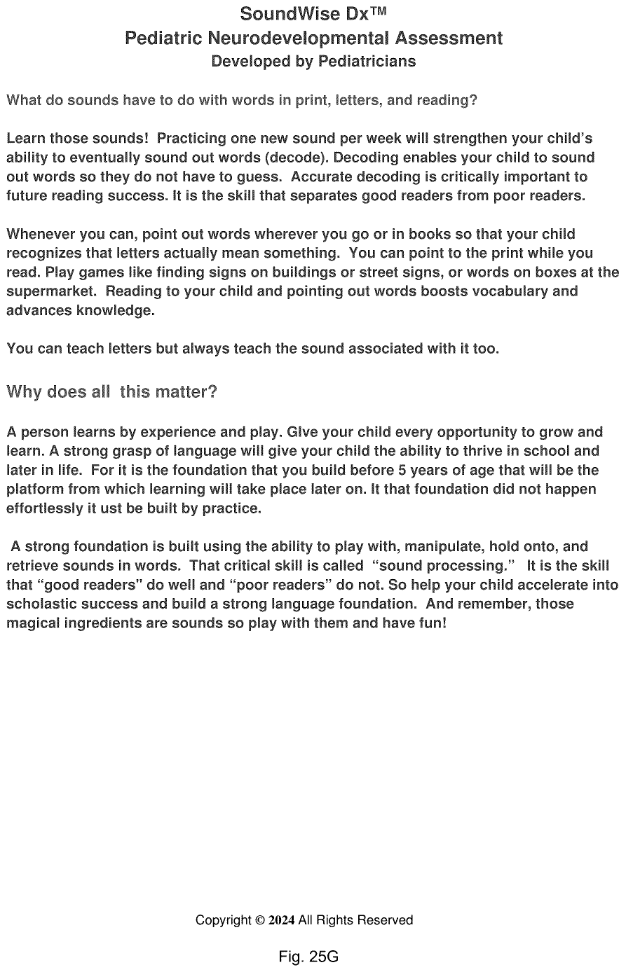

The system and method of the present disclosure may be configured to determine through an anxiety algorithm module and/or caregiver/teacher or authorized personnel to start Anxiety modules as well to start arranged telemedicine visits through the system and method of the present disclosure and/or in-person therapy. Also, the Caregiver/teacher will work on language development, social interaction, and sensory regulation by using a 6-Year-Old Developmental Milestone Information Sheet (DMIS) of the system and method of the present disclosure for age-appropriate activities. One example of a 6-Year-Old Developmental Milestone Information Sheet is shown in FIGS. 25A-25G. The system and method of the present disclosure may be configured to administer and score a diagnostic screen such as PHQ-9/SCARED/Columbia/Hamilton/GAD-7 or others for Anxiety that is appropriate for age. Based on the system and method of the present disclosure, Children will also be started on telemedicine services through the system and method of the present disclosure for anxiety.

Reasons for indications for medicine management for Anxiety is determined by the system and method of the present disclosure, physician, and input from caregiver/teacher. Pulse, Blood Pressure and EKG will be obtained by digital devices and are medically necessary before starting medication. Telemedicine services for anxiety though the system and method of the present disclosure must also be initiated and continued while the child is on medication for anxiety. All children diagnosed with Anxiety through the system and method of the present disclosure will also be evaluated for ADHD/ADD and/or Autism, if indicated.

Concerning low self-esteem, self-esteem should always be monitored closely in a child as this can lead to depression or other mental health disparities in the future. Low self-esteem can arise from a variety of reasons. The system and method of the present disclosure may include one or more modules that address building self-esteem and confidence in a child. Telemedicine visits will be recommended via the system and method of the present disclosure or a local provider.

In the fourth part of the method, a referral may also generate referring the individual for services in areas where the evaluation results were sufficient to improve the child's skills, even though the child's skills are proficient for his or her age. For example, if the child demonstrates that he or she is able to demonstrate social communication, interaction, eye contact and able to demonstrate joint attention back and forth with assessment tasks, it may be indicative that the child's skills are not deficient, and the child needs to only to continue to build and/or improve his or her skills. Further, not showing restrictive and repetition behaviors, such as flapping their hands, spinning a lot, being only interested in lining up toys or objects, having sensory issue such as being upset with loud noises and difficulty with change of routine may also be indicative that the child's skills are not deficient, and the child needs to only to continue to build and/or improve his or her skills. Indeed, if the child is able to maintain attention, is not easily distracted, follows directions, etc. it may also be indicative that the child's skills are not deficient, and the child needs to only continue to build and/or improve his or her skills. Likewise, if the child is able to complete the tests without crying or worrying about how they are doing on the tests, it may also be indicative that the child's skills are not deficient, and the child needs to only to continue to build and/or improve his or her skills.

In some embodiments, the fourth part of the method further includes observing behavioral engagement skills of the child throughout the testing process, such as, for example, during the first test of the fourth part of the method, during the second test of the fourth part of the method, during the third test of the fourth part of the method, during the fourth test of the fourth part of the method and/or during the fifth test of the fourth part of the method. In some embodiments, the observations made throughout the testing process of the fourth part of the method can include noting the child's demeanor, such as, for example, observations pertaining to whether the child is cooperative, uncooperative, refuses to interact, completed the tests independently, exhibited poor eye contact, smiled, cried, was tired, would not engage, was shy, was distractible, was anxious, needed redirection for completion, was happy and engaged, was an English language learner, was difficult to motivate, etc. In some embodiments, the observations made throughout the testing process of the fourth part of the method are included in the worksheet (FIG. 22A-22I or 23A-23I) and/or are used to generate the referral. In some embodiments, information pertaining to the answers provided in the questionnaire of the fourth part of the method (FIG. 21) is included in the worksheet and/or is used to generate the referral.

### Method—Part Five—Years Seven and Eight

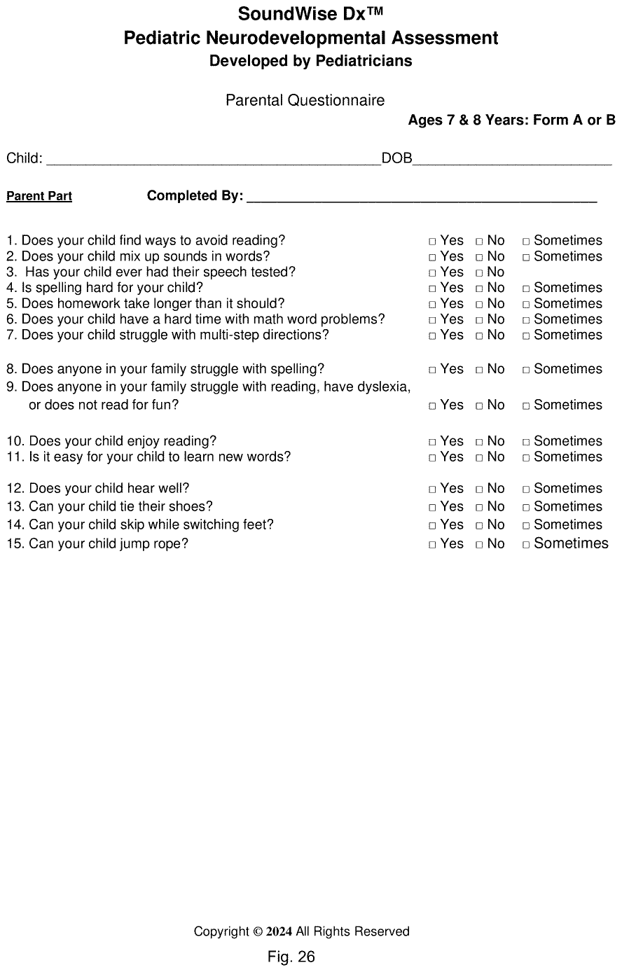

In a fifth part of the method, which should be administered after the fourth part of the method discussed above is administered to the child, when the child is approximately 7 or 8 years old, the child's parent's is required to fill out a questionnaire that includes various questions about the child. One embodiment of the questionnaire is shown in FIG. 26. As shown in FIG. 26, the questionnaire can include questions about the child's ability to read, ability to spell as well as the child's physical abilities, such as, for example, the child's hearing and vision. The questionnaire can also include questions relating to the child's ability to read, write, comprehend information, perform simple tasks, socialize, etc. For example, the questionnaire can include questions whether they can understand their child when the child speaks, whether the child avoids reading, mixes up sounds in words, has had their speech tested, whether the child has difficulty spelling, whether homework takes the child longer than it should, whether the child has difficulty with math word problems, whether the child struggles with multi-step directions, whether there is a family history of disorders, whether the child enjoys reading, whether it is easy for the child to learn new words, whether the child can tie their own shoes, etc. (See, FIG. 26).

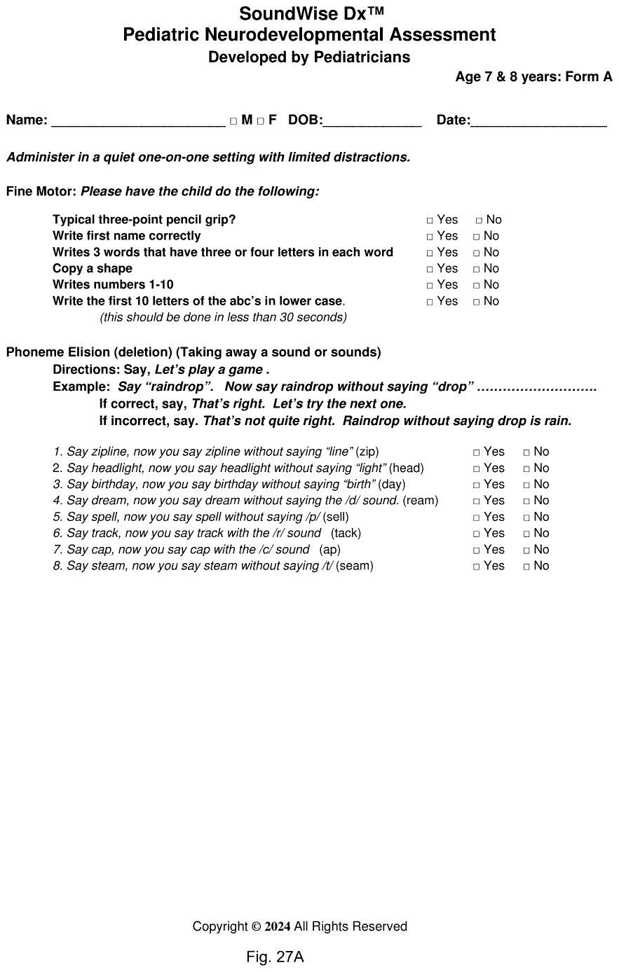

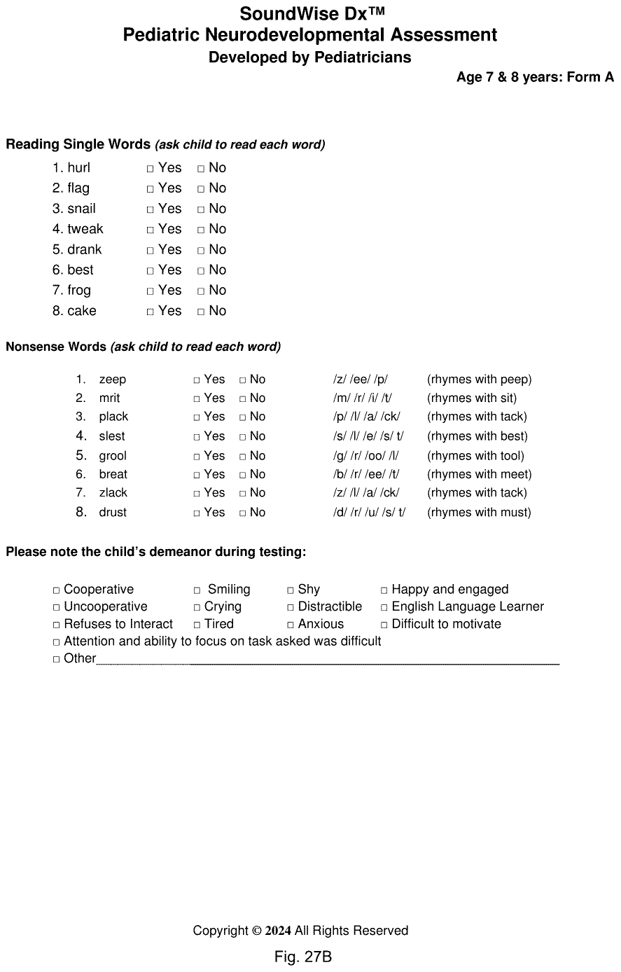

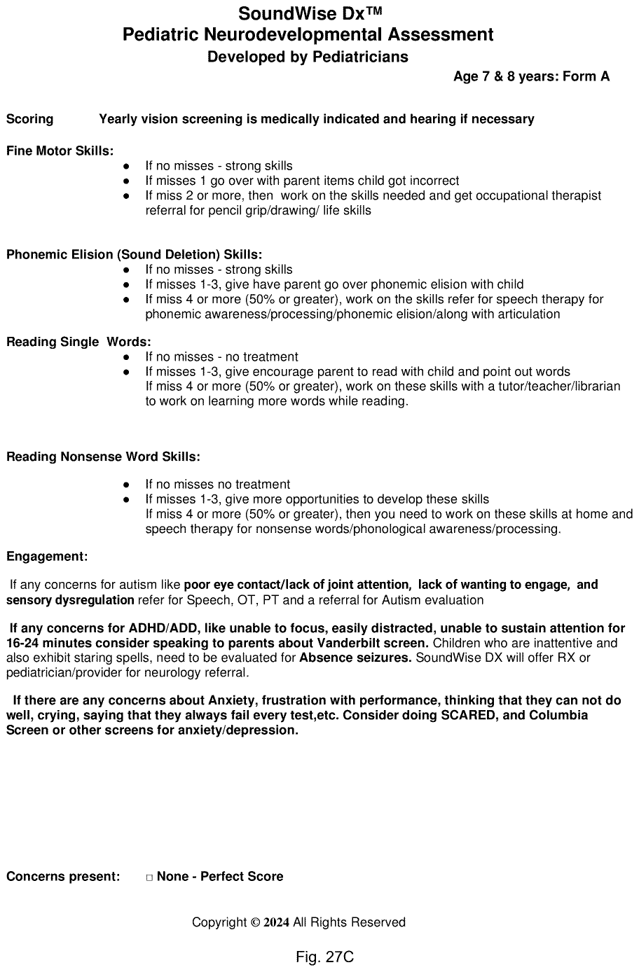

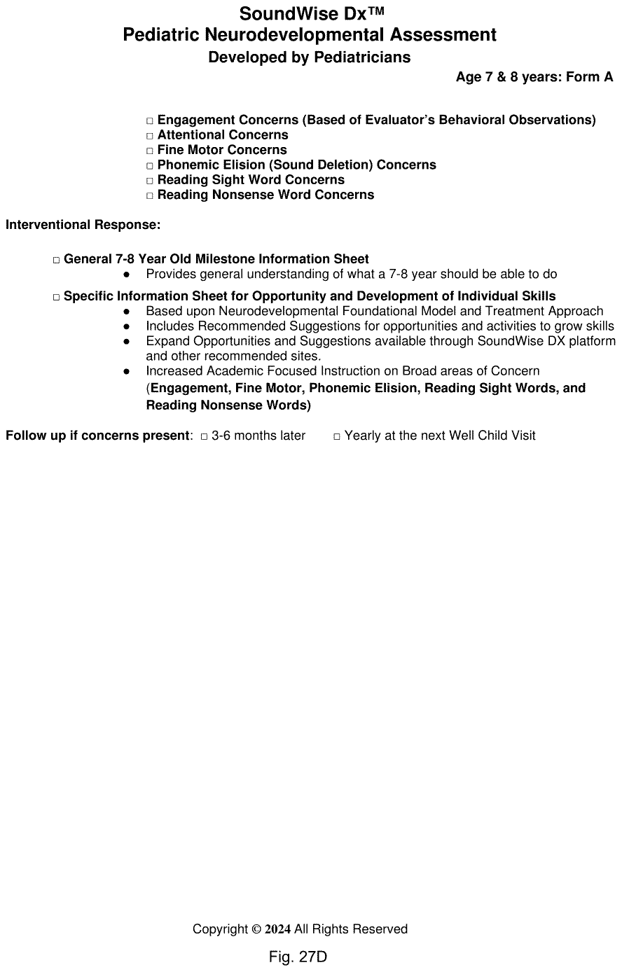

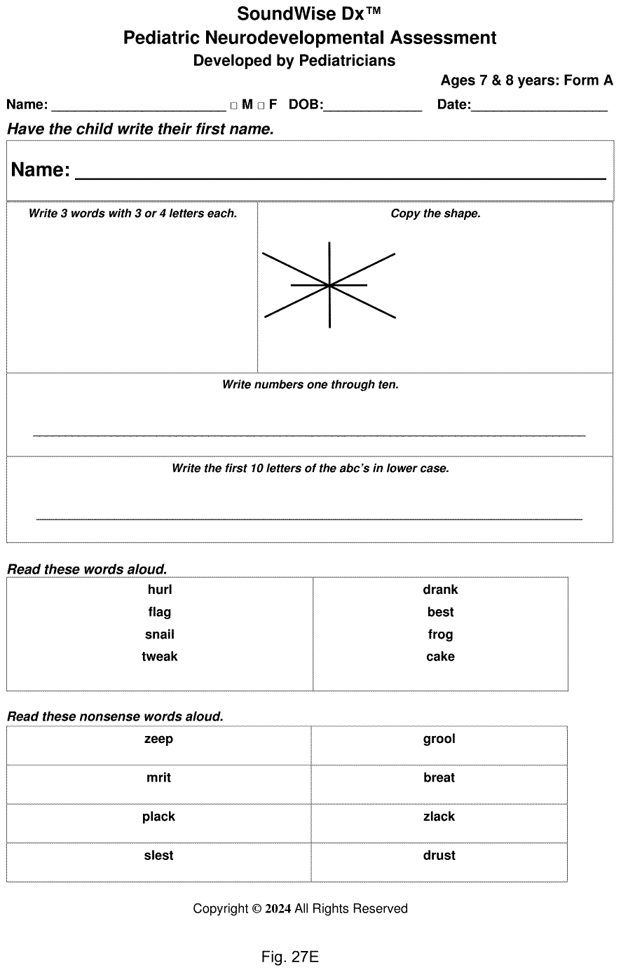

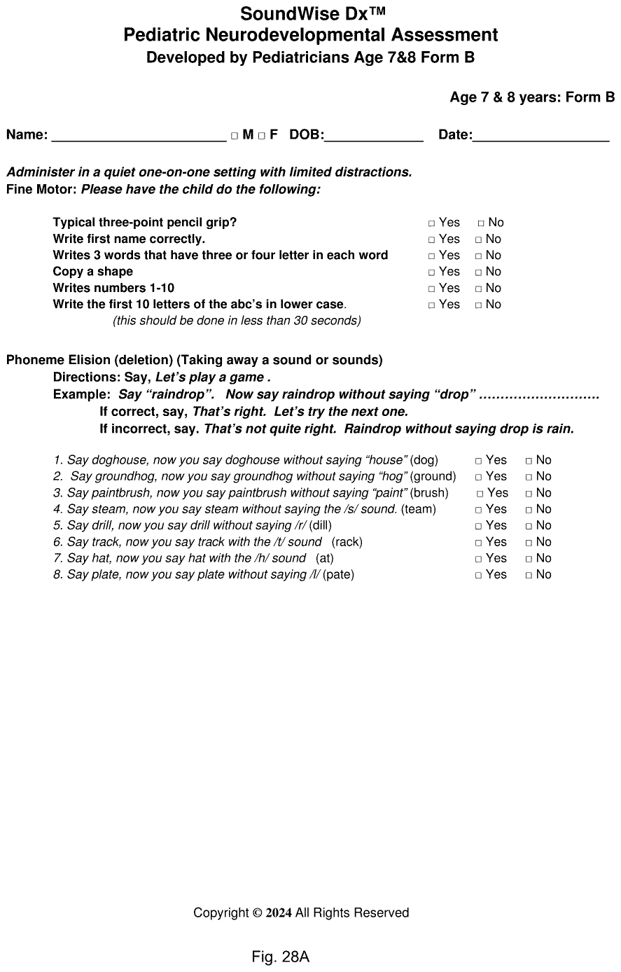

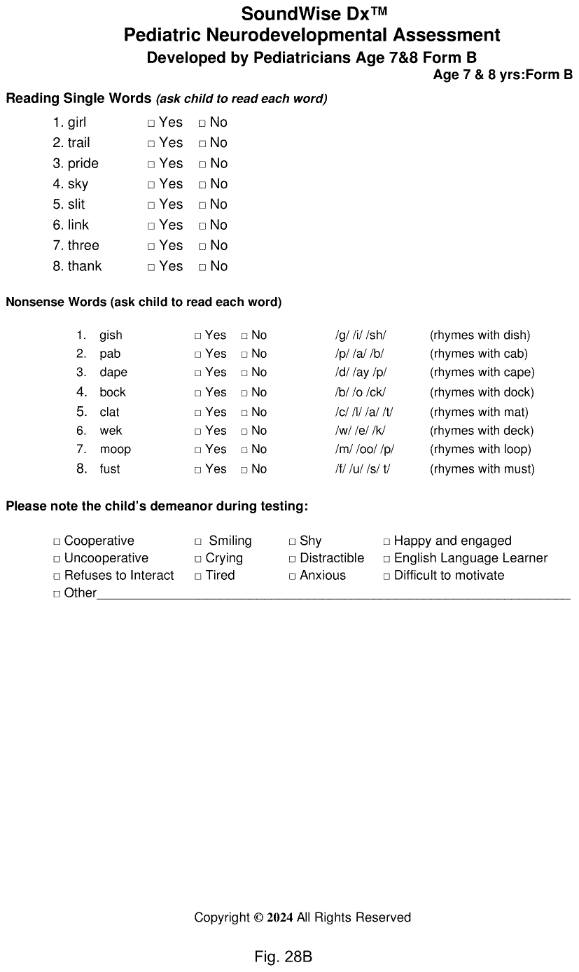

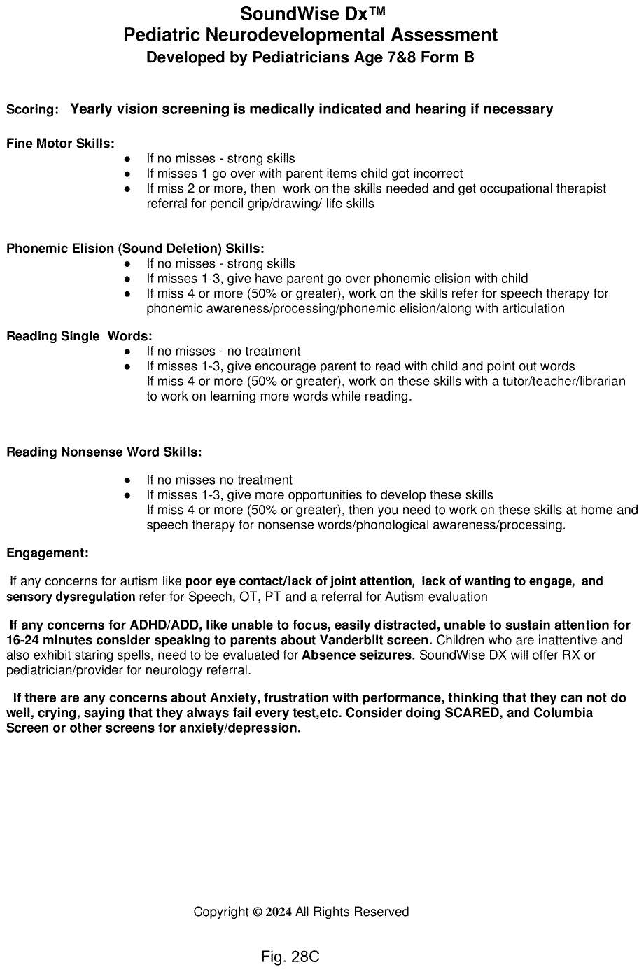

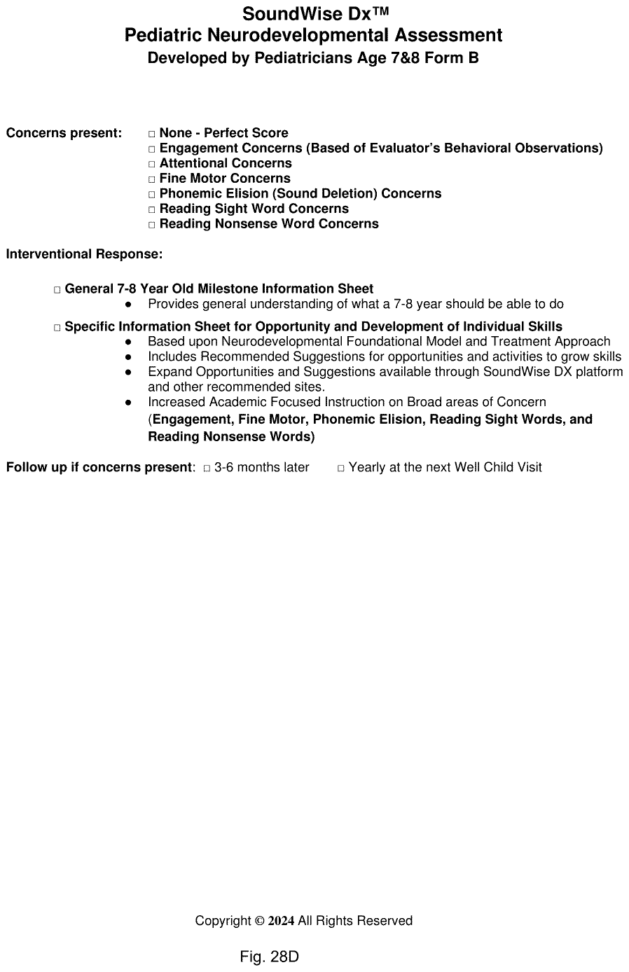

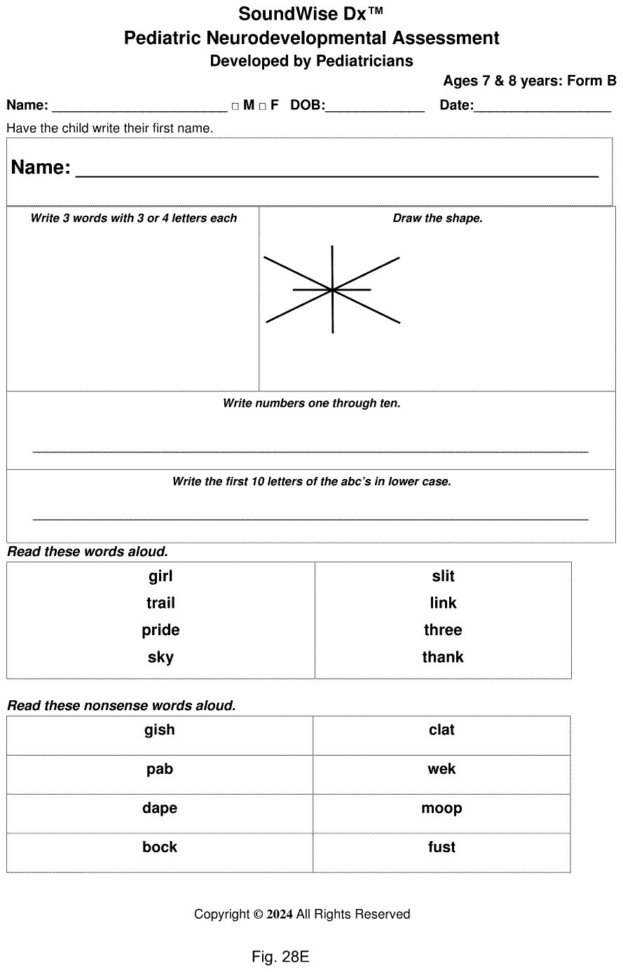

In the fifth part of the method, the child may be required to perform tests relating to fine motor skills, phoneme elision (deletion) skills, rhyming and/or reading skills (See, e.g., FIGS. 27A-27E; and FIGS. 28A-28E. For example, as shown in FIGS. 27A and 28A, the child may be required to perform a typical three-point pencil grip, write his or her first name, write three words that have three or four letters in each word, copy a shape, write numbers 1-10 and/or write the first 10 letters of the ABCs in lower case. In one embodiment, the child may also be required to read one or more single words (FIGS. 27A and 28A). In one embodiment, the child may also be required to read nonsense or nonreal words in which the child indicates whether or not the nonsense or nonreal word rhymes with an existing or proper word (FIGS. 27A and 28A).

In the fifth part of the method, the tests discussed above and shown in FIGS. 27A-22E and/or FIGS. 28A-28E relate to fine motor skills, phonemic elision (sound deletion) skills and reading skills (FIG. 27C and/or FIG. 28C). The child's behavior may be observed during these tests for considerations such as, for example, whether the child is cooperative, whether the child is uncooperative, whether the child refuses to interact, whether the child completed tasks independently, whether the child exhibited poor eye contact, whether the child smiled, whether the child cried, whether the child appeared tired, whether or not the child would engage, whether the child was shy, whether the child was distractible, whether the child was anxious, whether the child needed redirection to complete tasks, whether the child was happy and engaged, whether the child is an English language learner, whether the child was difficult whether the child's speech was understandable, etc., as shown in FIGS. 27B and 28B.

In the fifth part of the method, tests for fine motor skills, phonemic elision (sound deletion) skills and reading skills (FIG. 27C or 28C) are evaluated for the presence or absence of a disorder and/or school readiness. The ability of the child to perform the requested tasks is recorded to generate input data. The ability of the child to perform one or more of the requested tasks may be recorded in a fifth worksheet. One embodiment of the fourth worksheet is shown in FIGS. 27A-27E. Another embodiment of the fifth worksheet is shown in FIGS. 28A-28E.

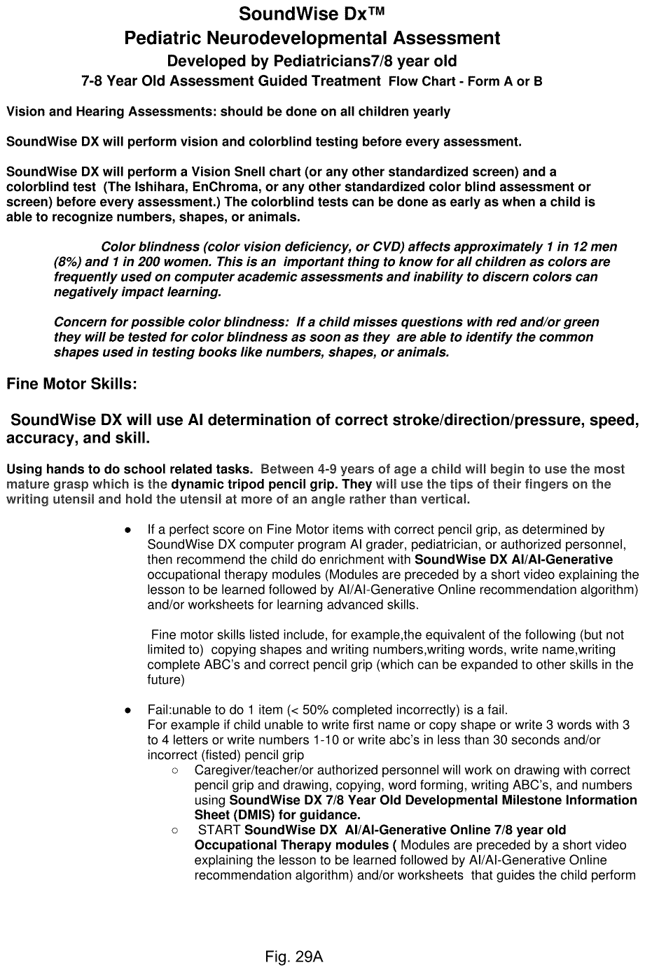

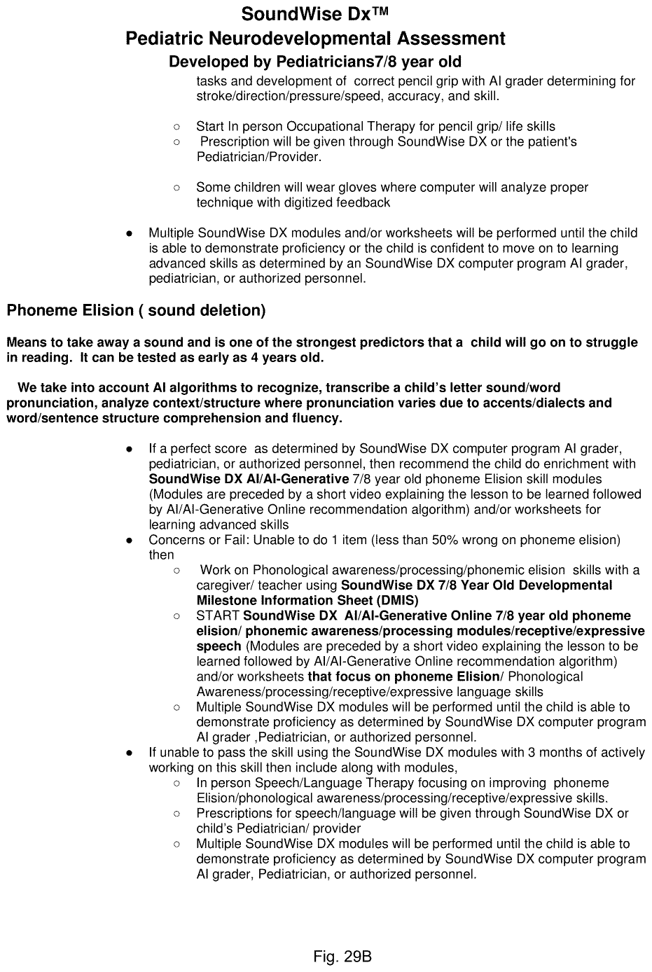

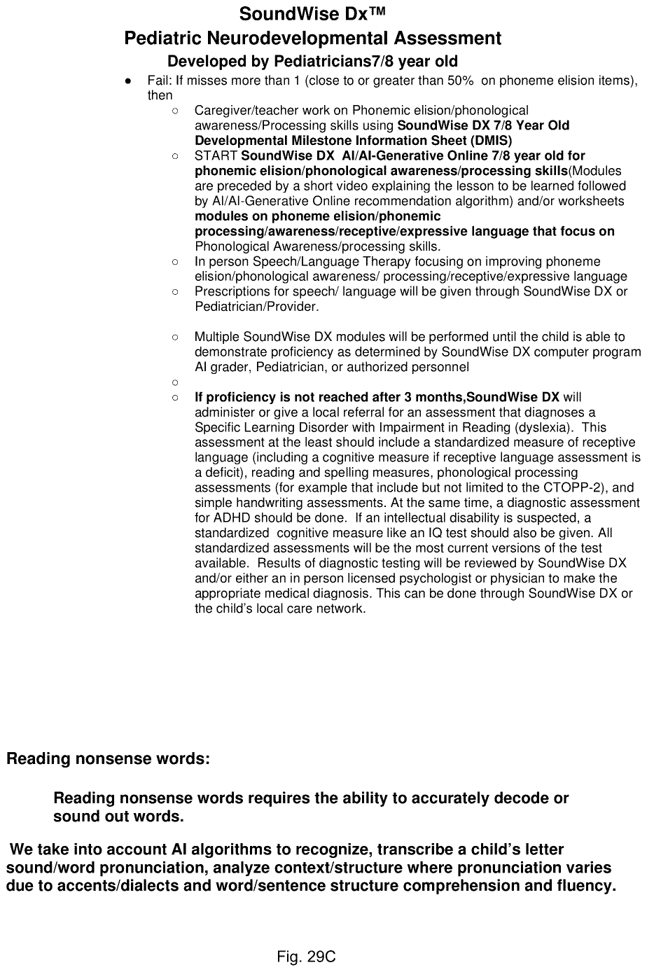

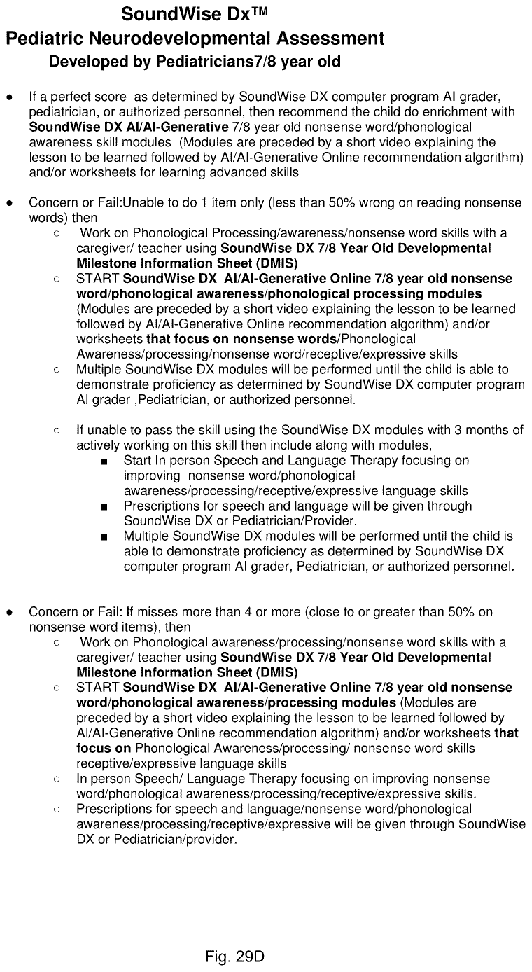

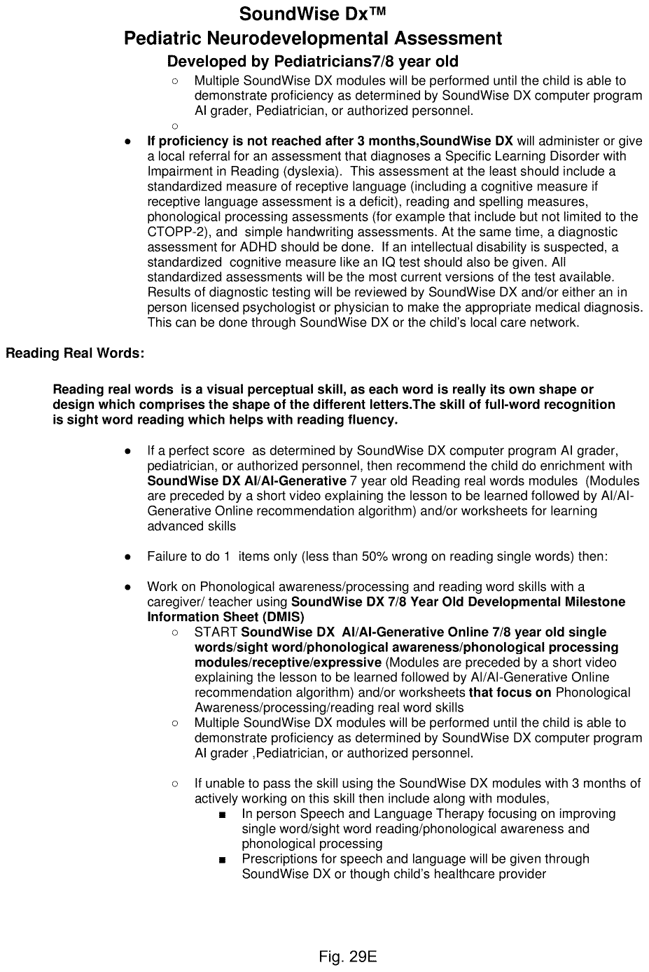

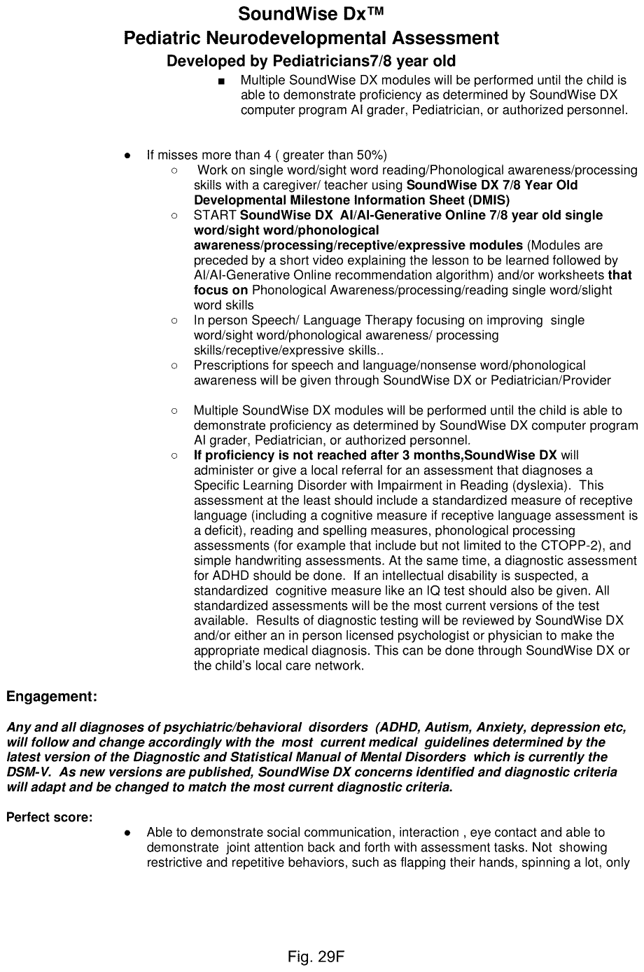

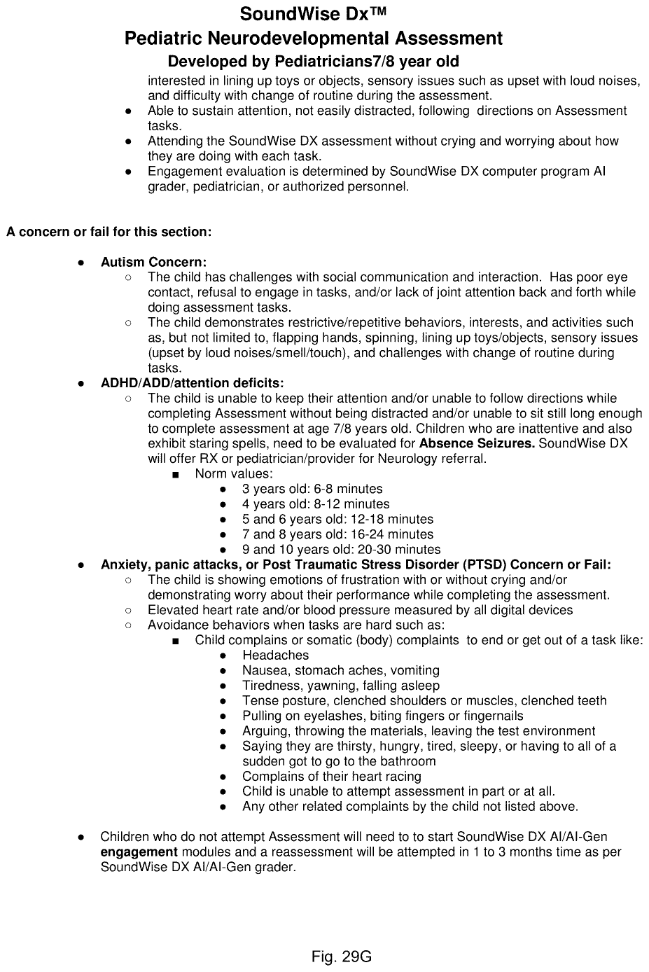

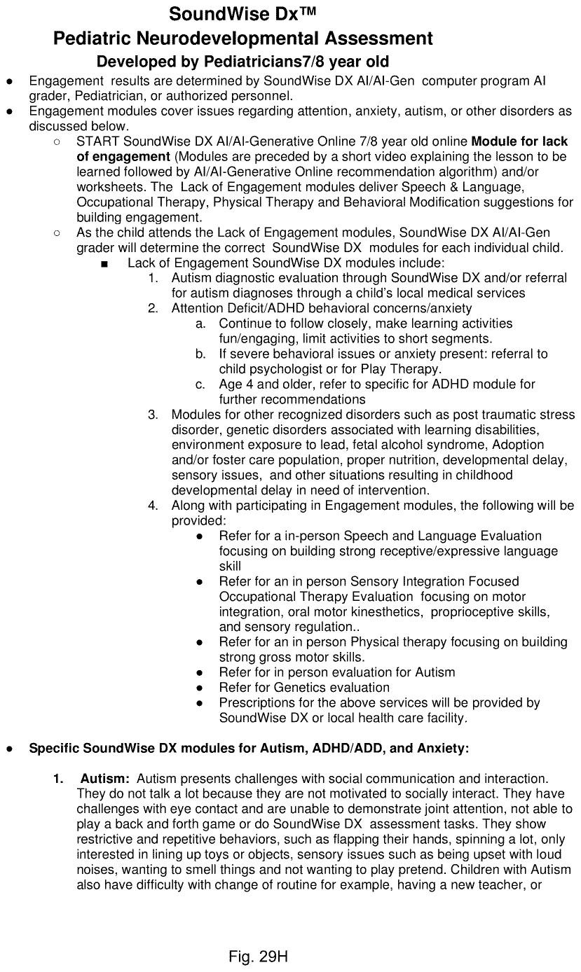

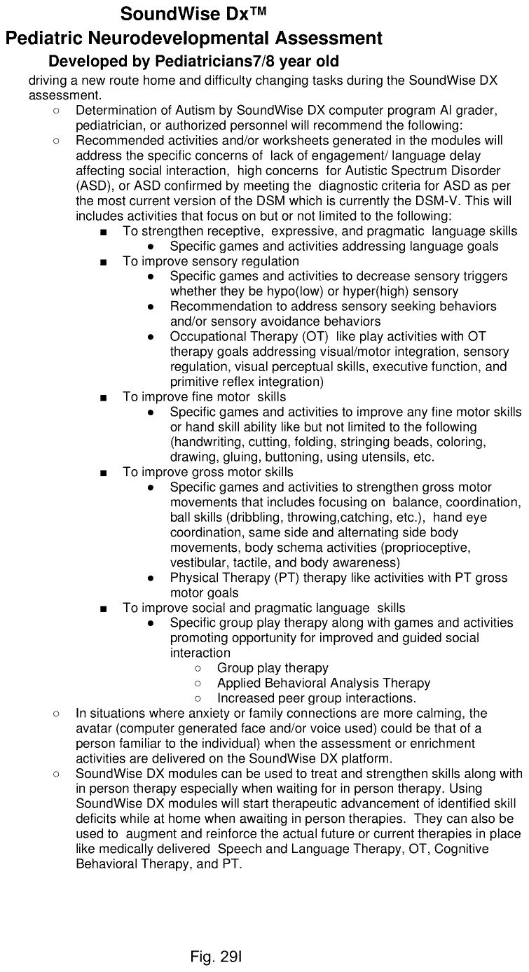

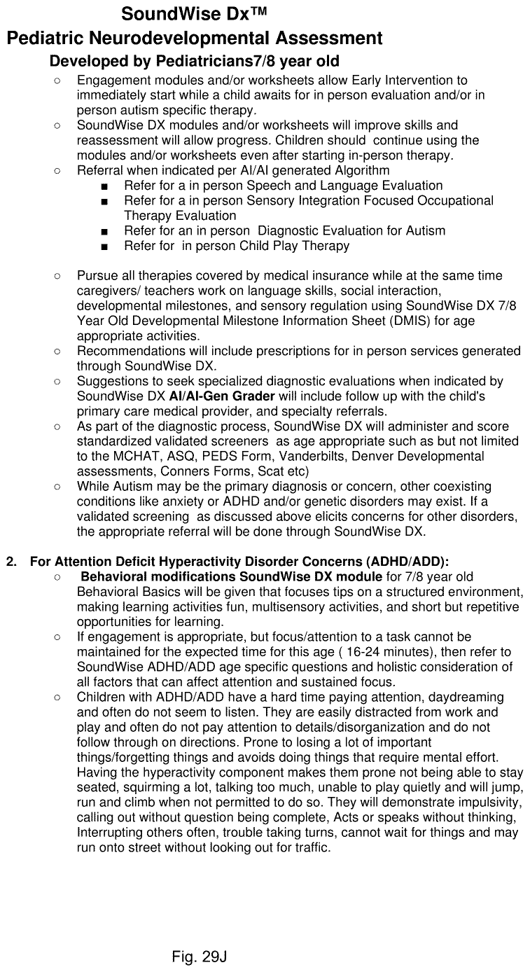

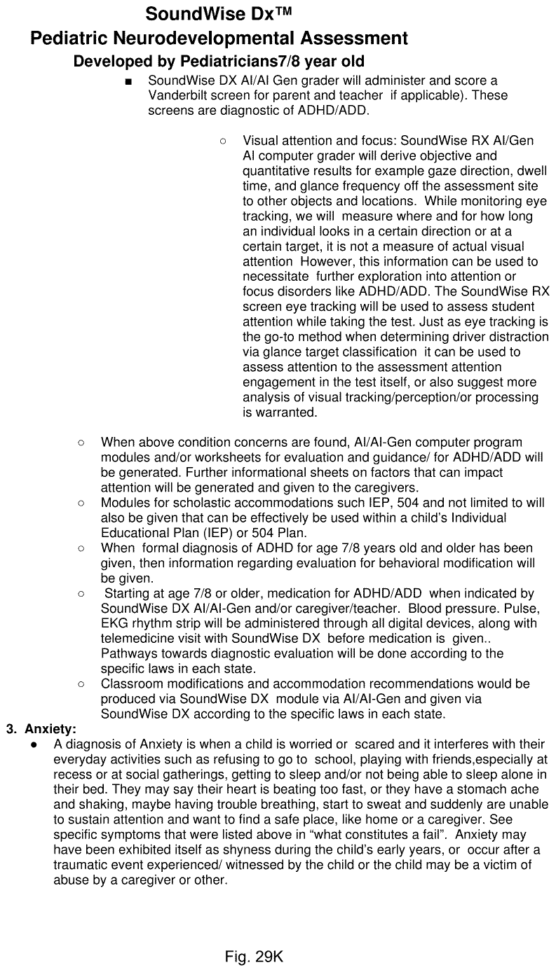

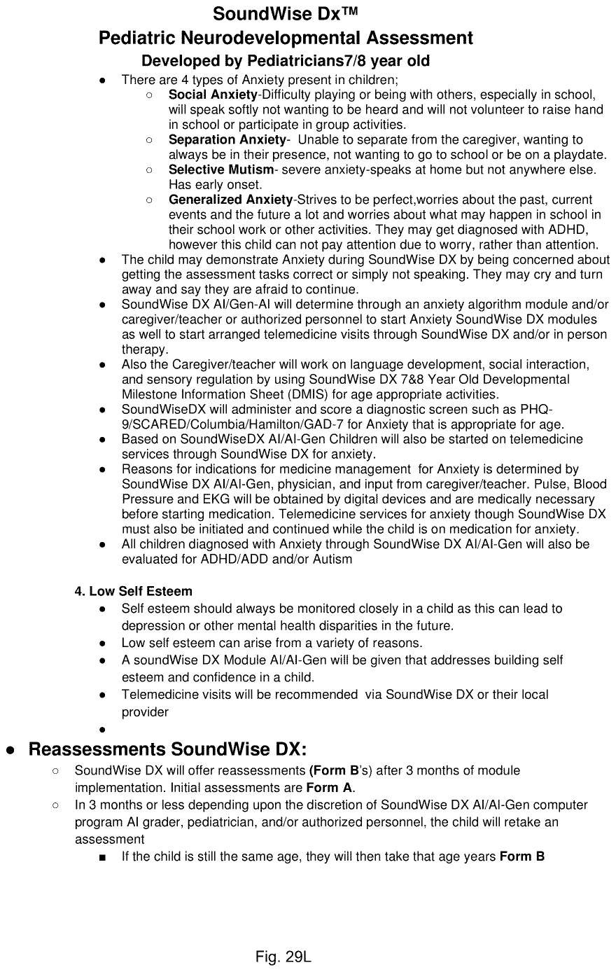

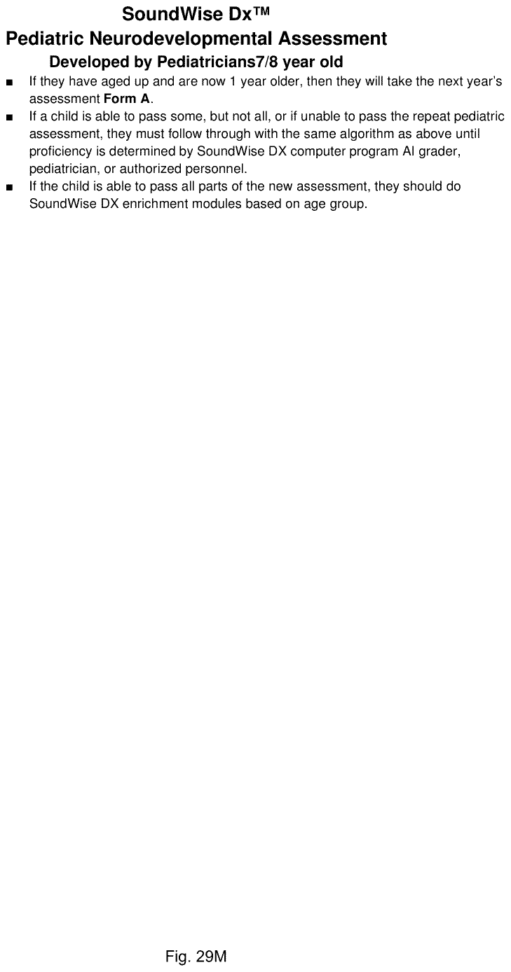

The fine motor skill input data may include whether or not the child was able to perform required tasks. The ability of the child to perform the required tasks may be recorded in the fifth worksheet. The fine motor input data is evaluated against an answer key to generate a fine motor evaluation result. The fine motor evaluation result is another determination with respect to the presence or absence of fine motor deficiency, after the fourth determination with respect the presence or absence of fine motor conducted during the fourth part of the method. In some embodiments, the fine motor evaluation result includes a score, such as, for example, a number score. In some embodiments, the fine motor evaluation result includes a written evaluation, such as, for example, words written in sentence or note form that explain what was observed by the practitioner. In some embodiments, the fine motor result is included in an assessment that evaluates at least each of the tests administered during the fourth part of the method. One embodiment of the assessment is shown in FIGS. 29A-29M.

In the fifth part of the method, a second test is administered in which the child has to do a phonemic elision (sound deletion) task to generate a phonemic elision task input data. The ability of the child to do a phonemic elision task may be recorded in the fourth worksheet (FIG. 27A-27E or FIG. 28A-28E). The phonemic elision input data is evaluated against an answer key to generate a phonemic elision evaluation result. The phonemic elision evaluation result is another determination with respect to the presence or absence of phonemic elision deficiency, after the fourth determination with respect the presence or absence of phonemic elision deficiency conducted during the fourth part of the method. In some embodiments, the phonemic elision evaluation result includes a score, such as, for example, a number score. In some embodiments, the phonemic elision evaluation result includes a written evaluation, such as, for example, words written in sentence or note form that explain what was observed by the practitioner. In some embodiments, the phonemic elision evaluation result is included in the assessment (FIGS. 29A-29M). In some embodiments, the phonemic elision evaluation result uses AI or other software to recognize and/or transcribe the child's letter sound/word pronunciation, analyze context/structure where pronunciation varies due to accents/dialects and word/sentence structure comprehension and fluency.

In the fifth part of the method, a third test is administered in which the child has to read sight words to generate a sight word reading input data. The ability of the child to do a sight word task may be recorded in the third worksheet (FIG. 27A-27E or FIG. 28A-28E). The sight word reading input data is evaluated against an answer key to generate a sight word reading evaluation result. The sight word reading evaluation result is a determination with respect to the presence or absence of sight word reading deficiency conducted during the fifth part of the method. In some embodiments, the sight word reading evaluation result includes a score, such as, for example, a number score. In some embodiments, the sight word reading evaluation result includes a written evaluation, such as, for example, words written in sentence or note form that explain what was observed by the practitioner. In some embodiments, the sight word reading evaluation result is included in the assessment (FIGS. 29A-29M). In some embodiments, the sight word reading evaluation result uses AI or other software to recognize and/or transcribe the child's letter sound/word pronunciation, analyze context/structure where pronunciation varies due to accents/dialects and word/sentence structure comprehension and fluency.

In one embodiment of the fifth part of the method, a fourth test is administered in which the child has to do a nonsense words exercise that compares nonsense words with real words to generate a nonsense word skill input data. The ability of the child to do a nonsense word skill may be recorded in the fifth worksheet (FIG. 27A-27E or FIG. 28A-28E). The nonsense word skill input data is evaluated against an answer key to generate a nonsense word skill evaluation result. The nonsense word skill evaluation result is another determination with respect to the presence or absence of nonsense word skills. In some embodiments, the nonsense word skill evaluation result includes a score, such as, for example, a number score. In some embodiments, the nonsense word skill evaluation result includes a written evaluation, such as, for example, words written in sentence or note form that explain what was observed by the practitioner. In some embodiments, the nonsense word skill evaluation result is included in the assessment (FIGS. 29A-29M). In some embodiments, the nonsense word skill evaluation result uses AI or other software to recognize and/or transcribe the child's letter sound/word pronunciation, analyze context/structure where pronunciation varies due to accents/dialects and word/sentence structure comprehension and fluency.

In the fifth part of the method, a referral is generated referring the individual for services in areas where the evaluation results were deficient to promote at least one of the skills tested in the fifth part of the method. In some embodiments, the assessment (FIGS. 29A-29M), which can include one or more of the test results in the fifth part of the method are evaluated to generate the referral. In particular, if the results indicate that the child has proficient fine motor skills, the child is not referred for services, such as, for example, occupational therapy to promote advancement in fine motor skills. If, however, the test results indicate that the child is deficient with his or her fine motor skills, the child is referred for services, such as, for example, occupational therapy to promote advancement in fine motor skills. Likewise, if the results indicate that the child has proficient phonemic elision skills, the child is not referred for services, such as, for example, speech therapy to promote advancement in phonemic elision skills. If, however, the test results indicate that the child is deficient with his or her phonemic elision skills, the child is referred for services, such as, for example, Speech therapy to promote advancement in phonemic elision skills. If the results indicate that the child has proficient sight word reading skills, the child is not referred for services, such as, for example, speech therapy to promote advancement in sight word reading skills. If, however, the test results indicate that the child is deficient with his or her sight word reading skills, the child is referred for services, such as, for example, Speech therapy to promote advancement in sight word reading skills. Lastly, if the results indicate that the child has proficient nonsense word skills, the child is not referred for services, such as, for example, Speech therapy to promote advancement in nonsense word skills. If, however, the test results indicate that the child is deficient with his or her nonsense word skills, the child is referred for services, such as, for example, Speech therapy to promote advancement in nonsense word skills.

In some embodiments, the fifth part of the method includes evaluating the child for ADHD, autism, anxiety, panic attacks, post-traumatic stress disorder (PTSD), depression, low self-esteem, etc. if the evaluation results in the fifth part of the method were deficient to promote at least one of the skills tested in the fifth part of the method.

The ADHD evaluation in the fifth part of the method can include giving focused tips on a structured environment, making learning activities fun, multisensory activities, and short but repetitive opportunities for learning. If engagement is appropriate, but focus/attention to a task cannot be maintained for the age expected time for the child's age, then the method may refer the child to take a test that includes ADHD/ADD age specific questions and consider all factors that can affect attention and sustained focus. In some embodiments, if the child exhibits signs of ADHD/ADD, the method can be adapted to administer and score a Vanderbilt screen for parent and teacher (if applicable). These screens are diagnostic of ADHD/ADD. Must rule out absence seizure with EEG or with another medical device.

Concerning autism, the fifth part of the method includes evaluating whether the child has challenges with social communication and interaction, has poor eye contact, refuses to engage in tasks, and/or lacks joint attention back and forth while doing assessment tasks. For example, if the child demonstrates restrictive/repetitive behaviors, interests, and activities such as, but not limited to, flapping hands, spinning, lining up toys/objects, sensory issues (upset by loud noises/smell/touch), and challenges with change of routine during tasks, the evaluation should include an autism evaluation.

If the autism evaluation of the present method may recommend activities and/or worksheets that are adapted to address the specific concerns of lack of engagement/language delay affecting social interaction, high concerns for Autistic Spectrum Disorder (ASD), or ASD confirmed. This will include activities that focus on strengthening receptive, expressive, and pragmatic language skills; specific games and activities addressing language goals. To improve sensory regulation, the autism evaluation of the present method may recommend specific games and activities to decrease sensory triggers whether they be hypo(low) or hyper(high) sensory. To address sensory seeking behaviors and/or sensory avoidance behaviors, the autism evaluation may include recommending Occupational Therapy (OT) like play activities with OT therapy goals addressing visual/motor integration, sensory regulation, visual perceptual skills, executive function, and primitive reflex integration.

The system and method of the present disclosure can be used to treat and strengthen skills along with in-person therapy especially when waiting for in-person therapy. The system and method of the present disclosure can recommend starting therapeutic advancement of identified skill deficits while at home when awaiting in-person therapies. Such therapeutic advancement can be used to augment and reinforce the actual future or current therapies in place like medically delivered Speech and Language Therapy, OT, Cognitive Behavioral Therapy, and PT. The system and method of the present disclosure can include engagement modules and/or worksheets that allow Early Intervention to immediately start while a child waits for in-person evaluation and/or in-person autism specific therapy. The system and method of the present disclosure can improve skills and reassessment will allow progress. Children should continue using the system and method of the present disclosure even after starting in-person therapy. Referral pursuant to the autism evaluation can include referring the child for an in-person Speech and Language Evaluation, referring the child for an in-person Sensory Integration Focused Occupational Therapy Evaluation, referring the child for an in-person Diagnostic Evaluation for Autism, referring the child for in-person Child Play Therapy.

Concerning anxiety, panic attacks, or PTSD, if the child is showing emotions of frustration with or without crying and/or demonstrating worry about their performance while completing the assessment, has an elevated heart rate and/or blood pressure and/or avoids behaviors when tasks are hard such as: complaining to end or get out of a task like, having headaches, having nausea, stomach aches, vomiting, having tiredness, yawning, falling asleep, having a tense posture, clenched shoulders or muscles, clenched teeth, pulling on eyelashes, biting fingers or fingernails, arguing, throwing the materials, leaving the test environment, saying they are thirsty, hungry, tired, sleepy, or having to all of a sudden got to go to the bathroom, complains of their heart racing or is unable to attempt to complete the tests in part or at all, the evaluation should include an anxiety, panic attack, or PTSD evaluation.

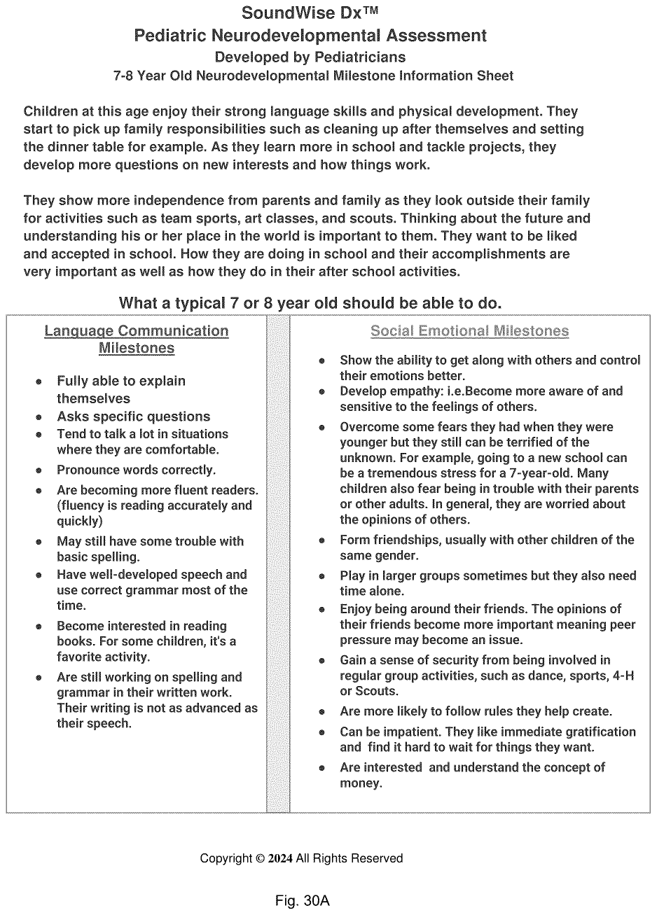

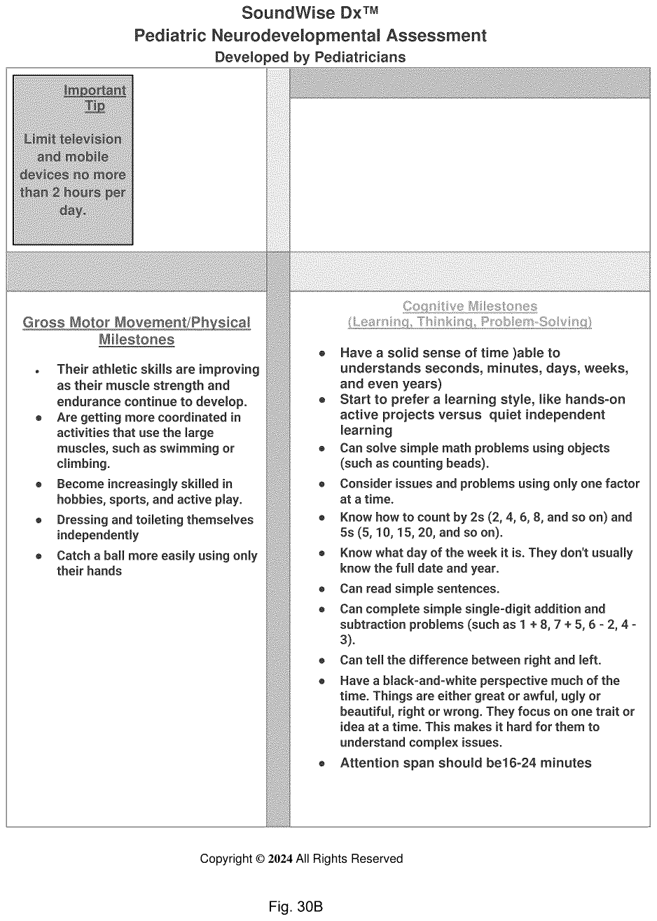

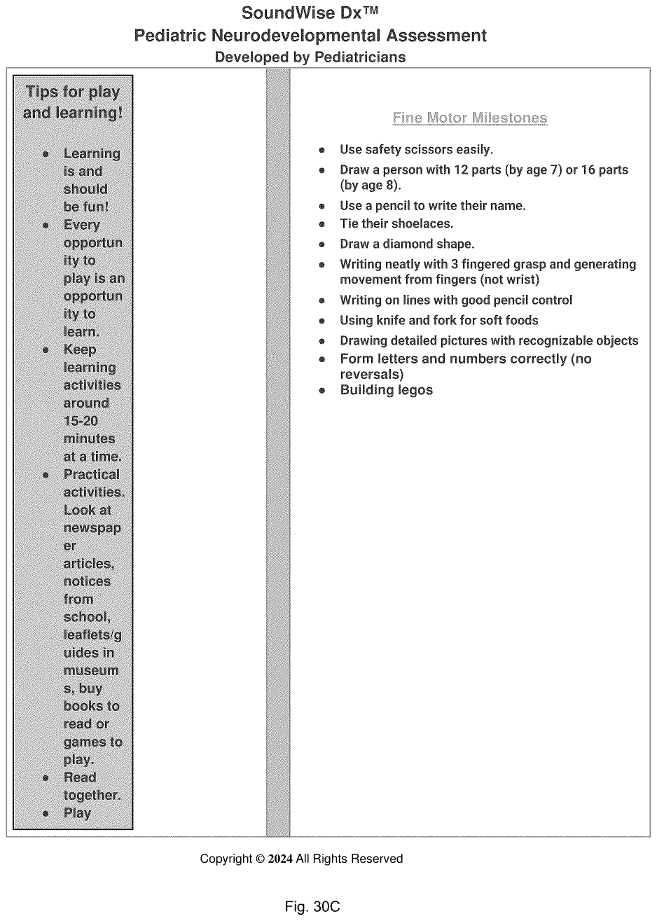

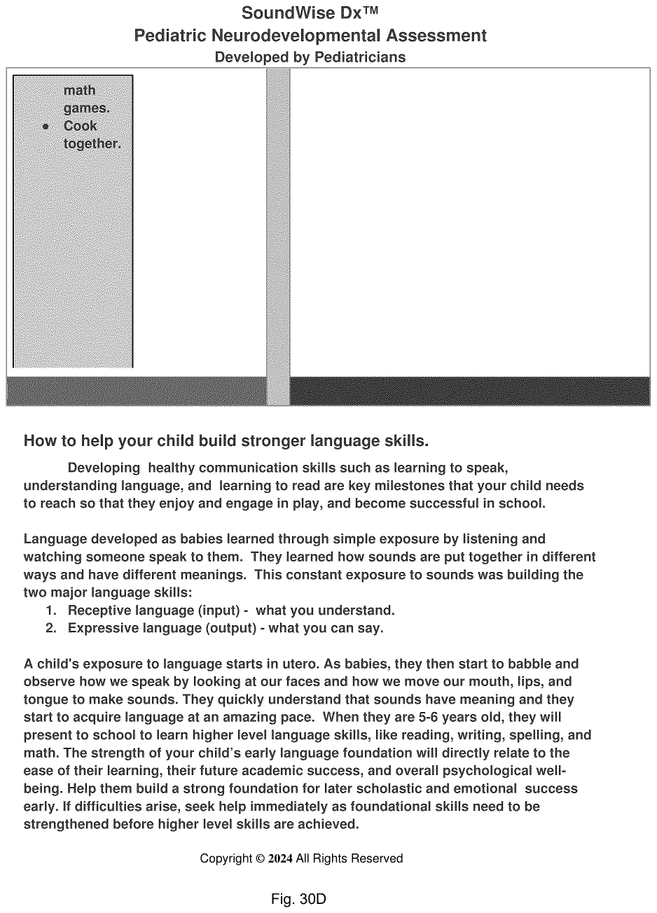

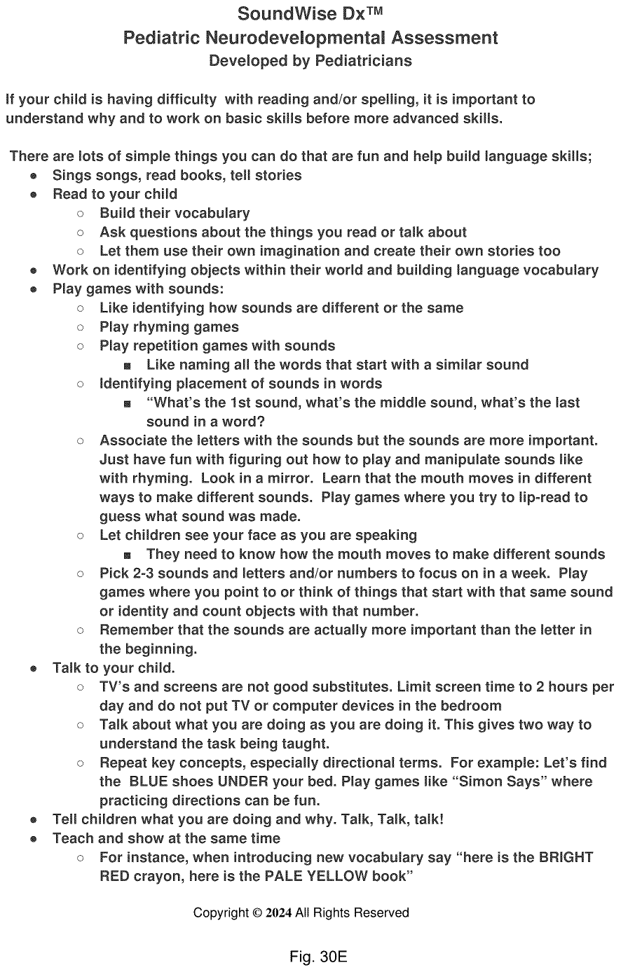

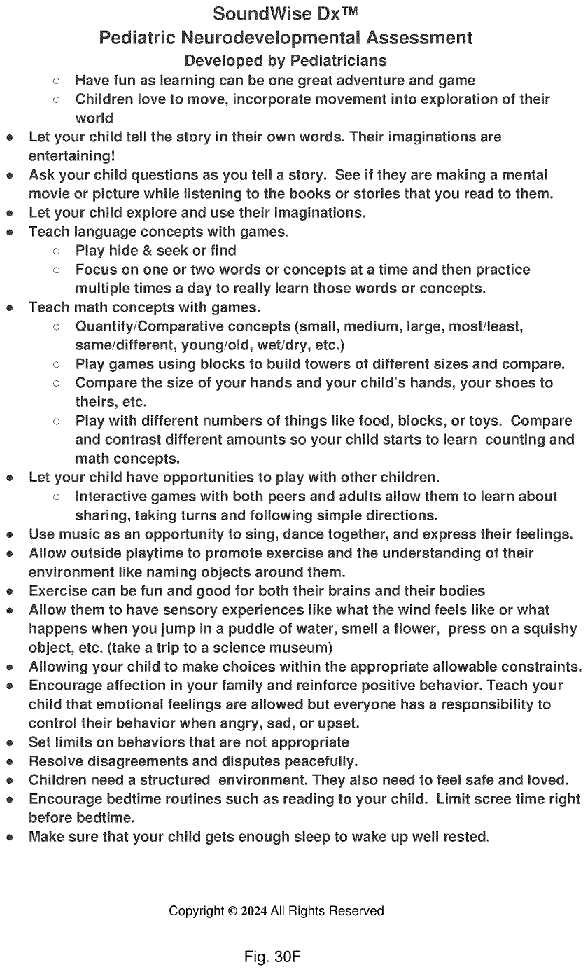

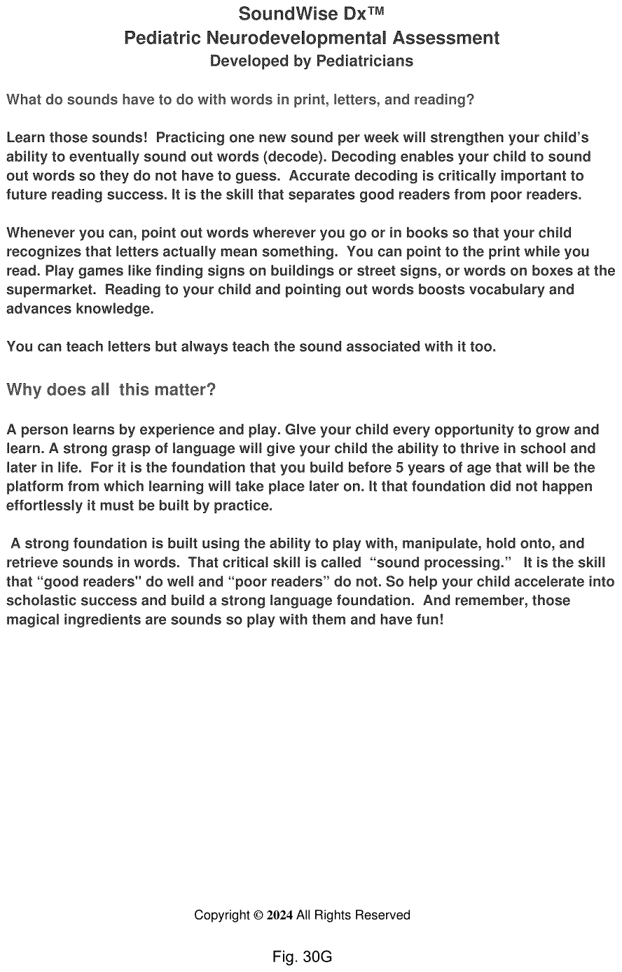

The system and method of the present disclosure may be configured to determine through an anxiety algorithm module and/or caregiver/teacher or authorized personnel to start Anxiety modules as well to start arranged telemedicine visits through the system and method of the present disclosure and/or in-person therapy. Also, the Caregiver/teacher will work on language development, social interaction, and sensory regulation by using a 7- and 8-Year-Old Developmental Milestone Information Sheet (DMIS) of the system and method of the present disclosure for age-appropriate activities. One example of a 7- and 8-Year-Old Developmental Milestone Information Sheet is shown in FIGS. 30A-30F. The system and method of the present disclosure may be configured to administer and score a diagnostic screen such as PHQ-9/SCARED/Columbia/Hamilton/GAD-7 or others for Anxiety that is appropriate for age. Based on the system and method of the present disclosure, Children will also be started on telemedicine services through the system and method of the present disclosure for anxiety.

Reasons for indications for medicine management for Anxiety is determined by the system and method of the present disclosure, physician, and input from caregiver/teacher. Pulse, Blood Pressure and EKG will be obtained by digital devices and are medically necessary before starting medication. Telemedicine services for anxiety though the system and method of the present disclosure must also be initiated and continued while the child is on medication for anxiety. All children diagnosed with Anxiety through the system and method of the present disclosure will also be evaluated for ADHD/ADD and/or Autism.

Concerning low self-esteem, self-esteem should always be monitored closely in a child as this can lead to depression or other mental health disparities in the future. Low self-esteem can arise from a variety of reasons. The system and method of the present disclosure may include one or more modules that address building self-esteem and confidence in a child. Telemedicine visits will be recommended via the system and method of the present disclosure or a local provider.

In the fifth part of the method, a referral may also generate referring the individual for services in areas where the evaluation results were sufficient to improve the child's skills, even though the child's skills are proficient for his or her age. For example, if the child demonstrates that he or she is able to demonstrate social communication, interaction, eye contact and able to demonstrate joint attention back and forth with assessment tasks, it may be indicative that the child's skills are not deficient, and the child needs to only to continue to build and/or improve his or her skills. Further, not showing restrictive and repetition behaviors, such as flapping their hands, spinning a lot, being only interested in lining up toys or objects, having sensory issue such as being upset with loud noises and difficulty with change of routine may also be indicative that the child's skills are not deficient, and the child needs to only to continue to build and/or improve his or her skills. Indeed, if the child is able to maintain attention, is not easily distracted, follows directions, etc. it may also be indicative that the child's skills are not deficient, and the child needs to only continue to build and/or improve his or her skills. Likewise, if the child is able to complete the tests without crying or worrying about how they are doing on the tests, it may also be indicative that the child's skills are not deficient, and the child needs to only to continue to build and/or improve his or her skills.

In some embodiments, the fifth part of the method further includes observing behavioral engagement skills of the child throughout the testing process, such as, for example, during the first test of the fifth part of the method, during the second test of the fifth part of the method, during the third test of the fifth part of the method and/or during the fourth test of the fifth part of the method. In some embodiments, the observations made throughout the testing process of the fifth part of the method can include noting the child's demeanor, such as, for example, observations pertaining to whether the child is cooperative, uncooperative, refuses to interact, completed the tests independently, exhibited poor eye contact, smiled, cried, was tired, would not engage, was shy, was distractible, was anxious, needed redirection for completion, was happy and engaged, was an English language learner, was difficult to motivate, etc. In some embodiments, the observations made throughout the testing process of the fifth part of the method are included in the worksheet (FIG. 27A-27E or 28A-28E) and/or are used to generate the referral. In some embodiments, information pertaining to the answers provided in the questionnaire of the fifth part of the method (FIG. 26) is included in the worksheet and/or is used to generate the referral.

### Method—Part Six—Years Nine+

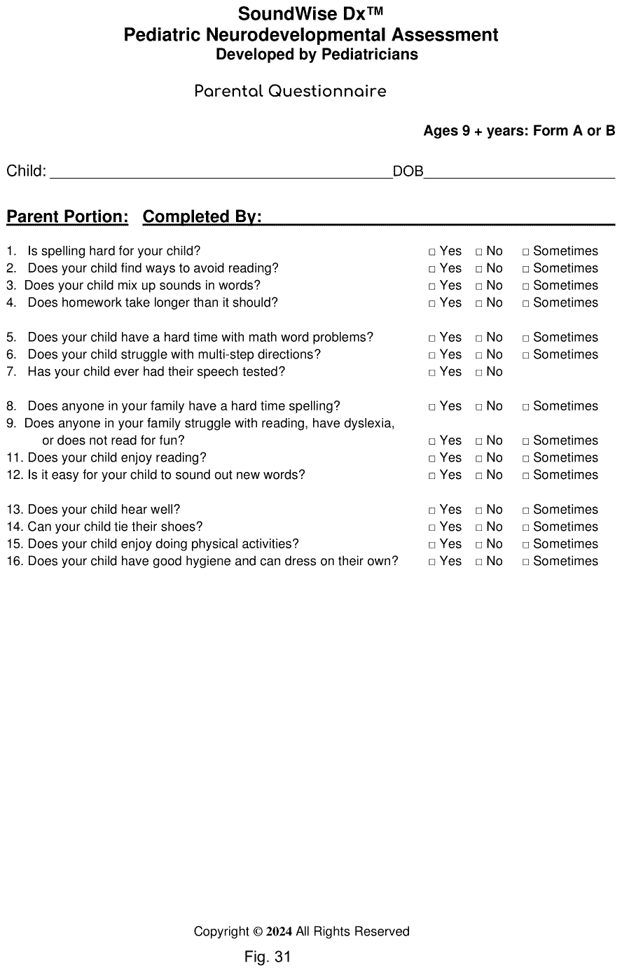

In a sixth part of the method, which should be administered after the fifth part of the method discussed above is administered to the child, when the child is approximately 9 years old or older, the child's parent's is required to fill out a questionnaire that includes various questions about the child. One embodiment of the questionnaire is shown in FIG. 31. As shown in FIG. 31, the questionnaire can include questions about the child's ability to read, ability to spell as well as the child's physical abilities, such as, for example, the child's hearing and vision. The questionnaire can also include questions relating to the child's ability to read, write, comprehend information, perform simple tasks, socialize, etc. For example, the questionnaire can include questions whether they can understand their child when the child speaks, whether the child avoids reading, mixes up sounds in words, has had their speech tested, whether the child has difficulty spelling, whether homework takes the child longer than it should, whether the child has difficulty with math word problems, whether the child struggles with multi-step directions, whether there is a family history of disorders, whether the child enjoys reading, whether it is easy for the child to sound out new words, whether the child can tie their own shoes, etc. (See, FIG. 31).

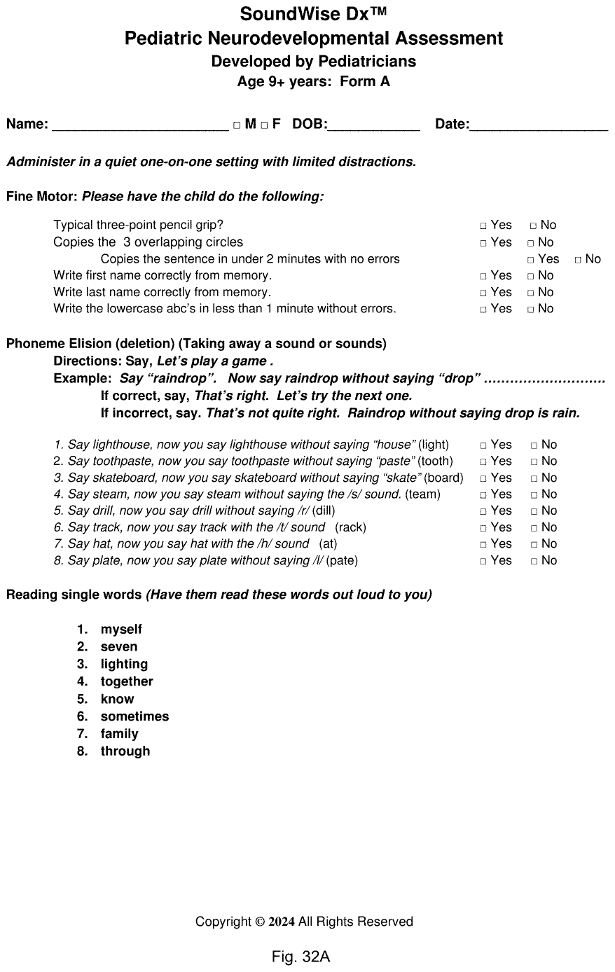

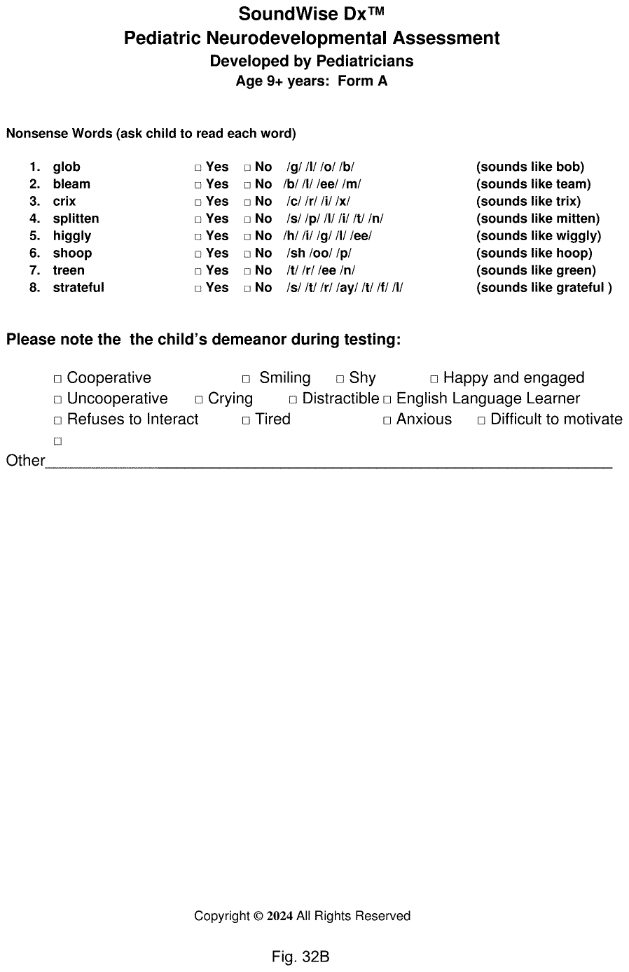

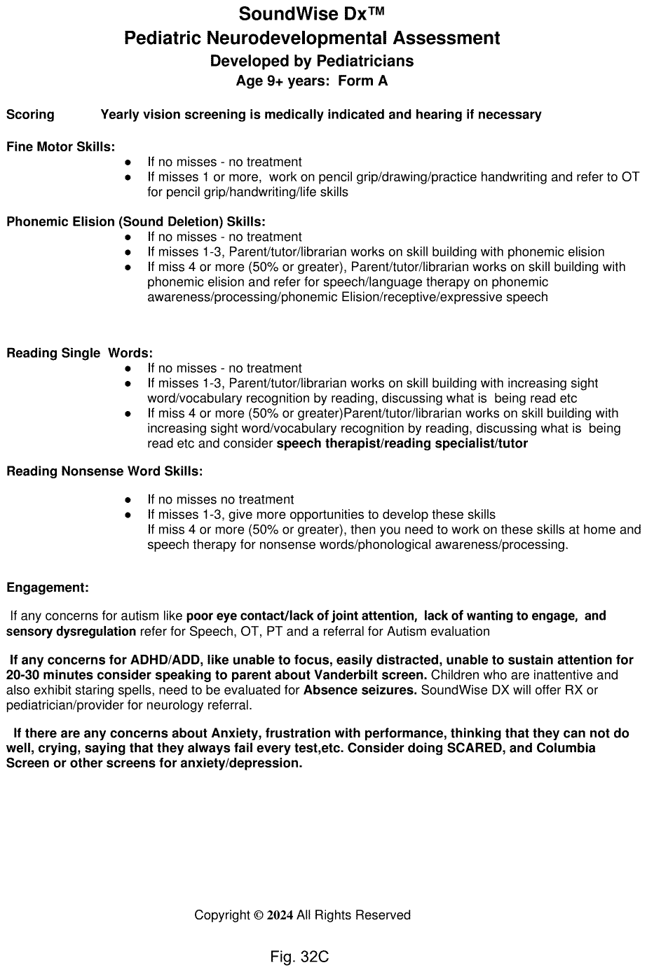

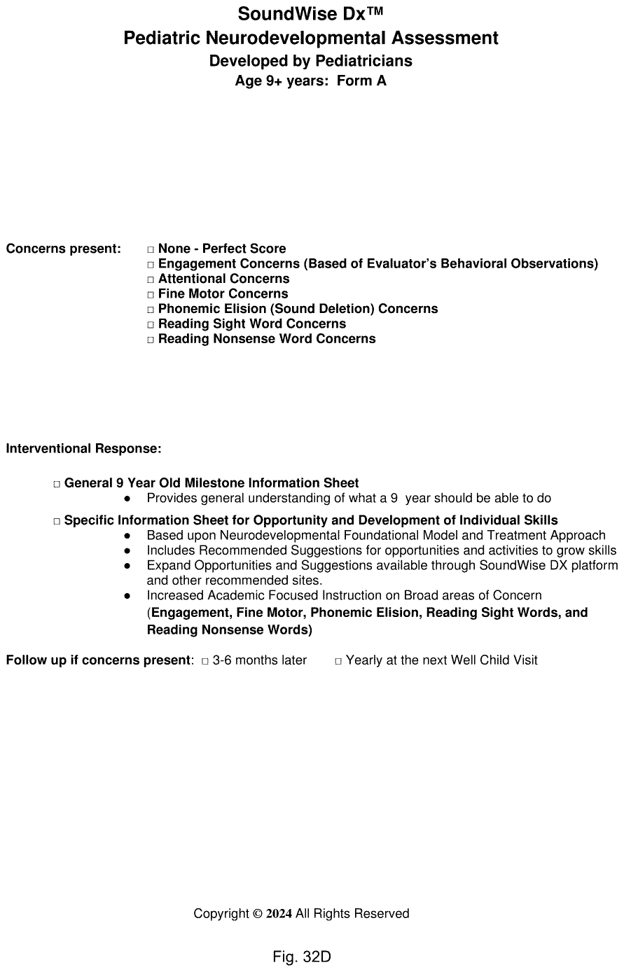

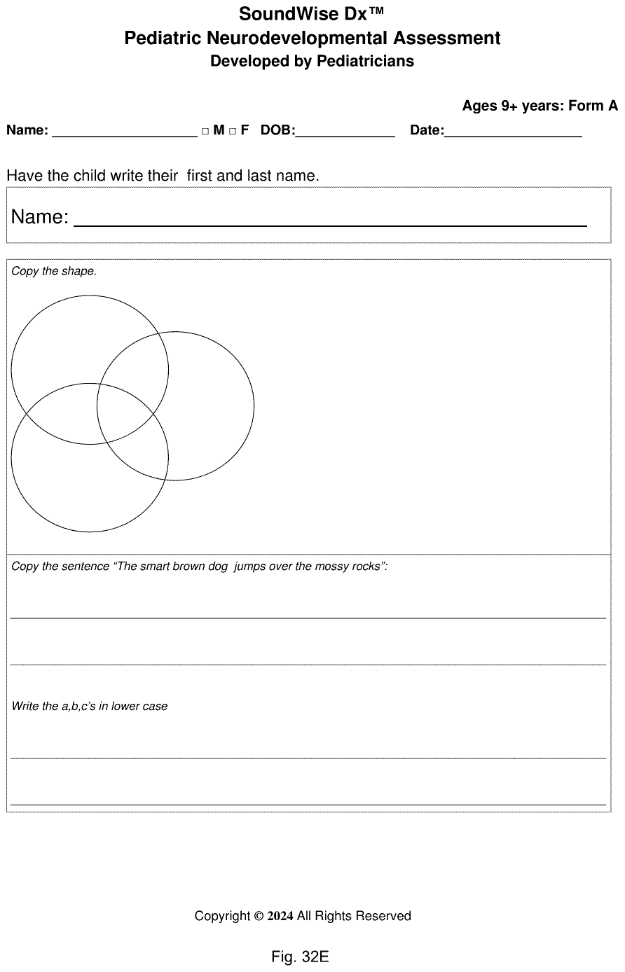

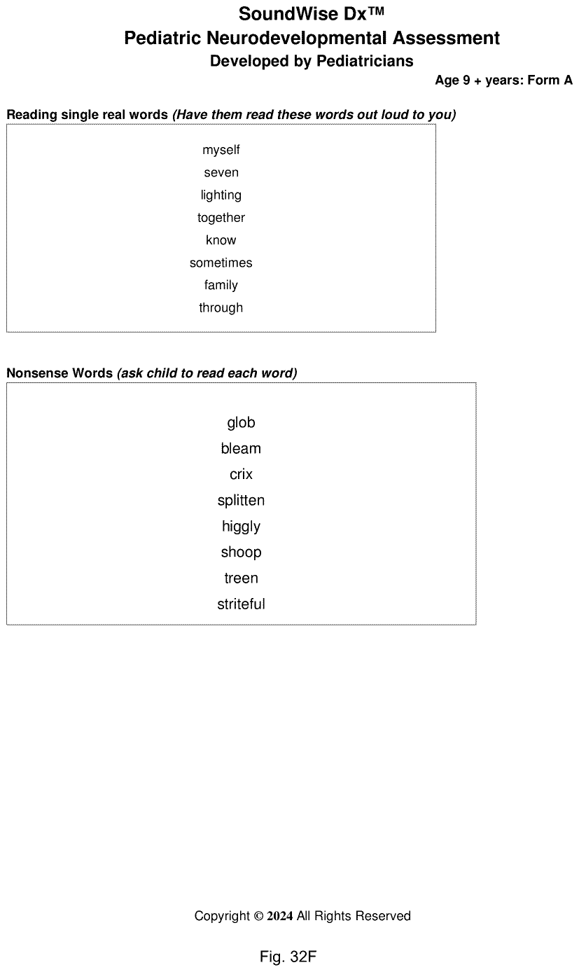

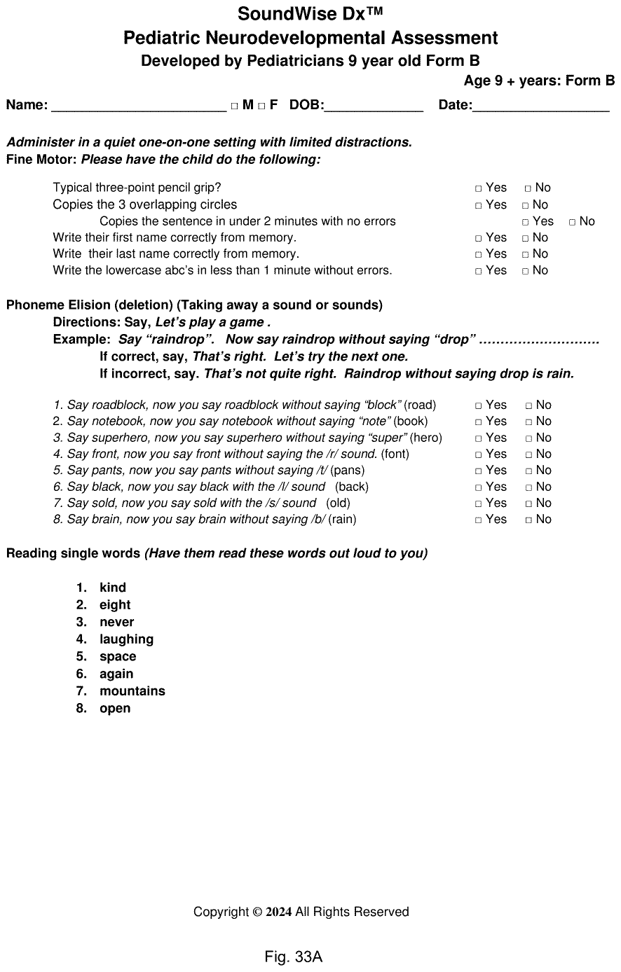

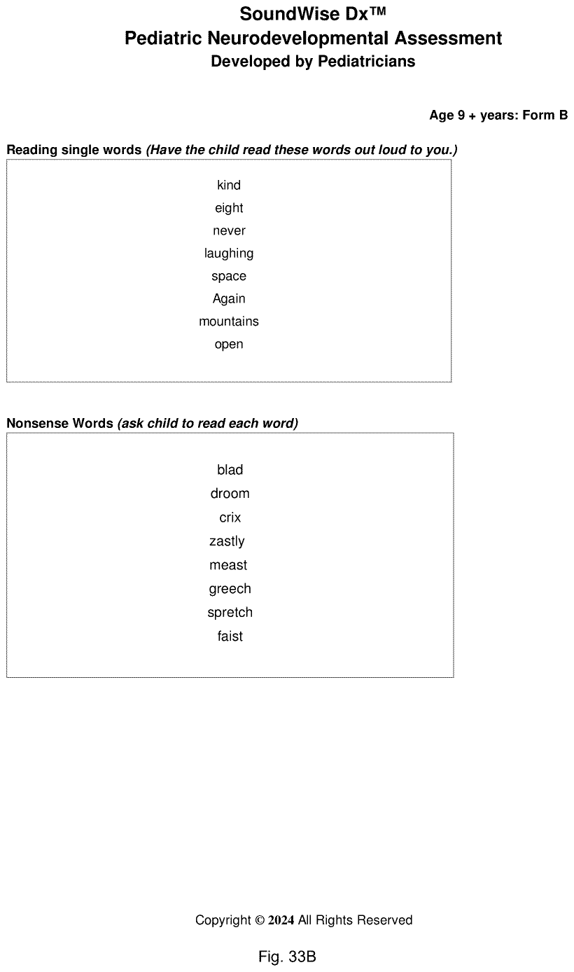

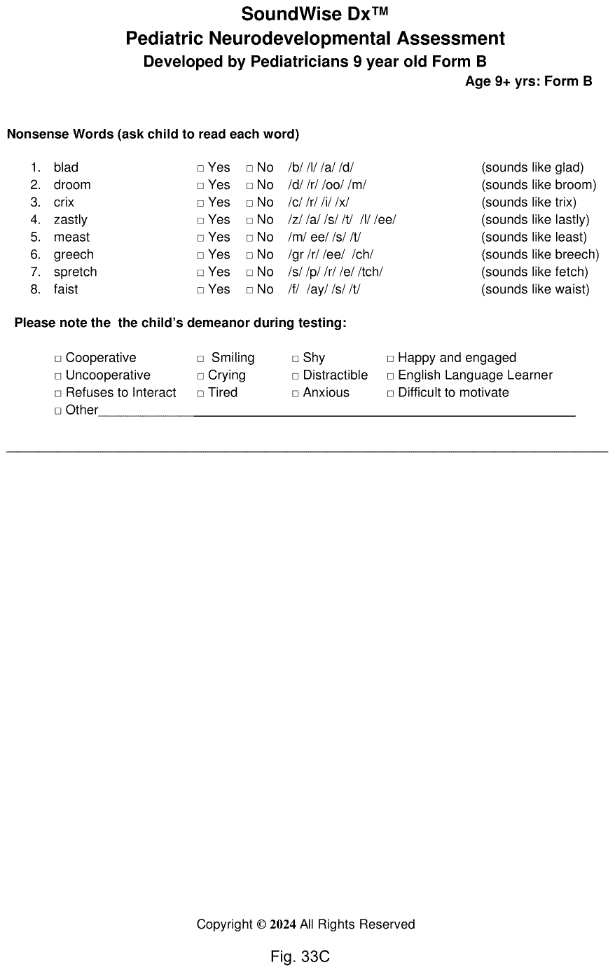

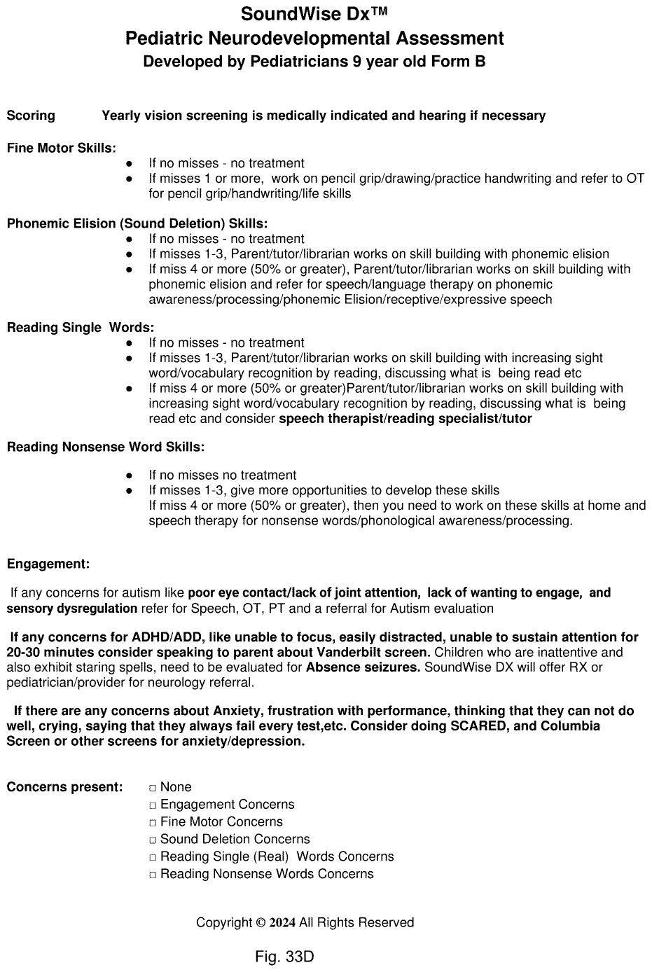

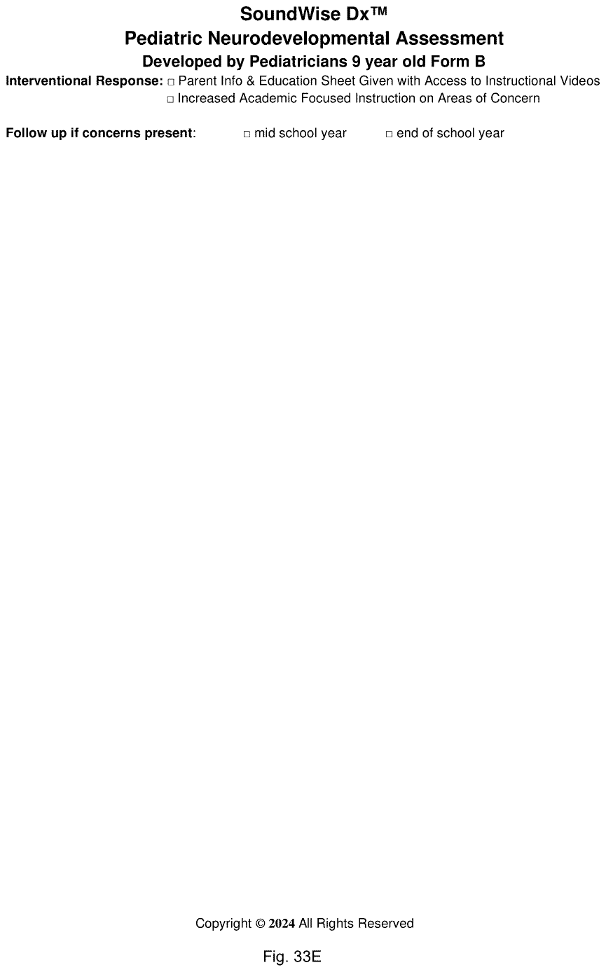

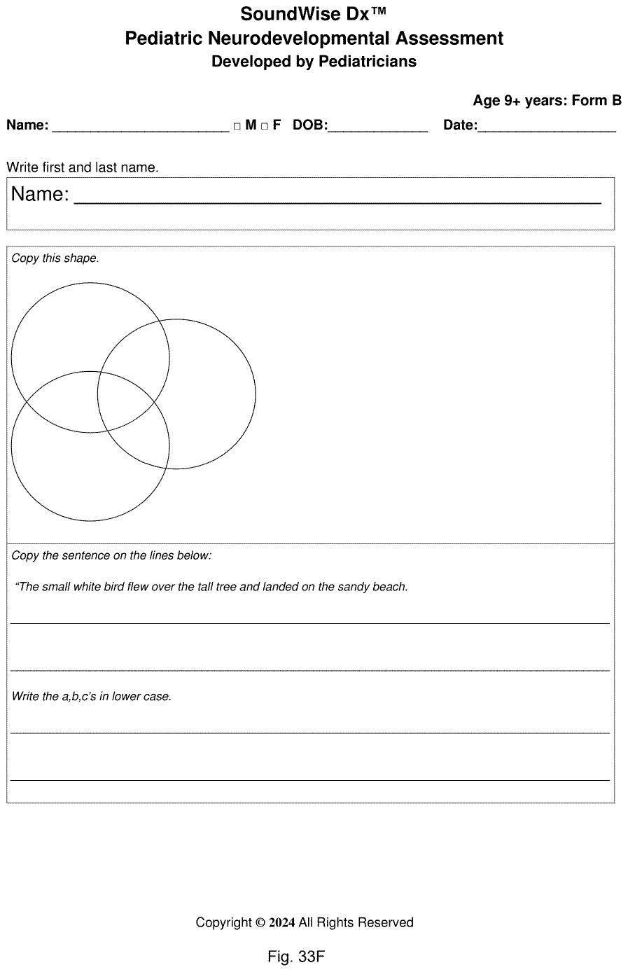

In the sixth part of the method, the child may be required to perform tests relating to fine motor skills, phoneme elision (deletion) skills, rhyming and/or reading skills (See, e.g., FIGS. 32A-32F; and FIGS. 33A-33E. For example, the child may be required to perform a typical three-point pencil grip, copy three overlapping circles, copy a sentence, write names from memory, write lower case ABC's. In one embodiment, the child may also be required to perform a phoneme elision (deletion) exercise in which the child is to say a word, such as, for example, “raindrop,” and the say raindrop without saying “drop,” for example. In one embodiment, the child may also be required to read one or more single words. In one embodiment, the child may also be required to read nonsense or nonreal words in which the child indicates whether or not the nonsense or nonreal word rhymes with an existing or proper word.

In the sixth part of the method, the tests discussed above and shown in FIGS. 32A-32F and/or FIGS. 33A-33E relate to fine motor skills, phonemic elision (sound deletion) skills and reading skills. The child's behavior may be observed during these tests for considerations such as, for example, whether the child is cooperative, whether the child is uncooperative, whether the child refuses to interact, whether the child completed tasks independently, whether the child exhibited poor eye contact, whether the child smiled, whether the child cried, whether the child appeared tired, whether or not the child would engage, whether the child was shy, whether the child was distractible, whether the child was anxious, whether the child needed redirection to complete tasks, whether the child was happy and engaged, whether the child is an English language learner, whether the child was difficult whether the child's speech was understandable, etc., as shown in FIGS. 32B and 33B.

In the sixth part of the method, tests for fine motor skills, phonemic elision (sound deletion) skills and reading skills (FIGS. 32C and 32D; or 33B and 33C) are evaluated for the presence or absence of a disorder and/or school readiness. The ability of the child to perform the requested tasks is recorded to generate input data. The ability of the child to perform one or more of the requested tasks may be recorded in a sixth worksheet. One embodiment of the fourth worksheet is shown in FIGS. 32A-32F. Another embodiment of the fifth worksheet is shown in FIGS. 33A-33E.

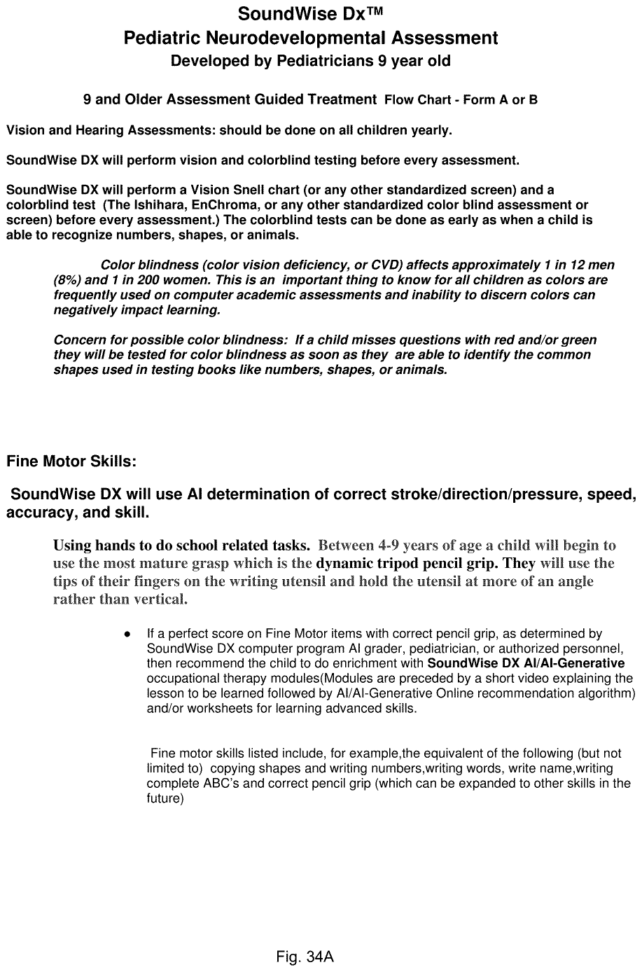

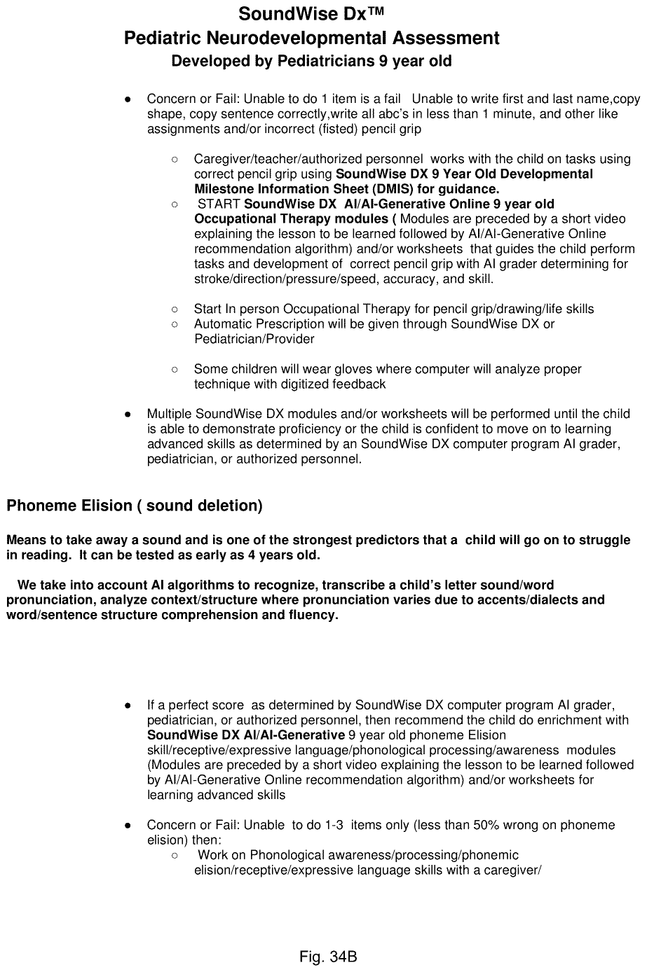

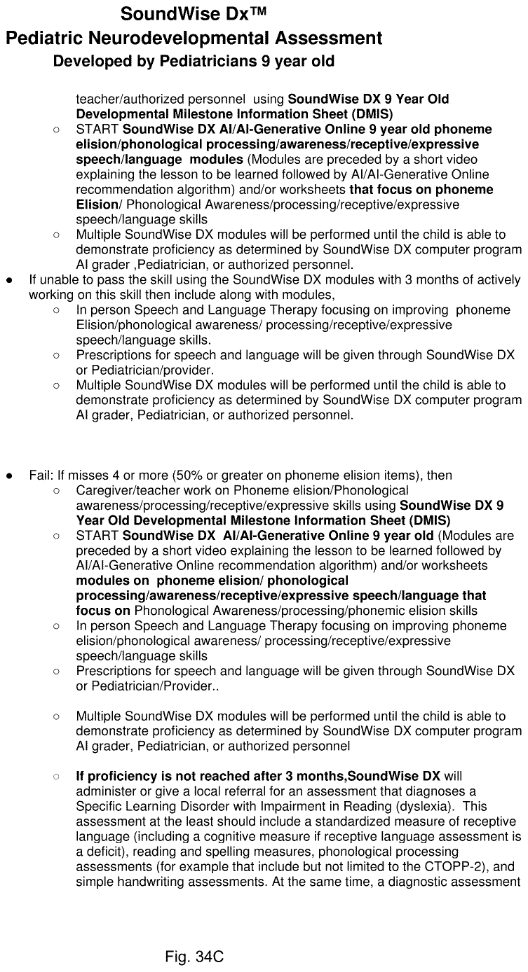

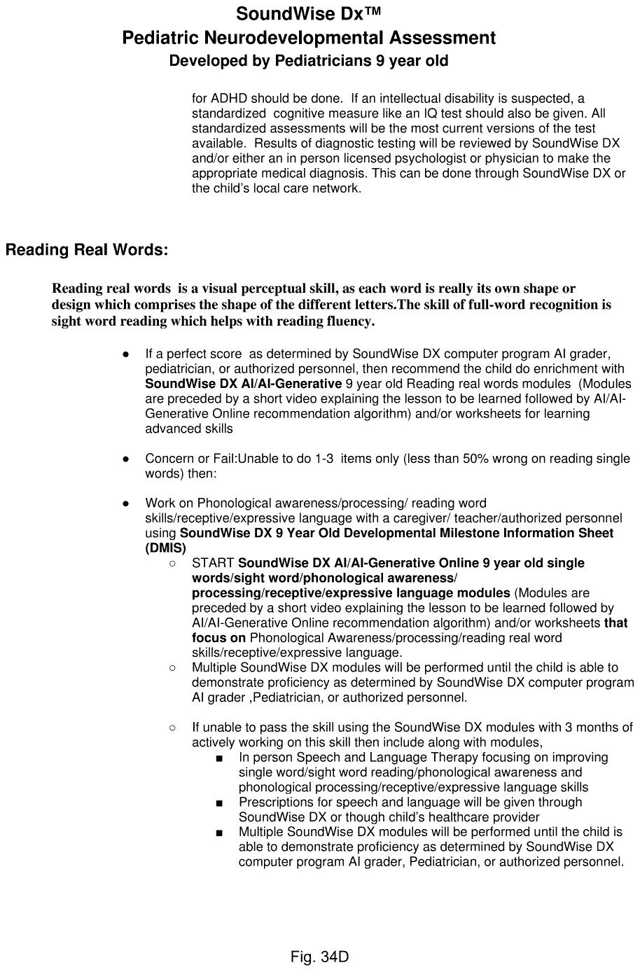

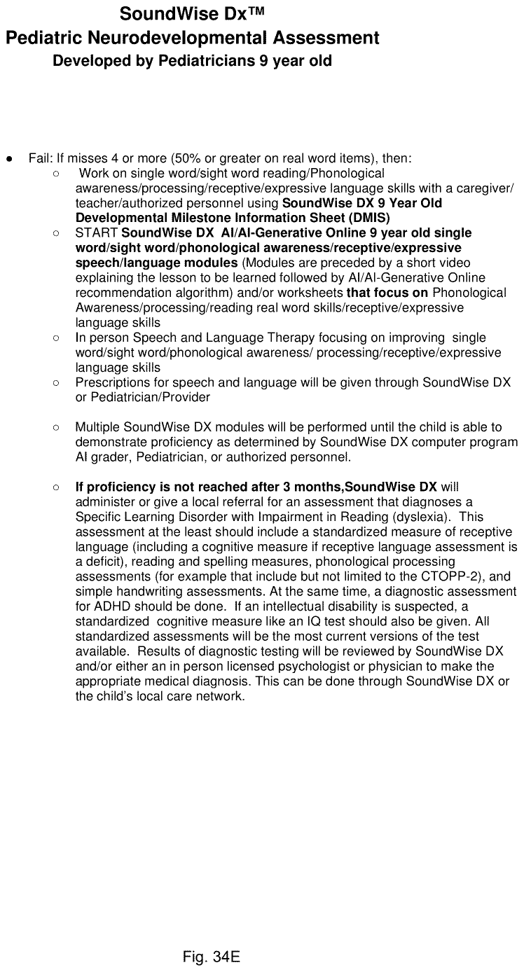

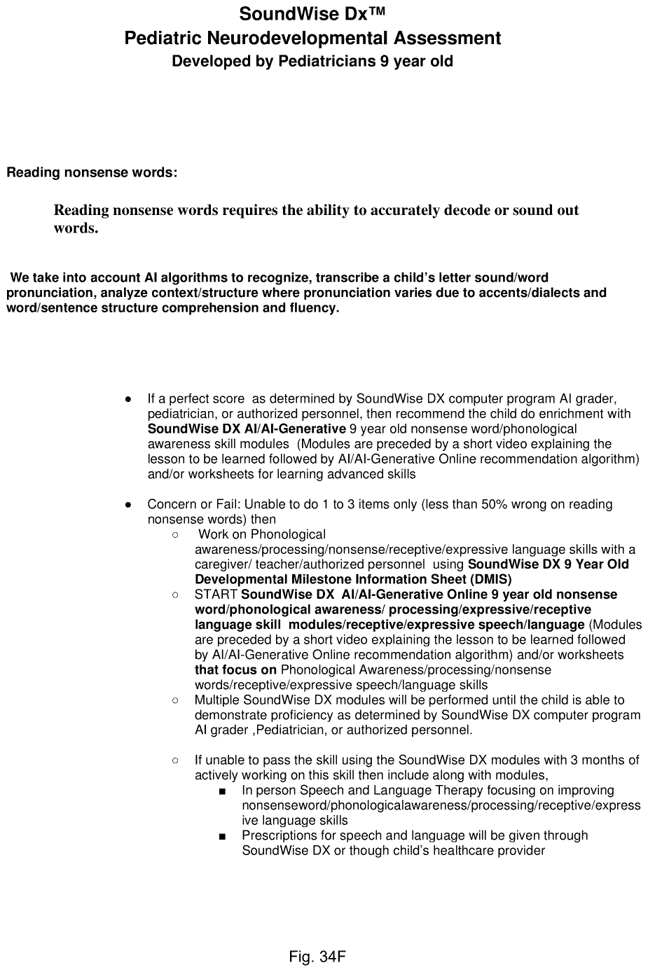

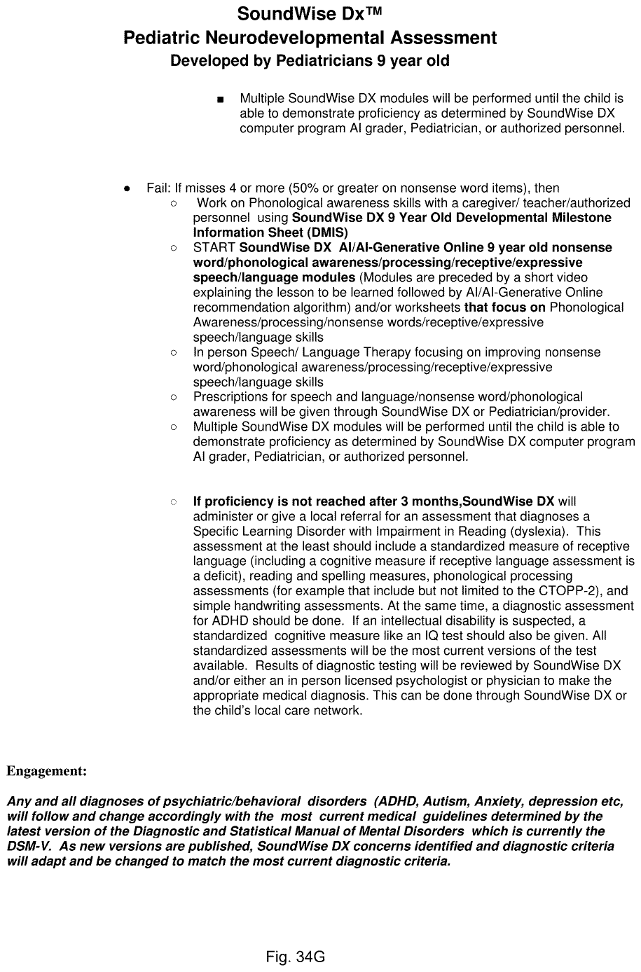

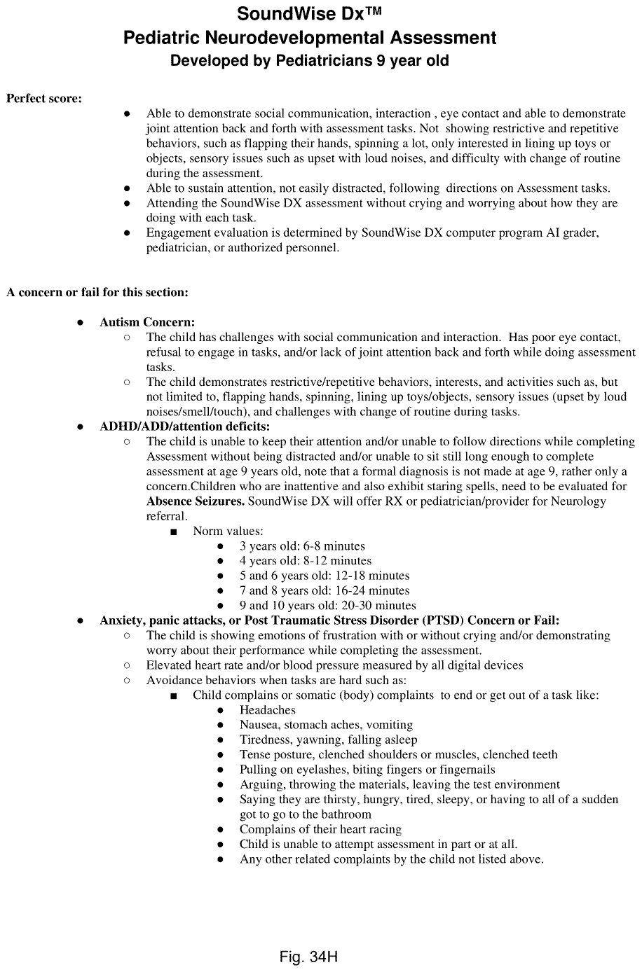

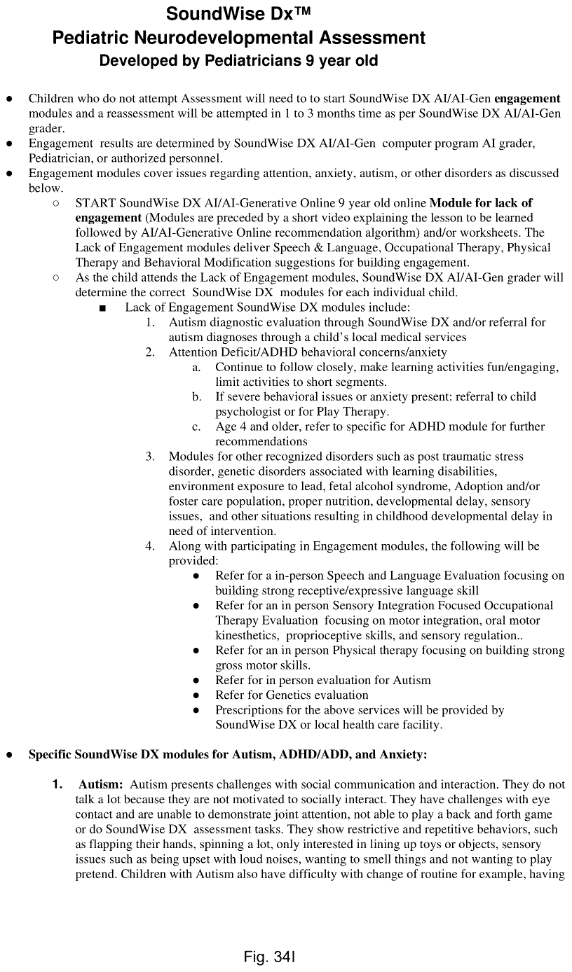

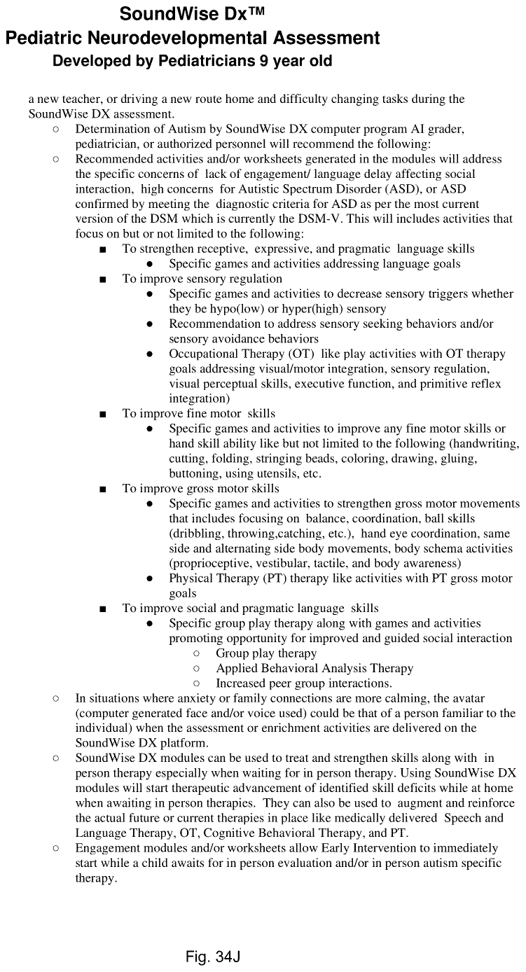

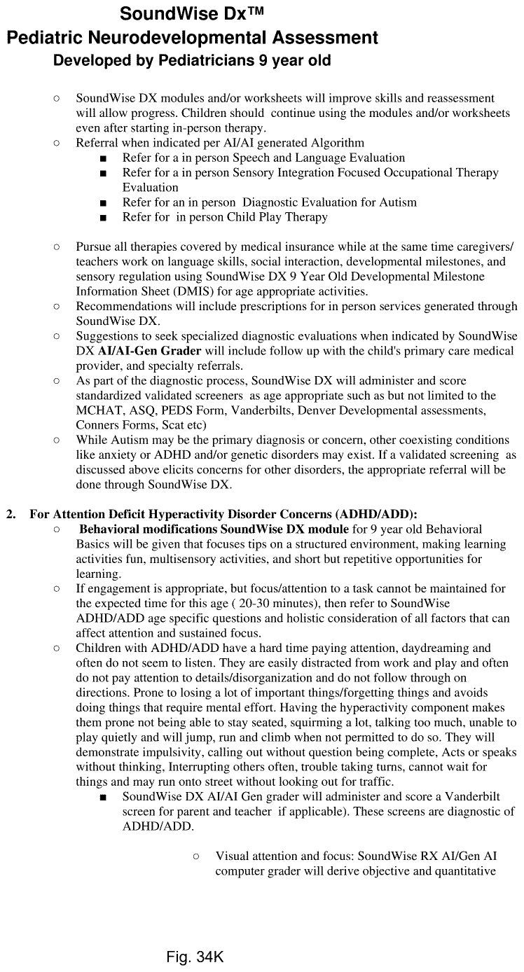

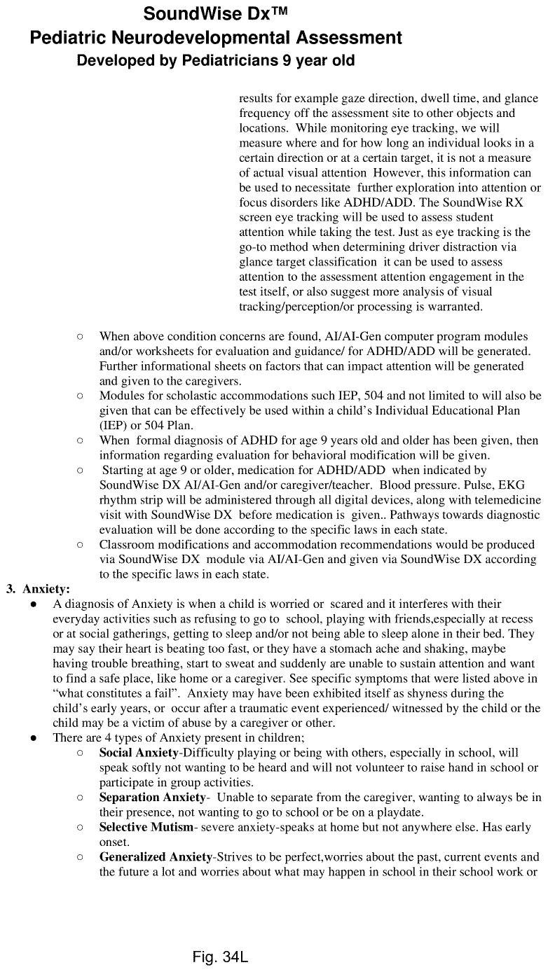

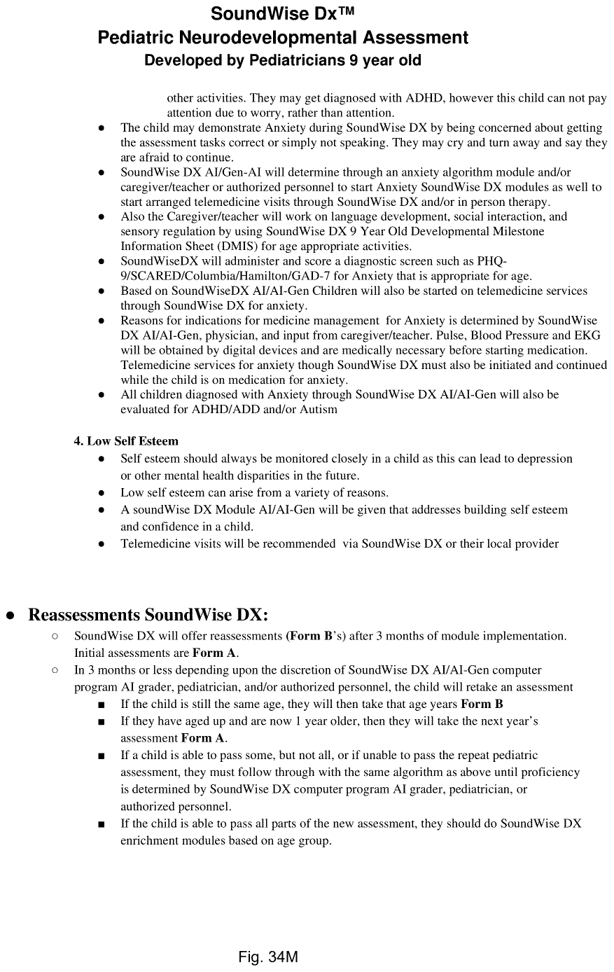

The fine motor skill input data may include whether or not the child was able to perform required tasks. The ability of the child to perform the required tasks may be recorded in the sixth worksheet. The fine motor input data is evaluated against an answer key to generate a fine motor evaluation result. The fine motor evaluation result is another determination with respect to the presence or absence of fine motor deficiency, after the fifth determination with respect the presence or absence of fine motor conducted during the fifth part of the method. In some embodiments, the fine motor evaluation result includes a score, such as, for example, a number score. In some embodiments, the fine motor evaluation result includes a written evaluation, such as, for example, words written in sentence or note form that explain what was observed by the practitioner. In some embodiments, the fine motor result is included in an assessment that evaluates at least each of the tests administered during the fourth part of the method. One embodiment of the assessment is shown in FIGS. 34A-34M.

In the sixth part of the method, a second test is administered in which the child has to do a phonemic elision (sound deletion) task to generate a phonemic elision task input data. The ability of the child to do a phonemic elision task may be recorded in the fourth worksheet (FIG. 32A-32F or FIG. 33A-33E). The phonemic elision input data is evaluated against an answer key to generate a phonemic elision evaluation result. The phonemic elision evaluation result is another determination with respect to the presence or absence of phonemic elision deficiency, after the determination with respect the presence or absence of phonemic elision deficiency conducted during the fifth part of the method. In some embodiments, the phonemic elision evaluation result includes a score, such as, for example, a number score. In some embodiments, the phonemic elision evaluation result includes a written evaluation, such as, for example, words written in sentence or note form that explain what was observed by the practitioner. In some embodiments, the phonemic elision evaluation result is included in the assessment (FIGS. 34A-34M). In some embodiments, the phonemic elision evaluation result uses AI or other software to recognize and/or transcribe the child's letter sound/word pronunciation, analyze context/structure where pronunciation varies due to accents/dialects and word/sentence structure comprehension and fluency.

In the sixth part of the method, a third test is administered in which the child has to read single words, such as, for example, sight words to generate a sight word reading input data. The ability of the child to do a sight word task may be recorded in the third worksheet (FIG. 32A-32F or FIG. 33A-33E). The sight word reading input data is evaluated against an answer key to generate a sight word reading evaluation result. The sight word reading evaluation result is a determination with respect to the presence or absence of sight word reading deficiency conducted during the sixth part of the method. In some embodiments, the sight word reading evaluation result includes a score, such as, for example, a number score. In some embodiments, the sight word reading evaluation result includes a written evaluation, such as, for example, words written in sentence or note form that explain what was observed by the practitioner. In some embodiments, the sight word reading evaluation result is included in the assessment (FIGS. 34A-34M). In some embodiments, the sight word reading evaluation result uses AI or other software to recognize and/or transcribe the child's letter sound/word pronunciation, analyze context/structure where pronunciation varies due to accents/dialects and word/sentence structure comprehension and fluency.

In one embodiment of the sixth part of the method, a fourth test is administered in which the child has to do a nonsense words exercise that compares nonsense words with real words to generate a nonsense word skill input data. The ability of the child to do a nonsense word skill may be recorded in the fifth worksheet (FIG. 32A-32F or FIG. 33A-33E). The nonsense word skill input data is evaluated against an answer key to generate a nonsense word skill evaluation result. The nonsense word skill evaluation result is another determination with respect to the presence or absence of nonsense word skills. In some embodiments, the nonsense word skill evaluation result includes a score, such as, for example, a number score. In some embodiments, the nonsense word skill evaluation result includes a written evaluation, such as, for example, words written in sentence or note form that explain what was observed by the practitioner. In some embodiments, the nonsense word skill evaluation result is included in the assessment (FIGS. 34A-34M). In some embodiments, the nonsense word skill evaluation result uses AI or other software to recognize and/or transcribe the child's letter sound/word pronunciation, analyze context/structure where pronunciation varies due to accents/dialects and word/sentence structure comprehension and fluency.

In the sixth part of the method, a referral is generated referring the individual for services in areas where the evaluation results were deficient to promote at least one of the skills tested in the fifth part of the method. In some embodiments, the assessment (FIGS. 34A-34M), which can include one or more of the test results in the sixth part of the method are evaluated to generate the referral. In particular, if the results indicate that the child has proficient fine motor skills, the child is not referred for services, such as, for example, occupational therapy to promote advancement in fine motor skills. If, however, the test results indicate that the child is deficient with his or her fine motor skills, the child is referred for services, such as, for example, occupational therapy to promote advancement in fine motor skills. Likewise, if the results indicate that the child has proficient phonemic elision skills, the child is not referred for services, such as, for example, Speech therapy to promote advancement in phonemic elision skills. If, however, the test results indicate that the child is deficient with his or her phonemic elision skills, the child is referred for services, such as, for example, Speech therapy to promote advancement in phonemic elision skills. If the results indicate that the child has proficient sight word reading skills, the child is not referred for services, such as, for example, speech therapy to promote advancement in sight word reading skills. If, however, the test results indicate that the child is deficient with his or her sight word reading skills, the child is referred for services, such as, for example, Speech therapy to promote advancement in sight word reading skills. Lastly, if the results indicate that the child has proficient nonsense word skills, the child is not referred for services, such as, for example, speech therapy to promote advancement in nonsense word skills. If, however, the test results indicate that the child is deficient with his or her nonsense word skills, the child is referred for services, such as, for example, speech therapy to promote advancement in nonsense word skills.

In some embodiments, the sixth part of the method includes evaluating the child for ADHD, autism, anxiety, panic attacks, post-traumatic stress disorder (PTSD), depression, low self-esteem, etc. if the evaluation results in the sixth part of the method were deficient to promote at least one of the skills tested in the sixth part of the method.

The ADHD evaluation in the sixth part of the method can include giving focused tips on a structured environment, making learning activities fun, multisensory activities, and short but repetitive opportunities for learning. If engagement is appropriate, but focus/attention to a task cannot be maintained for the age expected time for the child's age, then the method may refer the child to take a test that includes ADHD/ADD age specific questions and consider all factors that can affect attention and sustained focus. In some embodiments, if the child exhibits signs of ADHD/ADD, the method can be adapted to administer and score a Vanderbilt screen for parent and teacher (if applicable). These screens are diagnostic of ADHD/ADD. Absence seizure will be ruled out with EEG or another medical device.

Concerning autism, the sixth part of the method includes evaluating whether the child has challenges with social communication and interaction, has poor eye contact, refuses to engage in tasks, and/or lacks joint attention back and forth while doing assessment tasks. For example, if the child demonstrates restrictive/repetitive behaviors, interests, and activities such as, but not limited to, flapping hands, spinning, lining up toys/objects, sensory issues (upset by loud noises/smell/touch), and challenges with change of routine during tasks, the evaluation should include an autism evaluation.

If the autism evaluation of the present method may recommend activities and/or worksheets that are adapted to address the specific concerns of lack of engagement/language delay affecting social interaction, high concerns for Autistic Spectrum Disorder (ASD), or ASD confirmed. This will include activities that focus on strengthening receptive, expressive, and pragmatic language skills; specific games and activities addressing language goals. To improve sensory regulation, the autism evaluation of the present method may recommend specific games and activities to decrease sensory triggers whether they be hypo(low) or hyper(high) sensory. To address sensory seeking behaviors and/or sensory avoidance behaviors, the autism evaluation may include recommending Occupational Therapy (OT) like play activities with OT therapy goals addressing visual/motor integration, sensory regulation, visual perceptual skills, executive function, and primitive reflex integration.

The system and method of the present disclosure can be used to treat and strengthen skills along with in-person therapy especially when waiting for in-person therapy. The system and method of the present disclosure can recommend starting therapeutic advancement of identified skill deficits while at home when awaiting in-person therapies. Such therapeutic advancement can be used to augment and reinforce the actual future or current therapies in place like medically delivered Speech and Language Therapy, OT, Cognitive Behavioral Therapy, and PT. The system and method of the present disclosure can include engagement modules and/or worksheets that allow Early Intervention to immediately start while a child waits for in-person evaluation and/or in-person autism specific therapy. The system and method of the present disclosure can improve skills and reassessment will allow progress. Children should continue using the system and method of the present disclosure even after starting in-person therapy. Referral pursuant to the autism evaluation can include referring the child for an in-person Speech and Language Evaluation, referring the child for an in-person Sensory Integration Focused Occupational Therapy Evaluation, referring the child for an in-person Diagnostic Evaluation for Autism, referring the child for in-person Child Play Therapy.

Concerning anxiety, panic attacks, or PTSD, if the child is showing emotions of frustration with or without crying and/or demonstrating worry about their performance while completing the assessment, has an elevated heart rate and/or blood pressure and/or avoids behaviors when tasks are hard such as: complaining to end or get out of a task like, having headaches, having nausea, stomach aches, vomiting, having tiredness, yawning, falling asleep, having a tense posture, clenched shoulders or muscles, clenched teeth, pulling on eyelashes, biting fingers or fingernails, arguing, throwing the materials, leaving the test environment, saying they are thirsty, hungry, tired, sleepy, or having to all of a sudden got to go to the bathroom, complains of their heart racing, goes selectively mute or is unable to attempt to complete the tests in part or at all, the evaluation should include an anxiety, panic attack, or PTSD evaluation.

The system and method of the present disclosure may be configured to determine through an anxiety algorithm module and/or caregiver/teacher or authorized personnel to start Anxiety modules as well to start arranged telemedicine visits through the system and method of the present disclosure and/or in-person therapy. Also, the Caregiver/teacher will work on language development, social interaction, and sensory regulation by using a 9+ Year Old Developmental Milestone Information Sheet (DMIS) of the system and method of the present disclosure for age-appropriate activities. One example of a 9+ Year Old Developmental Milestone Information Sheet is shown in FIGS. 34A-34F. The system and method of the present disclosure may be configured to administer and score a diagnostic screen such as PHQ-9/SCARED/Columbia/Hamilton/GAD-7 for Anxiety that is appropriate for age. Based on the system and method of the present disclosure, Children will also be started on telemedicine services through the system and method of the present disclosure for anxiety.

Reasons for indications for medicine management for Anxiety is determined by the system and method of the present disclosure, physician, and input from caregiver/teacher. Pulse, Blood Pressure and EKG will be obtained by digital devices and are medically necessary before starting medication. Telemedicine services for anxiety though the system and method of the present disclosure must also be initiated and continued while the child is on medication for anxiety. All children diagnosed with Anxiety through the system and method of the present disclosure will also be evaluated for ADHD/ADD and/or Autism, if indicated.

Concerning low self-esteem, self-esteem should always be monitored closely in a child as this can lead to depression or other mental health disparities in the future. Low self-esteem can arise from a variety of reasons. The system and method of the present disclosure may include one or more modules that address building self-esteem and confidence in a child. Telemedicine visits will be recommended via the system and method of the present disclosure or a local provider.

In the sixth part of the method, a referral may also be generated referring the individual for services in areas where the evaluation results were sufficient to improve the child's skills, even though the child's skills are proficient for his or her age. For example, if the child demonstrates that he or she is able to demonstrate social communication, interaction, eye contact and able to demonstrate joint attention back and forth with assessment tasks, it may be indicative that the child's skills are not deficient, and the child needs to only to continue to build and/or improve his or her skills. Further, not showing restrictive and repetition behaviors, such as flapping their hands, spinning a lot, being only interested in lining up toys or objects, having sensory issue such as being upset with loud noises and difficulty with change of routine may also be indicative that the child's skills are not deficient, and the child needs to only to continue to build and/or improve his or her skills. Indeed, if the child is able to maintain attention, is not easily distracted, follows directions, etc. it may also be indicative that the child's skills are not deficient, and the child needs to only continue to build and/or improve his or her skills. Likewise, if the child is able to complete the tests without crying or worrying about how they are doing on the tests, it may also be indicative that the child's skills are not deficient, and the child needs to only to continue to build and/or improve his or her skills.

In some embodiments, the sixth part of the method further includes observing behavioral engagement skills of the child throughout the testing process, such as, for example, during the first test of the sixth part of the method, during the second test of the sixth part of the method, during the third test of the sixth part of the method and/or during the fourth test of the sixth part of the method. In some embodiments, the observations made throughout the testing process of the fifth part of the method can include noting the child's demeanor, such as, for example, observations pertaining to whether the child is cooperative, uncooperative, refuses to interact, completed the tests independently, exhibited poor eye contact, smiled, cried, was tired, would not engage, was shy, was distractible, was anxious, needed redirection for completion, was happy and engaged, was an English language learner, was difficult to motivate, etc. In some embodiments, the observations made throughout the testing process of the sixth part of the method are included in the worksheet (FIG. 32A-32F or 33A-33E) and/or are used to generate the referral. In some embodiments, information pertaining to the answers provided in the questionnaire of the sixth part of the method (FIG. 31) is included in the worksheet and/or is used to generate the referral.

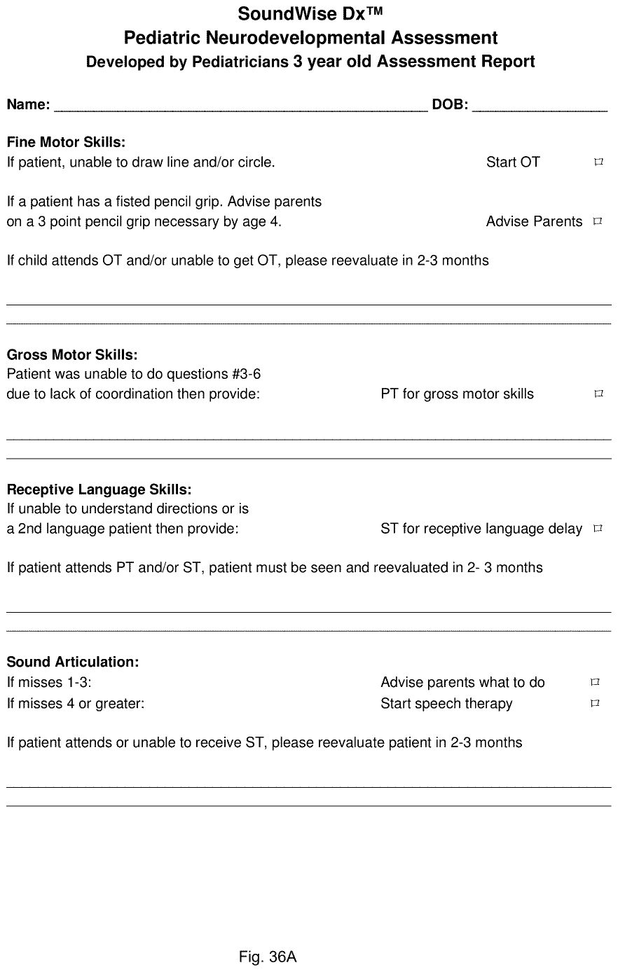

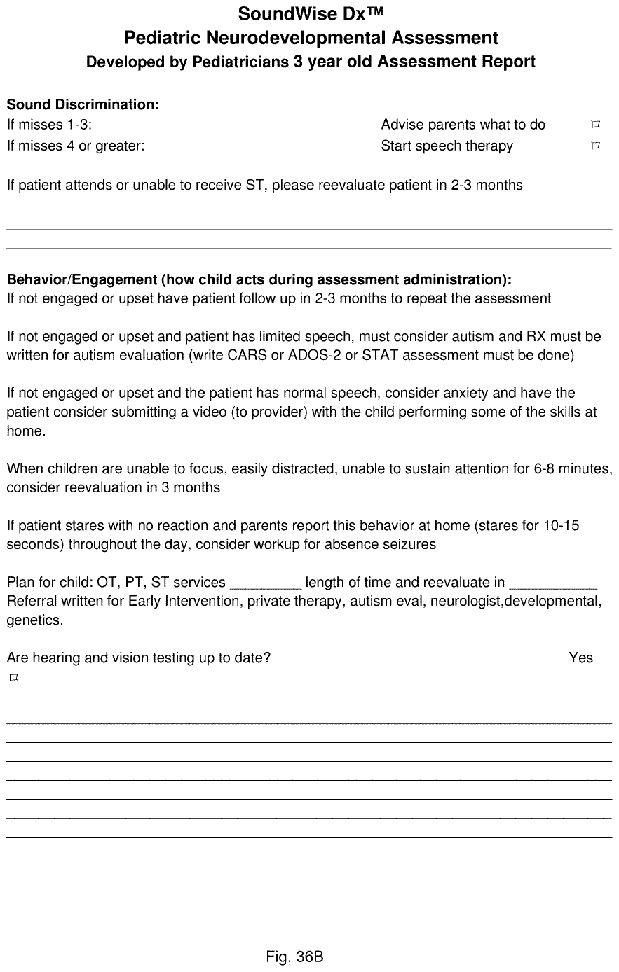

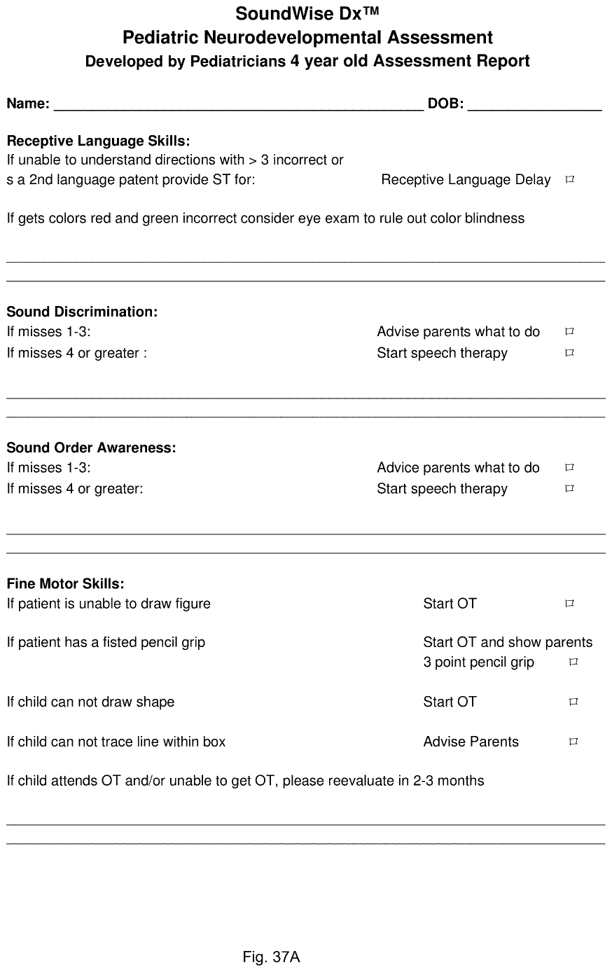

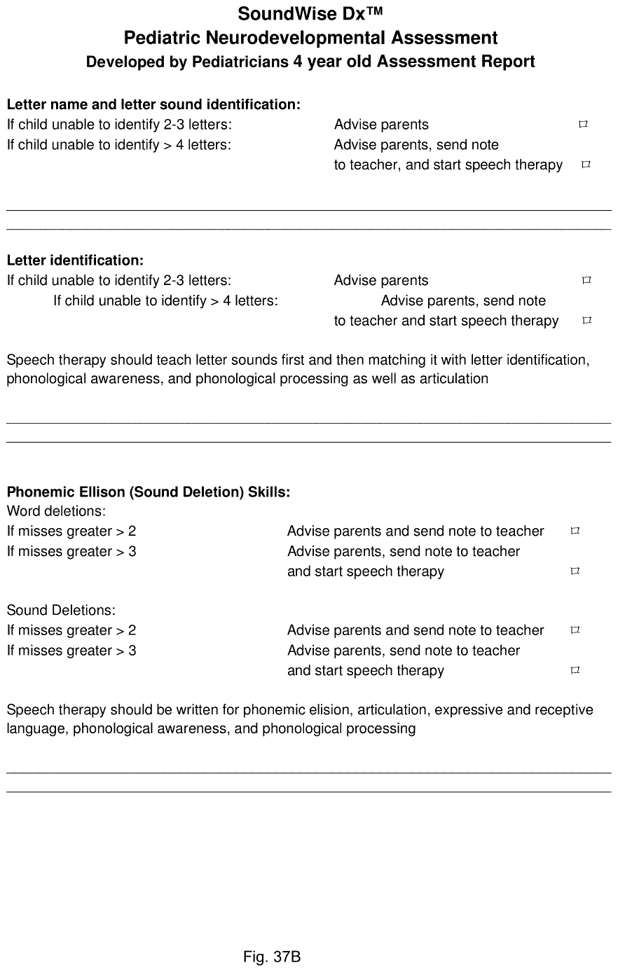

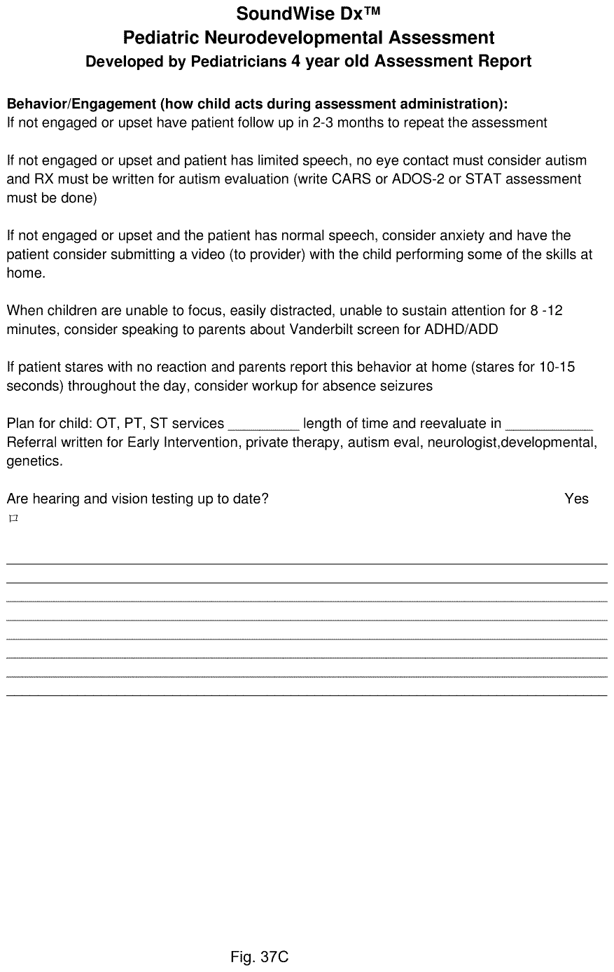

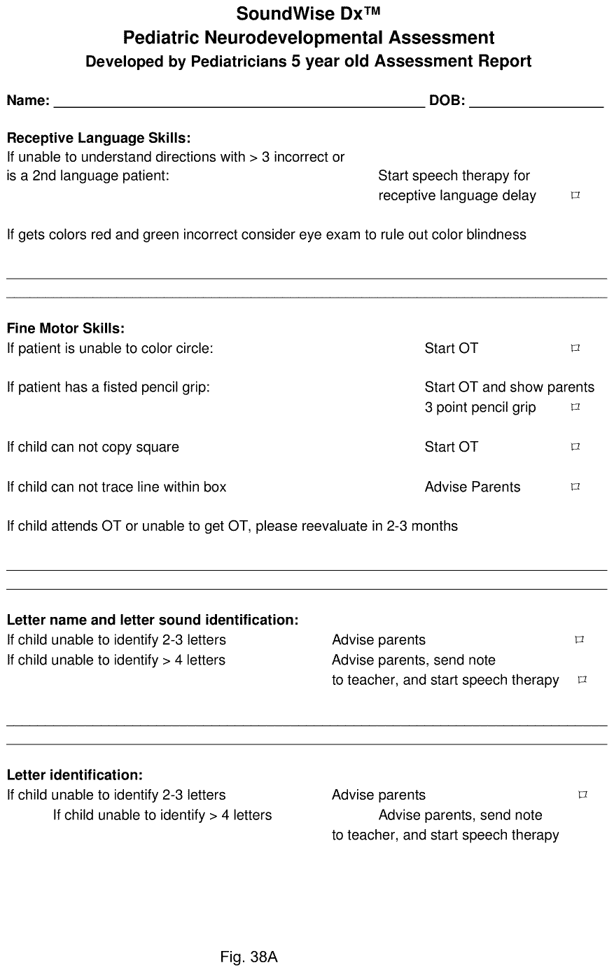

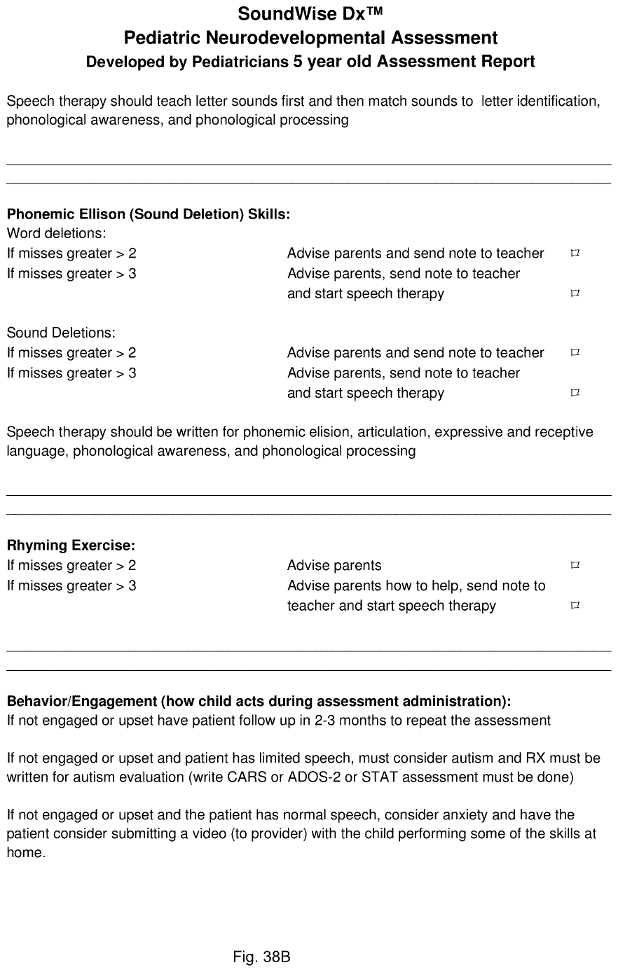

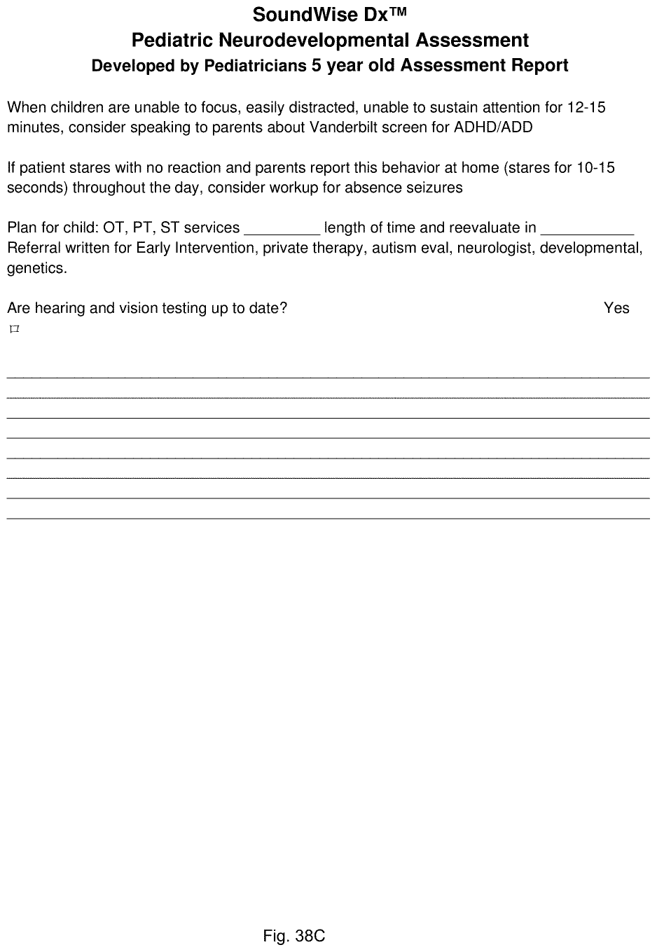

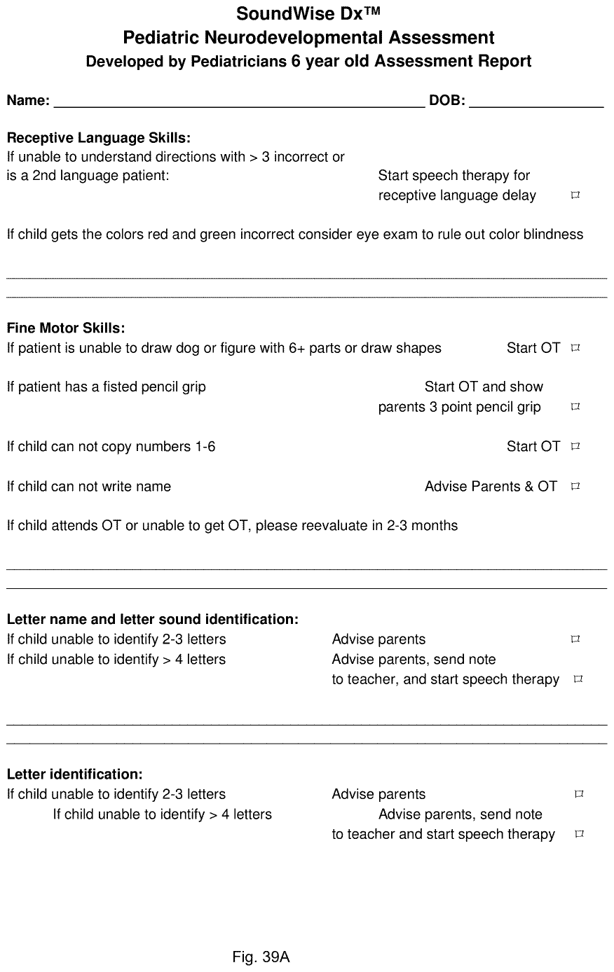

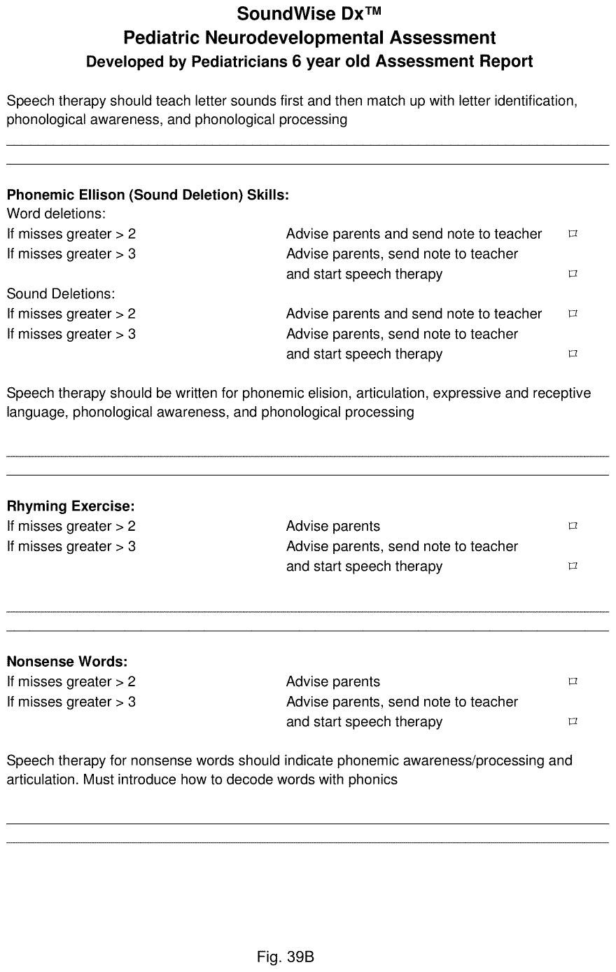

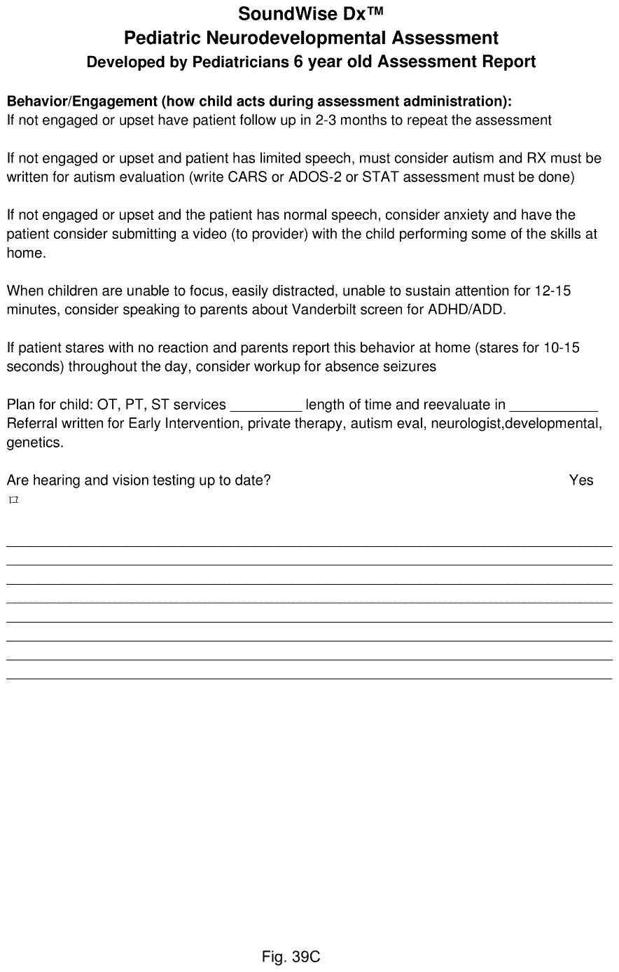

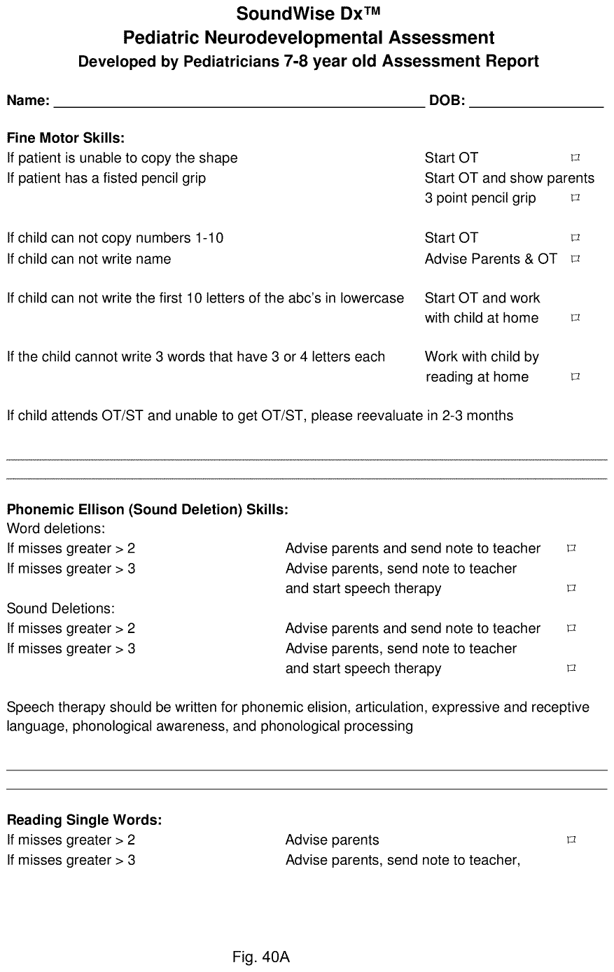

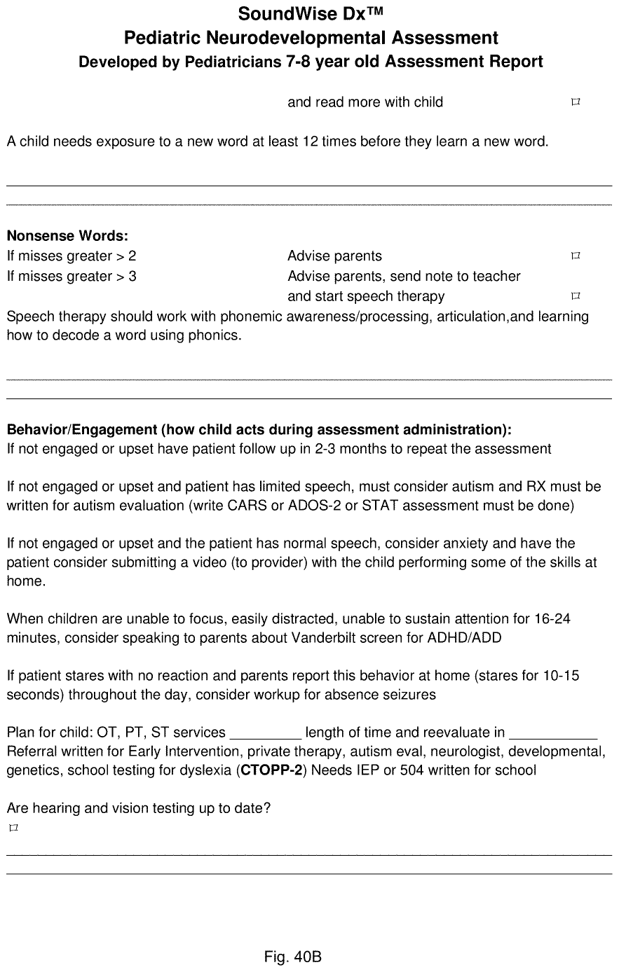

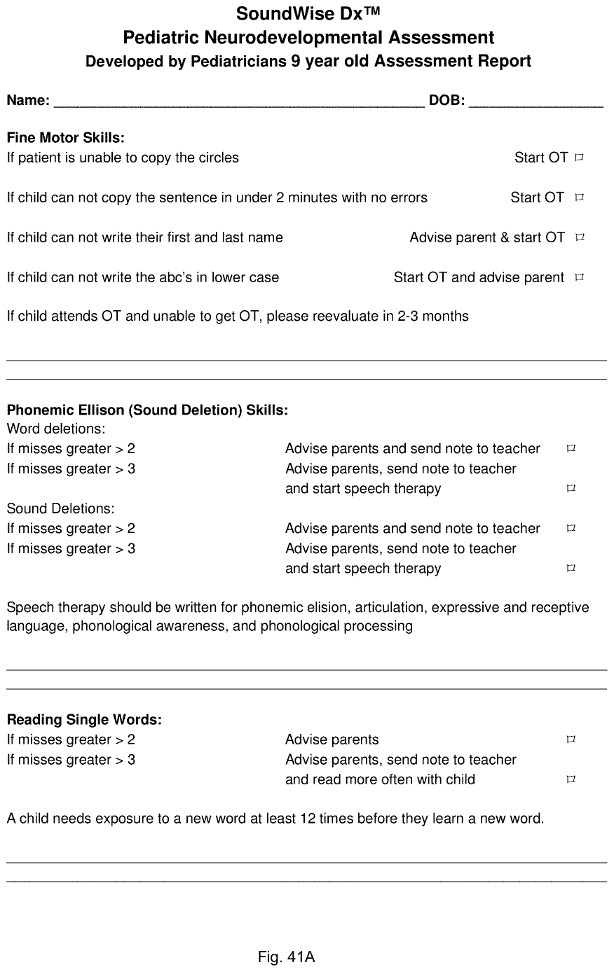

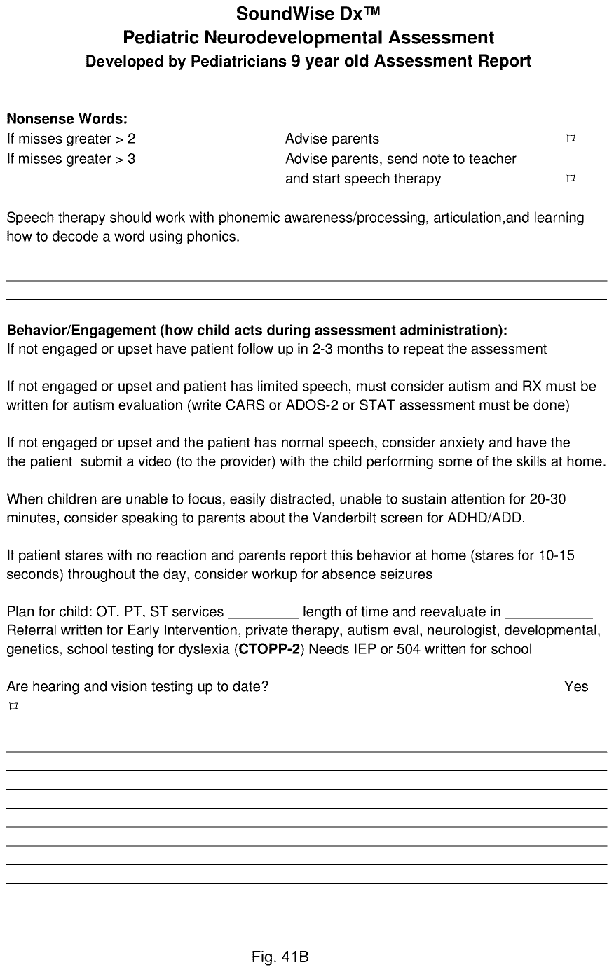

In some embodiments, one or more of the referrals described above may include an assessment report that refers the child to counseling, for example, based one or more of the evaluation results in a given part of the method. For example, in the first part of the method, the referral in the first part of the method may include an assessment report that assesses fine motor skills, gross motor skills, receptive language skills, sound articulation skills, sound discrimination skills and/or behavior/engagement. One example of an assessment report that can be used in the first step of the method is shown in FIGS. 36A-36B. In the second part of the method, the referral in the second part of the method may include an assessment report that assesses fine motor skills, receptive language skills, sound order awareness skills, sound discrimination skills, letter name and letter sound identification skills, letter identification skills and/or behavior/engagement, and/or social and emotional maturity. One example of an assessment report that can be used in the second step of the method is shown in FIGS. 37A-37B. In the third part of the method, the referral in the third part of the method may include an assessment report that assesses fine motor skills, receptive language skills, phonemic elision (sound deletion) skills, letter name and letter sound identification skills, letter identification skills, rhyming skills, behavior/engagement, and/or social-emotional maturity. One example of an assessment report that can be used in the third step of the method is shown in FIGS. 38A-38C. In the fourth part of the method, the referral in the fourth part of the method may include an assessment report that assesses fine motor skills, receptive language skills, phonemic elision (sound deletion) skills, letter name and letter sound identification skills, letter identification skills, rhyming skills, nonsense word skills, behavior/engagement, and/or social emotional maturity. One example of an assessment report that can be used in the fourth step of the method is shown in FIGS. 39A-39D. In the fifth part of the method, the referral in the fifth part of the method may include an assessment report that assesses fine motor skills, receptive language skills, phonemic elision (sound deletion) skills, letter name and letter sound identification skills, letter identification skills, rhyming skills, nonsense word skills, behavior/engagement and/or social emotional maturity. One example of an assessment report that can be used in the fifth step of the method is shown in FIGS. 40A-40B. In the sixth part of the method, the referral in the sixth part of the method may include an assessment report that assesses fine motor skills, receptive language skills, phonemic elision (sound deletion) skills, letter name and letter sound identification skills, letter identification skills, rhyming skills, nonsense word skills, social emotional maturity and/or behavior/engagement. One example of an assessment report that can be used in the sixth step of the method is shown in FIGS. 41A-41B. The assessments discussed above and shown in FIGS. 36A-41B may be used by a user of the system and method of the present disclosure to submit payments for insurance. For example, the user may use the assessments discussed above and shown in FIGS. 36A-41B to get paid by an insurance company for the tests, evaluations and/or assessments discussed herein.

In some embodiments, the method or methods described above may be executed or carried out by a computing system including a tangible computer-readable storage medium, also described herein as a storage machine, that holds machine-readable instructions executable by a logic machine (i.e. a processor or programmable control device) to provide, implement, perform, and/or enact the above-described methods, processes and/or tasks. When such methods and processes are implemented, the state of the storage machine may be changed to hold different data. For example, the storage machine may include memory devices such as various hard disk drives, CD, or DVD devices. The logic machine may execute machine-readable instructions via one or more physical information and/or logic processing devices. For example, the logic machine may be configured to execute instructions to perform tasks for a computer program. The logic machine may include one or more processors to execute the machine-readable instructions. The computing system may include a display subsystem to display a graphical user interface (GUI), or any visual element of the methods or processes described above. For example, the display subsystem, storage machine, and logic machine may be integrated such that the above method may be executed while visual elements of the disclosed system and/or method are displayed on a display screen for user consumption. The computing system may include an input subsystem that receives user input. The input subsystem may be configured to connect to and receive input from devices such as a mouse, keyboard or gaming controller. For example, a user input may indicate a request that certain task is to be executed by the computing system, such as requesting the computing system to display any of the above-described information or requesting that the user input updates or modifies existing stored information for processing. A communication subsystem may allow the methods described above to be executed or provided over a computer network. For example, the communication subsystem may be configured to enable the computing system to communicate with a plurality of personal computing devices. The communication subsystem may include wired and/or wireless communication devices to facilitate networked communication. The described methods or processes may be executed, provided, or implemented for a user or one or more computing devices via a computer-program product such as via an application programming interface (API).

In connection with the automated and/or computer-enabled versions of the methods discussed herein, in some embodiments, the evaluations conducted in relation to the tests administered during the first, second, third, fourth, fifth and/or sixths parts of the method may be recorded in worksheets, such as, for example, the worksheets shown in FIGS. 3, 4, 11A-D, 12A-D, 22A-I, 23A-J, 27A-E, 28A-E, 32A-F and 33A-F, as discussed above. Information on the worksheets may be input into an electronic device (logic machine) that includes a processor configured to operate AI software. The information that is input from the worksheets into the AI software may be utilized by the AI software to evaluate the information that is input from the worksheets against information captured by the AI software, such as, for example, an answer key. For example, in the first part of the method, the fine motor skill input data may be evaluated by the AI software against an answer key to generate the fine motor skill evaluation result. The fine motor skill evaluation result generated by the AI software may be used by the AI software to generate a referral, such as, for example, a referral to an occupational therapist to help the child with his or her fine motor skills. Likewise, in the first part of the method, the gross motor skill input data may be evaluated by the AI software against an answer key to generate the gross motor skill evaluation result. The gross motor skill evaluation result generated by the AI software may be used by the AI software to generate a referral, such as, for example, a referral to an occupational therapist to help the child with his or her gross motor skills. It is envisioned that the AI software can likewise be used to evaluate sound articulation input data, generate sound articulation evaluation results and provide a referral based on the sound articulation evaluation results; to evaluate sound discrimination input data, generate sound discrimination evaluation results and provide a referral based on the sound discrimination evaluation results; to evaluate receptive language input data, generate receptive language evaluation results and provide a referral based on the receptive language evaluation results; to evaluate sound order awareness input data, generate sound order awareness evaluation results and provide a referral based on the sound order awareness evaluation results; to evaluate letter naming and sound input data, generate letter naming and sound evaluation results and provide a referral based on the letter naming and sound evaluation results; to evaluate sound deletion input data, generate sound deletion results and provide a referral based on the sound deletion evaluation results; to evaluate rhyming input data, generate rhyming results and provide a referral based on the rhyming evaluation results; and to evaluate nonsense words input data, generate nonsense words evaluation results and provide a referral based on the nonsense words evaluation results.

The data used by the AI software to evaluate various input data can come from various sources. In some embodiments, the data used by the AI software to evaluate various input data can come from internet sources. data used by the AI software to evaluate various input data can be limited to what is found on a local area network that is owned and operated by the administrators of the disclosed method, for example. In some embodiments, data used by the AI software to evaluate various input data can come from any source, including, for example, a combination of internet and local area resources.

A portion of the disclosure of this patent document contains material which is subject to (copyright or mask work) protection. The (copyright or mask work) owner has no objection to the facsimile reproduction by anyone of the patent document or the patent disclosure, as it appears in the Patent and Trademark Office patent file or records, but otherwise reserves all (copyright or mask work) rights whatsoever.

It will be understood that various modifications may be made to the embodiments disclosed herein. For example, features of any one embodiment can be combined with features of any other embodiment. Therefore, the above description should not be construed as limiting, but merely as exemplification of the various embodiments. Those skilled in the art will envision other modifications within the scope and spirit of the claims appended hereto.

1-16. (canceled)

17. A computer-implemented method for monitoring neurodevelopment of an individual, the method comprising:
executing a processor to perform the steps of:
(a) causing the processor to instruct the individual to perform fine motor skills, letter naming and letter sound skills, sound deletion and rhyming tasks in a testing process;
(b) for a particular task of the tasks, (i) instructing the individual to perform a test relating to fine motor skills to generate a fine motor skill input data for processing, (ii) instructing the individual to perform a test relating to letter naming and letter sound skills to generate a letter naming and letter sound skill input data for processing, (iii) instructing the individual to perform a test relating to sound deletion to generate a sound deletion input data for processing, (iv) instructing the individual to perform a test relating to rhyming to generate a rhyming input data for processing;
(c) communicating with the individual to confirm the instruction to perform the test relating to fine motor skills and record a response time to execute the instruction to perform the test relating to fine motor skills onto a worksheet in the processor, simultaneously recording a second response time to execute the instruction to perform the test relating to letter naming and letter sound skills to execute the instruction to perform the test relating to letter naming and letter sound skills onto the worksheet of the processor, simultaneously recording a third response time to execute the instruction to perform the test relating to sound deletion to execute the instruction to perform the test relating to sound deletion onto the worksheet of the processor, and simultaneously recording a fourth response time to execute the instruction to perform the test relating to rhyming to perform the test relating to rhyming onto the worksheet of the processor;
(d) using the stored input data of the particular task from (b) in the processor to simultaneously evaluate the worksheet for the individual's fine motor skill task, the letter naming and letter sound skills task, the sound deletion task and the rhyming task to generate a score assessment for each task in 15 minutes;
(e) using the score assessment to determine a proposed treatment protocol for the respective fine motor skill, the letter naming and sound skill, the sound deletion skill and the rhyming skill;
(f) automatically generating a referral, by the processor sending the proposed treatment protocol for the individual to receive comprehensive services to promote at least one of the fine motor skills, the letter naming and sound skills, the sound deletion skills and the rhyming skills;
and
(f) delivering the referral to the individual or a caregiver of the individual.

18. The method recited in claim 17, further comprising observing behavioral engagement skills of the individual throughout the testing process.

19. The method recited in claim 17, wherein generating the referral includes performing one or more of the assessments selected from the group consisting of an anxiety assessment, an attention deficit/hyperactivity disorder (ADHD) assessment, an autism assessment, and a dyslexia assessment.

20. The method recited in claim 17, wherein the method is conducted using artificial intelligence (AI), pre-recorded programs on-line and/or is administered in-person.

21. The method recited in claim 17, wherein the test relating to fine motor skills includes performing a three-point pencil grip.

22. The method recited in claim 17, wherein the test relating to fine motor skills includes drawing an animal with six or more body parts.

23. The method recited in claim 17, wherein the test relating to fine motor skills includes copying numbers one through six.

24. The method recited in claim 17, wherein the test relating to fine motor skills includes writing the individual's name.

25. The method recited in claim 17, wherein the test relating to fine motor skills includes drawing shapes.

26. The method recited in claim 17, wherein the test relating to letter naming and letter sound skills includes identifying and naming letters of the alphabet.

27. The method recited in claim 17, wherein the test relating to letter naming and letter sound skills includes identifying corresponding sounds for letters.

28. The method recited in claim 17, wherein the test relating to sound deletion includes removing a specific sound from a spoken word and saying what remains.

29. The method recited in claim 17, wherein the test relating to rhyming includes identifying a rhyming pair.

30. The method recited in claim 17, wherein the test relating to rhyming includes producing words that rhyme with a given word.

31. The method recited in claim 17, wherein the test relating to rhyming includes identifying which word does not rhyme with other words.

32. The method recited in claim 17, wherein the processor executes machine-readable instructions.

33. The method recited in claim 17, further comprising playing with the individual in between performing the tasks.

34. The method recited in claim 17, further comprising teaching the individual in between performing the tasks.

35. The method recited in claim 17, wherein the processor includes only one processor.

36. The method recited in claim 17, wherein the processor includes a plurality of processors.
